SEQUENCE LISTING (1213 sequences)

SEQ ID NO: 1 - RNA, 19 nt, Artificial Sequence
  Description of Artificial Sequence  Target sequence/siNA sense region
aaggggagguaacccuggc

SEQ ID NO: 2 - RNA, 19 nt, Artificial Sequence
  Description of Artificial Sequence  Target sequence/siNA sense region
ccccuuuggucggggcccc

SEQ ID NO: 3 - RNA, 19 nt, Artificial Sequence
  Description of Artificial Sequence  Target sequence/siNA sense region
cgggcagccgcgcgccccu

SEQ ID NO: 4 - RNA, 19 nt, Artificial Sequence
  Description of Artificial Sequence  Target sequence/siNA sense region
uucccacggggcccuuuac

SEQ ID NO: 5 - RNA, 19 nt, Artificial Sequence
  Description of Artificial Sequence  Target sequence/siNA sense region
cugcgccgcgcgcccggcc

SEQ ID NO: 6 - RNA, 19 nt, Artificial Sequence
  Description of Artificial Sequence Target sequence/siNA sense region
ccccaccccucgcagcacc

SEQ ID NO: 7 - RNA, 19 nt, Artificial Sequence
  Description of Artificial Sequence  Target sequence/siNA sense region
cccgcgccccgcgcccucc

SEQ ID NO: 8 - RNA, 19 nt, Artificial Sequence
  Description of Artificial Sequence Target sequence/siNA sense region
ccagccggguccagccgga

SEQ ID NO: 9 - RNA, 19 nt, Artificial Sequence
  Description of Artificial Sequence Target sequence/siNA sense region
agccauggggccggagccg

SEQ ID NO: 10 - RNA, 19 nt, Artificial Sequence
  Description of Artificial Sequence Target sequence/siNA sense region
gcagugagcaccauggagc

SEQ ID NO: 11 - RNA, 19 nt, Artificial Sequence
  Description of Artificial Sequence Target sequence/siNA sense region
cuggcggccuugugccgcu

SEQ ID NO: 12 - RNA, 19 nt, Artificial Sequence
  Description of Artificial Sequence Target sequence/siNA sense region
ugggggcuccuccucgccc

SEQ ID NO: 13 - RNA, 19 nt, Artificial Sequence
  Description of Artificial Sequence  Target sequence/siNA sense region
cucuugccccccggagccg

SEQ ID NO: 14 - RNA, 19 nt, Artificial Sequence
  Description of Artificial Sequence Target sequence/siNA sense region
gcgagcacccaagugugca

SEQ ID NO: 15 - RNA, 19 nt, Artificial Sequence
  Description of Artificial Sequence  Target sequence/siNA sense region
accggcacagacaugaagc

SEQ ID NO: 16 - RNA, 19 nt, Artificial Sequence
  Description of Artificial Sequence  Target sequence/siNA sense region
cugcggcucccugccaguc

SEQ ID NO: 17 - RNA, 19 nt, Artificial Sequence
  Description of Artificial Sequence  Target sequence/siNA sense region
cccgagacccaccuggaca

SEQ ID NO: 18 - RNA, 19 nt, Artificial Sequence
  Description of Artificial Sequence  Target sequence/siNA sense region
augcuccgccaccucuacc

SEQ ID NO: 19 - RNA, 19 nt, Artificial Sequence
  Description of Artificial Sequence  Target sequence/siNA sense region
cagggcugccagguggugc

SEQ ID NO: 20 - RNA, 19 nt, Artificial Sequence
  Description of Artificial Sequence Target sequence/siNA sense region
cagggaaaccuggaacuca

SEQ ID NO: 21 - RNA, 19 nt, Artificial Sequence
  Description of Artificial Sequence Target sequence/siNA sense region
accuaccugcccaccaaug

SEQ ID NO: 22 - RNA, 19 nt, Artificial Sequence
  Description of Artificial Sequence Target sequence/siNA sense region
gccagccuguccuuccugc

SEQ ID NO: 23 - RNA, 19 nt, Artificial Sequence
  Description of Artificial Sequence Target sequence/siNA sense region
caggauauccaggaggugc

SEQ ID NO: 24 - RNA, 19 nt, Artificial Sequence
  Description of Artificial Sequence Target sequence/siNA sense region
cagggcuacgugcucaucg

SEQ ID NO: 25 - RNA, 19 nt, Artificial Sequence
  Description of Artificial Sequence Target sequence/siNA sense region
gcucacaaccaagugaggc

SEQ ID NO: 26 - RNA, 19 nt, Artificial Sequence
  Description of Artificial Sequence Target sequence/siNA sense region
caggucccacugcagaggc

SEQ ID NO: 27 - RNA, 19 nt, Artificial Sequence
  Description of Artificial Sequence Target sequence/siNA sense region
cugcggauugugcgaggca

SEQ ID NO: 28 - RNA, 19 nt, Artificial Sequence
  Description of Artificial Sequence Target sequence/siNA sense region
acccagcucuuugaggaca

SEQ ID NO: 29 - RNA, 19 nt, Artificial Sequence
  Description of Artificial Sequence Target sequence/siNA sense region
aacuaugcccuggccgugc

SEQ ID NO: 30 - RNA, 19 nt, Artificial Sequence
  Description of Artificial Sequence Target sequence/siNA sense region
cuagacaauggagacccgc

SEQ ID NO: 31 - RNA, 19 nt, Artificial Sequence
  Description of Artificial Sequence Target sequence/siNA sense region
cugaacaauaccaccccug

SEQ ID NO: 32 - RNA, 19 nt, Artificial Sequence
  Description of Artificial Sequence Target sequence/siNA sense region
gucacaggggccuccccag

SEQ ID NO: 33 - RNA, 19 nt, Artificial Sequence
  Description of Artificial Sequence Target sequence/siNA sense region
ggaggccugcgggagcugc

SEQ ID NO: 34 - RNA, 19 nt, Artificial Sequence
  Description of Artificial Sequence Target sequence/siNA sense region
cagcuucgaagccucacag

SEQ ID NO: 35 - RNA, 19 nt, Artificial Sequence
  Description of Artificial Sequence Target sequence/siNA sense region
gagaucuugaaaggagggg

SEQ ID NO: 36 - RNA, 19 nt, Artificial Sequence
  Description of Artificial Sequence Target sequence/siNA sense region
gucuugauccagcggaacc

SEQ ID NO: 37 - RNA, 19 nt, Artificial Sequence
  Description of Artificial Sequence Target sequence/siNA sense region
ccccagcucugcuaccagg

SEQ ID NO: 38 - RNA, 19 nt, Artificial Sequence
  Description of Artificial Sequence Target sequence/siNA sense region
gacacgauuuuguggaagg

SEQ ID NO: 39 - RNA, 19 nt, Artificial Sequence
  Description of Artificial Sequence Target sequence/siNA sense region
gacaucuuccacaagaaca

SEQ ID NO: 40 - RNA, 19 nt, Artificial Sequence
  Description of Artificial Sequence Target sequence/siNA sense region
aaccagcuggcucucacac

SEQ ID NO: 41 - RNA, 19 nt, Artificial Sequence
  Description of Artificial Sequence Target sequence/siNA sense region
cugauagacaccaaccgcu

SEQ ID NO: 42 - RNA, 19 nt, Artificial Sequence
  Description of Artificial Sequence Target sequence/siNA sense region
ucucgggccugccaccccu

SEQ ID NO: 43 - RNA, 19 nt, Artificial Sequence
  Description of Artificial Sequence Target sequence/siNA sense region
uguucuccgauguguaagg

SEQ ID NO: 44 - RNA, 19 nt, Artificial Sequence
  Description of Artificial Sequence Target sequence/siNA sense region
ggcucccgcugcuggggag

SEQ ID NO: 45 - RNA, 19 nt, Artificial Sequence
  Description of Artificial Sequence Target sequence/siNA sense region
gagaguucugaggauuguc

SEQ ID NO: 46 - RNA, 19 nt, Artificial Sequence
  Description of Artificial Sequence Target sequence/siNA sense region
cagagccugacgcgcacug

SEQ ID NO: 47 - RNA, 19 nt, Artificial Sequence
  Description of Artificial Sequence Target sequence/siNA sense region
gucugugccgguggcugug

SEQ ID NO: 48 - RNA, 19 nt, Artificial Sequence
  Description of Artificial Sequence Target sequence/siNA sense region
gcccgcugcaaggggccac

SEQ ID NO: 49 - RNA, 19 nt, Artificial Sequence
  Description of Artificial Sequence Target sequence/siNA sense region
cugcccacugacugcugcc

SEQ ID NO: 50 - RNA, 19 nt, Artificial Sequence
  Description of Artificial Sequence Target sequence/siNA sense region
caugagcagugugcugccg

SEQ ID NO: 51 - RNA, 19 nt, Artificial Sequence
  Description of Artificial Sequence Target sequence/siNA sense region
ggcugcacgggccccaagc

SEQ ID NO: 52 - RNA, 19 nt, Artificial Sequence
  Description of Artificial Sequence Target sequence/siNA sense region
cacucugacugccuggccu

SEQ ID NO: 53 - RNA, 19 nt, Artificial Sequence
  Description of Artificial Sequence Target sequence/siNA sense region
ugccuccacuucaaccaca

SEQ ID NO: 54 - RNA, 19 nt, Artificial Sequence
  Description of Artificial Sequence Target sequence/siNA sense region
aguggcaucugugagcugc

SEQ ID NO: 55 - RNA, 19 nt, Artificial Sequence
  Description of Artificial Sequence Target sequence/siNA sense region
cacugcccagcccugguca

SEQ ID NO: 56 - RNA, 19 nt, Artificial Sequence
  Description of Artificial Sequence Target sequence/siNA sense region
accuacaacacagacacgu

SEQ ID NO: 57 - RNA, 19 nt, Artificial Sequence
  Description of Artificial Sequence Target sequence/siNA sense region
uuugaguccaugcccaauc

SEQ ID NO: 58 - RNA, 19 nt, Artificial Sequence
  Description of Artificial Sequence Target sequence/siNA sense region
cccgagggccgguauacau

SEQ ID NO: 59 - RNA, 19 nt, Artificial Sequence
  Description of Artificial Sequence Target sequence/siNA sense region
uucggcgccagcuguguga

SEQ ID NO: 60 - RNA, 19 nt, Artificial Sequence
  Description of Artificial Sequence Target sequence/siNA sense region
acugccugucccuacaacu

SEQ ID NO: 61 - RNA, 19 nt, Artificial Sequence
  Description of Artificial Sequence Target sequence/siNA sense region
uaccuuucuacggacgugg

SEQ ID NO: 62 - RNA, 19 nt, Artificial Sequence
  Description of Artificial Sequence Target sequence/siNA sense region
ggauccugcacccucgucu

SEQ ID NO: 63 - RNA, 19 nt, Artificial Sequence
  Description of Artificial Sequence Target sequence/siNA sense region
ugcccccugcacaaccaag

SEQ ID NO: 64 - RNA, 19 nt, Artificial Sequence
  Description of Artificial Sequence Target sequence/siNA sense region
gaggugacagcagaggaug

SEQ ID NO: 65 - RNA, 19 nt, Artificial Sequence
  Description of Artificial Sequence Target sequence/siNA sense region
ggaacacagcggugugaga

SEQ ID NO: 66 - RNA, 19 nt, Artificial Sequence
  Description of Artificial Sequence Target sequence/siNA sense region
aagugcagcaagcccugug

SEQ ID NO: 67 - RNA, 19 nt, Artificial Sequence
  Description of Artificial Sequence Target sequence/siNA sense region
gcccgagugugcuaugguc

SEQ ID NO: 68 - RNA, 19 nt, Artificial Sequence
  Description of Artificial Sequence Target sequence/siNA sense region
cugggcauggagcacuugc

SEQ ID NO: 69 - RNA, 19 nt, Artificial Sequence
  Description of Artificial Sequence Target sequence/siNA sense region
cgagaggugagggcaguua

SEQ ID NO: 70 - RNA, 19 nt, Artificial Sequence
  Description of Artificial Sequence Target sequence/siNA sense region
accagugccaauauccagg

SEQ ID NO: 71 - RNA, 19 nt, Artificial Sequence
  Description of Artificial Sequence Target sequence/siNA sense region
gaguuugcuggcugcaaga

SEQ ID NO: 72 - RNA, 19 nt, Artificial Sequence
  Description of Artificial Sequence Target sequence/siNA sense region
aagaucuuugggagccugg

SEQ ID NO: 73 - RNA, 19 nt, Artificial Sequence
  Description of Artificial Sequence Target sequence/siNA sense region
gcauuucugccggagagcu

SEQ ID NO: 74 - RNA, 19 nt, Artificial Sequence
  Description of Artificial Sequence Target sequence/siNA sense region
uuugauggggacccagccu

SEQ ID NO: 75 - RNA, 19 nt, Artificial Sequence
  Description of Artificial Sequence Target sequence/siNA sense region
uccaacacugccccgcucc

SEQ ID NO: 76 - RNA, 19 nt, Artificial Sequence
  Description of Artificial Sequence Target sequence/siNA sense region
cagccagagcagcuccaag

SEQ ID NO: 77 - RNA, 19 nt, Artificial Sequence
  Description of Artificial Sequence Target sequence/siNA sense region
guguuugagacucuggaag

SEQ ID NO: 78 - RNA, 19 nt, Artificial Sequence
  Description of Artificial Sequence Target sequence/siNA sense region
gagaucacagguuaccuau

SEQ ID NO: 79 - RNA, 19 nt, Artificial Sequence
  Description of Artificial Sequence Target sequence/siNA sense region
uacaucucagcauggccgg

SEQ ID NO: 80 - RNA, 19 nt, Artificial Sequence
  Description of Artificial Sequence Target sequence/siNA sense region
gacagccugccugaccuca

SEQ ID NO: 81 - RNA, 19 nt, Artificial Sequence
  Description of Artificial Sequence Target sequence/siNA sense region
agcgucuuccagaaccugc

SEQ ID NO: 82 - RNA, 19 nt, Artificial Sequence
  Description of Artificial Sequence Target sequence/siNA sense region
caaguaauccggggacgaa

SEQ ID NO: 83 - RNA, 19 nt, Artificial Sequence
  Description of Artificial Sequence Target sequence/siNA sense region
auucugcacaauggcgccu

SEQ ID NO: 84 - RNA, 19 nt, Artificial Sequence
  Description of Artificial Sequence Target sequence/siNA sense region
uacucgcugacccugcaag

SEQ ID NO: 85 - RNA, 19 nt, Artificial Sequence
  Description of Artificial Sequence Target sequence/siNA sense region
gggcugggcaucagcuggc

SEQ ID NO: 86 - RNA, 19 nt, Artificial Sequence
  Description of Artificial Sequence Target sequence/siNA sense region
cuggggcugcgcucacuga

SEQ ID NO: 87 - RNA, 19 nt, Artificial Sequence
  Description of Artificial Sequence Target sequence/siNA sense region
agggaacugggcaguggac

SEQ ID NO: 88 - RNA, 19 nt, Artificial Sequence
  Description of Artificial Sequence Target sequence/siNA sense region
cuggcccucauccaccaua

SEQ ID NO: 89 - RNA, 19 nt, Artificial Sequence
  Description of Artificial Sequence Target sequence/siNA sense region
aacacccaccucugcuucg

SEQ ID NO: 90 - RNA, 19 nt, Artificial Sequence
  Description of Artificial Sequence Target sequence/siNA sense region
gugcacacggugcccuggg

SEQ ID NO: 91 - RNA, 19 nt, Artificial Sequence
  Description of Artificial Sequence Target sequence/siNA sense region
gaccagcucuuucggaacc

SEQ ID NO: 92 - RNA, 19 nt, Artificial Sequence
  Description of Artificial Sequence Target sequence/siNA sense region
ccgcaccaagcucugcucc

SEQ ID NO: 93 - RNA, 19 nt, Artificial Sequence
  Description of Artificial Sequence Target sequence/siNA sense region
cacacugccaaccggccag

SEQ ID NO: 94 - RNA, 19 nt, Artificial Sequence
  Description of Artificial Sequence Target sequence/siNA sense region
gaggacgagugugugggcg

SEQ ID NO: 95 - RNA, 19 nt, Artificial Sequence
  Description of Artificial Sequence Target sequence/siNA sense region
gagggccuggccugccacc

SEQ ID NO: 96 - RNA, 19 nt, Artificial Sequence
  Description of Artificial Sequence Target sequence/siNA sense region
cagcugugcgcccgagggc

SEQ ID NO: 97 - RNA, 19 nt, Artificial Sequence
  Description of Artificial Sequence Target sequence/siNA sense region
cacugcugggguccagggc

SEQ ID NO: 98 - RNA, 19 nt, Artificial Sequence
  Description of Artificial Sequence Target sequence/siNA sense region
cccacccagugugucaacu

SEQ ID NO: 99 - RNA, 19 nt, Artificial Sequence
  Description of Artificial Sequence Target sequence/siNA sense region
ugcagccaguuccuucggg

SEQ ID NO: 100 - RNA, 19 nt, Artificial Sequence
  Description of Artificial Sequence Target sequence/siNA sense region
ggccaggagugcguggagg

SEQ ID NO: 101 - RNA, 19 nt, Artificial Sequence
  Description of Artificial Sequence Target sequence/siNA sense region
gaaugccgaguacugcagg

SEQ ID NO: 102 - RNA, 19 nt, Artificial Sequence
  Description of Artificial Sequence Target sequence/siNA sense region
gggcuccccagggaguaug

SEQ ID NO: 103 - RNA, 19 nt, Artificial Sequence
  Description of Artificial Sequence Target sequence/siNA sense region
gugaaugccaggcacuguu

SEQ ID NO: 104 - RNA, 19 nt, Artificial Sequence
  Description of Artificial Sequence Target sequence/siNA sense region
uugccgugccacccugagu

SEQ ID NO: 105 - RNA, 19 nt, Artificial Sequence
  Description of Artificial Sequence Target sequence/siNA sense region
ugucagccccagaauggcu

SEQ ID NO: 106 - RNA, 19 nt, Artificial Sequence
  Description of Artificial Sequence Target sequence/siNA sense region
ucagugaccuguuuuggac

SEQ ID NO: 107 - RNA, 19 nt, Artificial Sequence
  Description of Artificial Sequence Target sequence/siNA sense region
ccggaggcugaccagugug

SEQ ID NO: 108 - RNA, 19 nt, Artificial Sequence
  Description of Artificial Sequence Target sequence/siNA sense region
guggccugugcccacuaua

SEQ ID NO: 109 - RNA, 19 nt, Artificial Sequence
  Description of Artificial Sequence Target sequence/siNA sense region
aaggacccucccuucugcg

SEQ ID NO: 110 - RNA, 19 nt, Artificial Sequence
  Description of Artificial Sequence Target sequence/siNA sense region
guggcccgcugccccagcg

SEQ ID NO: 111 - RNA, 19 nt, Artificial Sequence
  Description of Artificial Sequence Target sequence/siNA sense region
ggugugaaaccugaccucu

SEQ ID NO: 112 - RNA, 19 nt, Artificial Sequence
  Description of Artificial Sequence Target sequence/siNA sense region
uccuacaugcccaucugga

SEQ ID NO: 113 - RNA, 19 nt, Artificial Sequence
  Description of Artificial Sequence Target sequence/siNA sense region
aaguuuccagaugaggagg

SEQ ID NO: 114 - RNA, 19 nt, Artificial Sequence
  Description of Artificial Sequence Target sequence/siNA sense region
ggcgcaugccagccuugcc

SEQ ID NO: 115 - RNA, 19 nt, Artificial Sequence
  Description of Artificial Sequence Target sequence/siNA sense region
cccaucaacugcacccacu

SEQ ID NO: 116 - RNA, 19 nt, Artificial Sequence
  Description of Artificial Sequence Target sequence/siNA sense region
uccuguguggaccuggaug

SEQ ID NO: 117 - RNA, 19 nt, Artificial Sequence
  Description of Artificial Sequence Target sequence/siNA sense region
gacaagggcugccccgccg

SEQ ID NO: 118 - RNA, 19 nt, Artificial Sequence
  Description of Artificial Sequence Target sequence/siNA sense region
gagcagagagccagcccuc

SEQ ID NO: 119 - RNA, 19 nt, Artificial Sequence
  Description of Artificial Sequence Target sequence/siNA sense region
cugacguccaucaucucug

SEQ ID NO: 120 - RNA, 19 nt, Artificial Sequence
  Description of Artificial Sequence Target sequence/siNA sense region
gcggugguuggcauucugc

SEQ ID NO: 121 - RNA, 19 nt, Artificial Sequence
  Description of Artificial Sequence Target sequence/siNA sense region
cuggucguggucuuggggg

SEQ ID NO: 122 - RNA, 19 nt, Artificial Sequence
  Description of Artificial Sequence Target sequence/siNA sense region
guggucuuugggauccuca

SEQ ID NO: 123 - RNA, 19 nt, Artificial Sequence
  Description of Artificial Sequence Target sequence/siNA sense region
aucaagcgacggcagcaga

SEQ ID NO: 124 - RNA, 19 nt, Artificial Sequence
  Description of Artificial Sequence Target sequence/siNA sense region
aagauccggaaguacacga

SEQ ID NO: 125 - RNA, 19 nt, Artificial Sequence
  Description of Artificial Sequence Target sequence/siNA sense region
augcggagacugcugcagg

SEQ ID NO: 126 - RNA, 19 nt, Artificial Sequence
  Description of Artificial Sequence Target sequence/siNA sense region
gaaacggagcugguggagc

SEQ ID NO: 127 - RNA, 19 nt, Artificial Sequence
  Description of Artificial Sequence Target sequence/siNA sense region
ccgcugacaccuagcggag

SEQ ID NO: 128 - RNA, 19 nt, Artificial Sequence
  Description of Artificial Sequence Target sequence/siNA sense region
gcgaugcccaaccaggcgc

SEQ ID NO: 129 - RNA, 19 nt, Artificial Sequence
  Description of Artificial Sequence Target sequence/siNA sense region
cagaugcggauccugaaag

SEQ ID NO: 130 - RNA, 19 nt, Artificial Sequence
  Description of Artificial Sequence Target sequence/siNA sense region
gagacggagcugaggaagg

SEQ ID NO: 131 - RNA, 19 nt, Artificial Sequence
  Description of Artificial Sequence Target sequence/siNA sense region
gugaaggugcuuggaucug

SEQ ID NO: 132 - RNA, 19 nt, Artificial Sequence
  Description of Artificial Sequence Target sequence/siNA sense region
ggcgcuuuuggcacagucu

SEQ ID NO: 133 - RNA, 19 nt, Artificial Sequence
  Description of Artificial Sequence Target sequence/siNA sense region
uacaagggcaucuggaucc

SEQ ID NO: 134 - RNA, 19 nt, Artificial Sequence
  Description of Artificial Sequence Target sequence/siNA sense region
ccugauggggagaauguga

SEQ ID NO: 135 - RNA, 19 nt, Artificial Sequence
  Description of Artificial Sequence Target sequence/siNA sense region
aaaauuccaguggccauca

SEQ ID NO: 136 - RNA, 19 nt, Artificial Sequence
  Description of Artificial Sequence Target sequence/siNA sense region
aaaguguugagggaaaaca

SEQ ID NO: 137 - RNA, 19 nt, Artificial Sequence
  Description of Artificial Sequence Target sequence/siNA sense region
acaucccccaaagccaaca

SEQ ID NO: 138 - RNA, 19 nt, Artificial Sequence
  Description of Artificial Sequence Target sequence/siNA sense region
aaagaaaucuuagacgaag

SEQ ID NO: 139 - RNA, 19 nt, Artificial Sequence
  Description of Artificial Sequence Target sequence/siNA sense region
gcauacgugauggcuggug

SEQ ID NO: 140 - RNA, 19 nt, Artificial Sequence
  Description of Artificial Sequence Target sequence/siNA sense region
gugggcuccccauaugucu

SEQ ID NO: 141 - RNA, 19 nt, Artificial Sequence
  Description of Artificial Sequence Target sequence/siNA sense region
ucccgccuucugggcaucu

SEQ ID NO: 142 - RNA, 19 nt, Artificial Sequence
  Description of Artificial Sequence Target sequence/siNA sense region
ugccugacauccacggugc

SEQ ID NO: 143 - RNA, 19 nt, Artificial Sequence
  Description of Artificial Sequence Target sequence/siNA sense region
cagcuggugacacagcuua

SEQ ID NO: 144 - RNA, 19 nt, Artificial Sequence
  Description of Artificial Sequence Target sequence/siNA sense region
augcccuauggcugccucu

SEQ ID NO: 145 - RNA, 19 nt, Artificial Sequence
  Description of Artificial Sequence Target sequence/siNA sense region
uuagaccauguccgggaaa

SEQ ID NO: 146 - RNA, 19 nt, Artificial Sequence
  Description of Artificial Sequence Target sequence/siNA sense region
aaccgcggacgccugggcu

SEQ ID NO: 147 - RNA, 19 nt, Artificial Sequence
  Description of Artificial Sequence Target sequence/siNA sense region
ucccaggaccugcugaacu

SEQ ID NO: 148 - RNA, 19 nt, Artificial Sequence
  Description of Artificial Sequence Target sequence/siNA sense region
ugguguaugcagauugcca

SEQ ID NO: 149 - RNA, 19 nt, Artificial Sequence
  Description of Artificial Sequence Target sequence/siNA sense region
aaggggaugagcuaccugg

SEQ ID NO: 150 - RNA, 19 nt, Artificial Sequence
  Description of Artificial Sequence Target sequence/siNA sense region
gaggaugugcggcucguac

SEQ ID NO: 151 - RNA, 19 nt, Artificial Sequence
  Description of Artificial Sequence Target sequence/siNA sense region
cacagggacuuggccgcuc

SEQ ID NO: 152 - RNA, 19 nt, Artificial Sequence
  Description of Artificial Sequence Target sequence/siNA sense region
cggaacgugcuggucaaga

SEQ ID NO: 153 - RNA, 19 nt, Artificial Sequence
  Description of Artificial Sequence Target sequence/siNA sense region
agucccaaccaugucaaaa

SEQ ID NO: 154 - RNA, 19 nt, Artificial Sequence
  Description of Artificial Sequence Target sequence/siNA sense region
auuacagacuucgggcugg

SEQ ID NO: 155 - RNA, 19 nt, Artificial Sequence
  Description of Artificial Sequence Target sequence/siNA sense region
gcucggcugcuggacauug

SEQ ID NO: 156 - RNA, 19 nt, Artificial Sequence
  Description of Artificial Sequence Target sequence/siNA sense region
gacgagacagaguaccaug

SEQ ID NO: 157 - RNA, 19 nt, Artificial Sequence
  Description of Artificial Sequence Target sequence/siNA sense region
gcagaugggggcaaggugc

SEQ ID NO: 158 - RNA, 19 nt, Artificial Sequence
  Description of Artificial Sequence Target sequence/siNA sense region
cccaucaaguggauggcgc

SEQ ID NO: 159 - RNA, 19 nt, Artificial Sequence
  Description of Artificial Sequence Target sequence/siNA sense region
cuggaguccauucuccgcc

SEQ ID NO: 160 - RNA, 19 nt, Artificial Sequence
  Description of Artificial Sequence Target sequence/siNA sense region
cggcgguucacccaccaga

SEQ ID NO: 161 - RNA, 19 nt, Artificial Sequence
  Description of Artificial Sequence Target sequence/siNA sense region
agugauguguggaguuaug

SEQ ID NO: 162 - RNA, 19 nt, Artificial Sequence
  Description of Artificial Sequence Target sequence/siNA sense region
ggugugacugugugggagc

SEQ ID NO: 163 - RNA, 19 nt, Artificial Sequence
  Description of Artificial Sequence Target sequence/siNA sense region
cugaugacuuuuggggcca

SEQ ID NO: 164 - RNA, 19 nt, Artificial Sequence
  Description of Artificial Sequence Target sequence/siNA sense region
aaaccuuacgaugggaucc

SEQ ID NO: 165 - RNA, 19 nt, Artificial Sequence
  Description of Artificial Sequence Target sequence/siNA sense region
ccagcccgggagaucccug

SEQ ID NO: 166 - RNA, 19 nt, Artificial Sequence
  Description of Artificial Sequence Target sequence/siNA sense region
gaccugcuggaaaaggggg

SEQ ID NO: 167 - RNA, 19 nt, Artificial Sequence
  Description of Artificial Sequence Target sequence/siNA sense region
gagcggcugccccagcccc

SEQ ID NO: 168 - RNA, 19 nt, Artificial Sequence
  Description of Artificial Sequence Target sequence/siNA sense region
cccaucugcaccauugaug

SEQ ID NO: 169 - RNA, 19 nt, Artificial Sequence
  Description of Artificial Sequence Target sequence/siNA sense region
gucuacaugaucaugguca

SEQ ID NO: 170 - RNA, 19 nt, Artificial Sequence
  Description of Artificial Sequence Target sequence/siNA sense region
aaauguuggaugauugacu

SEQ ID NO: 171 - RNA, 19 nt, Artificial Sequence
  Description of Artificial Sequence Target sequence/siNA sense region
ucugaaugucggccaagau

SEQ ID NO: 172 - RNA, 19 nt, Artificial Sequence
  Description of Artificial Sequence Target sequence/siNA sense region
uuccgggaguuggugucug

SEQ ID NO: 173 - RNA, 19 nt, Artificial Sequence
  Description of Artificial Sequence Target sequence/siNA sense region
gaauucucccgcauggcca

SEQ ID NO: 174 - RNA, 19 nt, Artificial Sequence
  Description of Artificial Sequence Target sequence/siNA sense region
agggacccccagcgcuuug

SEQ ID NO: 175 - RNA, 19 nt, Artificial Sequence
  Description of Artificial Sequence Target sequence/siNA sense region
guggucauccagaaugagg

SEQ ID NO: 176 - RNA, 19 nt, Artificial Sequence
  Description of Artificial Sequence Target sequence/siNA sense region
gacuugggcccagccaguc

SEQ ID NO: 177 - RNA, 19 nt, Artificial Sequence
  Description of Artificial Sequence Target sequence/siNA sense region
cccuuggacagcaccuucu

SEQ ID NO: 178 - RNA, 19 nt, Artificial Sequence
  Description of Artificial Sequence Target sequence/siNA sense region
uaccgcucacugcuggagg

SEQ ID NO: 179 - RNA, 19 nt, Artificial Sequence
  Description of Artificial Sequence Target sequence/siNA sense region
gacgaugacaugggggacc

SEQ ID NO: 180 - RNA, 19 nt, Artificial Sequence
  Description of Artificial Sequence Target sequence/siNA sense region
cugguggaugcugaggagu

SEQ ID NO: 181 - RNA, 19 nt, Artificial Sequence
  Description of Artificial Sequence Target sequence/siNA sense region
uaucugguaccccagcagg

SEQ ID NO: 182 - RNA, 19 nt, Artificial Sequence
  Description of Artificial Sequence Target sequence/siNA sense region
ggcuucuucuguccagacc

SEQ ID NO: 183 - RNA, 19 nt, Artificial Sequence
  Description of Artificial Sequence Target sequence/siNA sense region
ccugccccgggcgcugggg

SEQ ID NO: 184 - RNA, 19 nt, Artificial Sequence
  Description of Artificial Sequence Target sequence/siNA sense region
ggcaugguccaccacaggc

SEQ ID NO: 185 - RNA, 19 nt, Artificial Sequence
  Description of Artificial Sequence Target sequence/siNA sense region
caccgcagcucaucuacca

SEQ ID NO: 186 - RNA, 19 nt, Artificial Sequence
  Description of Artificial Sequence Target sequence/siNA sense region
aggaguggcgguggggacc

SEQ ID NO: 187 - RNA, 19 nt, Artificial Sequence
  Description of Artificial Sequence Target sequence/siNA sense region
cugacacuagggcuggagc

SEQ ID NO: 188 - RNA, 19 nt, Artificial Sequence
  Description of Artificial Sequence Target sequence/siNA sense region
cccucugaagaggaggccc

SEQ ID NO: 189 - RNA, 19 nt, Artificial Sequence
  Description of Artificial Sequence Target sequence/siNA sense region
cccaggucuccacuggcac

SEQ ID NO: 190 - RNA, 19 nt, Artificial Sequence
  Description of Artificial Sequence Target sequence/siNA sense region
cccuccgaaggggcuggcu

SEQ ID NO: 191 - RNA, 19 nt, Artificial Sequence
  Description of Artificial Sequence Target sequence/siNA sense region
uccgauguauuugauggug

SEQ ID NO: 192 - RNA, 19 nt, Artificial Sequence
  Description of Artificial Sequence Target sequence/siNA sense region
gaccugggaaugggggcag

SEQ ID NO: 193 - RNA, 19 nt, Artificial Sequence
  Description of Artificial Sequence Target sequence/siNA sense region
gccaaggggcugcaaagcc

SEQ ID NO: 194 - RNA, 19 nt, Artificial Sequence
  Description of Artificial Sequence Target sequence/siNA sense region
cuccccacacaugacccca

SEQ ID NO: 195 - RNA, 19 nt, Artificial Sequence
  Description of Artificial Sequence Target sequence/siNA sense region
agcccucuacagcgguaca

SEQ ID NO: 196 - RNA, 19 nt, Artificial Sequence
  Description of Artificial Sequence Target sequence/siNA sense region
agugaggaccccacaguac

SEQ ID NO: 197 - RNA, 19 nt, Artificial Sequence
  Description of Artificial Sequence Target sequence/siNA sense region
ccccugcccucugagacug

SEQ ID NO: 198 - RNA, 19 nt, Artificial Sequence
  Description of Artificial Sequence Target sequence/siNA sense region
gauggcuacguugcccccc

SEQ ID NO: 199 - RNA, 19 nt, Artificial Sequence
  Description of Artificial Sequence Target sequence/siNA sense region
cugaccugcagcccccagc

SEQ ID NO: 200 - RNA, 19 nt, Artificial Sequence
  Description of Artificial Sequence Target sequence/siNA sense region
ccugaauaugugaaccagc

SEQ ID NO: 201 - RNA, 19 nt, Artificial Sequence
  Description of Artificial Sequence Target sequence/siNA sense region
ccagauguucggccccagc

SEQ ID NO: 202 - RNA, 19 nt, Artificial Sequence
  Description of Artificial Sequence Target sequence/siNA sense region
cccccuucgccccgagagg

SEQ ID NO: 203 - RNA, 19 nt, Artificial Sequence
  Description of Artificial Sequence Target sequence/siNA sense region
ggcccucugccugcugccc

SEQ ID NO: 204 - RNA, 19 nt, Artificial Sequence
  Description of Artificial Sequence Target sequence/siNA sense region
cgaccugcuggugccacuc

SEQ ID NO: 205 - RNA, 19 nt, Artificial Sequence
  Description of Artificial Sequence Target sequence/siNA sense region
cuggaaaggcccaagacuc

SEQ ID NO: 206 - RNA, 19 nt, Artificial Sequence
  Description of Artificial Sequence Target sequence/siNA sense region
cucuccccagggaagaaug

SEQ ID NO: 207 - RNA, 19 nt, Artificial Sequence
  Description of Artificial Sequence Target sequence/siNA sense region
ggggucgucaaagacguuu

SEQ ID NO: 208 - RNA, 19 nt, Artificial Sequence
  Description of Artificial Sequence Target sequence/siNA sense region
uuugccuuugggggugccg

SEQ ID NO: 209 - RNA, 19 nt, Artificial Sequence
  Description of Artificial Sequence Target sequence/siNA sense region
guggagaaccccgaguacu

SEQ ID NO: 210 - RNA, 19 nt, Artificial Sequence
  Description of Artificial Sequence Target sequence/siNA sense region
uugacaccccagggaggag

SEQ ID NO: 211 - RNA, 19 nt, Artificial Sequence
  Description of Artificial Sequence Target sequence/siNA sense region
gcugccccucagccccacc

SEQ ID NO: 212 - RNA, 19 nt, Artificial Sequence
  Description of Artificial Sequence Target sequence/siNA sense region
ccuccuccugccuucagcc

SEQ ID NO: 213 - RNA, 19 nt, Artificial Sequence
  Description of Artificial Sequence Target sequence/siNA sense region
ccagccuucgacaaccucu

SEQ ID NO: 214 - RNA, 19 nt, Artificial Sequence
  Description of Artificial Sequence Target sequence/siNA sense region
uauuacugggaccaggacc

SEQ ID NO: 215 - RNA, 19 nt, Artificial Sequence
  Description of Artificial Sequence Target sequence/siNA sense region
ccaccagagcggggggcuc

SEQ ID NO: 216 - RNA, 19 nt, Artificial Sequence
  Description of Artificial Sequence Target sequence/siNA sense region
ccacccagcaccuucaaag

SEQ ID NO: 217 - RNA, 19 nt, Artificial Sequence
  Description of Artificial Sequence Target sequence/siNA sense region
gggacaccuacggcagaga

SEQ ID NO: 218 - RNA, 19 nt, Artificial Sequence
  Description of Artificial Sequence Target sequence/siNA sense region
aacccagaguaccuggguc

SEQ ID NO: 219 - RNA, 19 nt, Artificial Sequence
  Description of Artificial Sequence Target sequence/siNA sense region
cuggacgugccagugugaa

SEQ ID NO: 220 - RNA, 19 nt, Artificial Sequence
  Description of Artificial Sequence Target sequence/siNA sense region
accagaaggccaaguccgc

SEQ ID NO: 221 - RNA, 19 nt, Artificial Sequence
  Description of Artificial Sequence Target sequence/siNA sense region
cagaagcccugaugugucc

SEQ ID NO: 222 - RNA, 19 nt, Artificial Sequence
  Description of Artificial Sequence Target sequence/siNA sense region
cucagggagcagggaaggc

SEQ ID NO: 223 - RNA, 19 nt, Artificial Sequence
  Description of Artificial Sequence Target sequence/siNA sense region
ccugacuucugcuggcauc

SEQ ID NO: 224 - RNA, 19 nt, Artificial Sequence
  Description of Artificial Sequence Target sequence/siNA sense region
caagaggugggagggcccu

SEQ ID NO: 225 - RNA, 19 nt, Artificial Sequence
  Description of Artificial Sequence Target sequence/siNA sense region
uccgaccacuuccagggga

SEQ ID NO: 226 - RNA, 19 nt, Artificial Sequence
  Description of Artificial Sequence Target sequence/siNA sense region
aaccugccaugccaggaac

SEQ ID NO: 227 - RNA, 19 nt, Artificial Sequence
  Description of Artificial Sequence Target sequence/siNA sense region
ccuguccuaaggaaccuuc

SEQ ID NO: 228 - RNA, 19 nt, Artificial Sequence
  Description of Artificial Sequence Target sequence/siNA sense region
ccuuccugcuugaguuccc

SEQ ID NO: 229 - RNA, 19 nt, Artificial Sequence
  Description of Artificial Sequence Target sequence/siNA sense region
cagauggcuggaagggguc

SEQ ID NO: 230 - RNA, 19 nt, Artificial Sequence
  Description of Artificial Sequence Target sequence/siNA sense region
ccagccucguuggaagagg

SEQ ID NO: 231 - RNA, 19 nt, Artificial Sequence
  Description of Artificial Sequence Target sequence/siNA sense region
gaacagcacuggggagucu

SEQ ID NO: 232 - RNA, 19 nt, Artificial Sequence
  Description of Artificial Sequence Target sequence/siNA sense region
uuuguggauucugaggccc

SEQ ID NO: 233 - RNA, 19 nt, Artificial Sequence
  Description of Artificial Sequence Target sequence/siNA sense region
cugcccaaugagacucuag

SEQ ID NO: 234 - RNA, 19 nt, Artificial Sequence
  Description of Artificial Sequence Target sequence/siNA sense region
ggguccaguggaugccaca

SEQ ID NO: 235 - RNA, 19 nt, Artificial Sequence
  Description of Artificial Sequence Target sequence/siNA sense region
agcccagcuuggcccuuuc

SEQ ID NO: 236 - RNA, 19 nt, Artificial Sequence
  Description of Artificial Sequence Target sequence/siNA sense region
ccuuccagauccuggguac

SEQ ID NO: 237 - RNA, 19 nt, Artificial Sequence
  Description of Artificial Sequence Target sequence/siNA sense region
cugaaagccuuagggaagc

SEQ ID NO: 238 - RNA, 19 nt, Artificial Sequence
  Description of Artificial Sequence Target sequence/siNA sense region
cuggccugagaggggaagc

SEQ ID NO: 239 - RNA, 19 nt, Artificial Sequence
  Description of Artificial Sequence Target sequence/siNA sense region
cggcccuaagggagugucu

SEQ ID NO: 240 - RNA, 19 nt, Artificial Sequence
  Description of Artificial Sequence Target sequence/siNA sense region
uaagaacaaaagcgaccca

SEQ ID NO: 241 - RNA, 19 nt, Artificial Sequence
  Description of Artificial Sequence Target sequence/siNA sense region
auucagagacugucccuga

SEQ ID NO: 242 - RNA, 19 nt, Artificial Sequence
  Description of Artificial Sequence Target sequence/siNA sense region
aaaccuaguacugcccccc

SEQ ID NO: 243 - RNA, 19 nt, Artificial Sequence
  Description of Artificial Sequence Target sequence/siNA sense region
caugaggaaggaacagcaa

SEQ ID NO: 244 - RNA, 19 nt, Artificial Sequence
  Description of Artificial Sequence Target sequence/siNA sense region
auggugucaguauccaggc

SEQ ID NO: 245 - RNA, 19 nt, Artificial Sequence
  Description of Artificial Sequence Target sequence/siNA sense region
cuuuguacagagugcuuuu

SEQ ID NO: 246 - RNA, 19 nt, Artificial Sequence
  Description of Artificial Sequence Target sequence/siNA sense region
ucuguuuaguuuuuacuuu

SEQ ID NO: 247 - RNA, 19 nt, Artificial Sequence
  Description of Artificial Sequence Target sequence/siNA sense region
uuuuuguuuuguuuuuuua

SEQ ID NO: 248 - RNA, 19 nt, Artificial Sequence
  Description of Artificial Sequence Target sequence/siNA sense region
aaagaugaaauaaagaccc

SEQ ID NO: 249 - RNA, 19 nt, Artificial Sequence
  Description of Artificial Sequence Target sequence/siNA sense region
aauaaagacccagggggag

SEQ ID NO: 250 - RNA, 19 nt, Artificial Sequence
  Description of Artificial Sequence siNA antisense region
gccaggguuaccuccccuu

SEQ ID NO: 251 - RNA, 19 nt, Artificial Sequence
  Description of Artificial Sequence siNA antisense region
ggggccccgaccaaagggg

SEQ ID NO: 252 - RNA, 19 nt, Artificial Sequence
  Description of Artificial Sequence siNA antisense region
aggggcgcgcggcugcccg

SEQ ID NO: 253 - RNA, 19 nt, Artificial Sequence
  Description of Artificial Sequence siNA antisense region
guaaagggccccgugggaa

SEQ ID NO: 254 - RNA, 19 nt, Artificial Sequence
  Description of Artificial Sequence siNA antisense region
ggccgggcgcgcggcgcag

SEQ ID NO: 255 - RNA, 19 nt, Artificial Sequence
  Description of Artificial Sequence siNA antisense region
ggugcugcgaggggugggg

SEQ ID NO: 256 - RNA, 19 nt, Artificial Sequence
  Description of Artificial Sequence siNA antisense region
ggagggcgcggggcgcggg

SEQ ID NO: 257 - RNA, 19 nt, Artificial Sequence
  Description of Artificial Sequence siNA antisense region
uccggcuggacccggcugg

SEQ ID NO: 258 - RNA, 19 nt, Artificial Sequence
  Description of Artificial Sequence siNA antisense region
cggcuccggccccauggcu

SEQ ID NO: 259 - RNA, 19 nt, Artificial Sequence
  Description of Artificial Sequence siNA antisense region
gcuccauggugcucacugc

SEQ ID NO: 260 - RNA, 19 nt, Artificial Sequence
  Description of Artificial Sequence siNA antisense region
agcggcacaaggccgccag

SEQ ID NO: 261 - RNA, 19 nt, Artificial Sequence
  Description of Artificial Sequence siNA antisense region
gggcgaggaggagccccca

SEQ ID NO: 262 - RNA, 19 nt, Artificial Sequence
  Description of Artificial Sequence siNA antisense region
cggcuccggggggcaagag

SEQ ID NO: 263 - RNA, 19 nt, Artificial Sequence
  Description of Artificial Sequence siNA antisense region
ugcacacuugggugcucgc

SEQ ID NO: 264 - RNA, 19 nt, Artificial Sequence
  Description of Artificial Sequence siNA antisense region
gcuucaugucugugccggu

SEQ ID NO: 265 - RNA, 19 nt, Artificial Sequence
  Description of Artificial Sequence siNA antisense region
gacuggcagggagccgcag

SEQ ID NO: 266 - RNA, 19 nt, Artificial Sequence
  Description of Artificial Sequence siNA antisense region
uguccaggugggucucggg

SEQ ID NO: 267 - RNA, 19 nt, Artificial Sequence
  Description of Artificial Sequence siNA antisense region
gguagagguggcggagcau

SEQ ID NO: 268 - RNA, 19 nt, Artificial Sequence
  Description of Artificial Sequence siNA antisense region
gcaccaccuggcagcccug

SEQ ID NO: 269 - RNA, 19 nt, Artificial Sequence
  Description of Artificial Sequence siNA antisense region
ugaguuccagguuucccug

SEQ ID NO: 270 - RNA, 19 nt, Artificial Sequence
  Description of Artificial Sequence siNA antisense region
cauuggugggcagguaggu

SEQ ID NO: 271 - RNA, 19 nt, Artificial Sequence
  Description of Artificial Sequence siNA antisense region
gcaggaaggacaggcuggc

SEQ ID NO: 272 - RNA, 19 nt, Artificial Sequence
  Description of Artificial Sequence siNA antisense region
gcaccuccuggauauccug

SEQ ID NO: 273 - RNA, 19 nt, Artificial Sequence
  Description of Artificial Sequence siNA antisense region
cgaugagcacguagcccug

SEQ ID NO: 274 - RNA, 19 nt, Artificial Sequence
  Description of Artificial Sequence siNA antisense region
gccucacuugguugugagc

SEQ ID NO: 275 - RNA, 19 nt, Artificial Sequence
  Description of Artificial Sequence siNA antisense region
gccucugcagugggaccug

SEQ ID NO: 276 - RNA, 19 nt, Artificial Sequence
  Description of Artificial Sequence siNA antisense region
ugccucgcacaauccgcag

SEQ ID NO: 277 - RNA, 19 nt, Artificial Sequence
  Description of Artificial Sequence siNA antisense region
uguccucaaagagcugggu

SEQ ID NO: 278 - RNA, 19 nt, Artificial Sequence
  Description of Artificial Sequence siNA antisense region
gcacggccagggcauaguu

SEQ ID NO: 279 - RNA, 19 nt, Artificial Sequence
  Description of Artificial Sequence siNA antisense region
gcgggucuccauugucuag

SEQ ID NO: 280 - RNA, 19 nt, Artificial Sequence
  Description of Artificial Sequence siNA antisense region
caggggugguauuguucag

SEQ ID NO: 281 - RNA, 19 nt, Artificial Sequence
  Description of Artificial Sequence siNA antisense region
cuggggaggccccugugac

SEQ ID NO: 282 - RNA, 19 nt, Artificial Sequence
  Description of Artificial Sequence siNA antisense region
gcagcucccgcaggccucc

SEQ ID NO: 283 - RNA, 19 nt, Artificial Sequence
  Description of Artificial Sequence siNA antisense region
cugugaggcuucgaagcug

SEQ ID NO: 284 - RNA, 19 nt, Artificial Sequence
  Description of Artificial Sequence siNA antisense region
ccccuccuuucaagaucuc

SEQ ID NO: 285 - RNA, 19 nt, Artificial Sequence
  Description of Artificial Sequence siNA antisense region
gguuccgcuggaucaagac

SEQ ID NO: 286 - RNA, 19 nt, Artificial Sequence
  Description of Artificial Sequence siNA antisense region
ccugguagcagagcugggg

SEQ ID NO: 287 - RNA, 19 nt, Artificial Sequence
  Description of Artificial Sequence siNA antisense region
ccuuccacaaaaucguguc

SEQ ID NO: 288 - RNA, 19 nt, Artificial Sequence
  Description of Artificial Sequence siNA antisense region
uguucuuguggaagauguc

SEQ ID NO: 289 - RNA, 19 nt, Artificial Sequence
  Description of Artificial Sequence siNA antisense region
gugugagagccagcugguu

SEQ ID NO: 290 - RNA, 19 nt, Artificial Sequence
  Description of Artificial Sequence siNA antisense region
agcgguuggugucuaucag

SEQ ID NO: 291 - RNA, 19 nt, Artificial Sequence
  Description of Artificial Sequence siNA antisense region
agggguggcaggcccgaga

SEQ ID NO: 292 - RNA, 19 nt, Artificial Sequence
  Description of Artificial Sequence siNA antisense region
ccuuacacaucggagaaca

SEQ ID NO: 293 - RNA, 19 nt, Artificial Sequence
  Description of Artificial Sequence siNA antisense region
cuccccagcagcgggagcc

SEQ ID NO: 294 - RNA, 19 nt, Artificial Sequence
  Description of Artificial Sequence siNA antisense region
gacaauccucagaacucuc

SEQ ID NO: 295 - RNA, 19 nt, Artificial Sequence
  Description of Artificial Sequence siNA antisense region
cagugcgcgucaggcucug

SEQ ID NO: 296 - RNA, 19 nt, Artificial Sequence
  Description of Artificial Sequence siNA antisense region
cacagccaccggcacagac

SEQ ID NO: 297 - RNA, 19 nt, Artificial Sequence
  Description of Artificial Sequence siNA antisense region
guggccccuugcagcgggc

SEQ ID NO: 298 - RNA, 19 nt, Artificial Sequence
  Description of Artificial Sequence siNA antisense region
ggcagcagucagugggcag

SEQ ID NO: 299 - RNA, 19 nt, Artificial Sequence
  Description of Artificial Sequence siNA antisense region
cggcagcacacugcucaug

SEQ ID NO: 300 - RNA, 19 nt, Artificial Sequence
  Description of Artificial Sequence siNA antisense region
gcuuggggcccgugcagcc

SEQ ID NO: 301 - RNA, 19 nt, Artificial Sequence
  Description of Artificial Sequence siNA antisense region
aggccaggcagucagagug

SEQ ID NO: 302 - RNA, 19 nt, Artificial Sequence
  Description of Artificial Sequence siNA antisense region
ugugguugaaguggaggca

SEQ ID NO: 303 - RNA, 19 nt, Artificial Sequence
  Description of Artificial Sequence siNA antisense region
gcagcucacagaugccacu

SEQ ID NO: 304 - RNA, 19 nt, Artificial Sequence
  Description of Artificial Sequence siNA antisense region
ugaccagggcugggcagug

SEQ ID NO: 305 - RNA, 19 nt, Artificial Sequence
  Description of Artificial Sequence siNA antisense region
acgugucuguguuguaggu

SEQ ID NO: 306 - RNA, 19 nt, Artificial Sequence
  Description of Artificial Sequence siNA antisense region
gauugggcauggacucaaa

SEQ ID NO: 307 - RNA, 19 nt, Artificial Sequence
  Description of Artificial Sequence siNA antisense region
auguauaccggcccucggg

SEQ ID NO: 308 - RNA, 19 nt, Artificial Sequence
  Description of Artificial Sequence siNA antisense region
ucacacagcuggcgccgaa

SEQ ID NO: 309 - RNA, 19 nt, Artificial Sequence
  Description of Artificial Sequence siNA antisense region
aguuguagggacaggcagu

SEQ ID NO: 310 - RNA, 19 nt, Artificial Sequence
  Description of Artificial Sequence siNA antisense region
ccacguccguagaaaggua

SEQ ID NO: 311 - RNA, 19 nt, Artificial Sequence
  Description of Artificial Sequence siNA antisense region
agacgagggugcaggaucc

SEQ ID NO: 312 - RNA, 19 nt, Artificial Sequence
  Description of Artificial Sequence siNA antisense region
cuugguugugcagggggca

SEQ ID NO: 313 - RNA, 19 nt, Artificial Sequence
  Description of Artificial Sequence siNA antisense region
cauccucugcugucaccuc

SEQ ID NO: 314 - RNA, 19 nt, Artificial Sequence
  Description of Artificial Sequence siNA antisense region
ucucacaccgcuguguucc

SEQ ID NO: 315 - RNA, 19 nt, Artificial Sequence
  Description of Artificial Sequence siNA antisense region
cacagggcuugcugcacuu

SEQ ID NO: 316 - RNA, 19 nt, Artificial Sequence
  Description of Artificial Sequence siNA antisense region
gaccauagcacacucgggc

SEQ ID NO: 317 - RNA, 19 nt, Artificial Sequence
  Description of Artificial Sequence siNA antisense region
gcaagugcuccaugcccag

SEQ ID NO: 318 - RNA, 19 nt, Artificial Sequence
  Description of Artificial Sequence siNA antisense region
uaacugcccucaccucucg

SEQ ID NO: 319 - RNA, 19 nt, Artificial Sequence
  Description of Artificial Sequence siNA antisense region
ccuggauauuggcacuggu

SEQ ID NO: 320 - RNA, 19 nt, Artificial Sequence
  Description of Artificial Sequence siNA antisense region
ucuugcagccagcaaacuc

SEQ ID NO: 321 - RNA, 19 nt, Artificial Sequence
  Description of Artificial Sequence siNA antisense region
ccaggcucccaaagaucuu

SEQ ID NO: 322 - RNA, 19 nt, Artificial Sequence
  Description of Artificial Sequence siNA antisense region
agcucuccggcagaaaugc

SEQ ID NO: 323 - RNA, 19 nt, Artificial Sequence
  Description of Artificial Sequence siNA antisense region
aggcuggguccccaucaaa

SEQ ID NO: 324 - RNA, 19 nt, Artificial Sequence
  Description of Artificial Sequence siNA antisense region
ggagcggggcaguguugga

SEQ ID NO: 325 - RNA, 19 nt, Artificial Sequence
  Description of Artificial Sequence siNA antisense region
cuuggagcugcucuggcug

SEQ ID NO: 326 - RNA, 19 nt, Artificial Sequence
  Description of Artificial Sequence siNA antisense region
cuuccagagucucaaacac

SEQ ID NO: 327 - RNA, 19 nt, Artificial Sequence
  Description of Artificial Sequence siNA antisense region
auagguaaccugugaucuc

SEQ ID NO: 328 - RNA, 19 nt, Artificial Sequence
  Description of Artificial Sequence siNA antisense region
ccggccaugcugagaugua

SEQ ID NO: 329 - RNA, 19 nt, Artificial Sequence
  Description of Artificial Sequence siNA antisense region
ugaggucaggcaggcuguc

SEQ ID NO: 330 - RNA, 19 nt, Artificial Sequence
  Description of Artificial Sequence siNA antisense region
gcagguucuggaagacgcu

SEQ ID NO: 331 - RNA, 19 nt, Artificial Sequence
  Description of Artificial Sequence siNA antisense region
uucguccccggauuacuug

SEQ ID NO: 332 - RNA, 19 nt, Artificial Sequence
  Description of Artificial Sequence siNA antisense region
aggcgccauugugcagaau

SEQ ID NO: 333 - RNA, 19 nt, Artificial Sequence
  Description of Artificial Sequence siNA antisense region
cuugcagggucagcgagua

SEQ ID NO: 334 - RNA, 19 nt, Artificial Sequence
  Description of Artificial Sequence siNA antisense region
gccagcugaugcccagccc

SEQ ID NO: 335 - RNA, 19 nt, Artificial Sequence
  Description of Artificial Sequence siNA antisense region
ucagugagcgcagccccag

SEQ ID NO: 336 - RNA, 19 nt, Artificial Sequence
  Description of Artificial Sequence siNA antisense region
guccacugcccaguucccu

SEQ ID NO: 337 - RNA, 19 nt, Artificial Sequence
  Description of Artificial Sequence siNA antisense region
uaugguggaugagggccag

SEQ ID NO: 338 - RNA, 19 nt, Artificial Sequence
  Description of Artificial Sequence siNA antisense region
cgaagcagagguggguguu

SEQ ID NO: 339 - RNA, 19 nt, Artificial Sequence
  Description of Artificial Sequence siNA antisense region
cccagggcaccgugugcac

SEQ ID NO: 340 - RNA, 19 nt, Artificial Sequence
  Description of Artificial Sequence siNA antisense region
gguuccgaaagagcugguc

SEQ ID NO: 341 - RNA, 19 nt, Artificial Sequence
  Description of Artificial Sequence siNA antisense region
ggagcagagcuuggugcgg

SEQ ID NO: 342 - RNA, 19 nt, Artificial Sequence
  Description of Artificial Sequence siNA antisense region
cuggccgguuggcagugug

SEQ ID NO: 343 - RNA, 19 nt, Artificial Sequence
  Description of Artificial Sequence siNA antisense region
cgcccacacacucguccuc

SEQ ID NO: 344 - RNA, 19 nt, Artificial Sequence
  Description of Artificial Sequence siNA antisense region
gguggcaggccaggcccuc

SEQ ID NO: 345 - RNA, 19 nt, Artificial Sequence
  Description of Artificial Sequence siNA antisense region
gcccucgggcgcacagcug

SEQ ID NO: 346 - RNA, 19 nt, Artificial Sequence
  Description of Artificial Sequence siNA antisense region
gcccuggaccccagcagug

SEQ ID NO: 347 - RNA, 19 nt, Artificial Sequence
  Description of Artificial Sequence siNA antisense region
aguugacacacuggguggg

SEQ ID NO: 348 - RNA, 19 nt, Artificial Sequence
  Description of Artificial Sequence siNA antisense region
cccgaaggaacuggcugca

SEQ ID NO: 349 - RNA, 19 nt, Artificial Sequence
  Description of Artificial Sequence siNA antisense region
ccuccacgcacuccuggcc

SEQ ID NO: 350 - RNA, 19 nt, Artificial Sequence
  Description of Artificial Sequence siNA antisense region
ccugcaguacucggcauuc

SEQ ID NO: 351 - RNA, 19 nt, Artificial Sequence
  Description of Artificial Sequence siNA antisense region
cauacucccuggggagccc

SEQ ID NO: 352 - RNA, 19 nt, Artificial Sequence
  Description of Artificial Sequence siNA antisense region
aacagugccuggcauucac

SEQ ID NO: 353 - RNA, 19 nt, Artificial Sequence
  Description of Artificial Sequence siNA antisense region
acucaggguggcacggcaa

SEQ ID NO: 354 - RNA, 19 nt, Artificial Sequence
  Description of Artificial Sequence siNA antisense region
agccauucuggggcugaca

SEQ ID NO: 355 - RNA, 19 nt, Artificial Sequence
  Description of Artificial Sequence siNA antisense region
guccaaaacaggucacuga

SEQ ID NO: 356 - RNA, 19 nt, Artificial Sequence
  Description of Artificial Sequence siNA antisense region
cacacuggucagccuccgg

SEQ ID NO: 357 - RNA, 19 nt, Artificial Sequence
  Description of Artificial Sequence siNA antisense region
uauagugggcacaggccac

SEQ ID NO: 358 - RNA, 19 nt, Artificial Sequence
  Description of Artificial Sequence siNA antisense region
cgcagaagggaggguccuu

SEQ ID NO: 359 - RNA, 19 nt, Artificial Sequence
  Description of Artificial Sequence siNA antisense region
cgcuggggcagcgggccac

SEQ ID NO: 360 - RNA, 19 nt, Artificial Sequence
  Description of Artificial Sequence siNA antisense region
agaggucagguuucacacc

SEQ ID NO: 361 - RNA, 19 nt, Artificial Sequence
  Description of Artificial Sequence siNA antisense region
uccagaugggcauguagga

SEQ ID NO: 362 - RNA, 19 nt, Artificial Sequence
  Description of Artificial Sequence siNA antisense region
ccuccucaucuggaaacuu

SEQ ID NO: 363 - RNA, 19 nt, Artificial Sequence
  Description of Artificial Sequence siNA antisense region
ggcaaggcuggcaugcgcc

SEQ ID NO: 364 - RNA, 19 nt, Artificial Sequence
  Description of Artificial Sequence siNA antisense region
agugggugcaguugauggg

SEQ ID NO: 365 - RNA, 19 nt, Artificial Sequence
  Description of Artificial Sequence siNA antisense region
cauccagguccacacagga

SEQ ID NO: 366 - RNA, 19 nt, Artificial Sequence
  Description of Artificial Sequence siNA antisense region
cggcggggcagcccuuguc

SEQ ID NO: 367 - RNA, 19 nt, Artificial Sequence
  Description of Artificial Sequence siNA antisense region
gagggcuggcucucugcuc

SEQ ID NO: 368 - RNA, 19 nt, Artificial Sequence
  Description of Artificial Sequence siNA antisense region
cagagaugauggacgucag

SEQ ID NO: 369 - RNA, 19 nt, Artificial Sequence
  Description of Artificial Sequence siNA antisense region
gcagaaugccaaccaccgc

SEQ ID NO: 370 - RNA, 19 nt, Artificial Sequence
  Description of Artificial Sequence siNA antisense region
cccccaagaccacgaccag

SEQ ID NO: 371 - RNA, 19 nt, Artificial Sequence
  Description of Artificial Sequence siNA antisense region
ugaggaucccaaagaccac

SEQ ID NO: 372 - RNA, 19 nt, Artificial Sequence
  Description of Artificial Sequence siNA antisense region
ucugcugccgucgcuugau

SEQ ID NO: 373 - RNA, 19 nt, Artificial Sequence
  Description of Artificial Sequence siNA antisense region
ucguguacuuccggaucuu

SEQ ID NO: 374 - RNA, 19 nt, Artificial Sequence
  Description of Artificial Sequence siNA antisense region
ccugcagcagucuccgcau

SEQ ID NO: 375 - RNA, 19 nt, Artificial Sequence
  Description of Artificial Sequence siNA antisense region
gcuccaccagcuccguuuc

SEQ ID NO: 376 - RNA, 19 nt, Artificial Sequence
  Description of Artificial Sequence siNA antisense region
cuccgcuaggugucagcgg

SEQ ID NO: 377 - RNA, 19 nt, Artificial Sequence
  Description of Artificial Sequence siNA antisense region
gcgccugguugggcaucgc

SEQ ID NO: 378 - RNA, 19 nt, Artificial Sequence
  Description of Artificial Sequence siNA antisense region
cuuucaggauccgcaucug

SEQ ID NO: 379 - RNA, 19 nt, Artificial Sequence
  Description of Artificial Sequence siNA antisense region
ccuuccucagcuccgucuc

SEQ ID NO: 380 - RNA, 19 nt, Artificial Sequence
  Description of Artificial Sequence siNA antisense region
cagauccaagcaccuucac

SEQ ID NO: 381 - RNA, 19 nt, Artificial Sequence
  Description of Artificial Sequence siNA antisense region
agacugugccaaaagcgcc

SEQ ID NO: 382 - RNA, 19 nt, Artificial Sequence
  Description of Artificial Sequence siNA antisense region
ggauccagaugcccuugua

SEQ ID NO: 383 - RNA, 19 nt, Artificial Sequence
  Description of Artificial Sequence siNA antisense region
ucacauucuccccaucagg

SEQ ID NO: 384 - RNA, 19 nt, Artificial Sequence
  Description of Artificial Sequence siNA antisense region
ugauggccacuggaauuuu

SEQ ID NO: 385 - RNA, 19 nt, Artificial Sequence
  Description of Artificial Sequence siNA antisense region
uguuuucccucaacacuuu

SEQ ID NO: 386 - RNA, 19 nt, Artificial Sequence
  Description of Artificial Sequence siNA antisense region
uguuggcuuugggggaugu

SEQ ID NO: 387 - RNA, 19 nt, Artificial Sequence
  Description of Artificial Sequence siNA antisense region
cuucgucuaagauuucuuu

SEQ ID NO: 388 - RNA, 19 nt, Artificial Sequence
  Description of Artificial Sequence siNA antisense region
caccagccaucacguaugc

SEQ ID NO: 389 - RNA, 19 nt, Artificial Sequence
  Description of Artificial Sequence siNA antisense region
agacauauggggagcccac

SEQ ID NO: 390 - RNA, 19 nt, Artificial Sequence
  Description of Artificial Sequence siNA antisense region
agaugcccagaaggcggga

SEQ ID NO: 391 - RNA, 19 nt, Artificial Sequence
  Description of Artificial Sequence siNA antisense region
gcaccguggaugucaggca

SEQ ID NO: 392 - RNA, 19 nt, Artificial Sequence
  Description of Artificial Sequence siNA antisense region
uaagcugugucaccagcug

SEQ ID NO: 393 - RNA, 19 nt, Artificial Sequence
  Description of Artificial Sequence siNA antisense region
agaggcagccauagggcau

SEQ ID NO: 394 - RNA, 19 nt, Artificial Sequence
  Description of Artificial Sequence siNA antisense region
uuucccggacauggucuaa

SEQ ID NO: 395 - RNA, 19 nt, Artificial Sequence
  Description of Artificial Sequence siNA antisense region
agcccaggcguccgcgguu

SEQ ID NO: 396 - RNA, 19 nt, Artificial Sequence
  Description of Artificial Sequence siNA antisense region
aguucagcagguccuggga

SEQ ID NO: 397 - RNA, 19 nt, Artificial Sequence
  Description of Artificial Sequence siNA antisense region
uggcaaucugcauacacca

SEQ ID NO: 398 - RNA, 19 nt, Artificial Sequence
  Description of Artificial Sequence siNA antisense region
ccagguagcucauccccuu

SEQ ID NO: 399 - RNA, 19 nt, Artificial Sequence
  Description of Artificial Sequence siNA antisense region
guacgagccgcacauccuc

SEQ ID NO: 400 - RNA, 19 nt, Artificial Sequence
  Description of Artificial Sequence siNA antisense region
gagcggccaagucccugug

SEQ ID NO: 401 - RNA, 19 nt, Artificial Sequence
  Description of Artificial Sequence siNA antisense region
ucuugaccagcacguuccg

SEQ ID NO: 402 - RNA, 19 nt, Artificial Sequence
  Description of Artificial Sequence siNA antisense region
uuuugacaugguugggacu

SEQ ID NO: 403 - RNA, 19 nt, Artificial Sequence
  Description of Artificial Sequence siNA antisense region
ccagcccgaagucuguaau

SEQ ID NO: 404 - RNA, 19 nt, Artificial Sequence
  Description of Artificial Sequence siNA antisense region
caauguccagcagccgagc

SEQ ID NO: 405 - RNA, 19 nt, Artificial Sequence
  Description of Artificial Sequence siNA antisense region
caugguacucugucucguc

SEQ ID NO: 406 - RNA, 19 nt, Artificial Sequence
  Description of Artificial Sequence siNA antisense region
gcaccuugcccccaucugc

SEQ ID NO: 407 - RNA, 19 nt, Artificial Sequence
  Description of Artificial Sequence siNA antisense region
gcgccauccacuugauggg

SEQ ID NO: 408 - RNA, 19 nt, Artificial Sequence
  Description of Artificial Sequence siNA antisense region
ggcggagaauggacuccag

SEQ ID NO: 409 - RNA, 19 nt, Artificial Sequence
  Description of Artificial Sequence siNA antisense region
ucuggugggugaaccgccg

SEQ ID NO: 410 - RNA, 19 nt, Artificial Sequence
  Description of Artificial Sequence siNA antisense region
cauaacuccacacaucacu

SEQ ID NO: 411 - RNA, 19 nt, Artificial Sequence
  Description of Artificial Sequence siNA antisense region
gcucccacacagucacacc

SEQ ID NO: 412 - RNA, 19 nt, Artificial Sequence
  Description of Artificial Sequence siNA antisense region
uggccccaaaagucaucag

SEQ ID NO: 413 - RNA, 19 nt, Artificial Sequence
  Description of Artificial Sequence siNA antisense region
ggaucccaucguaagguuu

SEQ ID NO: 414 - RNA, 19 nt, Artificial Sequence
  Description of Artificial Sequence siNA antisense region
cagggaucucccgggcugg

SEQ ID NO: 415 - RNA, 19 nt, Artificial Sequence
  Description of Artificial Sequence siNA antisense region
cccccuuuuccagcagguc

SEQ ID NO: 416 - RNA, 19 nt, Artificial Sequence
  Description of Artificial Sequence siNA antisense region
ggggcuggggcagccgcuc

SEQ ID NO: 417 - RNA, 19 nt, Artificial Sequence
  Description of Artificial Sequence siNA antisense region
caucaauggugcagauggg

SEQ ID NO: 418 - RNA, 19 nt, Artificial Sequence
  Description of Artificial Sequence siNA antisense region
ugaccaugaucauguagac

SEQ ID NO: 419 - RNA, 19 nt, Artificial Sequence
  Description of Artificial Sequence siNA antisense region
agucaaucauccaacauuu

SEQ ID NO: 420 - RNA, 19 nt, Artificial Sequence
  Description of Artificial Sequence siNA antisense region
aucuuggccgacauucaga

SEQ ID NO: 421 - RNA, 19 nt, Artificial Sequence
  Description of Artificial Sequence siNA antisense region
cagacaccaacucccggaa

SEQ ID NO: 422 - RNA, 19 nt, Artificial Sequence
  Description of Artificial Sequence siNA antisense region
uggccaugcgggagaauuc

SEQ ID NO: 423 - RNA, 19 nt, Artificial Sequence
  Description of Artificial Sequence siNA antisense region
caaagcgcugggggucccu

SEQ ID NO: 424 - RNA, 19 nt, Artificial Sequence
  Description of Artificial Sequence siNA antisense region
ccucauucuggaugaccac

SEQ ID NO: 425 - RNA, 19 nt, Artificial Sequence
  Description of Artificial Sequence siNA antisense region
gacuggcugggcccaaguc

SEQ ID NO: 426 - RNA, 19 nt, Artificial Sequence
  Description of Artificial Sequence siNA antisense region
agaaggugcuguccaaggg

SEQ ID NO: 427 - RNA, 19 nt, Artificial Sequence
  Description of Artificial Sequence siNA antisense region
ccuccagcagugagcggua

SEQ ID NO: 428 - RNA, 19 nt, Artificial Sequence
  Description of Artificial Sequence siNA antisense region
ggucccccaugucaucguc

SEQ ID NO: 429 - RNA, 19 nt, Artificial Sequence
  Description of Artificial Sequence siNA antisense region
acuccucagcauccaccag

SEQ ID NO: 430 - RNA, 19 nt, Artificial Sequence
  Description of Artificial Sequence siNA antisense region
ccugcugggguaccagaua

SEQ ID NO: 431 - RNA, 19 nt, Artificial Sequence
  Description of Artificial Sequence siNA antisense region
ggucuggacagaagaagcc

SEQ ID NO: 432 - RNA, 19 nt, Artificial Sequence
  Description of Artificial Sequence siNA antisense region
ccccagcgcccggggcagg

SEQ ID NO: 433 - RNA, 19 nt, Artificial Sequence
  Description of Artificial Sequence siNA antisense region
gccugugguggaccaugcc

SEQ ID NO: 434 - RNA, 19 nt, Artificial Sequence
  Description of Artificial Sequence siNA antisense region
ugguagaugagcugcggug

SEQ ID NO: 435 - RNA, 19 nt, Artificial Sequence
  Description of Artificial Sequence siNA antisense region
gguccccaccgccacuccu

SEQ ID NO: 436 - RNA, 19 nt, Artificial Sequence
  Description of Artificial Sequence siNA antisense region
gcuccagcccuagugucag

SEQ ID NO: 437 - RNA, 19 nt, Artificial Sequence
  Description of Artificial Sequence siNA antisense region
gggccuccucuucagaggg

SEQ ID NO: 438 - RNA, 19 nt, Artificial Sequence
  Description of Artificial Sequence siNA antisense region
gugccaguggagaccuggg

SEQ ID NO: 439 - RNA, 19 nt, Artificial Sequence
  Description of Artificial Sequence siNA antisense region
agccagccccuucggaggg

SEQ ID NO: 440 - RNA, 19 nt, Artificial Sequence
  Description of Artificial Sequence siNA antisense region
caccaucaaauacaucgga

SEQ ID NO: 441 - RNA, 19 nt, Artificial Sequence
  Description of Artificial Sequence siNA antisense region
cugcccccauucccagguc

SEQ ID NO: 442 - RNA, 19 nt, Artificial Sequence
  Description of Artificial Sequence siNA antisense region
ggcuuugcagccccuuggc

SEQ ID NO: 443 - RNA, 19 nt, Artificial Sequence
  Description of Artificial Sequence siNA antisense region
uggggucauguguggggag

SEQ ID NO: 444 - RNA, 19 nt, Artificial Sequence
  Description of Artificial Sequence siNA antisense region
uguaccgcuguagagggcu

SEQ ID NO: 445 - RNA, 19 nt, Artificial Sequence
  Description of Artificial Sequence siNA antisense region
guacugugggguccucacu

SEQ ID NO: 446 - RNA, 19 nt, Artificial Sequence
  Description of Artificial Sequence siNA antisense region
cagucucagagggcagggg

SEQ ID NO: 447 - RNA, 19 nt, Artificial Sequence
  Description of Artificial Sequence siNA antisense region
ggggggcaacguagccauc

SEQ ID NO: 448 - RNA, 19 nt, Artificial Sequence
  Description of Artificial Sequence siNA antisense region
gcugggggcugcaggucag

SEQ ID NO: 449 - RNA, 19 nt, Artificial Sequence
  Description of Artificial Sequence siNA antisense region
gcugguucacauauucagg

SEQ ID NO: 450 - RNA, 19 nt, Artificial Sequence
  Description of Artificial Sequence siNA antisense region
gcuggggccgaacaucugg

SEQ ID NO: 451 - RNA, 19 nt, Artificial Sequence
  Description of Artificial Sequence siNA antisense region
ccucucggggcgaaggggg

SEQ ID NO: 452 - RNA, 19 nt, Artificial Sequence
  Description of Artificial Sequence siNA antisense region
gggcagcaggcagagggcc

SEQ ID NO: 453 - RNA, 19 nt, Artificial Sequence
  Description of Artificial Sequence siNA antisense region
gaguggcaccagcaggucg

SEQ ID NO: 454 - RNA, 19 nt, Artificial Sequence
  Description of Artificial Sequence siNA antisense region
gagucuugggccuuuccag

SEQ ID NO: 455 - RNA, 19 nt, Artificial Sequence
  Description of Artificial Sequence siNA antisense region
cauucuucccuggggagag

SEQ ID NO: 456 - RNA, 19 nt, Artificial Sequence
  Description of Artificial Sequence siNA antisense region
aaacgucuuugacgacccc

SEQ ID NO: 457 - RNA, 19 nt, Artificial Sequence
  Description of Artificial Sequence siNA antisense region
cggcacccccaaaggcaaa

SEQ ID NO: 458 - RNA, 19 nt, Artificial Sequence
  Description of Artificial Sequence siNA antisense region
aguacucgggguucuccac

SEQ ID NO: 459 - RNA, 19 nt, Artificial Sequence
  Description of Artificial Sequence siNA antisense region
cuccucccuggggugucaa

SEQ ID NO: 460 - RNA, 19 nt, Artificial Sequence
  Description of Artificial Sequence siNA antisense region
gguggggcugaggggcagc

SEQ ID NO: 461 - RNA, 19 nt, Artificial Sequence
  Description of Artificial Sequence siNA antisense region
ggcugaaggcaggaggagg

SEQ ID NO: 462 - RNA, 19 nt, Artificial Sequence
  Description of Artificial Sequence siNA antisense region
agagguugucgaaggcugg

SEQ ID NO: 463 - RNA, 19 nt, Artificial Sequence
  Description of Artificial Sequence siNA antisense region
gguccuggucccaguaaua

SEQ ID NO: 464 - RNA, 19 nt, Artificial Sequence
  Description of Artificial Sequence siNA antisense region
gagccccccgcucuggugg

SEQ ID NO: 465 - RNA, 19 nt, Artificial Sequence
  Description of Artificial Sequence siNA antisense region
cuuugaaggugcugggugg

SEQ ID NO: 466 - RNA, 19 nt, Artificial Sequence
  Description of Artificial Sequence siNA antisense region
ucucugccguagguguccc

SEQ ID NO: 467 - RNA, 19 nt, Artificial Sequence
  Description of Artificial Sequence siNA antisense region
gacccagguacucuggguu

SEQ ID NO: 468 - RNA, 19 nt, Artificial Sequence
  Description of Artificial Sequence siNA antisense region
uucacacuggcacguccag

SEQ ID NO: 469 - RNA, 19 nt, Artificial Sequence
  Description of Artificial Sequence siNA antisense region
gcggacuuggccuucuggu

SEQ ID NO: 470 - RNA, 19 nt, Artificial Sequence
  Description of Artificial Sequence siNA antisense region
ggacacaucagggcuucug

SEQ ID NO: 471 - RNA, 19 nt, Artificial Sequence
  Description of Artificial Sequence siNA antisense region
gccuucccugcucccugag

SEQ ID NO: 472 - RNA, 19 nt, Artificial Sequence
  Description of Artificial Sequence siNA antisense region
gaugccagcagaagucagg

SEQ ID NO: 473 - RNA, 19 nt, Artificial Sequence
  Description of Artificial Sequence siNA antisense region
agggcccucccaccucuug

SEQ ID NO: 474 - RNA, 19 nt, Artificial Sequence
  Description of Artificial Sequence siNA antisense region
uccccuggaaguggucgga

SEQ ID NO: 475 - RNA, 19 nt, Artificial Sequence
  Description of Artificial Sequence siNA antisense region
guuccuggcauggcagguu

SEQ ID NO: 476 - RNA, 19 nt, Artificial Sequence
  Description of Artificial Sequence siNA antisense region
gaagguuccuuaggacagg

SEQ ID NO: 477 - RNA, 19 nt, Artificial Sequence
  Description of Artificial Sequence siNA antisense region
gggaacucaagcaggaagg

SEQ ID NO: 478 - RNA, 19 nt, Artificial Sequence
  Description of Artificial Sequence siNA antisense region
gaccccuuccagccaucug

SEQ ID NO: 479 - RNA, 19 nt, Artificial Sequence
  Description of Artificial Sequence siNA antisense region
ccucuuccaacgaggcugg

SEQ ID NO: 480 - RNA, 19 nt, Artificial Sequence
  Description of Artificial Sequence siNA antisense region
agacuccccagugcuguuc

SEQ ID NO: 481 - RNA, 19 nt, Artificial Sequence
  Description of Artificial Sequence siNA antisense region
gggccucagaauccacaaa

SEQ ID NO: 482 - RNA, 19 nt, Artificial Sequence
  Description of Artificial Sequence siNA antisense region
cuagagucucauugggcag

SEQ ID NO: 483 - RNA, 19 nt, Artificial Sequence
  Description of Artificial Sequence siNA antisense region
uguggcauccacuggaccc

SEQ ID NO: 484 - RNA, 19 nt, Artificial Sequence
  Description of Artificial Sequence siNA antisense region
gaaagggccaagcugggcu

SEQ ID NO: 485 - RNA, 19 nt, Artificial Sequence
  Description of Artificial Sequence siNA antisense region
guacccaggaucuggaagg

SEQ ID NO: 486 - RNA, 19 nt, Artificial Sequence
  Description of Artificial Sequence siNA antisense region
gcuucccuaaggcuuucag

SEQ ID NO: 487 - RNA, 19 nt, Artificial Sequence
  Description of Artificial Sequence siNA antisense region
gcuuccccucucaggccag

SEQ ID NO: 488 - RNA, 19 nt, Artificial Sequence
  Description of Artificial Sequence siNA antisense region
agacacucccuuagggccg

SEQ ID NO: 489 - RNA, 19 nt, Artificial Sequence
  Description of Artificial Sequence siNA antisense region
ugggucgcuuuuguucuua

SEQ ID NO: 490 - RNA, 19 nt, Artificial Sequence
  Description of Artificial Sequence siNA antisense region
ucagggacagucucugaau

SEQ ID NO: 491 - RNA, 19 nt, Artificial Sequence
  Description of Artificial Sequence siNA antisense region
ggggggcaguacuagguuu

SEQ ID NO: 492 - RNA, 19 nt, Artificial Sequence
  Description of Artificial Sequence siNA antisense region
uugcuguuccuuccucaug

SEQ ID NO: 493 - RNA, 19 nt, Artificial Sequence
  Description of Artificial Sequence siNA antisense region
gccuggauacugacaccau

SEQ ID NO: 494 - RNA, 19 nt, Artificial Sequence
  Description of Artificial Sequence siNA antisense region
aaaagcacucuguacaaag

SEQ ID NO: 495 - RNA, 19 nt, Artificial Sequence
  Description of Artificial Sequence siNA antisense region
aaaguaaaaacuaaacaga

SEQ ID NO: 496 - RNA, 19 nt, Artificial Sequence
  Description of Artificial Sequence siNA antisense region
uaaaaaaacaaaacaaaaa

SEQ ID NO: 497 - RNA, 19 nt, Artificial Sequence
  Description of Artificial Sequence siNA antisense region
gggucuuuauuucaucuuu

SEQ ID NO: 498 - RNA, 19 nt, Artificial Sequence
  Description of Artificial Sequence siNA antisense region
cucccccugggucuuuauu

SEQ ID NO: 499 - RNA, 21 nt, Artificial Sequence
  Description of Artificial Sequence siNA antisense region
uccauggugcucacugcggcu

SEQ ID NO: 500 - RNA, 21 nt, Artificial Sequence
  Description of Artificial Sequence  siNA sense region
agccgcagugagcaccaugga

SEQ ID NO: 501 - RNA, 21 nt, Artificial Sequence
  Description of Artificial Sequence  siNA sense inverted control
agguaccacgagugacgccga

SEQ ID NO: 502 - RNA, 21 nt, Artificial Sequence
  Description of Artificial Sequence siNA sense inverted control
ucggcgucacucgugguaccu

SEQ ID NO: 503 - RNA, 23 nt, Artificial Sequence
  Description of Artificial Sequence siNA antisense
uccauggugcucacugcggcuuu

SEQ ID NO: 504 - RNA, 23 nt, Artificial Sequence
  Description of Artificial Sequence  siNA sense
agccgcagugagcaccauggauu

SEQ ID NO: 505 - RNA, 23 nt, Artificial Sequence
  Description of Artificial Sequence  siNA antisense region
uccauggugcucacugcggcuuu

SEQ ID NO: 506 - RNA, 23 nt, Artificial Sequence
  Description of Artificial Sequence siNA sense region
agccgcagugagcaccauggauu

SEQ ID NO: 507 - RNA, 21 nt, Artificial Sequence
  Description of Artificial Sequence  siNA sense region
uggggucgucaaagacguunn

SEQ ID NO: 508 - RNA, 21 nt, Artificial Sequence
  Description of Artificial Sequence  siNA antisense region
aacgucuuugacgaccccann

SEQ ID NO: 509 - RNA, 21 nt, Artificial Sequence
  misc_feature (20)..(21): n stands for thymidine
uugcagaaacugcuggggunn

SEQ ID NO: 510 - RNA, 21 nt, Artificial Sequence
  Description of Artificial Sequence  siNA antisense inverted control
accccagcaguuucugcaann

SEQ ID NO: 511 - RNA, 21 nt, Artificial Sequence
  Description of Artificial Sequence  siNA sense region
ggugcuuggaucuggcgcunn

SEQ ID NO: 512 - RNA, 21 nt, Artificial Sequence
  Description of Artificial Sequence  siNA antisense region
agcgccagauccaagcaccnn

SEQ ID NO: 513 - RNA, 21 nt, Artificial Sequence
  Description of Artificial Sequence  siNA sense inverted control
ucgcggucuagguucguggnn

SEQ ID NO: 514 - RNA, 21 nt, Artificial Sequence
  Description of Artificial Sequence  siNA antisense inverted control
ccacgaaccuagaccgcgann

SEQ ID NO: 515 - RNA, 21 nt, Artificial Sequence
  Description of Artificial Sequence  siNA sense region
gaucuuugggagccuggcann

SEQ ID NO: 516 - RNA, 21 nt, Artificial Sequence
  Description of Artificial Sequence  siNA antisense region
ugccaggcucccaaagaucnn

SEQ ID NO: 517 - RNA, 21 nt, Artificial Sequence
  Description of Artificial Sequence  siNA sense inverted control
acgguccgaggguuucuagnn

SEQ ID NO: 518 - RNA, 21 nt, Artificial Sequence
  Description of Artificial Sequence  siNA antisense inverted control
cuagaaacccucggaccgunn

SEQ ID NO: 519 - RNA, 21 nt, Artificial Sequence
  Description of Artificial Sequence  siNA sense region
ggugcuuggaucuggcgcunn

SEQ ID NO: 520 - RNA, 21 nt, Artificial Sequence
  Description of Artificial Sequence  siNA antisense region
agcgccagauccaagcaccnn

SEQ ID NO: 521 - RNA, 21 nt, Artificial Sequence
  Description of Artificial Sequence  siNA sense region
ggugcuuggaucuggcgcunn

SEQ ID NO: 522 - RNA, 21 nt, Artificial Sequence
  Description of Artificial Sequence  siNA sense region
ggugcuuggaucuggcgcunn

SEQ ID NO: 523 - RNA, 21 nt, Artificial Sequence
  Description of Artificial Sequence  siNA antisense region
agcgccagauccaagcaccnn

SEQ ID NO: 524 - RNA, 21 nt, Artificial Sequence
  Description of Artificial Sequence  siNA antisense region
agcgccagauccaagcaccnn

SEQ ID NO: 525 - RNA, 21 nt, Artificial Sequence
  Description of Artificial Sequence  siNA antisense region
agcgccagauccaagcaccnn

SEQ ID NO: 526 - RNA, 21 nt, Artificial Sequence
  Description of Artificial Sequence  siNA sense inverted control
ucgcggucuagguucguggnn

SEQ ID NO: 527 - RNA, 21 nt, Artificial Sequence
  Description of Artificial Sequence  siNA sense inverted control
ucgcggucuagguucguggnn

SEQ ID NO: 528 - RNA, 21 nt, Artificial Sequence
  Description of Artificial Sequence  siNA sense inverted control
ucgcggucuagguucguggnn

SEQ ID NO: 529 - RNA, 21 nt, Artificial Sequence
  Description of Artificial Sequence  siNA antisense inverted control
ccacgaaccuagaccgcgann

SEQ ID NO: 530 - RNA, 21 nt, Artificial Sequence
  Description of Artificial Sequence  siNA antisense inverted control
ccacgaaccuagaccgcgann

SEQ ID NO: 531 - RNA, 21 nt, Artificial Sequence
  Description of Artificial Sequence  siNA antisense inverted control
ccacgaaccuagaccgcgann

SEQ ID NO: 532 - RNA, 21 nt, Artificial Sequence
  Description of Artificial Sequence  siNA antisense inverted control
ccacgaaccuagaccgcgann

SEQ ID NO: 533 - RNA, 23 nt, Artificial Sequence
  Description of Artificial Sequence  Target sequence/siNA sense region
uggucaccuacaacacagacacg

SEQ ID NO: 534 - RNA, 23 nt, Artificial Sequence
  Description of Artificial Sequence  Target sequence/siNA sense region
cagaauggcucagugaccuguuu

SEQ ID NO: 535 - RNA, 23 nt, Artificial Sequence
  Description of Artificial Sequence  Target sequence/siNA sense region
gcuuuguggucauccagaaugag

SEQ ID NO: 536 - RNA, 23 nt, Artificial Sequence
  Description of Artificial Sequence  Target sequence/siNA sense region
agcaccuucaaagggacaccuac

SEQ ID NO: 537 - RNA, 21 nt, Artificial Sequence
  Description of Artificial Sequence  siNA antisense region
gucaccuacaacacagacacg

SEQ ID NO: 538 - RNA, 21 nt, Artificial Sequence
  Description of Artificial Sequence  siNA antisense region
gaauggcucagugaccuguuu

SEQ ID NO: 539 - RNA, 21 nt, Artificial Sequence
  Description of Artificial Sequence  siNA antisense region
uuuguggucauccagaaugag

SEQ ID NO: 540 - RNA, 21 nt, Artificial Sequence
  Description of Artificial Sequence  siNA antisense region
caccuucaaagggacaccuac

SEQ ID NO: 541 - RNA, 21 nt, Artificial Sequence
  Description of Artificial Sequence  siNA antisense region
ugucuguguuguaggugacca

SEQ ID NO: 542 - RNA, 21 nt, Artificial Sequence
  Description of Artificial Sequence  siNA antisense region
acaggucacugagccauucug

SEQ ID NO: 543 - RNA, 21 nt, Artificial Sequence
  Description of Artificial Sequence  siNA antisense region
cauucuggaugaccacaaagc

SEQ ID NO: 544 - RNA, 21 nt, Artificial Sequence
  Description of Artificial Sequence siNA antisense region
aggugucccuuugaaggugcu

SEQ ID NO: 545 - RNA, 21 nt, Artificial Sequence
  Description of Artificial Sequence  siNA antisense region
gucaccuacaacacagacacg

SEQ ID NO: 546 - RNA, 21 nt, Artificial Sequence
  Description of Artificial Sequence  siNA antisense region
gaauggcucagugaccuguuu

SEQ ID NO: 547 - RNA, 21 nt, Artificial Sequence
  Description of Artificial Sequence  siNA antisense region
uuuguggucauccagaaugag

SEQ ID NO: 548 - RNA, 21 nt, Artificial Sequence
  Description of Artificial Sequence  siNA antisense region
caccuucaaagggacaccuac

SEQ ID NO: 549 - RNA, 21 nt, Artificial Sequence
  Description of Artificial Sequence  siNA antisense region
ugucuguguuguaggugacnn

SEQ ID NO: 550 - RNA, 21 nt, Artificial Sequence
  Description of Artificial Sequence  siNA antisense region
acaggucacugagccauucnn

SEQ ID NO: 551 - RNA, 21 nt, Artificial Sequence
  Description of Artificial Sequence  siNA antisense region
cauucuggaugaccacaaann

SEQ ID NO: 552 - RNA, 21 nt, Artificial Sequence
  Description of Artificial Sequence  siNA antisense region
aggugucccuuugaaggugnn

SEQ ID NO: 553 - RNA, 19 nt, Artificial Sequence
  Description of Artificial Sequence  Target sequence/siNA sense region
cgcgcugcgccggaguccc

SEQ ID NO: 554 - RNA, 19 nt, Artificial Sequence
  Description of Artificial Sequence  Target sequence/siNA sense region
cgagcuagccccggcgccg

SEQ ID NO: 555 - RNA, 19 nt, Artificial Sequence
  Description of Artificial Sequence  Target sequence/siNA sense region
gccgccgcccagaccggac

SEQ ID NO: 556 - RNA, 19 nt, Artificial Sequence
  Description of Artificial Sequence  Target sequence/siNA sense region
cgacaggccaccucgucgg

SEQ ID NO: 557 - RNA, 19 nt, Artificial Sequence
  Description of Artificial Sequence  Target sequence/siNA sense region
gcguccgcccgaguccccg

SEQ ID NO: 558 - RNA, 19 nt, Artificial Sequence
  Description of Artificial Sequence  Target sequence/siNA sense region
gccucgccgccaacgccac

SEQ ID NO: 559 - RNA, 19 nt, Artificial Sequence
  Description of Artificial Sequence  Target sequence/siNA sense region
caaccaccgcgcacggccc

SEQ ID NO: 560 - RNA, 19 nt, Artificial Sequence
  Description of Artificial Sequence  Target sequence/siNA sense region
cccugacuccguccaguau

SEQ ID NO: 561 - RNA, 19 nt, Artificial Sequence
  Description of Artificial Sequence  Target sequence/siNA sense region
uugaucgggagagccggag

SEQ ID NO: 562 - RNA, 19 nt, Artificial Sequence
  Description of Artificial Sequence  Target sequence/siNA sense region
gcgagcucuucggggagca

SEQ ID NO: 563 - RNA, 19 nt, Artificial Sequence
  Description of Artificial Sequence  Target sequence/siNA sense region
agcgaugcgacccuccggg

SEQ ID NO: 564 - RNA, 19 nt, Artificial Sequence
  Description of Artificial Sequence  Target sequence/siNA sense region
gacggccggggcagcgcuc

SEQ ID NO: 565 - RNA, 19 nt, Artificial Sequence
  Description of Artificial Sequence  Target sequence/siNA sense region
ccuggcgcugcuggcugcg

SEQ ID NO: 566 - RNA, 19 nt, Artificial Sequence
  Description of Artificial Sequence  Target sequence/siNA sense region
gcucugcccggcgagucgg

SEQ ID NO: 567 - RNA, 19 nt, Artificial Sequence
  Description of Artificial Sequence  Target sequence/siNA sense region
ggcucuggaggaaaagaaa

SEQ ID NO: 568 - RNA, 19 nt, Artificial Sequence
  Description of Artificial Sequence  Target sequence/siNA sense region
aguuugccaaggcacgagu

SEQ ID NO: 569 - RNA, 19 nt, Artificial Sequence
  Description of Artificial Sequence  Target sequence/siNA sense region
uaacaagcucacgcaguug

SEQ ID NO: 570 - RNA, 19 nt, Artificial Sequence
  Description of Artificial Sequence  Target sequence/siNA sense region
gggcacuuuugaagaucau

SEQ ID NO: 571 - RNA, 19 nt, Artificial Sequence
  Description of Artificial Sequence  Target sequence/siNA sense region
uuuucucagccuccagagg

SEQ ID NO: 572 - RNA, 19 nt, Artificial Sequence
  Description of Artificial Sequence  Target sequence/siNA sense region
gauguucaauaacugugag

SEQ ID NO: 573 - RNA, 19 nt, Artificial Sequence
  Description of Artificial Sequence  Target sequence/siNA sense region
ggugguccuugggaauuug

SEQ ID NO: 574 - RNA, 19 nt, Artificial Sequence
  Description of Artificial Sequence  Target sequence/siNA sense region
ggaaauuaccuaugugcag

SEQ ID NO: 575 - RNA, 19 nt, Artificial Sequence
  Description of Artificial Sequence  Target sequence/siNA sense region
gaggaauuaugaucuuucc

SEQ ID NO: 576 - RNA, 19 nt, Artificial Sequence
  Description of Artificial Sequence  Target sequence/siNA sense region
cuucuuaaagaccauccag

SEQ ID NO: 577 - RNA, 19 nt, Artificial Sequence
  Description of Artificial Sequence  Target sequence/siNA sense region
ggagguggcugguuauguc

SEQ ID NO: 578 - RNA, 19 nt, Artificial Sequence
  Description of Artificial Sequence  Target sequence/siNA sense region
ccucauugcccucaacaca

SEQ ID NO: 579 - RNA, 19 nt, Artificial Sequence
  Description of Artificial Sequence  Target sequence/siNA sense region
aguggagcgaauuccuuug

SEQ ID NO: 580 - RNA, 19 nt, Artificial Sequence
  Description of Artificial Sequence  Target sequence/siNA sense region
ggaaaaccugcagaucauc

SEQ ID NO: 581 - RNA, 19 nt, Artificial Sequence
  Description of Artificial Sequence  Target sequence/siNA sense region
cagaggaaauauguacuac

SEQ ID NO: 582 - RNA, 19 nt, Artificial Sequence
  Description of Artificial Sequence  Target sequence/siNA sense region
cgaaaauuccuaugccuua

SEQ ID NO: 583 - RNA, 19 nt, Artificial Sequence
  Description of Artificial Sequence  Target sequence/siNA sense region
agcagucuuaucuaacuau

SEQ ID NO: 584 - RNA, 19 nt, Artificial Sequence
  Description of Artificial Sequence  Target sequence/siNA sense region
ugaugcaaauaaaaccgga

SEQ ID NO: 585 - RNA, 19 nt, Artificial Sequence
  Description of Artificial Sequence  Target sequence/siNA sense region
acugaaggagcugcccaug

SEQ ID NO: 586 - RNA, 19 nt, Artificial Sequence
  Description of Artificial Sequence  Target sequence/siNA sense region
gagaaauuuacaggaaauc

SEQ ID NO: 587 - RNA, 19 nt, Artificial Sequence
  Description of Artificial Sequence  Target sequence/siNA sense region
ccugcauggcgccgugcgg

SEQ ID NO: 588 - RNA, 19 nt, Artificial Sequence
  Description of Artificial Sequence  Target sequence/siNA sense region
guucagcaacaacccugcc

SEQ ID NO: 589 - RNA, 19 nt, Artificial Sequence
  Description of Artificial Sequence  Target sequence/siNA sense region
ccugugcaacguggagagc

SEQ ID NO: 590 - RNA, 19 nt, Artificial Sequence
  Description of Artificial Sequence  Target sequence/siNA sense region
cauccaguggcgggacaua

SEQ ID NO: 591 - RNA, 19 nt, Artificial Sequence
  Description of Artificial Sequence  Target sequence/siNA sense region
agucagcagugacuuucuc

SEQ ID NO: 592 - RNA, 19 nt, Artificial Sequence
  Description of Artificial Sequence  Target sequence/siNA sense region
cagcaacaugucgauggac

SEQ ID NO: 593 - RNA, 19 nt, Artificial Sequence
  Description of Artificial Sequence  Target sequence/siNA sense region
cuuccagaaccaccugggc

SEQ ID NO: 594 - RNA, 19 nt, Artificial Sequence
  Description of Artificial Sequence  Target sequence/siNA sense region
cagcugccaaaagugugau

SEQ ID NO: 595 - RNA, 19 nt, Artificial Sequence
  Description of Artificial Sequence  Target sequence/siNA sense region
uccaagcugucccaauggg

SEQ ID NO: 596 - RNA, 19 nt, Artificial Sequence
  Description of Artificial Sequence  Target sequence/siNA sense region
gagcugcuggggugcagga

SEQ ID NO: 597 - RNA, 19 nt, Artificial Sequence
  Description of Artificial Sequence  Target sequence/siNA sense region
agaggagaacugccagaaa

SEQ ID NO: 598 - RNA, 19 nt, Artificial Sequence
  Description of Artificial Sequence  Target sequence/siNA sense region
acugaccaaaaucaucugu

SEQ ID NO: 599 - RNA, 19 nt, Artificial Sequence
  Description of Artificial Sequence  Target sequence/siNA sense region
ugcccagcagugcuccggg

SEQ ID NO: 600 - RNA, 19 nt, Artificial Sequence
  Description of Artificial Sequence  Target sequence/siNA sense region
gcgcugccguggcaagucc

SEQ ID NO: 601 - RNA, 19 nt, Artificial Sequence
  Description of Artificial Sequence  Target sequence/siNA sense region
ccccagugacugcugccac

SEQ ID NO: 602 - RNA, 19 nt, Artificial Sequence
  Description of Artificial Sequence  Target sequence/siNA sense region
caaccagugugcugcaggc

SEQ ID NO: 603 - RNA, 19 nt, Artificial Sequence
  Description of Artificial Sequence  Target sequence/siNA sense region
cugcacaggcccccgggag

SEQ ID NO: 604 - RNA, 19 nt, Artificial Sequence
  Description of Artificial Sequence  Target sequence/siNA sense region
gagcgacugccuggucugc

SEQ ID NO: 605 - RNA, 19 nt, Artificial Sequence
  Description of Artificial Sequence  Target sequence/siNA sense region
ccgcaaauuccgagacgaa

SEQ ID NO: 606 - RNA, 19 nt, Artificial Sequence
  Description of Artificial Sequence  Target sequence/siNA sense region
agccacgugcaaggacacc

SEQ ID NO: 607 - RNA, 19 nt, Artificial Sequence
  Description of Artificial Sequence  Target sequence/siNA sense region
cugccccccacucaugcuc

SEQ ID NO: 608 - RNA, 19 nt, Artificial Sequence
  Description of Artificial Sequence  Target sequence/siNA sense region
cuacaaccccaccacguac

SEQ ID NO: 609 - RNA, 19 nt, Artificial Sequence
  Description of Artificial Sequence  Target sequence/siNA sense region
ccagauggaugugaacccc

SEQ ID NO: 610 - RNA, 19 nt, Artificial Sequence
  Description of Artificial Sequence  Target sequence/siNA sense region
cgagggcaaauacagcuuu

SEQ ID NO: 611 - RNA, 19 nt, Artificial Sequence
  Description of Artificial Sequence  Target sequence/siNA sense region
uggugccaccugcgugaag

SEQ ID NO: 612 - RNA, 19 nt, Artificial Sequence
  Description of Artificial Sequence  Target sequence/siNA sense region
gaaguguccccguaauuau

SEQ ID NO: 613 - RNA, 19 nt, Artificial Sequence
  Description of Artificial Sequence  Target sequence/siNA sense region
uguggugacagaucacggc

SEQ ID NO: 614 - RNA, 19 nt, Artificial Sequence
  Description of Artificial Sequence  Target sequence/siNA sense region
cucgugcguccgagccugu

SEQ ID NO: 615 - RNA, 19 nt, Artificial Sequence
  Description of Artificial Sequence  Target sequence/siNA sense region
uggggccgacagcuaugag

SEQ ID NO: 616 - RNA, 19 nt, Artificial Sequence
  Description of Artificial Sequence  Target sequence/siNA sense region
gauggaggaagacggcguc

SEQ ID NO: 617 - RNA, 19 nt, Artificial Sequence
  Description of Artificial Sequence  Target sequence/siNA sense region
ccgcaaguguaagaagugc

SEQ ID NO: 618 - RNA, 19 nt, Artificial Sequence
  Description of Artificial Sequence  Target sequence/siNA sense region
cgaagggccuugccgcaaa

SEQ ID NO: 619 - RNA, 19 nt, Artificial Sequence
  Description of Artificial Sequence  Target sequence/siNA sense region
aguguguaacggaauaggu

SEQ ID NO: 620 - RNA, 19 nt, Artificial Sequence
  Description of Artificial Sequence  Target sequence/siNA sense region
uauuggugaauuuaaagac

SEQ ID NO: 621 - RNA, 19 nt, Artificial Sequence
  Description of Artificial Sequence  Target sequence/siNA sense region
cucacucuccauaaaugcu

SEQ ID NO: 622 - RNA, 19 nt, Artificial Sequence
  Description of Artificial Sequence  Target sequence/siNA sense region
uacgaauauuaaacacuuc

SEQ ID NO: 623 - RNA, 19 nt, Artificial Sequence
  Description of Artificial Sequence  Target sequence/siNA sense region
caaaaacugcaccuccauc

SEQ ID NO: 624 - RNA, 19 nt, Artificial Sequence
  Description of Artificial Sequence  Target sequence/siNA sense region
caguggcgaucuccacauc

SEQ ID NO: 625 - RNA, 19 nt, Artificial Sequence
  Description of Artificial Sequence  Target sequence/siNA sense region
ccugccgguggcauuuagg

SEQ ID NO: 626 - RNA, 19 nt, Artificial Sequence
  Description of Artificial Sequence  Target sequence/siNA sense region
gggugacuccuucacacau

SEQ ID NO: 627 - RNA, 19 nt, Artificial Sequence
  Description of Artificial Sequence  Target sequence/siNA sense region
uacuccuccucuggaucca

SEQ ID NO: 628 - RNA, 19 nt, Artificial Sequence
  Description of Artificial Sequence  Target sequence/siNA sense region
acaggaacuggauauucug

SEQ ID NO: 629 - RNA, 19 nt, Artificial Sequence
  Description of Artificial Sequence  Target sequence/siNA sense region
gaaaaccguaaaggaaauc

SEQ ID NO: 630 - RNA, 19 nt, Artificial Sequence
  Description of Artificial Sequence  Target sequence/siNA sense region
cacaggguuuuugcugauu

SEQ ID NO: 631 - RNA, 19 nt, Artificial Sequence
  Description of Artificial Sequence  Target sequence/siNA sense region
ucaggcuuggccugaaaac

SEQ ID NO: 632 - RNA, 19 nt, Artificial Sequence
  Description of Artificial Sequence  Target sequence/siNA sense region
caggacggaccuccaugcc

SEQ ID NO: 633 - RNA, 19 nt, Artificial Sequence
  Description of Artificial Sequence  Target sequence/siNA sense region
cuuugagaaccuagaaauc

SEQ ID NO: 634 - RNA, 19 nt, Artificial Sequence
  Description of Artificial Sequence  Target sequence/siNA sense region
cauacgcggcaggaccaag

SEQ ID NO: 635 - RNA, 19 nt, Artificial Sequence
  Description of Artificial Sequence  Target sequence/siNA sense region
gcaacauggucaguuuucu

SEQ ID NO: 636 - RNA, 19 nt, Artificial Sequence
  Description of Artificial Sequence  Target sequence/siNA sense region
ucuugcagucgucagccug

SEQ ID NO: 637 - RNA, 19 nt, Artificial Sequence
  Description of Artificial Sequence  Target sequence/siNA sense region
gaacauaacauccuuggga

SEQ ID NO: 638 - RNA, 19 nt, Artificial Sequence
  Description of Artificial Sequence  Target sequence/siNA sense region
auuacgcucccucaaggag

SEQ ID NO: 639 - RNA, 19 nt, Artificial Sequence
  Description of Artificial Sequence  Target sequence/siNA sense region
gauaagugauggagaugug

SEQ ID NO: 640 - RNA, 19 nt, Artificial Sequence
  Description of Artificial Sequence  Target sequence/siNA sense region
gauaauuucaggaaacaaa

SEQ ID NO: 641 - RNA, 19 nt, Artificial Sequence
  Description of Artificial Sequence  Target sequence/siNA sense region
aaauuugugcuaugcaaau

SEQ ID NO: 642 - RNA, 19 nt, Artificial Sequence
  Description of Artificial Sequence  Target sequence/siNA sense region
uacaauaaacuggaaaaaa

SEQ ID NO: 643 - RNA, 19 nt, Artificial Sequence
  Description of Artificial Sequence  Target sequence/siNA sense region
acuguuugggaccuccggu

SEQ ID NO: 644 - RNA, 19 nt, Artificial Sequence
  Description of Artificial Sequence  Target sequence/siNA sense region
ucagaaaaccaaaauuaua

SEQ ID NO: 645 - RNA, 19 nt, Artificial Sequence
  Description of Artificial Sequence  Target sequence/siNA sense region
aagcaacagaggugaaaac

SEQ ID NO: 646 - RNA, 19 nt, Artificial Sequence
  Description of Artificial Sequence  Target sequence/siNA sense region
cagcugcaaggccacaggc

SEQ ID NO: 647 - RNA, 19 nt, Artificial Sequence
  Description of Artificial Sequence  Target sequence/siNA sense region
ccaggucugccaugccuug

SEQ ID NO: 648 - RNA, 19 nt, Artificial Sequence
  Description of Artificial Sequence  Target sequence/siNA sense region
gugcucccccgagggcugc

SEQ ID NO: 649 - RNA, 19 nt, Artificial Sequence
  Description of Artificial Sequence  Target sequence/siNA sense region
cuggggcccggagcccagg

SEQ ID NO: 650 - RNA, 19 nt, Artificial Sequence
  Description of Artificial Sequence  Target sequence/siNA sense region
ggacugcgucucuugccgg

SEQ ID NO: 651 - RNA, 19 nt, Artificial Sequence
  Description of Artificial Sequence  Target sequence/siNA sense region
gaaugucagccgaggcagg

SEQ ID NO: 652 - RNA, 19 nt, Artificial Sequence
  Description of Artificial Sequence  Target sequence/siNA sense region
ggaaugcguggacaagugc

SEQ ID NO: 653 - RNA, 19 nt, Artificial Sequence
  Description of Artificial Sequence  Target sequence/siNA sense region
caagcuucuggagggugag

SEQ ID NO: 654 - RNA, 19 nt, Artificial Sequence
  Description of Artificial Sequence  Target sequence/siNA sense region
gccaagggaguuuguggag

SEQ ID NO: 655 - RNA, 19 nt, Artificial Sequence
  Description of Artificial Sequence  Target sequence/siNA sense region
gaacucugagugcauacag

SEQ ID NO: 656 - RNA, 19 nt, Artificial Sequence
  Description of Artificial Sequence  Target sequence/siNA sense region
gugccacccagagugccug

SEQ ID NO: 657 - RNA, 19 nt, Artificial Sequence
  Description of Artificial Sequence  Target sequence/siNA sense region
gccucaggccaugaacauc

SEQ ID NO: 658 - RNA, 19 nt, Artificial Sequence
  Description of Artificial Sequence  Target sequence/siNA sense region
caccugcacaggacgggga

SEQ ID NO: 659 - RNA, 19 nt, Artificial Sequence
  Description of Artificial Sequence  Target sequence/siNA sense region
accagacaacuguauccag

SEQ ID NO: 660 - RNA, 19 nt, Artificial Sequence
  Description of Artificial Sequence  Target sequence/siNA sense region
gugugcccacuacauugac

SEQ ID NO: 661 - RNA, 19 nt, Artificial Sequence
  Description of Artificial Sequence  Target sequence/siNA sense region
cggcccccacugcgucaag

SEQ ID NO: 662 - RNA, 19 nt, Artificial Sequence
  Description of Artificial Sequence  Target sequence/siNA sense region
gaccugcccggcaggaguc

SEQ ID NO: 663 - RNA, 19 nt, Artificial Sequence
  Description of Artificial Sequence  Target sequence/siNA sense region
caugggagaaaacaacacc

SEQ ID NO: 664 - RNA, 19 nt, Artificial Sequence
  Description of Artificial Sequence  Target sequence/siNA sense region
ccuggucuggaaguacgca

SEQ ID NO: 665 - RNA, 19 nt, Artificial Sequence
  Description of Artificial Sequence  Target sequence/siNA sense region
agacgccggccaugugugc

SEQ ID NO: 666 - RNA, 19 nt, Artificial Sequence
  Description of Artificial Sequence  Target sequence/siNA sense region
ccaccugugccauccaaac

SEQ ID NO: 667 - RNA, 19 nt, Artificial Sequence
  Description of Artificial Sequence  Target sequence/siNA sense region
cugcaccuacggaugcacu

SEQ ID NO: 668 - RNA, 19 nt, Artificial Sequence
  Description of Artificial Sequence  Target sequence/siNA sense region
ugggccaggucuugaaggc

SEQ ID NO: 669 - RNA, 19 nt, Artificial Sequence
  Description of Artificial Sequence  Target sequence/siNA sense region
cuguccaacgaaugggccu

SEQ ID NO: 670 - RNA, 19 nt, Artificial Sequence
  Description of Artificial Sequence  Target sequence/siNA sense region
uaagaucccguccaucgcc

SEQ ID NO: 671 - RNA, 19 nt, Artificial Sequence
  Description of Artificial Sequence  Target sequence/siNA sense region
cacugggauggugggggcc

SEQ ID NO: 672 - RNA, 19 nt, Artificial Sequence
  Description of Artificial Sequence  Target sequence/siNA sense region
ccuccucuugcugcuggug

SEQ ID NO: 673 - RNA, 19 nt, Artificial Sequence
  Description of Artificial Sequence  Target sequence/siNA sense region
gguggcccuggggaucggc

SEQ ID NO: 674 - RNA, 19 nt, Artificial Sequence
  Description of Artificial Sequence  Target sequence/siNA sense region
ccucuucaugcgaaggcgc

SEQ ID NO: 675 - RNA, 19 nt, Artificial Sequence
  Description of Artificial Sequence  Target sequence/siNA sense region
ccacaucguucggaagcgc

SEQ ID NO: 676 - RNA, 19 nt, Artificial Sequence
  Description of Artificial Sequence  Target sequence/siNA sense region
cacgcugcggaggcugcug

SEQ ID NO: 677 - RNA, 19 nt, Artificial Sequence
  Description of Artificial Sequence  Target sequence/siNA sense region
gcaggagagggagcuugug

SEQ ID NO: 678 - RNA, 19 nt, Artificial Sequence
  Description of Artificial Sequence  Target sequence/siNA sense region
ggagccucuuacacccagu

SEQ ID NO: 679 - RNA, 19 nt, Artificial Sequence
  Description of Artificial Sequence  Target sequence/siNA sense region
uggagaagcucccaaccaa

SEQ ID NO: 680 - RNA, 19 nt, Artificial Sequence
  Description of Artificial Sequence  Target sequence/siNA sense region
agcucucuugaggaucuug

SEQ ID NO: 681 - RNA, 19 nt, Artificial Sequence
  Description of Artificial Sequence  Target sequence/siNA sense region
gaaggaaacugaauucaaa

SEQ ID NO: 682 - RNA, 19 nt, Artificial Sequence
  Description of Artificial Sequence  Target sequence/siNA sense region
aaagaucaaagugcugggc

SEQ ID NO: 683 - RNA, 19 nt, Artificial Sequence
  Description of Artificial Sequence  Target sequence/siNA sense region
cuccggugcguucggcacg

SEQ ID NO: 684 - RNA, 19 nt, Artificial Sequence
  Description of Artificial Sequence  Target sequence/siNA sense region
gguguauaagggacucugg

SEQ ID NO: 685 - RNA, 19 nt, Artificial Sequence
  Description of Artificial Sequence  Target sequence/siNA sense region
gaucccagaaggugagaaa

SEQ ID NO: 686 - RNA, 19 nt, Artificial Sequence
  Description of Artificial Sequence  Target sequence/siNA sense region
aguuaaaauucccgucgcu

SEQ ID NO: 687 - RNA, 19 nt, Artificial Sequence
  Description of Artificial Sequence  Target sequence/siNA sense region
uaucaaggaauuaagagaa

SEQ ID NO: 688 - RNA, 19 nt, Artificial Sequence
  Description of Artificial Sequence  Target sequence/siNA sense region
agcaacaucuccgaaagcc

SEQ ID NO: 689 - RNA, 19 nt, Artificial Sequence
  Description of Artificial Sequence  Target sequence/siNA sense region
caacaaggaaauccucgau

SEQ ID NO: 690 - RNA, 19 nt, Artificial Sequence
  Description of Artificial Sequence  Target sequence/siNA sense region
ugaagccuacgugauggcc

SEQ ID NO: 691 - RNA, 19 nt, Artificial Sequence
  Description of Artificial Sequence  Target sequence/siNA sense region
cagcguggacaacccccac

SEQ ID NO: 692 - RNA, 19 nt, Artificial Sequence
  Description of Artificial Sequence  Target sequence/siNA sense region
cgugugccgccugcugggc

SEQ ID NO: 693 - RNA, 19 nt, Artificial Sequence
  Description of Artificial Sequence  Target sequence/siNA sense region
caucugccucaccuccacc

SEQ ID NO: 694 - RNA, 19 nt, Artificial Sequence
  Description of Artificial Sequence  Target sequence/siNA sense region
cgugcaacucaucacgcag

SEQ ID NO: 695 - RNA, 19 nt, Artificial Sequence
  Description of Artificial Sequence  Target sequence/siNA sense region
gcucaugcccuucggcugc

SEQ ID NO: 696 - RNA, 19 nt, Artificial Sequence
  Description of Artificial Sequence  Target sequence/siNA sense region
ccuccuggacuauguccgg

SEQ ID NO: 697 - RNA, 19 nt, Artificial Sequence
  Description of Artificial Sequence  Target sequence/siNA sense region
ggaacacaaagacaauauu

SEQ ID NO: 698 - RNA, 19 nt, Artificial Sequence
  Description of Artificial Sequence  Target sequence/siNA sense region
uggcucccaguaccugcuc

SEQ ID NO: 699 - RNA, 19 nt, Artificial Sequence
  Description of Artificial Sequence  Target sequence/siNA sense region
caacuggugugugcagauc

SEQ ID NO: 700 - RNA, 19 nt, Artificial Sequence
  Description of Artificial Sequence  Target sequence/siNA sense region
cgcaaagggcaugaacuac

SEQ ID NO: 701 - RNA, 19 nt, Artificial Sequence
  Description of Artificial Sequence  Target sequence/siNA sense region
cuuggaggaccgucgcuug

SEQ ID NO: 702 - RNA, 19 nt, Artificial Sequence
  Description of Artificial Sequence  Target sequence/siNA sense region
ggugcaccgcgaccuggca

SEQ ID NO: 703 - RNA, 19 nt, Artificial Sequence
  Description of Artificial Sequence  Target sequence/siNA sense region
agccaggaacguacuggug

SEQ ID NO: 704 - RNA, 19 nt, Artificial Sequence
  Description of Artificial Sequence  Target sequence/siNA sense region
gaaaacaccgcagcauguc

SEQ ID NO: 705 - RNA, 19 nt, Artificial Sequence
  Description of Artificial Sequence  Target sequence/siNA sense region
caagaucacagauuuuggg

SEQ ID NO: 706 - RNA, 19 nt, Artificial Sequence
  Description of Artificial Sequence  Target sequence/siNA sense region
gcuggccaaacugcugggu

SEQ ID NO: 707 - RNA, 19 nt, Artificial Sequence
  Description of Artificial Sequence  Target sequence/siNA sense region
ugcggaagagaaagaauac

SEQ ID NO: 708 - RNA, 19 nt, Artificial Sequence
  Description of Artificial Sequence  Target sequence/siNA sense region
ccaugcagaaggaggcaaa

SEQ ID NO: 709 - RNA, 19 nt, Artificial Sequence
  Description of Artificial Sequence  Target sequence/siNA sense region
agugccuaucaaguggaug

SEQ ID NO: 710 - RNA, 19 nt, Artificial Sequence
  Description of Artificial Sequence  Target sequence/siNA sense region
ggcauuggaaucaauuuua

SEQ ID NO: 711 - RNA, 19 nt, Artificial Sequence
  Description of Artificial Sequence  Target sequence/siNA sense region
acacagaaucuauacccac

SEQ ID NO: 712 - RNA, 19 nt, Artificial Sequence
  Description of Artificial Sequence  Target sequence/siNA sense region
ccagagugaugucuggagc

SEQ ID NO: 713 - RNA, 19 nt, Artificial Sequence
  Description of Artificial Sequence  Target sequence/siNA sense region
cuacggggugaccguuugg

SEQ ID NO: 714 - RNA, 19 nt, Artificial Sequence
  Description of Artificial Sequence  Target sequence/siNA sense region
ggaguugaugaccuuugga

SEQ ID NO: 715 - RNA, 19 nt, Artificial Sequence
  Description of Artificial Sequence  Target sequence/siNA sense region
auccaagccauaugacgga

SEQ ID NO: 716 - RNA, 19 nt, Artificial Sequence
  Description of Artificial Sequence  Target sequence/siNA sense region
aaucccugccagcgagauc

SEQ ID NO: 717 - RNA, 19 nt, Artificial Sequence
  Description of Artificial Sequence  Target sequence/siNA sense region
cuccuccauccuggagaaa

SEQ ID NO: 718 - RNA, 19 nt, Artificial Sequence
  Description of Artificial Sequence  Target sequence/siNA sense region
aggagaacgccucccucag

SEQ ID NO: 719 - RNA, 19 nt, Artificial Sequence
  Description of Artificial Sequence  Target sequence/siNA sense region
gccacccauauguaccauc

SEQ ID NO: 720 - RNA, 19 nt, Artificial Sequence
  Description of Artificial Sequence  Target sequence/siNA sense region
cgaugucuacaugaucaug

SEQ ID NO: 721 - RNA, 19 nt, Artificial Sequence
  Description of Artificial Sequence  Target sequence/siNA sense region
ggucaagugcuggaugaua

SEQ ID NO: 722 - RNA, 19 nt, Artificial Sequence
  Description of Artificial Sequence  Target sequence/siNA sense region
agacgcagauagucgccca

SEQ ID NO: 723 - RNA, 19 nt, Artificial Sequence
  Description of Artificial Sequence  Target sequence/siNA sense region
aaaguuccgugaguugauc

SEQ ID NO: 724 - RNA, 19 nt, Artificial Sequence
  Description of Artificial Sequence  Target sequence/siNA sense region
caucgaauucuccaaaaug

SEQ ID NO: 725 - RNA, 19 nt, Artificial Sequence
  Description of Artificial Sequence  Target sequence/siNA sense region
ggcccgagacccccagcgc

SEQ ID NO: 726 - RNA, 19 nt, Artificial Sequence
  Description of Artificial Sequence  Target sequence/siNA sense region
cuaccuugucauucagggg

SEQ ID NO: 727 - RNA, 19 nt, Artificial Sequence
  Description of Artificial Sequence  Target sequence/siNA sense region
ggaugaaagaaugcauuug

SEQ ID NO: 728 - RNA, 19 nt, Artificial Sequence
  Description of Artificial Sequence  Target sequence/siNA sense region
gccaaguccuacagacucc

SEQ ID NO: 729 - RNA, 19 nt, Artificial Sequence
  Description of Artificial Sequence  Target sequence/siNA sense region
caacuucuaccgugcccug

SEQ ID NO: 730 - RNA, 19 nt, Artificial Sequence
  Description of Artificial Sequence  Target sequence/siNA sense region
gauggaugaagaagacaug

SEQ ID NO: 731 - RNA, 19 nt, Artificial Sequence
  Description of Artificial Sequence  Target sequence/siNA sense region
ggacgacgugguggaugcc

SEQ ID NO: 732 - RNA, 19 nt, Artificial Sequence
  Description of Artificial Sequence  Target sequence/siNA sense region
cgacgaguaccucauccca

SEQ ID NO: 733 - RNA, 19 nt, Artificial Sequence
  Description of Artificial Sequence  Target sequence/siNA sense region
acagcagggcuucuucagc

SEQ ID NO: 734 - RNA, 19 nt, Artificial Sequence
  Description of Artificial Sequence  Target sequence/siNA sense region
cagccccuccacgucacgg

SEQ ID NO: 735 - RNA, 19 nt, Artificial Sequence
  Description of Artificial Sequence  Target sequence/siNA sense region
gacuccccuccugagcucu

SEQ ID NO: 736 - RNA, 19 nt, Artificial Sequence
  Description of Artificial Sequence  Target sequence/siNA sense region
ucugagugcaaccagcaac

SEQ ID NO: 737 - RNA, 19 nt, Artificial Sequence
  Description of Artificial Sequence  Target sequence/siNA sense region
caauuccaccguggcuugc

SEQ ID NO: 738 - RNA, 19 nt, Artificial Sequence
  Description of Artificial Sequence  Target sequence/siNA sense region
cauugauagaaaugggcug

SEQ ID NO: 739 - RNA, 19 nt, Artificial Sequence
  Description of Artificial Sequence  Target sequence/siNA sense region
gcaaagcugucccaucaag

SEQ ID NO: 740 - RNA, 19 nt, Artificial Sequence
  Description of Artificial Sequence  Target sequence/siNA sense region
ggaagacagcuucuugcag

SEQ ID NO: 741 - RNA, 19 nt, Artificial Sequence
  Description of Artificial Sequence  Target sequence/siNA sense region
gcgauacagcucagacccc

SEQ ID NO: 742 - RNA, 19 nt, Artificial Sequence
  Description of Artificial Sequence  Target sequence/siNA sense region
cacaggcgccuugacugag

SEQ ID NO: 743 - RNA, 19 nt, Artificial Sequence
  Description of Artificial Sequence  Target sequence/siNA sense region
ggacagcauagacgacacc

SEQ ID NO: 744 - RNA, 19 nt, Artificial Sequence
  Description of Artificial Sequence  Target sequence/siNA sense region
cuuccucccagugccugaa

SEQ ID NO: 745 - RNA, 19 nt, Artificial Sequence
  Description of Artificial Sequence  Target sequence/siNA sense region
auacauaaaccaguccguu

SEQ ID NO: 746 - RNA, 19 nt, Artificial Sequence
  Description of Artificial Sequence  Target sequence/siNA sense region
ucccaaaaggcccgcuggc

SEQ ID NO: 747 - RNA, 19 nt, Artificial Sequence
  Description of Artificial Sequence  Target sequence/siNA sense region
cucugugcagaauccuguc

SEQ ID NO: 748 - RNA, 19 nt, Artificial Sequence
  Description of Artificial Sequence  Target sequence/siNA sense region
cuaucacaaucagccucug

SEQ ID NO: 749 - RNA, 19 nt, Artificial Sequence
  Description of Artificial Sequence  Target sequence/siNA sense region
gaaccccgcgcccagcaga

SEQ ID NO: 750 - RNA, 19 nt, Artificial Sequence
  Description of Artificial Sequence  Target sequence/siNA sense region
agacccacacuaccaggac

SEQ ID NO: 751 - RNA, 19 nt, Artificial Sequence
  Description of Artificial Sequence  Target sequence/siNA sense region
cccccacagcacugcagug

SEQ ID NO: 752 - RNA, 19 nt, Artificial Sequence
  Description of Artificial Sequence  Target sequence/siNA sense region
gggcaaccccgaguaucuc

SEQ ID NO: 753 - RNA, 19 nt, Artificial Sequence
  Description of Artificial Sequence  Target sequence/siNA sense region
caacacuguccagcccacc

SEQ ID NO: 754 - RNA, 19 nt, Artificial Sequence
  Description of Artificial Sequence  Target sequence/siNA sense region
cugugucaacagcacauuc

SEQ ID NO: 755 - RNA, 19 nt, Artificial Sequence
  Description of Artificial Sequence  Target sequence/siNA sense region
cgacagcccugcccacugg

SEQ ID NO: 756 - RNA, 19 nt, Artificial Sequence
  Description of Artificial Sequence  Target sequence/siNA sense region
ggcccagaaaggcagccac

SEQ ID NO: 757 - RNA, 19 nt, Artificial Sequence
  Description of Artificial Sequence  Target sequence/siNA sense region
ccaaauuagccuggacaac

SEQ ID NO: 758 - RNA, 19 nt, Artificial Sequence
  Description of Artificial Sequence  Target sequence/siNA sense region
cccugacuaccagcaggac

SEQ ID NO: 759 - RNA, 19 nt, Artificial Sequence
  Description of Artificial Sequence  Target sequence/siNA sense region
cuucuuucccaaggaagcc

SEQ ID NO: 760 - RNA, 19 nt, Artificial Sequence
  Description of Artificial Sequence  Target sequence/siNA sense region
caagccaaauggcaucuuu

SEQ ID NO: 761 - RNA, 19 nt, Artificial Sequence
  Description of Artificial Sequence  Target sequence/siNA sense region
uaagggcuccacagcugaa

SEQ ID NO: 762 - RNA, 19 nt, Artificial Sequence
  Description of Artificial Sequence  Target sequence/siNA sense region
aaaugcagaauaccuaagg

SEQ ID NO: 763 - RNA, 19 nt, Artificial Sequence
  Description of Artificial Sequence  Target sequence/siNA sense region
ggucgcgccacaaagcagu

SEQ ID NO: 764 - RNA, 19 nt, Artificial Sequence
  Description of Artificial Sequence  Target sequence/siNA sense region
ugaauuuauuggagcauga

SEQ ID NO: 765 - RNA, 19 nt, Artificial Sequence
  Description of Artificial Sequence  Target sequence/siNA sense region
accacggaggauaguauga

SEQ ID NO: 766 - RNA, 19 nt, Artificial Sequence
  Description of Artificial Sequence  Target sequence/siNA sense region
agcccuaaaaauccagacu

SEQ ID NO: 767 - RNA, 19 nt, Artificial Sequence
  Description of Artificial Sequence  Target sequence/siNA sense region
ucuuucgauacccaggacc

SEQ ID NO: 768 - RNA, 19 nt, Artificial Sequence
  Description of Artificial Sequence  Target sequence/siNA sense region
caagccacagcagguccuc

SEQ ID NO: 769 - RNA, 19 nt, Artificial Sequence
  Description of Artificial Sequence  Target sequence/siNA sense region
ccaucccaacagccaugcc

SEQ ID NO: 770 - RNA, 19 nt, Artificial Sequence
  Description of Artificial Sequence  Target sequence/siNA sense region
ccgcauuagcucuuagacc

SEQ ID NO: 771 - RNA, 19 nt, Artificial Sequence
  Description of Artificial Sequence  Target sequence/siNA sense region
ccacagacugguuuugcaa

SEQ ID NO: 772 - RNA, 19 nt, Artificial Sequence
  Description of Artificial Sequence  Target sequence/siNA sense region
acguuuacaccgacuagcc

SEQ ID NO: 773 - RNA, 19 nt, Artificial Sequence
  Description of Artificial Sequence  Target sequence/siNA sense region
caggaaguacuuccaccuc

SEQ ID NO: 774 - RNA, 19 nt, Artificial Sequence
  Description of Artificial Sequence  Target sequence/siNA sense region
cgggcacauuuugggaagu

SEQ ID NO: 775 - RNA, 19 nt, Artificial Sequence
  Description of Artificial Sequence  Target sequence/siNA sense region
uugcauuccuuugucuuca

SEQ ID NO: 776 - RNA, 19 nt, Artificial Sequence
  Description of Artificial Sequence  Target sequence/siNA sense region
aaacugugaagcauuuaca

SEQ ID NO: 777 - RNA, 19 nt, Artificial Sequence
  Description of Artificial Sequence  Target sequence/siNA sense region
agaaacgcauccagcaaga

SEQ ID NO: 778 - RNA, 19 nt, Artificial Sequence
  Description of Artificial Sequence  Target sequence/siNA sense region
aauauugucccuuugagca

SEQ ID NO: 779 - RNA, 19 nt, Artificial Sequence
  Description of Artificial Sequence  Target sequence/siNA sense region
agaaauuuaucuuucaaag

SEQ ID NO: 780 - RNA, 19 nt, Artificial Sequence
  Description of Artificial Sequence  Target sequence/siNA sense region
gagguauauuugaaaaaaa

SEQ ID NO: 781 - RNA, 19 nt, Artificial Sequence
  Description of Artificial Sequence  Target sequence/siNA sense region
aaaaaaaaaguauauguga

SEQ ID NO: 782 - RNA, 19 nt, Artificial Sequence
  Description of Artificial Sequence  Target sequence/siNA sense region
aggauuuuuauugauuggg

SEQ ID NO: 783 - RNA, 19 nt, Artificial Sequence
  Description of Artificial Sequence  Target sequence/siNA sense region
ggaucuuggaguuuuucau

SEQ ID NO: 784 - RNA, 19 nt, Artificial Sequence
  Description of Artificial Sequence  Target sequence/siNA sense region
uugucgcuauugauuuuua

SEQ ID NO: 785 - RNA, 19 nt, Artificial Sequence
  Description of Artificial Sequence  Target sequence/siNA sense region
acuucaaugggcucuucca

SEQ ID NO: 786 - RNA, 19 nt, Artificial Sequence
  Description of Artificial Sequence  Target sequence/siNA sense region
aacaaggaagaagcuugcu

SEQ ID NO: 787 - RNA, 19 nt, Artificial Sequence
  Description of Artificial Sequence  Target sequence/siNA sense region
ugguagcacuugcuacccu

SEQ ID NO: 788 - RNA, 19 nt, Artificial Sequence
  Description of Artificial Sequence  Target sequence/siNA sense region
ugaguucauccaggcccaa

SEQ ID NO: 789 - RNA, 19 nt, Artificial Sequence
  Description of Artificial Sequence  Target sequence/siNA sense region
acugugagcaaggagcaca

SEQ ID NO: 790 - RNA, 19 nt, Artificial Sequence
  Description of Artificial Sequence  Target sequence/siNA sense region
aagccacaagucuuccaga

SEQ ID NO: 791 - RNA, 19 nt, Artificial Sequence
  Description of Artificial Sequence  Target sequence/siNA sense region
aggaugcuugauuccagug

SEQ ID NO: 792 - RNA, 19 nt, Artificial Sequence
  Description of Artificial Sequence  Target sequence/siNA sense region
gguucugcuucaaggcuuc

SEQ ID NO: 793 - RNA, 19 nt, Artificial Sequence
  Description of Artificial Sequence  Target sequence/siNA sense region
ccacugcaaaacacuaaag

SEQ ID NO: 794 - RNA, 19 nt, Artificial Sequence
  Description of Artificial Sequence  Target sequence/siNA sense region
gauccaagaaggccuucau

SEQ ID NO: 795 - RNA, 19 nt, Artificial Sequence
  Description of Artificial Sequence  Target sequence/siNA sense region
uggccccagcaggccggau

SEQ ID NO: 796 - RNA, 19 nt, Artificial Sequence
  Description of Artificial Sequence  Target sequence/siNA sense region
ucgguacuguaucaaguca

SEQ ID NO: 797 - RNA, 19 nt, Artificial Sequence
  Description of Artificial Sequence  Target sequence/siNA sense region
auggcagguacaguaggau

SEQ ID NO: 798 - RNA, 19 nt, Artificial Sequence
  Description of Artificial Sequence  Target sequence/siNA sense region
uaagccacucugucccuuc

SEQ ID NO: 799 - RNA, 19 nt, Artificial Sequence
  Description of Artificial Sequence  Target sequence/siNA sense region
ccugggcaaagaagaaacg

SEQ ID NO: 800 - RNA, 19 nt, Artificial Sequence
  Description of Artificial Sequence  Target sequence/siNA sense region
ggaggggaugaauucuucc

SEQ ID NO: 801 - RNA, 19 nt, Artificial Sequence
  Description of Artificial Sequence  Target sequence/siNA sense region
cuuagacuuacuuuuguaa

SEQ ID NO: 802 - RNA, 19 nt, Artificial Sequence
  Description of Artificial Sequence  Target sequence/siNA sense region
aaaauguccccacgguacu

SEQ ID NO: 803 - RNA, 19 nt, Artificial Sequence
  Description of Artificial Sequence  Target sequence/siNA sense region
uuacuccccacugauggac

SEQ ID NO: 804 - RNA, 19 nt, Artificial Sequence
  Description of Artificial Sequence  Target sequence/siNA sense region
ccagugguuuccagucaug

SEQ ID NO: 805 - RNA, 19 nt, Artificial Sequence
  Description of Artificial Sequence  Target sequence/siNA sense region
gagcguuagacugacuugu

SEQ ID NO: 806 - RNA, 19 nt, Artificial Sequence
  Description of Artificial Sequence  Target sequence/siNA sense region
uuugucuuccauuccauug

SEQ ID NO: 807 - RNA, 19 nt, Artificial Sequence
  Description of Artificial Sequence  Target sequence/siNA sense region
guuuugaaacucaguaugc

SEQ ID NO: 808 - RNA, 19 nt, Artificial Sequence
  Description of Artificial Sequence  Target sequence/siNA sense region
ccgccccugucuugcuguc

SEQ ID NO: 809 - RNA, 19 nt, Artificial Sequence
  Description of Artificial Sequence  Target sequence/siNA sense region
caugaaaucagcaagagag

SEQ ID NO: 810 - RNA, 19 nt, Artificial Sequence
  Description of Artificial Sequence  Target sequence/siNA sense region
ggaugacacaucaaauaau

SEQ ID NO: 811 - RNA, 19 nt, Artificial Sequence
  Description of Artificial Sequence  Target sequence/siNA sense region
uaacucggauuccagccca

SEQ ID NO: 812 - RNA, 19 nt, Artificial Sequence
  Description of Artificial Sequence  Target sequence/siNA sense region
acauuggauucaucagcau

SEQ ID NO: 813 - RNA, 19 nt, Artificial Sequence
  Description of Artificial Sequence  Target sequence/siNA sense region
uuuggaccaauagcccaca

SEQ ID NO: 814 - RNA, 19 nt, Artificial Sequence
  Description of Artificial Sequence  Target sequence/siNA sense region
agcugagaauguggaauac

SEQ ID NO: 815 - RNA, 19 nt, Artificial Sequence
  Description of Artificial Sequence  Target sequence/siNA sense region
ccuaaggauaacaccgcuu

SEQ ID NO: 816 - RNA, 19 nt, Artificial Sequence
  Description of Artificial Sequence  Target sequence/siNA sense region
uuuguucucgcaaaaacgu

SEQ ID NO: 817 - RNA, 19 nt, Artificial Sequence
  Description of Artificial Sequence  Target sequence/siNA sense region
uaucuccuaauuugaggcu

SEQ ID NO: 818 - RNA, 19 nt, Artificial Sequence
  Description of Artificial Sequence  Target sequence/siNA sense region
ucagaugaaaugcaucagg

SEQ ID NO: 819 - RNA, 19 nt, Artificial Sequence
  Description of Artificial Sequence  Target sequence/siNA sense region
guccuuuggggcauagauc

SEQ ID NO: 820 - RNA, 19 nt, Artificial Sequence
  Description of Artificial Sequence  Target sequence/siNA sense region
cagaagacuacaaaaauga

SEQ ID NO: 821 - RNA, 19 nt, Artificial Sequence
  Description of Artificial Sequence  Target sequence/siNA sense region
aagcugcucugaaaucucc

SEQ ID NO: 822 - RNA, 19 nt, Artificial Sequence
  Description of Artificial Sequence  Target sequence/siNA sense region
cuuuagccaucaccccaac

SEQ ID NO: 823 - RNA, 19 nt, Artificial Sequence
  Description of Artificial Sequence  Target sequence/siNA sense region
ccccccaaaauuaguuugu

SEQ ID NO: 824 - RNA, 19 nt, Artificial Sequence
  Description of Artificial Sequence  Target sequence/siNA sense region
uguuacuuauggaagauag

SEQ ID NO: 825 - RNA, 19 nt, Artificial Sequence
  Description of Artificial Sequence  Target sequence/siNA sense region
guuuucuccuuuuacuuca

SEQ ID NO: 826 - RNA, 19 nt, Artificial Sequence
  Description of Artificial Sequence  Target sequence/siNA sense region
acuucaaaagcuuuuuacu

SEQ ID NO: 827 - RNA, 19 nt, Artificial Sequence
  Description of Artificial Sequence  Target sequence/siNA sense region
ucaaagaguauauguuccc

SEQ ID NO: 828 - RNA, 19 nt, Artificial Sequence
  Description of Artificial Sequence  Target sequence/siNA sense region
cuccaggucagcugccccc

SEQ ID NO: 829 - RNA, 19 nt, Artificial Sequence
  Description of Artificial Sequence  Target sequence/siNA sense region
caaacccccuccuuacgcu

SEQ ID NO: 830 - RNA, 19 nt, Artificial Sequence
  Description of Artificial Sequence  Target sequence/siNA sense region
uuugucacacaaaaagugu

SEQ ID NO: 831 - RNA, 19 nt, Artificial Sequence
  Description of Artificial Sequence  Target sequence/siNA sense region
ucucugccuugagucaucu

SEQ ID NO: 832 - RNA, 19 nt, Artificial Sequence
  Description of Artificial Sequence  Target sequence/siNA sense region
uauucaagcacuuacagcu

SEQ ID NO: 833 - RNA, 19 nt, Artificial Sequence
  Description of Artificial Sequence  Target sequence/siNA sense region
ucuggccacaacagggcau

SEQ ID NO: 834 - RNA, 19 nt, Artificial Sequence
  Description of Artificial Sequence  Target sequence/siNA sense region
uuuuacaggugcgaaugac

SEQ ID NO: 835 - RNA, 19 nt, Artificial Sequence
  Description of Artificial Sequence  Target sequence/siNA sense region
caguagcauuaugaguagu

SEQ ID NO: 836 - RNA, 19 nt, Artificial Sequence
  Description of Artificial Sequence  Target sequence/siNA sense region
ugugaauucagguaguaaa

SEQ ID NO: 837 - RNA, 19 nt, Artificial Sequence
  Description of Artificial Sequence  Target sequence/siNA sense region
auaugaaacuaggguuuga

SEQ ID NO: 838 - RNA, 19 nt, Artificial Sequence
  Description of Artificial Sequence  Target sequence/siNA sense region
aaauugauaaugcuuucac

SEQ ID NO: 839 - RNA, 19 nt, Artificial Sequence
  Description of Artificial Sequence  Target sequence/siNA sense region
caacauuugcagauguuuu

SEQ ID NO: 840 - RNA, 19 nt, Artificial Sequence
  Description of Artificial Sequence  Target sequence/siNA sense region
uagaaggaaaaaaguuccu

SEQ ID NO: 841 - RNA, 19 nt, Artificial Sequence
  Description of Artificial Sequence  Target sequence/siNA sense region
uuccuaaaauaauuucucu

SEQ ID NO: 842 - RNA, 19 nt, Artificial Sequence
  Description of Artificial Sequence  Target sequence/siNA sense region
uacaauuggaagauuggaa

SEQ ID NO: 843 - RNA, 19 nt, Artificial Sequence
  Description of Artificial Sequence  Target sequence/siNA sense region
agauucagcuaguuaggag

SEQ ID NO: 844 - RNA, 19 nt, Artificial Sequence
  Description of Artificial Sequence  Target sequence/siNA sense region
gcccauuuuuuccuaaucu

SEQ ID NO: 845 - RNA, 19 nt, Artificial Sequence
  Description of Artificial Sequence  Target sequence/siNA sense region
ugugugugcccuguaaccu

SEQ ID NO: 846 - RNA, 19 nt, Artificial Sequence
  Description of Artificial Sequence  Target sequence/siNA sense region
ugacugguuaacagcaguc

SEQ ID NO: 847 - RNA, 19 nt, Artificial Sequence
  Description of Artificial Sequence  Target sequence/siNA sense region
ccuuuguaaacaguguuuu

SEQ ID NO: 848 - RNA, 19 nt, Artificial Sequence
  Description of Artificial Sequence  Target sequence/siNA sense region
uaaacucuccuagucaaua

SEQ ID NO: 849 - RNA, 19 nt, Artificial Sequence
  Description of Artificial Sequence  Target sequence/siNA sense region
auccaccccauccaauuua

SEQ ID NO: 850 - RNA, 19 nt, Artificial Sequence
  Description of Artificial Sequence  Target sequence/siNA sense region
aucaaggaagaaaugguuc

SEQ ID NO: 851 - RNA, 19 nt, Artificial Sequence
  Description of Artificial Sequence  Target sequence/siNA sense region
cagaaaauauuuucagccu

SEQ ID NO: 852 - RNA, 19 nt, Artificial Sequence
  Description of Artificial Sequence  Target sequence/siNA sense region
uacaguuauguucagucac

SEQ ID NO: 853 - RNA, 19 nt, Artificial Sequence
  Description of Artificial Sequence  Target sequence/siNA sense region
cacacacauacaaaauguu

SEQ ID NO: 854 - RNA, 19 nt, Artificial Sequence
  Description of Artificial Sequence  Target sequence/siNA sense region
uccuuuugcuuuuaaagua

SEQ ID NO: 855 - RNA, 19 nt, Artificial Sequence
  Description of Artificial Sequence  Target sequence/siNA sense region
aauuuuugacucccagauc

SEQ ID NO: 856 - RNA, 19 nt, Artificial Sequence
  Description of Artificial Sequence  Target sequence/siNA sense region
cagucagagccccuacagc

SEQ ID NO: 857 - RNA, 19 nt, Artificial Sequence
  Description of Artificial Sequence  Target sequence/siNA sense region
cauuguuaagaaaguauuu

SEQ ID NO: 858 - RNA, 19 nt, Artificial Sequence
  Description of Artificial Sequence  Target sequence/siNA sense region
ugauuuuugucucaaugaa

SEQ ID NO: 859 - RNA, 19 nt, Artificial Sequence
  Description of Artificial Sequence  Target sequence/siNA sense region
aaauaaaacuauauucauu

SEQ ID NO: 860 - RNA, 19 nt, Artificial Sequence
  Description of Artificial Sequence  siNA antisense region
gggacuccggcgcagcgcg

SEQ ID NO: 861 - RNA, 19 nt, Artificial Sequence
  Description of Artificial Sequence  siNA antisense region
cggcgccggggcuagcucg

SEQ ID NO: 862 - RNA, 19 nt, Artificial Sequence
  Description of Artificial Sequence  siNA antisense region
guccggucugggcggcggc

SEQ ID NO: 863 - RNA, 19 nt, Artificial Sequence
  Description of Artificial Sequence  siNA antisense region
ccgacgagguggccugucg

SEQ ID NO: 864 - RNA, 19 nt, Artificial Sequence
  Description of Artificial Sequence  siNA antisense region
cggggacucgggcggacgc

SEQ ID NO: 865 - RNA, 19 nt, Artificial Sequence
  Description of Artificial Sequence  siNA antisense region
guggcguuggcggcgaggc

SEQ ID NO: 866 - RNA, 19 nt, Artificial Sequence
  Description of Artificial Sequence  siNA antisense region
gggccgugcgcggugguug

SEQ ID NO: 867 - RNA, 19 nt, Artificial Sequence
  Description of Artificial Sequence  siNA antisense region
auacuggacggagucaggg

SEQ ID NO: 868 - RNA, 19 nt, Artificial Sequence
  Description of Artificial Sequence  siNA antisense region
cuccggcucucccgaucaa

SEQ ID NO: 869 - RNA, 19 nt, Artificial Sequence
  Description of Artificial Sequence  siNA antisense region
ugcuccccgaagagcucgc

SEQ ID NO: 870 - RNA, 19 nt, Artificial Sequence
  Description of Artificial Sequence  siNA antisense region
cccggagggucgcaucgcu

SEQ ID NO: 871 - RNA, 19 nt, Artificial Sequence
  Description of Artificial Sequence  siNA antisense region
gagcgcugccccggccguc

SEQ ID NO: 872 - RNA, 19 nt, Artificial Sequence
  Description of Artificial Sequence  siNA antisense region
cgcagccagcagcgccagg

SEQ ID NO: 873 - RNA, 19 nt, Artificial Sequence
  Description of Artificial Sequence  siNA antisense region
ccgacucgccgggcagagc

SEQ ID NO: 874 - RNA, 19 nt, Artificial Sequence
  Description of Artificial Sequence  siNA antisense region
uuucuuuuccuccagagcc

SEQ ID NO: 875 - RNA, 19 nt, Artificial Sequence
  Description of Artificial Sequence  siNA antisense region
acucgugccuuggcaaacu

SEQ ID NO: 876 - RNA, 19 nt, Artificial Sequence
  Description of Artificial Sequence  siNA antisense region
caacugcgugagcuuguua

SEQ ID NO: 877 - RNA, 19 nt, Artificial Sequence
  Description of Artificial Sequence  siNA antisense region
augaucuucaaaagugccc

SEQ ID NO: 878 - RNA, 19 nt, Artificial Sequence
  Description of Artificial Sequence  siNA antisense region
ccucuggaggcugagaaaa

SEQ ID NO: 879 - RNA, 19 nt, Artificial Sequence
  Description of Artificial Sequence  siNA antisense region
cucacaguuauugaacauc

SEQ ID NO: 880 - RNA, 19 nt, Artificial Sequence
  Description of Artificial Sequence  siNA antisense region
caaauucccaaggaccacc

SEQ ID NO: 881 - RNA, 19 nt, Artificial Sequence
  Description of Artificial Sequence  siNA antisense region
cugcacauagguaauuucc

SEQ ID NO: 882 - RNA, 19 nt, Artificial Sequence
  Description of Artificial Sequence  siNA antisense region
ggaaagaucauaauuccuc

SEQ ID NO: 883 - RNA, 19 nt, Artificial Sequence
  Description of Artificial Sequence  siNA antisense region
cuggauggucuuuaagaag

SEQ ID NO: 884 - RNA, 19 nt, Artificial Sequence
  Description of Artificial Sequence  siNA antisense region
gacauaaccagccaccucc

SEQ ID NO: 885 - RNA, 19 nt, Artificial Sequence
  Description of Artificial Sequence  siNA antisense region
uguguugagggcaaugagg

SEQ ID NO: 886 - RNA, 19 nt, Artificial Sequence
  Description of Artificial Sequence  siNA antisense region
caaaggaauucgcuccacu

SEQ ID NO: 887 - RNA, 19 nt, Artificial Sequence
  Description of Artificial Sequence  siNA antisense region
gaugaucugcagguuuucc

SEQ ID NO: 888 - RNA, 19 nt, Artificial Sequence
  Description of Artificial Sequence  siNA antisense region
guaguacauauuuccucug

SEQ ID NO: 889 - RNA, 19 nt, Artificial Sequence
  Description of Artificial Sequence  siNA antisense region
uaaggcauaggaauuuucg

SEQ ID NO: 890 - RNA, 19 nt, Artificial Sequence
  Description of Artificial Sequence  siNA antisense region
auaguuagauaagacugcu

SEQ ID NO: 891 - RNA, 19 nt, Artificial Sequence
  Description of Artificial Sequence  siNA antisense region
uccgguuuuauuugcauca

SEQ ID NO: 892 - RNA, 19 nt, Artificial Sequence
  Description of Artificial Sequence  siNA antisense region
caugggcagcuccuucagu

SEQ ID NO: 893 - RNA, 19 nt, Artificial Sequence
  Description of Artificial Sequence  siNA antisense region
gauuuccuguaaauuucuc

SEQ ID NO: 894 - RNA, 19 nt, Artificial Sequence
  Description of Artificial Sequence  siNA antisense region
ccgcacggcgccaugcagg

SEQ ID NO: 895 - RNA, 19 nt, Artificial Sequence
  Description of Artificial Sequence  siNA antisense region
ggcaggguuguugcugaac

SEQ ID NO: 896 - RNA, 19 nt, Artificial Sequence
  Description of Artificial Sequence  siNA antisense region
gcucuccacguugcacagg

SEQ ID NO: 897 - RNA, 19 nt, Artificial Sequence
  Description of Artificial Sequence  siNA antisense region
uaugucccgccacuggaug

SEQ ID NO: 898 - RNA, 19 nt, Artificial Sequence
  Description of Artificial Sequence  siNA antisense region
gagaaagucacugcugacu

SEQ ID NO: 899 - RNA, 19 nt, Artificial Sequence
  Description of Artificial Sequence  siNA antisense region
guccaucgacauguugcug

SEQ ID NO: 900 - RNA, 19 nt, Artificial Sequence
  Description of Artificial Sequence  siNA antisense region
gcccaggugguucuggaag

SEQ ID NO: 901 - RNA, 19 nt, Artificial Sequence
  Description of Artificial Sequence  siNA antisense region
aucacacuuuuggcagcug

SEQ ID NO: 902 - RNA, 19 nt, Artificial Sequence
  Description of Artificial Sequence  siNA antisense region
cccauugggacagcuugga

SEQ ID NO: 903 - RNA, 19 nt, Artificial Sequence
  Description of Artificial Sequence  siNA antisense region
uccugcaccccagcagcuc

SEQ ID NO: 904 - RNA, 19 nt, Artificial Sequence
  Description of Artificial Sequence  siNA antisense region
uuucuggcaguucuccucu

SEQ ID NO: 905 - RNA, 19 nt, Artificial Sequence
  Description of Artificial Sequence  siNA antisense region
acagaugauuuuggucagu

SEQ ID NO: 906 - RNA, 19 nt, Artificial Sequence
  Description of Artificial Sequence  siNA antisense region
cccggagcacugcugggca

SEQ ID NO: 907 - RNA, 19 nt, Artificial Sequence
  Description of Artificial Sequence  siNA antisense region
ggacuugccacggcagcgc

SEQ ID NO: 908 - RNA, 19 nt, Artificial Sequence
  Description of Artificial Sequence  siNA antisense region
guggcagcagucacugggg

SEQ ID NO: 909 - RNA, 19 nt, Artificial Sequence
  Description of Artificial Sequence  siNA antisense region
gccugcagcacacugguug

SEQ ID NO: 910 - RNA, 19 nt, Artificial Sequence
  Description of Artificial Sequence  siNA antisense region
cucccgggggccugugcag

SEQ ID NO: 911 - RNA, 19 nt, Artificial Sequence
  Description of Artificial Sequence  siNA antisense region
gcagaccaggcagucgcuc

SEQ ID NO: 912 - RNA, 19 nt, Artificial Sequence
  Description of Artificial Sequence  siNA antisense region
uucgucucggaauuugcgg

SEQ ID NO: 913 - RNA, 19 nt, Artificial Sequence
  Description of Artificial Sequence  siNA antisense region
gguguccuugcacguggcu

SEQ ID NO: 914 - RNA, 19 nt, Artificial Sequence
  Description of Artificial Sequence  siNA antisense region
gagcaugaguggggggcag

SEQ ID NO: 915 - RNA, 19 nt, Artificial Sequence
  Description of Artificial Sequence  siNA antisense region
guacguggugggguuguag

SEQ ID NO: 916 - RNA, 19 nt, Artificial Sequence
  Description of Artificial Sequence  siNA antisense region
gggguucacauccaucugg

SEQ ID NO: 917 - RNA, 19 nt, Artificial Sequence
  Description of Artificial Sequence  siNA antisense region
aaagcuguauuugcccucg

SEQ ID NO: 918 - RNA, 19 nt, Artificial Sequence
  Description of Artificial Sequence  siNA antisense region
cuucacgcagguggcacca

SEQ ID NO: 919 - RNA, 19 nt, Artificial Sequence
  Description of Artificial Sequence  siNA antisense region
auaauuacggggacacuuc

SEQ ID NO: 920 - RNA, 19 nt, Artificial Sequence
  Description of Artificial Sequence  siNA antisense region
gccgugaucugucaccaca

SEQ ID NO: 921 - RNA, 19 nt, Artificial Sequence
  Description of Artificial Sequence  siNA antisense region
acaggcucggacgcacgag

SEQ ID NO: 922 - RNA, 19 nt, Artificial Sequence
  Description of Artificial Sequence  siNA antisense region
cucauagcugucggcccca

SEQ ID NO: 923 - RNA, 19 nt, Artificial Sequence
  Description of Artificial Sequence  siNA antisense region
gacgccgucuuccuccauc

SEQ ID NO: 924 - RNA, 19 nt, Artificial Sequence
  Description of Artificial Sequence  siNA antisense region
gcacuucuuacacuugcgg

SEQ ID NO: 925 - RNA, 19 nt, Artificial Sequence
  Description of Artificial Sequence  siNA antisense region
uuugcggcaaggcccuucg

SEQ ID NO: 926 - RNA, 19 nt, Artificial Sequence
  Description of Artificial Sequence  siNA antisense region
accuauuccguuacacacu

SEQ ID NO: 927 - RNA, 19 nt, Artificial Sequence
  Description of Artificial Sequence  siNA antisense region
gucuuuaaauucaccaaua

SEQ ID NO: 928 - RNA, 19 nt, Artificial Sequence
  Description of Artificial Sequence  siNA antisense region
agcauuuauggagagugag

SEQ ID NO: 929 - RNA, 19 nt, Artificial Sequence
  Description of Artificial Sequence  siNA antisense region
gaaguguuuaauauucgua

SEQ ID NO: 930 - RNA, 19 nt, Artificial Sequence
  Description of Artificial Sequence  siNA antisense region
gauggaggugcaguuuuug

SEQ ID NO: 931 - RNA, 19 nt, Artificial Sequence
  Description of Artificial Sequence  siNA antisense region
gauguggagaucgccacug

SEQ ID NO: 932 - RNA, 19 nt, Artificial Sequence
  Description of Artificial Sequence  siNA antisense region
ccuaaaugccaccggcagg

SEQ ID NO: 933 - RNA, 19 nt, Artificial Sequence
  Description of Artificial Sequence  siNA antisense region
augugugaaggagucaccc

SEQ ID NO: 934 - RNA, 19 nt, Artificial Sequence
  Description of Artificial Sequence  siNA antisense region
uggauccagaggaggagua

SEQ ID NO: 935 - RNA, 19 nt, Artificial Sequence
  Description of Artificial Sequence  siNA antisense region
cagaauauccaguuccugu

SEQ ID NO: 936 - RNA, 19 nt, Artificial Sequence
  Description of Artificial Sequence  siNA antisense region
gauuuccuuuacgguuuuc

SEQ ID NO: 937 - RNA, 19 nt, Artificial Sequence
  Description of Artificial Sequence  siNA antisense region
aaucagcaaaaacccugug

SEQ ID NO: 938 - RNA, 19 nt, Artificial Sequence
  Description of Artificial Sequence  siNA antisense region
guuuucaggccaagccuga

SEQ ID NO: 939 - RNA, 19 nt, Artificial Sequence
  Description of Artificial Sequence  siNA antisense region
ggcauggagguccguccug

SEQ ID NO: 940 - RNA, 19 nt, Artificial Sequence
  Description of Artificial Sequence  siNA antisense region
gauuucuagguucucaaag

SEQ ID NO: 941 - RNA, 19 nt, Artificial Sequence
  Description of Artificial Sequence  siNA antisense region
cuugguccugccgcguaug

SEQ ID NO: 942 - RNA, 19 nt, Artificial Sequence
  Description of Artificial Sequence  siNA antisense region
agaaaacugaccauguugc

SEQ ID NO: 943 - RNA, 19 nt, Artificial Sequence
  Description of Artificial Sequence  siNA antisense region
caggcugacgacugcaaga

SEQ ID NO: 944 - RNA, 19 nt, Artificial Sequence
  Description of Artificial Sequence  siNA antisense region
ucccaaggauguuauguuc

SEQ ID NO: 945 - RNA, 19 nt, Artificial Sequence
  Description of Artificial Sequence  siNA antisense region
cuccuugagggagcguaau

SEQ ID NO: 946 - RNA, 19 nt, Artificial Sequence
  Description of Artificial Sequence  siNA antisense region
cacaucuccaucacuuauc

SEQ ID NO: 947 - RNA, 19 nt, Artificial Sequence
  Description of Artificial Sequence  siNA antisense region
uuuguuuccugaaauuauc

SEQ ID NO: 948 - RNA, 19 nt, Artificial Sequence
  Description of Artificial Sequence  siNA antisense region
auuugcauagcacaaauuu

SEQ ID NO: 949 - RNA, 19 nt, Artificial Sequence
  Description of Artificial Sequence  siNA antisense region
uuuuuuccaguuuauugua

SEQ ID NO: 950 - RNA, 19 nt, Artificial Sequence
  Description of Artificial Sequence  siNA antisense region
accggaggucccaaacagu

SEQ ID NO: 951 - RNA, 19 nt, Artificial Sequence
  Description of Artificial Sequence  siNA antisense region
uauaauuuugguuuucuga

SEQ ID NO: 952 - RNA, 19 nt, Artificial Sequence
  Description of Artificial Sequence  siNA antisense region
guuuucaccucuguugcuu

SEQ ID NO: 953 - RNA, 19 nt, Artificial Sequence
  Description of Artificial Sequence  siNA antisense region
gccuguggccuugcagcug

SEQ ID NO: 954 - RNA, 19 nt, Artificial Sequence
  Description of Artificial Sequence  siNA antisense region
caaggcauggcagaccugg

SEQ ID NO: 955 - RNA, 19 nt, Artificial Sequence
  Description of Artificial Sequence  siNA antisense region
gcagcccucgggggagcac

SEQ ID NO: 956 - RNA, 19 nt, Artificial Sequence
  Description of Artificial Sequence  siNA antisense region
ccugggcuccgggccccag

SEQ ID NO: 957 - RNA, 19 nt, Artificial Sequence
  Description of Artificial Sequence  siNA antisense region
ccggcaagagacgcagucc

SEQ ID NO: 958 - RNA, 19 nt, Artificial Sequence
  Description of Artificial Sequence  siNA antisense region
ccugccucggcugacauuc

SEQ ID NO: 959 - RNA, 19 nt, Artificial Sequence
  Description of Artificial Sequence  siNA antisense region
gcacuuguccacgcauucc

SEQ ID NO: 960 - RNA, 19 nt, Artificial Sequence
  Description of Artificial Sequence  siNA antisense region
cucacccuccagaagcuug

SEQ ID NO: 961 - RNA, 19 nt, Artificial Sequence
  Description of Artificial Sequence  siNA antisense region
cuccacaaacucccuuggc

SEQ ID NO: 962 - RNA, 19 nt, Artificial Sequence
  Description of Artificial Sequence  siNA antisense region
cuguaugcacucagaguuc

SEQ ID NO: 963 - RNA, 19 nt, Artificial Sequence
  Description of Artificial Sequence  siNA antisense region
caggcacucuggguggcac

SEQ ID NO: 964 - RNA, 19 nt, Artificial Sequence
  Description of Artificial Sequence  siNA antisense region
gauguucauggccugaggc

SEQ ID NO: 965 - RNA, 19 nt, Artificial Sequence
  Description of Artificial Sequence  siNA antisense region
uccccguccugugcaggug

SEQ ID NO: 966 - RNA, 19 nt, Artificial Sequence
  Description of Artificial Sequence  siNA antisense region
cuggauacaguugucuggu

SEQ ID NO: 967 - RNA, 19 nt, Artificial Sequence
  Description of Artificial Sequence  siNA antisense region
gucaauguagugggcacac

SEQ ID NO: 968 - RNA, 19 nt, Artificial Sequence
  Description of Artificial Sequence  siNA antisense region
cuugacgcagugggggccg

SEQ ID NO: 969 - RNA, 19 nt, Artificial Sequence
  Description of Artificial Sequence  siNA antisense region
gacuccugccgggcagguc

SEQ ID NO: 970 - RNA, 19 nt, Artificial Sequence
  Description of Artificial Sequence  siNA antisense region
gguguuguuuucucccaug

SEQ ID NO: 971 - RNA, 19 nt, Artificial Sequence
  Description of Artificial Sequence  siNA antisense region
ugcguacuuccagaccagg

SEQ ID NO: 972 - RNA, 19 nt, Artificial Sequence
  Description of Artificial Sequence  siNA antisense region
gcacacauggccggcgucu

SEQ ID NO: 973 - RNA, 19 nt, Artificial Sequence
  Description of Artificial Sequence  siNA antisense region
guuuggauggcacaggugg

SEQ ID NO: 974 - RNA, 19 nt, Artificial Sequence
  Description of Artificial Sequence  siNA antisense region
agugcauccguaggugcag

SEQ ID NO: 975 - RNA, 19 nt, Artificial Sequence
  Description of Artificial Sequence  siNA antisense region
gccuucaagaccuggccca

SEQ ID NO: 976 - RNA, 19 nt, Artificial Sequence
  Description of Artificial Sequence  siNA antisense region
aggcccauucguuggacag

SEQ ID NO: 977 - RNA, 19 nt, Artificial Sequence
  Description of Artificial Sequence  siNA antisense region
ggcgauggacgggaucuua

SEQ ID NO: 978 - RNA, 19 nt, Artificial Sequence
  Description of Artificial Sequence  siNA antisense region
ggcccccaccaucccagug

SEQ ID NO: 979 - RNA, 19 nt, Artificial Sequence
  Description of Artificial Sequence  siNA antisense region
caccagcagcaagaggagg

SEQ ID NO: 980 - RNA, 19 nt, Artificial Sequence
  Description of Artificial Sequence  siNA antisense region
gccgauccccagggccacc

SEQ ID NO: 981 - RNA, 19 nt, Artificial Sequence
  Description of Artificial Sequence  siNA antisense region
gcgccuucgcaugaagagg

SEQ ID NO: 982 - RNA, 19 nt, Artificial Sequence
  Description of Artificial Sequence  siNA antisense region
gcgcuuccgaacgaugugg

SEQ ID NO: 983 - RNA, 19 nt, Artificial Sequence
  Description of Artificial Sequence  siNA antisense region
cagcagccuccgcagcgug

SEQ ID NO: 984 - RNA, 19 nt, Artificial Sequence
  Description of Artificial Sequence  siNA antisense region
cacaagcucccucuccugc

SEQ ID NO: 985 - RNA, 19 nt, Artificial Sequence
  Description of Artificial Sequence  siNA antisense region
acuggguguaagaggcucc

SEQ ID NO: 986 - RNA, 19 nt, Artificial Sequence
  Description of Artificial Sequence  siNA antisense region
uugguugggagcuucucca

SEQ ID NO: 987 - RNA, 19 nt, Artificial Sequence
  Description of Artificial Sequence  siNA antisense region
caagauccucaagagagcu

SEQ ID NO: 988 - RNA, 19 nt, Artificial Sequence
  Description of Artificial Sequence  siNA antisense region
uuugaauucaguuuccuuc

SEQ ID NO: 989 - RNA, 19 nt, Artificial Sequence
  Description of Artificial Sequence  siNA antisense region
gcccagcacuuugaucuuu

SEQ ID NO: 990 - RNA, 19 nt, Artificial Sequence
  Description of Artificial Sequence  siNA antisense region
cgugccgaacgcaccggag

SEQ ID NO: 991 - RNA, 19 nt, Artificial Sequence
  Description of Artificial Sequence  siNA antisense region
ccagagucccuuauacacc

SEQ ID NO: 992 - RNA, 19 nt, Artificial Sequence
  Description of Artificial Sequence  siNA antisense region
uuucucaccuucugggauc

SEQ ID NO: 993 - RNA, 19 nt, Artificial Sequence
  Description of Artificial Sequence  siNA antisense region
agcgacgggaauuuuaacu

SEQ ID NO: 994 - RNA, 19 nt, Artificial Sequence
  Description of Artificial Sequence  siNA antisense region
uucucuuaauuccuugaua

SEQ ID NO: 995 - RNA, 19 nt, Artificial Sequence
  Description of Artificial Sequence  siNA antisense region
ggcuuucggagauguugcu

SEQ ID NO: 996 - RNA, 19 nt, Artificial Sequence
  Description of Artificial Sequence  siNA antisense region
aucgaggauuuccuuguug

SEQ ID NO: 997 - RNA, 19 nt, Artificial Sequence
  Description of Artificial Sequence  siNA antisense region
ggccaucacguaggcuuca

SEQ ID NO: 998 - RNA, 19 nt, Artificial Sequence
  Description of Artificial Sequence  siNA antisense region
guggggguuguccacgcug

SEQ ID NO: 999 - RNA, 19 nt, Artificial Sequence
  Description of Artificial Sequence  siNA antisense region
gcccagcaggcggcacacg

SEQ ID NO: 1000 - RNA, 19 nt, Artificial Sequence
  Description of Artificial Sequence  siNA antisense region
gguggaggugaggcagaug

SEQ ID NO: 1001 - RNA, 19 nt, Artificial Sequence
  Description of Artificial Sequence  siNA antisense region
cugcgugaugaguugcacg

SEQ ID NO: 1002 - RNA, 19 nt, Artificial Sequence
  Description of Artificial Sequence  siNA antisense region
gcagccgaagggcaugagc

SEQ ID NO: 1003 - RNA, 19 nt, Artificial Sequence
  Description of Artificial Sequence  siNA antisense region
ccggacauaguccaggagg

SEQ ID NO: 1004 - RNA, 19 nt, Artificial Sequence
  Description of Artificial Sequence  siNA antisense region
aauauugucuuuguguucc

SEQ ID NO: 1005 - RNA, 19 nt, Artificial Sequence
  Description of Artificial Sequence  siNA antisense region
gagcagguacugggagcca

SEQ ID NO: 1006 - RNA, 19 nt, Artificial Sequence
  Description of Artificial Sequence  siNA antisense region
gaucugcacacaccaguug

SEQ ID NO: 1007 - RNA, 19 nt, Artificial Sequence
  Description of Artificial Sequence  siNA antisense region
guaguucaugcccuuugcg

SEQ ID NO: 1008 - RNA, 19 nt, Artificial Sequence
  Description of Artificial Sequence  siNA antisense region
caagcgacgguccuccaag

SEQ ID NO: 1009 - RNA, 19 nt, Artificial Sequence
  Description of Artificial Sequence  siNA antisense region
ugccaggucgcggugcacc

SEQ ID NO: 1010 - RNA, 19 nt, Artificial Sequence
  Description of Artificial Sequence  siNA antisense region
caccaguacguuccuggcu

SEQ ID NO: 1011 - RNA, 19 nt, Artificial Sequence
  Description of Artificial Sequence  siNA antisense region
gacaugcugcgguguuuuc

SEQ ID NO: 1012 - RNA, 19 nt, Artificial Sequence
  Description of Artificial Sequence  siNA antisense region
cccaaaaucugugaucuug

SEQ ID NO: 1013 - RNA, 19 nt, Artificial Sequence
  Description of Artificial Sequence  siNA antisense region
acccagcaguuuggccagc

SEQ ID NO: 1014 - RNA, 19 nt, Artificial Sequence
  Description of Artificial Sequence  siNA antisense region
guauucuuucucuuccgca

SEQ ID NO: 1015 - RNA, 19 nt, Artificial Sequence
  Description of Artificial Sequence  siNA antisense region
uuugccuccuucugcaugg

SEQ ID NO: 1016 - RNA, 19 nt, Artificial Sequence
  Description of Artificial Sequence  siNA antisense region
cauccacuugauaggcacu

SEQ ID NO: 1017 - RNA, 19 nt, Artificial Sequence
  Description of Artificial Sequence  siNA antisense region
uaaaauugauuccaaugcc

SEQ ID NO: 1018 - RNA, 19 nt, Artificial Sequence
  Description of Artificial Sequence  siNA antisense region
guggguauagauucugugu

SEQ ID NO: 1019 - RNA, 19 nt, Artificial Sequence
  Description of Artificial Sequence  siNA antisense region
gcuccagacaucacucugg

SEQ ID NO: 1020 - RNA, 19 nt, Artificial Sequence
  Description of Artificial Sequence  siNA antisense region
ccaaacggucaccccguag

SEQ ID NO: 1021 - RNA, 19 nt, Artificial Sequence
  Description of Artificial Sequence  siNA antisense region
uccaaaggucaucaacucc

SEQ ID NO: 1022 - RNA, 19 nt, Artificial Sequence
  Description of Artificial Sequence  siNA antisense region
uccgucauauggcuuggau

SEQ ID NO: 1023 - RNA, 19 nt, Artificial Sequence
  Description of Artificial Sequence  siNA antisense region
gaucucgcuggcagggauu

SEQ ID NO: 1024 - RNA, 19 nt, Artificial Sequence
  Description of Artificial Sequence  siNA antisense region
uuucuccaggauggaggag

SEQ ID NO: 1025 - RNA, 19 nt, Artificial Sequence
  Description of Artificial Sequence  siNA antisense region
cugagggaggcguucuccu

SEQ ID NO: 1026 - RNA, 19 nt, Artificial Sequence
  Description of Artificial Sequence  siNA antisense region
gaugguacauauggguggc

SEQ ID NO: 1027 - RNA, 19 nt, Artificial Sequence
  Description of Artificial Sequence  siNA antisense region
caugaucauguagacaucg

SEQ ID NO: 1028 - RNA, 19 nt, Artificial Sequence
  Description of Artificial Sequence  siNA antisense region
uaucauccagcacuugacc

SEQ ID NO: 1029 - RNA, 19 nt, Artificial Sequence
  Description of Artificial Sequence  siNA antisense region
ugggcgacuaucugcgucu

SEQ ID NO: 1030 - RNA, 19 nt, Artificial Sequence
  Description of Artificial Sequence  siNA antisense region
gaucaacucacggaacuuu

SEQ ID NO: 1031 - RNA, 19 nt, Artificial Sequence
  Description of Artificial Sequence  siNA antisense region
cauuuuggagaauucgaug

SEQ ID NO: 1032 - RNA, 19 nt, Artificial Sequence
  Description of Artificial Sequence  siNA antisense region
gcgcugggggucucgggcc

SEQ ID NO: 1033 - RNA, 19 nt, Artificial Sequence
  Description of Artificial Sequence  siNA antisense region
ccccugaaugacaagguag

SEQ ID NO: 1034 - RNA, 19 nt, Artificial Sequence
  Description of Artificial Sequence  siNA antisense region
caaaugcauucuuucaucc

SEQ ID NO: 1035 - RNA, 19 nt, Artificial Sequence
  Description of Artificial Sequence  siNA antisense region
ggagucuguaggacuuggc

SEQ ID NO: 1036 - RNA, 19 nt, Artificial Sequence
  Description of Artificial Sequence  siNA antisense region
cagggcacgguagaaguug

SEQ ID NO: 1037 - RNA, 19 nt, Artificial Sequence
  Description of Artificial Sequence  siNA antisense region
caugucuucuucauccauc

SEQ ID NO: 1038 - RNA, 19 nt, Artificial Sequence
  Description of Artificial Sequence  siNA antisense region
ggcauccaccacgucgucc

SEQ ID NO: 1039 - RNA, 19 nt, Artificial Sequence
  Description of Artificial Sequence  siNA antisense region
ugggaugagguacucgucg

SEQ ID NO: 1040 - RNA, 19 nt, Artificial Sequence
  Description of Artificial Sequence  siNA antisense region
gcugaagaagcccugcugu

SEQ ID NO: 1041 - RNA, 19 nt, Artificial Sequence
  Description of Artificial Sequence  siNA antisense region
ccgugacguggaggggcug

SEQ ID NO: 1042 - RNA, 19 nt, Artificial Sequence
  Description of Artificial Sequence  siNA antisense region
agagcucaggaggggaguc

SEQ ID NO: 1043 - RNA, 19 nt, Artificial Sequence
  Description of Artificial Sequence  siNA antisense region
guugcugguugcacucaga

SEQ ID NO: 1044 - RNA, 19 nt, Artificial Sequence
  Description of Artificial Sequence  siNA antisense region
gcaagccacgguggaauug

SEQ ID NO: 1045 - RNA, 19 nt, Artificial Sequence
  Description of Artificial Sequence  siNA antisense region
cagcccauuucuaucaaug

SEQ ID NO: 1046 - RNA, 19 nt, Artificial Sequence
  Description of Artificial Sequence  siNA antisense region
cuugaugggacagcuuugc

SEQ ID NO: 1047 - RNA, 19 nt, Artificial Sequence
  Description of Artificial Sequence  siNA antisense region
cugcaagaagcugucuucc

SEQ ID NO: 1048 - RNA, 19 nt, Artificial Sequence
  Description of Artificial Sequence  siNA antisense region
ggggucugagcuguaucgc

SEQ ID NO: 1049 - RNA, 19 nt, Artificial Sequence
  Description of Artificial Sequence  siNA antisense region
cucagucaaggcgccugug

SEQ ID NO: 1050 - RNA, 19 nt, Artificial Sequence
  Description of Artificial Sequence  siNA antisense region
ggugucgucuaugcugucc

SEQ ID NO: 1051 - RNA, 19 nt, Artificial Sequence
  Description of Artificial Sequence  siNA antisense region
uucaggcacugggaggaag

SEQ ID NO: 1052 - RNA, 19 nt, Artificial Sequence
  Description of Artificial Sequence  siNA antisense region
aacggacugguuuauguau

SEQ ID NO: 1053 - RNA, 19 nt, Artificial Sequence
  Description of Artificial Sequence  siNA antisense region
gccagcgggccuuuuggga

SEQ ID NO: 1054 - RNA, 19 nt, Artificial Sequence
  Description of Artificial Sequence  siNA antisense region
gacaggauucugcacagag

SEQ ID NO: 1055 - RNA, 19 nt, Artificial Sequence
  Description of Artificial Sequence  siNA antisense region
cagaggcugauugugauag

SEQ ID NO: 1056 - RNA, 19 nt, Artificial Sequence
  Description of Artificial Sequence  siNA antisense region
ucugcugggcgcgggguuc

SEQ ID NO: 1057 - RNA, 19 nt, Artificial Sequence
  Description of Artificial Sequence  siNA antisense region
guccugguagugugggucu

SEQ ID NO: 1058 - RNA, 19 nt, Artificial Sequence
  Description of Artificial Sequence  siNA antisense region
cacugcagugcuguggggg

SEQ ID NO: 1059 - RNA, 19 nt, Artificial Sequence
  Description of Artificial Sequence  siNA antisense region
gagauacucgggguugccc

SEQ ID NO: 1060 - RNA, 19 nt, Artificial Sequence
  Description of Artificial Sequence  siNA antisense region
ggugggcuggacaguguug

SEQ ID NO: 1061 - RNA, 19 nt, Artificial Sequence
  Description of Artificial Sequence  siNA antisense region
gaaugugcuguugacacag

SEQ ID NO: 1062 - RNA, 19 nt, Artificial Sequence
  Description of Artificial Sequence  siNA antisense region
ccagugggcagggcugucg

SEQ ID NO: 1063 - RNA, 19 nt, Artificial Sequence
  Description of Artificial Sequence  siNA antisense region
guggcugccuuucugggcc

SEQ ID NO: 1064 - RNA, 19 nt, Artificial Sequence
  Description of Artificial Sequence  siNA antisense region
guuguccaggcuaauuugg

SEQ ID NO: 1065 - RNA, 19 nt, Artificial Sequence
  Description of Artificial Sequence  siNA antisense region
guccugcugguagucaggg

SEQ ID NO: 1066 - RNA, 19 nt, Artificial Sequence
  Description of Artificial Sequence  siNA antisense region
ggcuuccuugggaaagaag

SEQ ID NO: 1067 - RNA, 19 nt, Artificial Sequence
  Description of Artificial Sequence  siNA antisense region
aaagaugccauuuggcuug

SEQ ID NO: 1068 - RNA, 19 nt, Artificial Sequence
  Description of Artificial Sequence  siNA antisense region
uucagcuguggagcccuua

SEQ ID NO: 1069 - RNA, 19 nt, Artificial Sequence
  Description of Artificial Sequence  siNA antisense region
ccuuagguauucugcauuu

SEQ ID NO: 1070 - RNA, 19 nt, Artificial Sequence
  Description of Artificial Sequence  siNA antisense region
acugcuuuguggcgcgacc

SEQ ID NO: 1071 - RNA, 19 nt, Artificial Sequence
  Description of Artificial Sequence  siNA antisense region
ucaugcuccaauaaauuca

SEQ ID NO: 1072 - RNA, 19 nt, Artificial Sequence
  Description of Artificial Sequence  siNA antisense region
ucauacuauccuccguggu

SEQ ID NO: 1073 - RNA, 19 nt, Artificial Sequence
  Description of Artificial Sequence  siNA antisense region
agucuggauuuuuagggcu

SEQ ID NO: 1074 - RNA, 19 nt, Artificial Sequence
  Description of Artificial Sequence  siNA antisense region
gguccuggguaucgaaaga

SEQ ID NO: 1075 - RNA, 19 nt, Artificial Sequence
  Description of Artificial Sequence  siNA antisense region
gaggaccugcuguggcuug

SEQ ID NO: 1076 - RNA, 19 nt, Artificial Sequence
  Description of Artificial Sequence  siNA antisense region
ggcauggcuguugggaugg

SEQ ID NO: 1077 - RNA, 19 nt, Artificial Sequence
  Description of Artificial Sequence  siNA antisense region
ggucuaagagcuaaugcgg

SEQ ID NO: 1078 - RNA, 19 nt, Artificial Sequence
  Description of Artificial Sequence  siNA antisense region
uugcaaaaccagucugugg

SEQ ID NO: 1079 - RNA, 19 nt, Artificial Sequence
  Description of Artificial Sequence  siNA antisense region
ggcuagucgguguaaacgu

SEQ ID NO: 1080 - RNA, 19 nt, Artificial Sequence
  Description of Artificial Sequence  siNA antisense region
gagguggaaguacuuccug

SEQ ID NO: 1081 - RNA, 19 nt, Artificial Sequence
  Description of Artificial Sequence  siNA antisense region
acuucccaaaaugugcccg

SEQ ID NO: 1082 - RNA, 19 nt, Artificial Sequence
  Description of Artificial Sequence  siNA antisense region
ugaagacaaaggaaugcaa

SEQ ID NO: 1083 - RNA, 19 nt, Artificial Sequence
  Description of Artificial Sequence  siNA antisense region
uguaaaugcuucacaguuu

SEQ ID NO: 1084 - RNA, 19 nt, Artificial Sequence
  Description of Artificial Sequence  siNA antisense region
ucuugcuggaugcguuucu

SEQ ID NO: 1085 - RNA, 19 nt, Artificial Sequence
  Description of Artificial Sequence  siNA antisense region
ugcucaaagggacaauauu

SEQ ID NO: 1086 - RNA, 19 nt, Artificial Sequence
  Description of Artificial Sequence  siNA antisense region
cuuugaaagauaaauuucu

SEQ ID NO: 1087 - RNA, 19 nt, Artificial Sequence
  Description of Artificial Sequence  siNA antisense region
uuuuuuucaaauauaccuc

SEQ ID NO: 1088 - RNA, 19 nt, Artificial Sequence
  Description of Artificial Sequence  siNA antisense region
ucacauauacuuuuuuuuu

SEQ ID NO: 1089 - RNA, 19 nt, Artificial Sequence
  Description of Artificial Sequence  siNA antisense region
cccaaucaauaaaaauccu

SEQ ID NO: 1090 - RNA, 19 nt, Artificial Sequence
  Description of Artificial Sequence  siNA antisense region
augaaaaacuccaagaucc

SEQ ID NO: 1091 - RNA, 19 nt, Artificial Sequence
  Description of Artificial Sequence  siNA antisense region
uaaaaaucaauagcgacaa

SEQ ID NO: 1092 - RNA, 19 nt, Artificial Sequence
  Description of Artificial Sequence  siNA antisense region
uggaagagcccauugaagu

SEQ ID NO: 1093 - RNA, 19 nt, Artificial Sequence
  Description of Artificial Sequence  siNA antisense region
agcaagcuucuuccuuguu

SEQ ID NO: 1094 - RNA, 19 nt, Artificial Sequence
  Description of Artificial Sequence  siNA antisense region
aggguagcaagugcuacca

SEQ ID NO: 1095 - RNA, 19 nt, Artificial Sequence
  Description of Artificial Sequence  siNA antisense region
uugggccuggaugaacuca

SEQ ID NO: 1096 - RNA, 19 nt, Artificial Sequence
  Description of Artificial Sequence  siNA antisense region
ugugcuccuugcucacagu

SEQ ID NO: 1097 - RNA, 19 nt, Artificial Sequence
  Description of Artificial Sequence  siNA antisense region
ucuggaagacuuguggcuu

SEQ ID NO: 1098 - RNA, 19 nt, Artificial Sequence
  Description of Artificial Sequence  siNA antisense region
cacuggaaucaagcauccu

SEQ ID NO: 1099 - RNA, 19 nt, Artificial Sequence
  Description of Artificial Sequence  siNA antisense region
gaagccuugaagcagaacc

SEQ ID NO: 1100 - RNA, 19 nt, Artificial Sequence
  Description of Artificial Sequence  siNA antisense region
cuuuaguguuuugcagugg

SEQ ID NO: 1101 - RNA, 19 nt, Artificial Sequence
  Description of Artificial Sequence  siNA antisense region
augaaggccuucuuggauc

SEQ ID NO: 1102 - RNA, 19 nt, Artificial Sequence
  Description of Artificial Sequence  siNA antisense region
auccggccugcuggggcca

SEQ ID NO: 1103 - RNA, 19 nt, Artificial Sequence
  Description of Artificial Sequence  siNA antisense region
ugacuugauacaguaccga

SEQ ID NO: 1104 - RNA, 19 nt, Artificial Sequence
  Description of Artificial Sequence  siNA antisense region
auccuacuguaccugccau

SEQ ID NO: 1105 - RNA, 19 nt, Artificial Sequence
  Description of Artificial Sequence  siNA antisense region
gaagggacagaguggcuua

SEQ ID NO: 1106 - RNA, 19 nt, Artificial Sequence
  Description of Artificial Sequence  siNA antisense region
cguuucuucuuugcccagg

SEQ ID NO: 1107 - RNA, 19 nt, Artificial Sequence
  Description of Artificial Sequence  siNA antisense region
ggaagaauucauccccucc

SEQ ID NO: 1108 - RNA, 19 nt, Artificial Sequence
  Description of Artificial Sequence  siNA antisense region
uuacaaaaguaagucuaag

SEQ ID NO: 1109 - RNA, 19 nt, Artificial Sequence
  Description of Artificial Sequence  siNA antisense region
aguaccguggggacauuuu

SEQ ID NO: 1110 - RNA, 19 nt, Artificial Sequence
  Description of Artificial Sequence  siNA antisense region
guccaucaguggggaguaa

SEQ ID NO: 1111 - RNA, 19 nt, Artificial Sequence
  Description of Artificial Sequence  siNA antisense region
caugacuggaaaccacugg

SEQ ID NO: 1112 - RNA, 19 nt, Artificial Sequence
  Description of Artificial Sequence  siNA antisense region
acaagucagucuaacgcuc

SEQ ID NO: 1113 - RNA, 19 nt, Artificial Sequence
  Description of Artificial Sequence  siNA antisense region
caauggaauggaagacaaa

SEQ ID NO: 1114 - RNA, 19 nt, Artificial Sequence
  Description of Artificial Sequence  siNA antisense region
gcauacugaguuucaaaac

SEQ ID NO: 1115 - RNA, 19 nt, Artificial Sequence
  Description of Artificial Sequence  siNA antisense region
gacagcaagacaggggcgg

SEQ ID NO: 1116 - RNA, 19 nt, Artificial Sequence
  Description of Artificial Sequence  siNA antisense region
cucucuugcugauuucaug

SEQ ID NO: 1117 - RNA, 19 nt, Artificial Sequence
  Description of Artificial Sequence  siNA antisense region
auuauuugaugugucaucc

SEQ ID NO: 1118 - RNA, 19 nt, Artificial Sequence
  Description of Artificial Sequence  siNA antisense region
ugggcuggaauccgaguua

SEQ ID NO: 1119 - RNA, 19 nt, Artificial Sequence
  Description of Artificial Sequence  siNA antisense region
augcugaugaauccaaugu

SEQ ID NO: 1120 - RNA, 19 nt, Artificial Sequence
  Description of Artificial Sequence  siNA antisense region
ugugggcuauugguccaaa

SEQ ID NO: 1121 - RNA, 19 nt, Artificial Sequence
  Description of Artificial Sequence  siNA antisense region
guauuccacauucucagcu

SEQ ID NO: 1122 - RNA, 19 nt, Artificial Sequence
  Description of Artificial Sequence  siNA antisense region
aagcgguguuauccuuagg

SEQ ID NO: 1123 - RNA, 19 nt, Artificial Sequence
  Description of Artificial Sequence  siNA antisense region
acguuuuugcgagaacaaa

SEQ ID NO: 1124 - RNA, 19 nt, Artificial Sequence
  Description of Artificial Sequence  siNA antisense region
agccucaaauuaggagaua

SEQ ID NO: 1125 - RNA, 19 nt, Artificial Sequence
  Description of Artificial Sequence  siNA antisense region
ccugaugcauuucaucuga

SEQ ID NO: 1126 - RNA, 19 nt, Artificial Sequence
  Description of Artificial Sequence  siNA antisense region
gaucuaugccccaaaggac

SEQ ID NO: 1127 - RNA, 19 nt, Artificial Sequence
  Description of Artificial Sequence  siNA antisense region
ucauuuuuguagucuucug

SEQ ID NO: 1128 - RNA, 19 nt, Artificial Sequence
  Description of Artificial Sequence  siNA antisense region
ggagauuucagagcagcuu

SEQ ID NO: 1129 - RNA, 19 nt, Artificial Sequence
  Description of Artificial Sequence  siNA antisense region
guuggggugauggcuaaag

SEQ ID NO: 1130 - RNA, 19 nt, Artificial Sequence
  Description of Artificial Sequence  siNA antisense region
acaaacuaauuuugggggg

SEQ ID NO: 1131 - RNA, 19 nt, Artificial Sequence
  Description of Artificial Sequence  siNA antisense region
cuaucuuccauaaguaaca

SEQ ID NO: 1132 - RNA, 19 nt, Artificial Sequence
  Description of Artificial Sequence  siNA antisense region
ugaaguaaaaggagaaaac

SEQ ID NO: 1133 - RNA, 19 nt, Artificial Sequence
  Description of Artificial Sequence  siNA antisense region
aguaaaaagcuuuugaagu

SEQ ID NO: 1134 - RNA, 19 nt, Artificial Sequence
  Description of Artificial Sequence  siNA antisense region
gggaacauauacucuuuga

SEQ ID NO: 1135 - RNA, 19 nt, Artificial Sequence
  Description of Artificial Sequence  siNA antisense region
gggggcagcugaccuggag

SEQ ID NO: 1136 - RNA, 19 nt, Artificial Sequence
  Description of Artificial Sequence  siNA antisense region
agcguaaggaggggguuug

SEQ ID NO: 1137 - RNA, 19 nt, Artificial Sequence
  Description of Artificial Sequence  siNA antisense region
acacuuuuugugugacaaa

SEQ ID NO: 1138 - RNA, 19 nt, Artificial Sequence
  Description of Artificial Sequence  siNA antisense region
agaugacucaaggcagaga

SEQ ID NO: 1139 - RNA, 19 nt, Artificial Sequence
  Description of Artificial Sequence  siNA antisense region
agcuguaagugcuugaaua

SEQ ID NO: 1140 - RNA, 19 nt, Artificial Sequence
  Description of Artificial Sequence  siNA antisense region
augcccuguuguggccaga

SEQ ID NO: 1141 - RNA, 19 nt, Artificial Sequence
  Description of Artificial Sequence  siNA antisense region
gucauucgcaccuguaaaa

SEQ ID NO: 1142 - RNA, 19 nt, Artificial Sequence
  Description of Artificial Sequence  siNA antisense region
acuacucauaaugcuacug

SEQ ID NO: 1143 - RNA, 19 nt, Artificial Sequence
  Description of Artificial Sequence  siNA antisense region
uuuacuaccugaauucaca

SEQ ID NO: 1144 - RNA, 19 nt, Artificial Sequence
  Description of Artificial Sequence  siNA antisense region
ucaaacccuaguuucauau

SEQ ID NO: 1145 - RNA, 19 nt, Artificial Sequence
  Description of Artificial Sequence  siNA antisense region
gugaaagcauuaucaauuu

SEQ ID NO: 1146 - RNA, 19 nt, Artificial Sequence
  Description of Artificial Sequence  siNA antisense region
aaaacaucugcaaauguug

SEQ ID NO: 1147 - RNA, 19 nt, Artificial Sequence
  Description of Artificial Sequence  siNA antisense region
aggaacuuuuuuccuucua

SEQ ID NO: 1148 - RNA, 19 nt, Artificial Sequence
  Description of Artificial Sequence  siNA antisense region
agagaaauuauuuuaggaa

SEQ ID NO: 1149 - RNA, 19 nt, Artificial Sequence
  Description of Artificial Sequence  siNA antisense region
uuccaaucuuccaauugua

SEQ ID NO: 1150 - RNA, 19 nt, Artificial Sequence
  Description of Artificial Sequence  siNA antisense region
cuccuaacuagcugaaucu

SEQ ID NO: 1151 - RNA, 19 nt, Artificial Sequence
  Description of Artificial Sequence  siNA antisense region
agauuaggaaaaaaugggc

SEQ ID NO: 1152 - RNA, 19 nt, Artificial Sequence
  Description of Artificial Sequence  siNA antisense region
agguuacagggcacacaca

SEQ ID NO: 1153 - RNA, 19 nt, Artificial Sequence
  Description of Artificial Sequence  siNA antisense region
gacugcuguuaaccaguca

SEQ ID NO: 1154 - RNA, 19 nt, Artificial Sequence
  Description of Artificial Sequence  siNA antisense region
aaaacacuguuuacaaagg

SEQ ID NO: 1155 - RNA, 19 nt, Artificial Sequence
  Description of Artificial Sequence  siNA antisense region
uauugacuaggagaguuua

SEQ ID NO: 1156 - RNA, 19 nt, Artificial Sequence
  Description of Artificial Sequence  siNA antisense region
uaaauuggaugggguggau

SEQ ID NO: 1157 - RNA, 19 nt, Artificial Sequence
  Description of Artificial Sequence  siNA antisense region
gaaccauuucuuccuugau

SEQ ID NO: 1158 - RNA, 19 nt, Artificial Sequence
  Description of Artificial Sequence  siNA antisense region
aggcugaaaauauuuucug

SEQ ID NO: 1159 - RNA, 19 nt, Artificial Sequence
  Description of Artificial Sequence  siNA antisense region
gugacugaacauaacugua

SEQ ID NO: 1160 - RNA, 19 nt, Artificial Sequence
  Description of Artificial Sequence  siNA antisense region
aacauuuuguaugugugug

SEQ ID NO: 1161 - RNA, 19 nt, Artificial Sequence
  Description of Artificial Sequence  siNA antisense region
uacuuuaaaagcaaaagga

SEQ ID NO: 1162 - RNA, 19 nt, Artificial Sequence
  Description of Artificial Sequence  siNA antisense region
gaucugggagucaaaaauu

SEQ ID NO: 1163 - RNA, 19 nt, Artificial Sequence
  Description of Artificial Sequence  siNA antisense region
gcuguaggggcucugacug

SEQ ID NO: 1164 - RNA, 19 nt, Artificial Sequence
  Description of Artificial Sequence  siNA antisense region
aaauacuuucuuaacaaug

SEQ ID NO: 1165 - RNA, 19 nt, Artificial Sequence
  Description of Artificial Sequence  siNA antisense region
uucauugagacaaaaauca

SEQ ID NO: 1166 - RNA, 19 nt, Artificial Sequence
  Description of Artificial Sequence  siNA antisense region
aaugaauauaguuuuauuu

SEQ ID NO: 1167 - RNA, 23 nt, Artificial Sequence
  Description of Artificial Sequence  Targe t sequence/siNA sense region
gagaacugccagaaacugaccaa

SEQ ID NO: 1168 - RNA, 23 nt, Artificial Sequence
  Description of Artificial Sequence  Targe t sequence/siNA sense region
aaaggaaaucacaggguuuuugc

SEQ ID NO: 1169 - RNA, 23 nt, Artificial Sequence
  Description of Artificial Sequence  Targe t sequence/siNA sense region
aaguuccgugaguugaucaucga

SEQ ID NO: 1170 - RNA, 23 nt, Artificial Sequence
  Description of Artificial Sequence  Targe t sequence/siNA sense region
ugccaaguccuacagacuccaac

SEQ ID NO: 1171 - RNA, 21 nt, Artificial Sequence
  Description of Artificial Sequence  siNA antisense region
gaacugccagaaacugaccaa

SEQ ID NO: 1172 - RNA, 21 nt, Artificial Sequence
  Description of Artificial Sequence  siNA antisense region
aggaaaucacaggguuuuugc

SEQ ID NO: 1173 - RNA, 21 nt, Artificial Sequence
  Description of Artificial Sequence  siNA antisense region
guuccgugaguugaucaucga

SEQ ID NO: 1174 - RNA, 21 nt, Artificial Sequence
  Description of Artificial Sequence  siNA antisense region
ccaaguccuacagacuccaac

SEQ ID NO: 1175 - RNA, 21 nt, Artificial Sequence
  Description of Artificial Sequence  siNA antisense region
ggucaguuucuggcaguucuc

SEQ ID NO: 1176 - RNA, 21 nt, Artificial Sequence
  Description of Artificial Sequence  siNA antisense region
aaaaacccugugauuuccuuu

SEQ ID NO: 1177 - RNA, 21 nt, Artificial Sequence
  Description of Artificial Sequence  siNA antisense region
gaugaucaacucacggaacuu

SEQ ID NO: 1178 - RNA, 21 nt, Artificial Sequence
  Description of Artificial Sequence  siNA antisense region
uggagucuguaggacuuggca

SEQ ID NO: 1179 - RNA, 21 nt, Artificial Sequence
  Description of Artificial Sequence  siNA antisense region
gaacugccagaaacugaccaa

SEQ ID NO: 1180 - RNA, 21 nt, Artificial Sequence
  Description of Artificial Sequence  siNA antisense region
aggaaaucacaggguuuuugc

SEQ ID NO: 1181 - RNA, 21 nt, Artificial Sequence
  Description of Artificial Sequence  siNA antisense region
guuccgugaguugaucaucga

SEQ ID NO: 1182 - RNA, 21 nt, Artificial Sequence
  Description of Artificial Sequence  siNA antisense region
ccaaguccuacagacuccaac

SEQ ID NO: 1183 - RNA, 21 nt, Artificial Sequence
  Description of Artificial Sequence  siNA antisense region
ggucaguuucuggcaguucnn

SEQ ID NO: 1184 - RNA, 21 nt, Artificial Sequence
  Description of Artificial Sequence  siNA antisense region
aaaaacccugugauuuccunn

SEQ ID NO: 1185 - RNA, 21 nt, Artificial Sequence
  Description of Artificial Sequence  siNA antisense region
gaugaucaacucacggaacnn

SEQ ID NO: 1186 - RNA, 21 nt, Artificial Sequence
  Description of Artificial Sequence  siNA antisense region
uggagucuguaggacuuggnn

SEQ ID NO: 1187 - RNA, 21 nt, Artificial Sequence
  Description of Artificial Sequence  siNA sense region
nnnnnnnnnnnnnnnnnnnnn

SEQ ID NO: 1188 - RNA, 21 nt, Artificial Sequence
  Description of Artificial Sequence  siNA antisense region
nnnnnnnnnnnnnnnnnnnnn

SEQ ID NO: 1189 - RNA, 21 nt, Artificial Sequence
  Description of Artificial Sequence  siNA sense region
nnnnnnnnnnnnnnnnnnnnn

SEQ ID NO: 1190 - RNA, 21 nt, Artificial Sequence
  Description of Artificial Sequence  siNA antisense region
nnnnnnnnnnnnnnnnnnnnn

SEQ ID NO: 1191 - RNA, 21 nt, Artificial Sequence
  Description of Artificial Sequence  siNA sense region
nnnnnnnnnnnnnnnnnnnnn

SEQ ID NO: 1192 - RNA, 21 nt, Artificial Sequence
  Description of Artificial Sequence  siNA sense region
nnnnnnnnnnnnnnnnnnnnn

SEQ ID NO: 1193 - RNA, 21 nt, Artificial Sequence
  Description of Artificial Sequence  siNA antisense region
nnnnnnnnnnnnnnnnnnnnn

SEQ ID NO: 1194 - RNA, 21 nt, Artificial Sequence
  Description of Artificial Sequence  siNA antisense region
nnnnnnnnnnnnnnnnnnnnn

SEQ ID NO: 1195 - RNA, 21 nt, Artificial Sequence
  Description of Artificial Sequence  siNA antisense region
nnnnnnnnnnnnnnnnnnnnn

SEQ ID NO: 1196 - RNA, 21 nt, Artificial Sequence
  Description of Artificial Sequence  siNA sense region
augcuccgccaccucuaccnn

SEQ ID NO: 1197 - RNA, 21 nt, Artificial Sequence
  Description of Artificial Sequence  siNA antisense region
gguagagguggcggagcaunn

SEQ ID NO: 1198 - RNA, 21 nt, Artificial Sequence
  Description of Artificial Sequence  siNA sense region
augcuccgccaccucuaccnn

SEQ ID NO: 1199 - RNA, 21 nt, Artificial Sequence
  Description of Artificial Sequence  siNA antisense region
gguagagguggcggagcaunn

SEQ ID NO: 1200 - RNA, 21 nt, Artificial Sequence
  Description of Artificial Sequence  siNA sense region
augcuccgccaccucuaccnn

SEQ ID NO: 1201 - RNA, 21 nt, Artificial Sequence
  Description of Artificial Sequence  siNA sense region
augcuccgccaccucuaccnn

SEQ ID NO: 1202 - RNA, 21 nt, Artificial Sequence
  Description of Artificial Sequence  siNA antisense region
gguagagguggcggagcaunn

SEQ ID NO: 1203 - RNA, 21 nt, Artificial Sequence
  Description of Artificial Sequence  siNA antisense region
gguagagguggcggagcaunn

SEQ ID NO: 1204 - RNA, 21 nt, Artificial Sequence
  Description of Artificial Sequence  siNA antisense region
gguagagguggcggagcaunn

SEQ ID NO: 1205 - RNA, 21 nt, Artificial Sequence
  Description of Artificial Sequence  siNA sense region
uuugucuuccauuccauugnn

SEQ ID NO: 1206 - RNA, 21 nt, Artificial Sequence
  Description of Artificial Sequence  siNA antisense region
caauggaauggaagacaaann

SEQ ID NO: 1207 - RNA, 21 nt, Artificial Sequence
  Description of Artificial Sequence  siNA sense region
uuugucuuccauuccauugnn

SEQ ID NO: 1208 - RNA, 21 nt, Artificial Sequence
  Description of Artificial Sequence  siNA antisense region
caauggaauggaagacaaann

SEQ ID NO: 1209 - RNA, 21 nt, Artificial Sequence
  Description of Artificial Sequence  siNA sense region
uuugucuuccauuccauugnn

SEQ ID NO: 1210 - RNA, 21 nt, Artificial Sequence
  Description of Artificial Sequence  siNA sense region
uuugucuuccauuccauugnn

SEQ ID NO: 1211 - RNA, 21 nt, Artificial Sequence
  Description of Artificial Sequence  siNA antisense region
caauggaauggaagacaaann

SEQ ID NO: 1212 - RNA, 21 nt, Artificial Sequence
  Description of Artificial Sequence  siNA antisense region
caauggaauggaagacaaann

SEQ ID NO: 1213 - RNA, 21 nt, Artificial Sequence
  Description of Artificial Sequence  siNA antisense region
caauggaauggaagacaaann

(19) United States
(12) Patent Application Publication
(10) Pub. No.: US 2003/0170891 A1
(43) Pub. Date: Sep. 11, 2003

(54) RNA interference mediated inhibition of epidermal growth factor receptor gene expression using short interfering nucleic acid (siNA)

(76) Inventor: James A. McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/251,117

(22) Filed: Sep. 19, 2002

Related U.S. Application Data
(63) Continuation-in-part of application No. 09/916,466, filed on Jul. 25, 2001.
(63) Continuation-in-part of application No. 10/163,552, filed on Jun. 6, 2002.
(60) Provisional application No. 60/358,580, filed on Feb. 20, 2002.
(60) Provisional application No. 60/393,924, filed on Jul. 3, 2002.
(60) Provisional application No. 60/296,249, filed on Jun. 6, 2001.

(51) Int. Cl. A61K 48/00; C07H 21/02; A61K 31/675; A61K 31/52; C07D 473/24

(52) U.S. Cl. 435/366; 514/44; 514/81; 514/263.21; 544/276; 544/277; 536/23.1; 544/243; 544/244

(57) ABSTRACT

The present invention concerns methods and reagents useful in modulating EGFR (HER1, HER2, HER3, and/or HER4) gene expression in a variety of applications, including use in therapeutic, diagnostic, agricultural, target validation, and genomic discovery applications. Specifically, the invention relates to short interfering nucleic acid (siNA) or short interfering RNA (siRNA) molecules capable of mediating RNA interference (RNAi) against epidermal growth factor receptor targets.

### PRIORITY

This application claims the benefit of U.S. application Ser. Nos. 60/358,580, filed Feb. 20, 2002, and 60/393,924, filed Jul. 3, 2002. This application also claims priority to U.S. application Ser. No. 09/916,466, filed Jul. 25, 2001 and to U.S. application Ser. No. 10/163,552, filed Jun. 6, 2002, which claims the benefit of U.S. application Ser. No. 60/296,249, filed Jun. 6, 2001.

### BACKGROUND OF THE INVENTION

The present invention concerns methods and reagents useful in modulating epidermal growth factor receptor (EGFR) gene expression in a variety of applications, including use in therapeutic, diagnostic, target validation, and genomic discovery applications. Specifically, the invention relates to short interfering nucleic acid molecules (siNA) capable of mediating RNA interference (RNAi) against HER1, HER2, HER3 and HER4 expression.

The following is a discussion of relevant art pertaining to RNAi. The discussion is provided only for understanding of the invention that follows. The summary is not an admission that any of the work described below is prior art to the claimed invention.

RNA interference refers to the process of sequence-specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J, 20, 6877) has revealed certain requirements for siRNA length, structure, chemical composition, and sequence that are essential to mediate efficient RNAi activity. These studies have shown that 21-nucleotide siRNA duplexes are most active when containing 3′-terminal di-nucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end o of the siRNA guide sequence (Elbashir et al., 2001, EMBO J, 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-terminal nucleotide overhanging segments of a 21-mer siRNA duplex having two 2 nucleotide 3′-overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity (Elbashir et al., 2001, EMBO J., 20, 6877). In addition, Elbashir et al., supra, also report that substitution of siRNA with 2′-O-methyl nucleotides completely abolishes RNAi activity. Li et al., International PCT Publication No. WO 00/44914, and Beach et al, International PCT Publication No. WO 01/68836, both suggest that siRNA “may include modifications to either the phosphate-sugar back bone or the nucleoside . . . to include at least one of a nitrogen or sulfur heteroatom”, however, neither application teaches to what extent these modifications are tolerated in siRNA molecules nor provides any examples of such modified siRNA. Kreutzer and Limmer, Canadian Patent Application No. 2,359,180, also describe certain chemical modifications for use in dsRNA constructs in order to counteract activation of double-stranded-RNA-dependent protein kinase PKR, specifically 2′-amino or 2′-O-methyl nucleotides, and nucleotides containing a 2′-O or 4′-C methylene bridge. However, Kreutzer and Limmer similarly fail to show to what extent these modifications are tolerated in siRNA molecules nor do they provide any examples of such modified siRNA.”

Parrish et al., 2000, Molecular Cell, 6, 1977-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that “RNAs with two [phosphorothioate] modified bases also had substantial decreases in effectiveness as RNAi triggers (data not shown); [phosphorothioate] modification of more than two residues greatly destabilized the RNAs in vitro and we were not able to assay interference activities.” Id. at 1081. The authors also tested certain modifications at the 2′-position of the nucleotide sugar in the long siRNA transcripts and observed that substituting deoxynucleotides for ribonucleotides “produced a substantial decrease in interference activity,” especially in the case of Uridine to Thymidine and/or Cytidine to deoxy-Cytidine substitutions. Id. In addition, the authors tested certain base modifications, including substituting in sense and antisense strands of the siRNA, 4-thiouracil, 5-bromouracil, 5-iodouracil, 3-(aminoallyl)uracil for uracil, and inosine for guanosine. They found that whereas 4-thiouracil and 5-bromouracil were all well tolerated, inosine “produced a substantial decrease in interference activity” when incorporated in either strand. Incorporation of 5-iodouracil and 3-(aminoallyl)uracil in the antisense strand resulted in substantial decrease in RNAi activity as well.

Beach et al., International PCT Publication No. WO 01/68836, describe specific methods for attenuating gene expression using endogenously derived dsRNA. Tuschl et al., International PCT Publication No. WO 01/75164, describe a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due “to the danger of activating interferon response”. Li et al., International PCT Publication No. WO 00/44914, describes the use of specific dsRNAs for use in attenuating the expression of certain target genes. Zernicka-Goetz et al., International PCT Publication No. WO 01/36646, describe certain methods for inhibiting the expression of particular genes in mammalian cells using certain dsRNA molecules. Fire et al., International PCT Publication No. WO 99/32619, describe particular methods for introducing certain dsRNA molecules into cells for use in inhibiting gene expression. Plaetinck et al., International PCT Publication No. WO 00/01846, describe certain methods for identifying specific genes responsible for conferring a particular phenotype in a cell using specific dsRNA molecules. Mello et al., International PCT Publication No. WO 01/29058, describe the identification of specific genes involved in dsRNA mediated RNAi. Deschamps Depaillette et al., International PCT Publication No. WO 99/07409, describe specific compositions consisting of particular dsRNA molecules combined with certain anti-viral agents. Waterhouse et al., International PCT Publication No. 99/53050, describe certain methods for decreasing the phenotypic expression of a nucleic acid in plant cells. Driscoll et al., International PCT Publication No. WO 01/49844, describe specific DNA constructs for use in facilitating gene silencing in targeted organisms. Parrish et al., 2000, Molecular Cell, 6, 1977-1087, describe specific chemically-modified siRNA constructs targeting the unc-22 gene of C. elegans. Grossniklaus, International PCT Publication No. WO 01/38551, describes certain methods for regulating polycomb gene expression in plants. Churikov et al., International PCT Publication No. WO 01/42443, describe certain methods for modifying genetic characteristics of an organism. Cogoni et al., International PCT Publication No. WO 01/53475, describe certain methods for isolating a Neurospora silencing gene and uses thereof. Reed et al., International PCT Publication No. WO 01/68836, describe certain methods for gene silencing in plants. Honer et al., International PCT Publication No. WO 01/70944, describe certain methods of drug screening using transgenic nematodes as Parkinson's disease models. Deak et al., International PCT Publication No. WO 01/72774, describe certain Drosophila derived gene products. Arndt et al., International PCT Publication No. WO 01/92513 describe certain methods for mediating gene suppression by using factors that enhance RNAi. Tuschl et al., International PCT Publication No. WO 02/44321, describe certain synthetic siRNA constructs. Pachuk et al., International PCT Publication No. WO 00/63364, and Satishchandran et al., International PCT Publication No. WO 01/04313 describe certain methods and compositions for inhibiting the function of certain polynucleotide sequences. Echeverri et al., International PCT Publication No. WO 02/38805, describe certain C. elegans genes identified via RNAi. Kreutzer et al., International PCT Publication Nos. WO 02/055692 and WO 02/055693, describe certain methods for inhibiting gene expression using RNAi.

The epidermal growth factor receptor (EGFR) is a 170 kDa transmembrane glycoprotein consisting of an extracellular ‘ligand’ binding domain, a transmembrane region and an intracellular domain with tyrosine kinase activity (Kung et al., 1994). The binding of growth factors to the EGFR results in down regulation of the ligand-receptor complex, autophosphorylation of the receptor and other protein substrates, leading ultimately to DNA synthesis and cell division. The external ligand binding domain is stimulated by EGF and also by TGFa, amphiregulin and some viral growth factors (Modjtahedi & Dean, 1994).

One of the striking characteristics of the EGFR gene (c-erbB1), located on chromosome 7, is it's homology to the avian erythroblastosis virus oncogene (v-erbB), which induces malignancies in chickens. The v-erbB gene codes for a truncated product that lacks the extracellular ligand binding domain. The tyrosine kinase domain of the EGFR has been found to have 97% homology to the v-erbB transforming protein (Downward et al., 1984).

Recent studies have shown that the EGFR is overexpressed in a number of malignant human tissues when compared to their normal tissue counterparts (for review see Khazaie et al., 1993). An important finding has been the discovery that the gene for the receptor is both amplified and overexpressed in a number of cancer cells. Overexpression of the EGFR is often accompanied by the co-expression of the growth factors EGF and TGF∝, suggesting that an autocrine pathway for control of growth may play a major part in the progression of tumors (Sporn & Roberts, 1985). It is now widely believed that this is a mechanism by which tumor cells can escape normal physiological control.

Growth factors and their receptors appear to have an important role in the development of human brain tumors. A high incidence of overexpression, amplification, deletion and structural rearrangement of the gene coding for the EGFR has been found in biopsies of brain tumors (Ostrowski et al., 1994). In fact the amplification of the EGFR gene in glioblastoma multiforme tumors is one of the most consistent genetic alterations known, with the EGFR being overexpressed in approximately 40% of malignant gliomas (Black, 1991). It has also been demonstrated that in 50% of glioblastomas, amplification of the EGFR gene is accompanied by the co-expression of mRNA for at least one or both of the growth factors EGF and TNFα (Ekstrand et al., 1991).

The amplified genes are frequently rearranged and associated with polymorphism leading to abnormal protein products (Wong et al., 1994). The rearrangements that have been characterized usually show deletions of part of the extracellular domain, resulting in the production of an EGFR protein that is smaller in size. Three classes of deletion mutant EGF receptor genes have been identified in glioblastoma tumors. Type I mutants lack the majority of the external domain, including the ligand binding site, type II mutants have a deletion in the domain adjacent to the membrane but can still bind ligands and type III, which is the most common and found in 17% of glioblastomas, have a deletion of 267 amino acids spanning domains I and II of the EGFR.

In addition to glioblastomas, abnormal EGFR expression has also been reported in a number of squamous epidermoid cancers and breast cancers (reviewed in Kung et al, 1994; Modjtahedi & Dean, 1994). Interestingly, evidence also suggests that many patients with tumors that over-express the EGFR have a poorer prognosis than those who do not (Khazaie et al., 1993). Consequently, therapeutic strategies which can potentially inhibit or reduce the aberrant expression of the EGFR receptor are of great interest as potential anti-cancer agents.

### SUMMARY OF THE INVENTION

This invention relates to compounds, compositions, and methods useful for modulating epidermal growth factor receptor (EGFR) function and/or gene expression in a cell by RNA interference (RNAi) using short interfering nucleic acid (siNA). In particular, the instant invention features siNA molecules and methods to modulate the expression of an epidermal growth factor receptor (EGFR), such as HER1, HER2, HER3 and HER4. A siNA of the invention can be unmodified or chemically-modified. A siNA of the instant invention can be chemically synthesized, expressed from a vector or enzymatically synthesized. The instant invention also features various chemically-modified synthetic short interfering nucleic acid (siNA) molecules capable of modulating EGFR gene expression in cells by RNA inference (RNAi). The use of chemically-modified siNA is expected to improve various properties of native siNA molecules through increased resistance to nuclease degradation in vivo and/or improved cellular uptake. The siNA molecules of the instant invention provide useful reagents and methods for a variety of therapeutic, diagnostic, agricultural, target validation, genomic discovery, genetic engineering and pharmacogenomic applications.

In one embodiment, the invention features one or more siNA molecules and methods that independently or in combination modulate the expression of gene(s) encoding epidermal growth factor receptors. Specifically, the present invention features siNA molecules that modulate the expression of EGFR genes HER1 (for example Genbank Accession No. NM_{—}005228), HER2 (erbB2/neu) (for example Genbank Accession No. NM_{—}004448), HER3 (for example Genbank Accession No. NM_{—}001982), and HER4 (for example Genbank Accession No. NM_{—}005235).

The description below of the various aspects and embodiments is provided with reference to the exemplary epidermal growth receptor (EGFR) genes HER1, HER2, HER3, and HER4, collectively referred to hereinafter as EGFR. However, the various aspects and embodiments are also directed to other genes which express EGFR proteins and other receptors involved in oncogenesis. Those additional genes can be analyzed for target sites using the methods described for EGFR. Thus, the inhibition and the effects of such inhibition of the other genes can be performed as described herein.

In one embodiment, the invention features a siNA molecule that down regulates expression of an epidermal growth factor receptor (EGFR) gene by RNA interference. The EGFR gene can comprise, for example, HER1 sequence, HER2 sequence, HER3 sequence, or HER4 sequence and/or any combination thereof.

In one embodiment, the invention features a siNA molecule having RNAi activity against HER2 RNA, wherein the siNA molecule comprises a sequence complementary to any RNA having HER2 encoding sequence, for example Genbank Accession No. NM_{—}004448. In another embodiment, the invention features a siNA molecule comprising a sequence selected from the group consisting of SEQ ID NOs: 1-552 and 1187-1204. The sequences shown in SEQ ID NOs: 1-552 and 1187-1204 are not limiting. A siNA molecule of the invention can comprise any contiguous HER2 sequences (e.g., about 19 contiguous HER2 nucleotides. In another embodiment, the invention features a siNA molecule having RNAi activity against HER1 RNA, wherein the siNA molecule comprises a sequence complementary to any RNA having HER1 encoding sequence, for example Genbank Accession No. NM_{—}005228. In another embodiment, the invention features a siNA molecule comprising a sequence selected from the group consisting of SEQ ID NOs: 553-1186, 1187-1195, and 1205-1213. The sequences shown in SEQ ID NOs: 553-1186, 1187-1195, and 1205-1213 are not limiting. A siNA molecule of the invention can comprise any contiguous HER1 sequences (e.g., about 19 contiguous HER1 nucleotides. In yet another embodiment, the invention features a siNA molecule comprising a sequence complementary to a sequence comprising Genbank Accession Nos. NM_{—}005228 (HER1), NM_{—}004448 (HER2), NM_{—}001982 (HER3), and/or NM_{—}005235 (HER4).

In one embodiment, a siNA molecule of the invention has RNAi activity that modulates expression of RNA encoded by an EGFR gene, for example, a HER1, HER2, HER3, or HER4 gene and any combination thereof.

In one embodiment of the invention a siNA molecule is adapted for use to treat cancer. A siNA molecule can comprise a sense region and an antisense region, wherein said antisense region can comprise sequence complementary to an RNA sequence encoding EGFR and the sense region can comprise sequence complementary to the antisense region. A siNA molecule can be assembled from two nucleic acid fragments wherein one fragment can comprise the sense region and the second fragment can comprise the antisense region of said siNA molecule. The sense region and antisense region can be covalently connected via a linker molecule. The linker molecule can be a polynucleotide or non-nucleotide linker. The sense region of a siNA molecule of the invention can comprise a 3′-terminal overhang and the antisense region can comprise a 3′-terminal overhang. The 3′-terminal overhangs each can comprise about 2 nucleotides. The antisense region 3′-terminal nucleotide overhang can be complementary to RNA encoding EGFR. The sense region can comprise a terminal cap moiety at the 5′-end, 3′-end, or both 5′ and 3′ ends of the sense region.

In one embodiment, nucleic acid molecules of the invention that act as mediators of the RNA interference gene silencing response are double-stranded RNA molecules. In another embodiment, the siNA molecules of the invention consist of duplexes containing about 19 base pairs between oligonucleotides comprising about 19 to about 25 nucleotides. In yet another embodiment, siNA molecules of the invention comprise duplexes with overhanging ends of about 1 to about 3 nucleotides, for example about 21 nucleotide duplexes with about 19 base pairs and about 2 nucleotide 3′-overhangs.

In one embodiment, the invention features one or more chemically-modified siNA constructs having specificity for EGFR expressing nucleic acid molecules. Non-limiting examples of such chemical modifications include without limitation phosphorothioate internucleotide linkages, 2′-O-methyl ribonucleotides, 2′-O-methyl modified pyrimidine nucleotides, 2′-deoxy-2′-fluoro ribonucleotides, 2′-deoxy-2-fluoro modified pyrimidine nucleotides, “universal base” nucleotides, 5-C-methyl nucleotides, and inverted deoxy abasic residue incorporation. These chemical modifications, when used in various siNA constructs, are shown to preserve RNAi activity in cells while at the same time, dramatically increasing the serum stability of these compounds. Furthermore, contrary to the data published by Parrish et al., supra, applicant demonstrates that multiple (greater than one) phosphorothioate substitutions are well tolerated and confer substantial increases in serum stability for modified siNA constructs.

The antisense region of a siNA molecule of the invention can comprise a phosphorothioate internucleotide linkage at the 3′ end of said antisense region. The antisense region can comprise between about one and about five phosphorothioate internucleotide linkages at the 5′ end of said antisense region. The 3′-terminal nucleotide overhangs of a siNA molecule of the invention can comprise ribonucleotides or deoxyribonucleotides that are chemically-modified at a nucleic acid sugar, base, or backbone. The 3′-terminal nucleotide overhangs can comprise one or more universal base ribonucleotides. The 3′-terminal nucleotide overhangs can comprise one or more acyclic nucleotides.

In a non-limiting example, the introduction of chemically-modified nucleotides into nucleic acid molecules will provide a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example when compared to an all RNA nucleic acid molecule, the overall activity of the modified nucleic acid molecule can be greater than the native molecule due to improved stability and/or delivery of the molecule. Unlike native unmodified siNA, chemically-modified siNA can also minimize the possibility of activating interferon activity in humans.

One embodiment of the invention provides an expression vector comprising a nucleic acid sequence encoding at least one siNA molecule of the invention in a manner that allows expression of the nucleic acid molecule. Another embodiment of the invention provides a mammalian cell comprising such an expression vector. The mammalian cell can be a human cell. The siNA molecule of the expression vector can comprise a sense region and an antisense region and the antisense region can comprise sequence complementary to a RNA sequence encoding EGFR and the sense region can comprise sequence complementary to the antisense region. The siNA molecule can comprise two distinct strands having complementarity sense and antisense regions. The siNA molecule can comprise a single-strand having complementary sense and antisense regions.

In one embodiment, the invention features a chemically-modified short interfering nucleic acid (siNA) molecule capable of mediating RNA interference (RNAi) against EGFR inside a cell, wherein the chemical modification comprises one or more nucleotides comprising a backbone modified internucleotide linkage, for example, at a 3′ terminal nucleotide overhang, having Formula I:

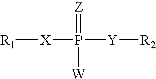

wherein each R1 and R2 is independently any nucleotide, non-nucleotide, or polynucleotide which can be naturally occurring or chemically-modified, each X and Y is independently O, S, N, alkyl, or substituted alkyl, each Z and W is independently O, S, N, alkyl, substituted alkyl, O-alkyl, S-alkyl, alkaryl, or aralkyl.

The chemically-modified internucleotide linkages having Formula I, for example wherein any Z, W, X, and/or Y independently comprises a sulphur atom, can be present in one or both oligonucleotide strands of the siNA duplex, for example in the sense strand, the antisense strand, or both strands. The siNA molecules of the invention can comprise one or more chemically-modified internucleotide linkages having Formula I at the 3′-end, the 5′-end, or both of the 3′ and 5′-ends of the sense strand, the antisense strand, or both strands. For example, an exemplary siNA molecule of the invention can comprise between about 1 and about 5 chemically-modified internucleotide linkages having Formula I at the 5′-end of the sense strand, the antisense strand, or both strands. In another non-limiting example, an exemplary siNA molecule of the invention can comprise one or more pyrimidine nucleotides with chemically-modified internucleotide linkages having Formula I in the sense strand, the antisense strand, or both of the strands. In yet another non-limiting example, an exemplary siNA molecule of the invention can comprise one or more purine nucleotides with chemically-modified internucleotide linkages having Formula I in the sense strand, antisense strand, or both strands. In another embodiment, a siNA molecule of the invention having internucleotide linkage(s) of Formula I also comprises a chemically-modified nucleotide or non-nucleotide having any of Formulae II, III, V, VI, or VII.

In one embodiment, the invention features a chemically-modified short interfering nucleic acid (siNA) molecule capable of mediating RNA interference (RNAi) against EGFR inside a cell, wherein the chemical modification comprises one or more nucleotides or non-nucleotides at, for example, a 3′ terminal nucleotide overhang, having Formula II:

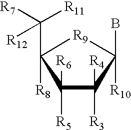

wherein each R3, R4, R5, R6, R7, R8, R10, R 11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be employed to form a stable duplex with RNA or a non-nucleosidic base such as phenyl, naphthyl, 3-nitropyrrole, 5-nitroindole, nebularine, pyridone, pyridinone, or any other non-naturally occurring universal base that can be employed to form a stable duplex with RNA.

The chemically-modified nucleotide or non-nucleotide of Formula II can be present in one or both oligonucleotide strands of the siNA duplex, for example in the sense strand, the antisense strand, or both strands. The siNA molecules of the invention can comprise one or more chemically-modified nucleotide or non-nucleotide of Formula II at the 3′-end, the 5′-end, or both of the 3′ and 5′-ends of the sense strand, antisense strand, or both strands. For example, an exemplary siNA molecule of the invention can comprise between about 1 and about 5 chemically-modified nucleotide or non-nucleotide of Formula II at the 5′-end of the sense strand, the antisense strand, or both of the strands. In anther non-limiting example, an exemplary siNA molecule of the invention can comprise between about 1 and about 5 chemically-modified nucleotide or non-nucleotide of Formula II at the 3′-end of the sense strand, the antisense strand, or both strands.

In one embodiment, the invention features a chemically-modified short interfering nucleic acid (siNA) molecule capable of mediating RNA interference (RNAi) against EGFR inside a cell, wherein the chemical modification comprises one or more nucleotides or non-nucleotides having Formula III:

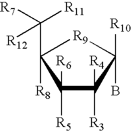

wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be employed to form a stable duplex with RNA or a non-nucleosidic base such as phenyl, naphthyl, 3-nitropyrrole, 5-nitroindole, nebularine, pyridone, pyridinone, or any other non-naturally occurring universal base that can be employed to form a stable duplex with RNA.

The chemically-modified nucleotide or non-nucleotide of Formula III can be present in one or both oligonucleotide strands of the siNA duplex, for example in the sense strand, the antisense strand, or both strands. The siNA molecules of the invention can comprise one or more chemically-modified nucleotide or non-nucleotide of Formula III at the 3′-end, the 5′-end, or both of the 3′ and 5′-ends of the sense strand, the antisense strand, or both strands. For example, an exemplary siNA molecule of the invention can comprise between about 1 and about 5 chemically-modified nucleotide or non-nucleotide of Formula III at the 5′-end of the sense strand, the antisense strand, or both strands. In anther non-limiting example, an exemplary siNA molecule of the invention can comprise between about 1 and about 5 chemically-modified nucleotide or non-nucleotide of Formula III at the 3′-end of the sense strand, the antisense strand, or both strands.

In another embodiment, a siNA molecule of the invention comprises a nucleotide having Formulae II or III, wherein the nucleotide having Formulae II or III is in an inverted configuration. For example, the nucleotide having Formulae II or III is connected to the siNA construct in a 3′-3′, 3′-2′, 2′-3′, or 5′-5′ configuration, such as at the 3′-end, the 5′-end, or both of the 3′ and 5′ ends of one or both siNA strands.

In one embodiment, the invention features a chemically-modified short interfering nucleic acid (siNA) molecule capable of mediating RNA interference (RNAi) against EGFR inside a cell, wherein the chemical modification comprises a 5′-terminal phosphate group having Formula IV:

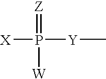

wherein each X and Y is independently O, S, N, alkyl, substituted alkyl, or alkylhalo; each Z and W is independently O, S, N, alkyl, substituted alkyl, O-alkyl, S-alkyl, alkaryl, aralkyl, or alkylhalo; and wherein W, X, Y and Z are not O.

In one embodiment, the invention features a siNA molecule having a 5′-terminal phosphate group having Formula IV on the target-complementary strand, for example a strand complementary to EGFR RNA, wherein the siNA molecule comprises an all RNA siNA molecule. In another embodiment, the invention features a siNA molecule having a 5′-terminal phosphate group having Formula IV on the target-complementary strand wherein the siNA molecule also comprises 1-3 nucleotide 3′-terminal nucleotide overhangs having between about 1 and about 4 deoxyribonucleotides on the 3′-end of one or both strands. In another embodiment, a 5′-terminal phosphate group having Formula IV is present on the target-complementary strand of a siNA molecule of the invention, for example a siNA molecule having chemical modifications having Formulae I, Formula II Formula III, Formula IV, Formula V, Formula VI, and/or Formula VII.

In one embodiment, the invention features a chemically-modified short interfering nucleic acid (siNA) molecule capable of mediating RNA interference (RNAi) against EGFR inside a cell, wherein the chemical modification comprises one or more phosphorothioate internucleotide linkages. For example, in a non-limiting example, the invention features a chemically-modified short interfering nucleic acid (siNA) having about 1, 2, 3, 4, 5, 6, 7, or 8 phosphorothioate internucleotide linkages in one siNA strand. In yet another embodiment, the invention features a chemically-modified short interfering nucleic acid (siNA) individually having about 1, 2, 3, 4, 5, 6, 7, or 8 phosphorothioate internucleotide linkages in both siNA strands. The phosphorothioate internucleotide linkages can be present in one or both oligonucleotide strands of the siNA duplex, for example in the sense strand, the antisense strand, or both strands. The siNA molecules of the invention can comprise one or more phosphorothioate internucleotide linkages at the 3′-end, the 5′-end, or both of the 3′ and 5′-ends of the sense strand, antisense strand, or both strands. For example, an exemplary siNA molecule of the invention can comprise between about 1 and about 5 phosphorothioate internucleotide linkages at the 5′-end of the sense strand, the antisense strand, or both strands. In another non-limiting example, an exemplary siNA molecule of the invention can comprise one or more pyrimidine phosphorothioate internucleotide linkages in the sense strand, the antisense strand, or both strands. In yet another non-limiting example, an exemplary siNA molecule of the invention can comprise one or more purine phosphorothioate internucleotide linkages in the sense strand, the antisense strand, or both strands.

In one embodiment, the invention features a siNA molecule, wherein the sense strand comprises one or more, for example about 1, 2, 3, 4, 5, 6, 7, 8, 9, or 10 phosphorothioate internucleotide linkages, and/or one or more 2′-deoxy, 2′-O-methyl, 2′-deoxy-2′-fluoro, and/or one or more universal base modified nucleotides, and optionally a terminal cap molecule at the 3′ end, the 5′ end, or both of the 3′ and 5′-ends of the sense strand; and wherein the antisense strand comprises any of between 1 and 10, specifically about 1, 2, 3, 4, 5, 6, 7, 8, 9, or 10 phosphorothioate internucleotide linkages, and/or one or more 2′-deoxy, 2′-O-methyl, 2′-deoxy-2′-fluoro, and/or one or more universal base modified nucleotides, and optionally a terminal cap molecule at the 3′ end, the 5′ end, or both of the 3′ and 5′-ends of the antisense strand. In another embodiment, one or more, for example about 1, 2, 3, 4, 5, 6, 7, 8, 9, or 10 pyrimidine nucleotides of the sense and/or antisense siNA stand are chemically-modified with 2′-deoxy, 2′-O-methyl and/or 2′-deoxy-2′-fluoro nucleotides, with or without one or more, for example about 1, 2, 3, 4, 5, 6, 7, 8, 9, or 10 phosphorothioate internucleotide linkages and/or a terminal cap molecule at the 3′ end, the 5′ end, or both of the 3′ and 5′-ends, being present in the same or different strand.

In another embodiment, the invention features a siNA molecule, wherein the sense strand comprises between 1 and 5, specifically about 1, 2, 3, 4, or 5 phosphorothioate internucleotide linkages, and/or one or more 2′-deoxy, 2′-O-methyl, 2′-deoxy-2′-fluoro, and/or one or more universal base modified nucleotides, and optionally a terminal cap molecule at the 3′ end, the 5′ end, or both of the 3′ and 5′-ends of the sense strand; and wherein the antisense strand comprises any of between 1 and 5, specifically about 1, 2, 3, 4, or 5 phosphorothioate internucleotide linkages, and/or one or more 2′-deoxy, 2′-O-methyl, 2′-deoxy-2′-fluoro, and/or one or more universal base modified nucleotides, and optionally a terminal cap molecule at the 3′ end, the 5′ end, or both of the 3′ and 5′-ends of the antisense strand. In another embodiment, one or more, for example about 1, 2, 3, 4, 5, 6, 7, 8, 9, or 10 or more, pyrimidine nucleotides of the sense and/or antisense siNA stand are chemically-modified with 2′-deoxy, 2′-O-methyl and/or 2′-deoxy-2′-fluoro nucleotides, with or without between 1 and 5, for example about 1, 2, 3, 4, or 5 phosphorothioate internucleotide linkages and/or a terminal cap molecule at the 3′ end, the 5′ end, or both of the 3′ and 5′-ends, being present in the same or different strand.

In one embodiment, the invention features a siNA molecule, wherein the antisense strand comprises one or more, for example about 1, 2, 3, 4, 5, 6, 7, 8, 9, or 10 phosphorothioate internucleotide linkages, and/or between one or more 2′-deoxy, 2′-O-methyl, 2′-deoxy-2′-fluoro, and/or one or more universal base modified nucleotides, and optionally a terminal cap molecule at the 3′ end, the 5′ end, or both of the 3′ and 5′-ends of the sense strand; and wherein the antisense strand comprises any of between 1 and 10, specifically about 1, 2, 3, 4, 5, 6, 7, 8, 9, or 10 phosphorothioate internucleotide linkages, and/or one or more 2′-deoxy, 2′-O-methyl, 2′-deoxy-2′-fluoro, and/or one or more universal base modified nucleotides, and optionally a terminal cap molecule at the 3′ end, the 5′ end, or both of the 3′ and 5′-ends of the antisense strand. In another embodiment, one or more, for example about 1, 2, 3, 4, 5, 6, 7, 8, 9, or 10 pyrimidine nucleotides of the sense and/or antisense siNA stand are chemically-modified with 2′-deoxy, 2′-O-methyl and/or 2′-deoxy-2′-fluoro nucleotides, with or without one or more, for example about 1, 2, 3, 4, 5, 6, 7, 8, 9, or 10 phosphorothioate internucleotide linkages and/or a terminal cap molecule at the 3′ end, the 5′ end, or both of the 3′ and 5′-ends, being present in the same or different strand.

In another embodiment, the invention features a siNA molecule, wherein the antisense strand comprises between 1 and 5, specifically about 1, 2, 3, 4, or 5 phosphorothioate internucleotide linkages, and/or one or more 2′-deoxy, 2′-O-methyl, 2′-deoxy-2′-fluoro, and/or one or more universal base modified nucleotides, and optionally a terminal cap molecule at the 3′ end, the 5′ end, or both of the 3′ and 5′-ends of the sense strand; and wherein the antisense strand comprises any of between 1 and 5, specifically about 1, 2, 3, 4, or 5 phosphorothioate internucleotide linkages, and/or one or more 2′-deoxy, 2′-O-methyl, 2′-deoxy-2′-fluoro, and/or one or more universal base modified nucleotides, and optionally a terminal cap molecule at the 3′ end, the 5′ end, or both of the 3′ and 5′-ends of the antisense strand. In another embodiment, one or more, for example about 1, 2, 3, 4, 5, 6, 7, 8, 9, or 10 pyrimidine nucleotides of the sense and/or antisense siNA stand are chemically-modified with 2′-deoxy, 2′-O-methyl and/or 2′-deoxy-2′-fluoro nucleotides, with or without between 1 and 5, for example about 1, 2, 3, 4, or 5 phosphorothioate internucleotide linkages and/or a terminal cap molecule at the 3′ end, the 5′ end, or both of the 3′ and 5′-ends, being present in the same or different strand.

In one embodiment, the invention features a chemically-modified short interfering nucleic acid (siNA) molecule having between about 1 and 5, specifically 1, 2, 3, 4, or 5 phosphorothioate internucleotide linkages in each strand of the siNA molecule.

In another embodiment, the invention features a siNA molecule comprising 2′-5′ internucleotide linkages. The 2′-5′ internucleotide linkage(s) can be at the 5′-end, 3′-end, or both 5′ and 3′ ends of one or both siNA sequence strands. In addition, the 2′-5′ internucleotide linkage(s) can be present at various other positions within one or both siNA sequence strands, for example, every internucleotide linkage of a pyrimidine nucleotide in one or both strands of the siNA molecule can comprise a 2′-5′ internucleotide linkage, or every internucleotide linkage of a purine nucleotide in one or both strands of the siNA molecule can comprise a 2′-5′ internucleotide linkage.

In another embodiment, a chemically-modified siNA molecule of the invention comprises a duplex having two strands, one or both of which can be chemically-modified, wherein each strand is between about 18 and about 27 nucleotides in length, wherein the duplex has between about 18 and about 23 base pairs, and wherein the chemical modification comprises a structure having any of Formulae I-VII. For example, an exemplary chemically-modified siNA molecule of the invention comprises a duplex having two strands, one or both of which can be chemically-modified with a chemical modification having any of Formulae I-VII, wherein each strand consists of about 21 nucleotides, each having two about 2 nucleotide 3′-terminal nucleotide overhangs, and wherein the duplex has 19 base pairs.

In another embodiment, a siNA molecule of the invention comprises a single-stranded hairpin structure, wherein the siNA is between about 36 and about 70 nucleotides in length having between about 18 and about 23 base pairs, and wherein the siNA can include a chemical modification comprising a structure having any of Formulae I-VII. For example, an exemplary chemically-modified siNA molecule of the invention comprises a linear oligonucleotide having between 42 and 50 nucleotides that is chemically-modified with a chemical modification having any of Formulae I-VII, wherein the linear oligonucleotide forms a hairpin structure having 19 base pairs and a 2 nucleotide 3′-terminal nucleotide overhang.

In another embodiment, a linear hairpin siNA molecule of the invention contains a stem loop motif, wherein the loop portion of the siNA molecule is biodegradable. For example, a linear hairpin siNA molecule of the invention is designed such that degradation of the loop portion of the siNA molecule in vivo can generate a double-stranded siNA molecule with 3′-terminal overhangs, such as 3′-terminal nucleotide overhangs comprising about 2 nucleotides.

In another embodiment, a siNA molecule of the invention comprises a circular nucleic acid molecule, wherein the siNA is between about 38 and about 70 nucleotides in length having between about 18 and about 23 base pairs, and wherein the siNA can include a chemical modification, which comprises a structure having any of Formulae I-VII. For example, an exemplary chemically-modified siNA molecule of the invention comprises a circular oligonucleotide having between 42 and 50 nucleotides that is chemically-modified with a chemical modification having any of Formulae I-VII, wherein the circular oligonucleotide forms a dumbbell-shaped structure having 19 base pairs and 2 loops.

In another embodiment, a circular siNA molecule of the invention contains two loop motifs, wherein one or both loop portions of the siNA molecule is biodegradable. For example, a circular siNA molecule of the invention is designed such that degradation of the loop portions of the siNA molecule in vivo can generate a double-stranded siNA molecule with 3′-terminal overhangs, such as 3′-terminal nucleotide overhangs comprising about 2 nucleotides.

In one embodiment, a siNA molecule of the invention comprises one or more abasic residues, for example a compound having Formula V:

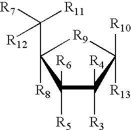

wherein each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2.

In one embodiment, a siNA molecule of the invention comprises one or more inverted abasic residues, for example a compound having Formula VI:

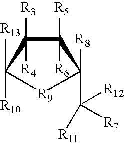

wherein each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and either R2, R3, R8 or R13 serve as points of attachment to the siNA molecule of the invention.

In another embodiment, a siNA molecule of the invention comprises at least one (e.g., about 1, 2, 3, 4, 5, 6, 7, 8, 9, 10, or more) substituted polyalkyl moieties, for example a compound having Formula VII:

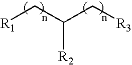

wherein each n is independently an integer from 1 to 12, each R1, R2 and R3 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I, and either R1, R2 or R3 serve as points of attachment to the siNA molecule of the invention.

In another embodiment, the invention features a compound having Formula VII, wherein R1 and R2 are hydroxyl (OH) groups, n=1, and R3 comprises O and is the point of attachment to the 3′-end, the 5-end, or both 3′ and 5′-ends of one or both strands of a double-stranded siNA molecule of the invention or to a single-stranded siNA molecule of the invention. This modification is referred to herein as “glyceryl” (for example modification 6 in FIG. 19).

In another embodiment, a moiety having any of Formulae V, VI or VII of the invention is at the 3′-end, the 5′-end, or both of the 3′ and 5′-ends of a siNA molecule of the invention. For example, a moiety having Formulae V, VI or VII can be present at the 3′-end, the 5′-end, or both of the 3′ and 5′-ends of the antisense strand, the sense strand, or both antisense and sense strands of the siNA molecule. In addition, a moiety having Formulae V, VI or VII can be present at the 3′-end or the 5′-end of a hairpin siNA molecule as described herein.

In another embodiment, a siNA molecule of the invention comprises an abasic residue having Formula V or VI, wherein the abasic residue having Formula V or VI is connected to the siNA construct in a 3′-3′, 3′-2′, 2′-3′, or 5′-5′ configuration, such as at the 3′-end, 5′-end, or both 3′ and ‘5’-ends of one or both siNA strands.

In one embodiment, a siNA molecule of the invention comprises one or more (e.g., about 1, 2, 3, 4, 5, 6, 7, 8, 9, 10, or more) locked nucleic acid (LNA) nucleotides, for example at the 5′-end, 3′-end, 5′ and 3′-end, or any combination thereof, of the siNA molecule.

In another embodiment, a siNA molecule of the invention comprises one or more (e.g., about 1, 2, 3, 4, 5, 6, 7, 8, 9, 10, or more) acyclic nucleotides, for example at the 5′-end, 3′-end, 5′ and 3′-end, or any combination thereof, of the siNA molecule.

In one embodiment, the invention features a chemically-modified short interfering nucleic acid (siNA) molecule of the invention capable of mediating RNA interference (RNAi) against EGFR inside a cell or reconstituted in vitro system, wherein the chemically-modified siNA comprises a sense region, where any (e.g., one or more or all) pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides).

In one embodiment, the invention features a chemically-modified short interfering nucleic acid (siNA) molecule of the invention capable of mediating RNA interference (RNAi) against EGFR inside a cell or reconstituted in vitro system, wherein the chemically-modified siNA comprises a sense region, where any (e.g., one or more or all) pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where any (e.g., one or more or all) purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides), wherein any nucleotides comprising a 3′-terminal nucleotide overhang that are present in said sense region are 2′-deoxy nucleotides.

In one embodiment, the invention features a chemically-modified short interfering nucleic acid (siNA) molecule of the invention capable of mediating RNA interference (RNAi) against EGFR inside a cell or reconstituted in vitro system, wherein the chemically-modified siNA comprises an antisense region, where any (e.g., one or more or all) pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any (e.g., one or more or all) purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides).

In one embodiment, the invention features a chemically-modified short interfering nucleic acid (siNA) molecule of the invention capable of mediating RNA interference (RNAi) against EGFR inside a cell or reconstituted in vitro system, wherein the chemically-modified siNA comprises a sense region, where one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where one or more purine nucleotides present in the sense region are 2′-deoxy purine nucleotides (e.g., wherein all purine nucleotides are 2′-deoxy purine nucleotides or alternately a plurality of purine nucleotides are 2′-deoxy purine nucleotides), and inverted deoxy abasic modifications that are optionally present at the 3′-end, the 5′-end, or both of the 3′ and 5′-ends of the sense region, the sense region optionally further comprising a 3′-terminal nucleotide overhang having between about 1 and about 4 (e.g, about 1, 2, 3, or 4) 2′-deoxyribonucleotides; and wherein the chemically-modified short interfering nucleic acid molecule comprises an antisense region, where one or more pyrimidine nucleotides present in the antisense region are 2′-deoxy-2′-fluoro substituted pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein one or more purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), and a terminal cap modification, such as any modification described herein or shown in FIG. 10, that is optionally present at the 3′-end, the 5′-end, or both of the 3′ and 5′-ends of the antisense sequence, the antisense region optionally further comprising a 3′-terminal overhang having between about 1 and about 4 (e.g, about 1, 2, 3, or 4) 2′-deoxynucleotides, wherein the overhang nucleotides can further comprise one or more (e.g., 1, 2, 3, or 4 ) phosphorothioate internucleotide linkages. Non-limiting examples of these chemically-modified siNAs are shown in FIGS. 4 and 5 herein.

In one embodiment, the invention features a chemically-modified short interfering nucleic acid (siNA) molecule of the invention capable of mediating RNA interference (RNAi) against EGFR inside a cell or reconstituted in vitro system, wherein the siNA comprises a sense region, where one or more pyrimidine nucleotides present in the sense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and where one or more purine nucleotides present in the sense region are purine ribonucleotides (e.g., wherein all purine nucleotides are purine ribonucleotides or alternately a plurality of purine nucleotides are purine ribonucleotides), and inverted deoxy abasic modifications that are optionally present at the 3′-end, the 5′-end, or both of the 3′ and 5′-ends of the sense region, the sense region optionally further comprising a 3′-terminal nucleotide overhang having between about 1 and about 4 (e.g, about 1, 2, 3, or 4) 2′-deoxyribonucleotides; and wherein the siNA comprises an antisense region, where one or more pyrimidine nucleotides present in the antisense region are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the antisense region are 2′-O-methyl purine nucleotides (e.g., wherein all purine nucleotides are 2′-O-methyl purine nucleotides or alternately a plurality of purine nucleotides are 2′-O-methyl purine nucleotides), and a terminal cap modification, such as any modification described herein or shown in FIG. 10, that is optionally present at the 3′-end, the 5′-end, or both of the 3′ and 5′-ends of the antisense sequence, the antisense region optionally further comprising a 3′-terminal nucleotide overhang having between about 1 and about 4 (e.g, about 1, 2, 3, or 4) 2′-deoxynucleotides, wherein the overhang nucleotides can further comprise one or more (e.g., 1, 2, 3, or 4 ) phosphorothioate internucleotide linkages. Non-limiting examples of these chemically-modified siNAs are shown in FIGS. 4 and 5 herein.

In one embodiment, the invention features a chemically-modified short interfering nucleic acid (siNA) molecule capable of mediating RNA interference (RNAi) against EGFR inside a cell, wherein the chemical modification comprises a conjugate covalently attached to the siNA molecule. In another embodiment, the conjugate is covalently attached to the siNA molecule via a biodegradable linker. In one embodiment, the conjugate molecule is attached at the 3′-end of either the sense strand, the antisense strand, or both strands of the siNA. In another embodiment, the conjugate molecule is attached at the 5′-end of either the sense strand, the antisense strand, or both strands of the siNA. In yet another embodiment, the conjugate molecule is attached to both the 3′-end and the 5′-end of either the sense strand, the antisense strand, or both strands of the siNA, or any combination thereof. In one embodiment, a conjugate molecule of the invention comprises a molecule that facilitates delivery of a siNA molecule into a biological system such as a cell. In another embodiment, the conjugate molecule attached to the siNA is a poly ethylene glycol, human serum albumin, or a ligand for a cellular receptor that can mediate cellular uptake. Examples of specific conjugate molecules contemplated by the instant invention that can be attached to siNA molecules are described in Vargeese et al., U.S. Ser. No. 60/311,865, incorporated by reference herein.

In one embodiment, the invention features a siNA molecule capable of mediating RNA interference (RNAi) against EGFR inside a cell, wherein one or both strands of the siNA comprise ribonucleotides at positions within the siNA that are critical for siNA mediated RNAi in a cell. All other positions within the siNA can include chemically-modified nucleotides and/or non-nucleotides such as nucleotides and or non-nucleotides having any of Formulae I, II, III, IV, V, VI, or VII or any combination thereof to the extent that the ability of the siNA molecule to support RNAi activity in a cell is maintained.

In one embodiment, the invention features a method for modulating the expression of a EGFR gene within a cell, comprising: (a) synthesizing a siNA molecule of the invention, which can be chemically-modified, wherein one of the siNA strands includes a sequence complementary to RNA of the EGFR gene; and (b) introducing the siNA molecule into a cell under conditions suitable to modulate the expression of the EGFR gene in the cell.

In one embodiment, the invention features a method for modulating the expression of a EGFR gene within a cell, comprising: (a) synthesizing a siNA molecule of the invention, which can be chemically-modified, wherein one of the siNA strands includes a sequence complementary to RNA of the EGFR gene and wherein the sense strand sequence of the siNA is identical to the complementary sequence of the EGFR RNA; and (b) introducing the siNA molecule into a cell under conditions suitable to modulate the expression of the EGFR gene in the cell.

In another embodiment, the invention features a method for modulating the expression of more than one EGFR gene within a cell, comprising: (a) synthesizing siNA molecules of the invention, which can be chemically-modified, wherein one of the siNA strands includes a sequence complementary to RNA of the EGFR genes; and (b) introducing the siNA molecules into a cell under conditions suitable to modulate the expression of the EGFR genes in the cell.

In another embodiment, the invention features a method for modulating the expression of more than one EGFR gene within a cell, comprising: (a) synthesizing a siNA molecule of the invention, which can be chemically-modified, wherein one of the siNA strands includes a sequence complementary to RNA of the EGFR gene and wherein the sense strand sequence of the siNA is identical to the complementary sequence of the EGFR RNA; and (b) introducing the siNA molecules into a cell under conditions suitable to modulate the expression of the EGFR genes in the cell.

In one embodiment, the invention features a method of modulating the expression of a EGFR gene in a tissue explant, comprising: (a) synthesizing a siNA molecule of the invention, which can be chemically-modified, wherein one of the siNA strands includes a sequence complementary to RNA of the EGFR gene; (b) introducing the siNA molecule into a cell of the tissue explant derived from a particular organism under conditions suitable to modulate the expression of the EGFR gene in the tissue explant, and (c) optionally introducing the tissue explant back into the organism the tissue was derived from or into another organism under conditions suitable to modulate the expression of the EGFR gene in that organism.

In one embodiment, the invention features a method of modulating the expression of a EGFR gene in a tissue explant, comprising: (a) synthesizing a siNA molecule of the invention, which can be chemically-modified, wherein one of the siNA strands includes a sequence complementary to RNA of the EGFR gene and wherein the sense strand sequence of the siNA is identical to the complementary sequence of the EGFR RNA; (b) introducing the siNA molecule into a cell of the tissue explant derived from a particular organism under conditions suitable to modulate the expression of the EGFR gene in the tissue explant, and (c) optionally introducing the tissue explant back into the organism the tissue was derived from or into another organism under conditions suitable to modulate the expression of the EGFR gene in that organism.

In another embodiment, the invention features a method of modulating the expression of more than one EGFR gene in a tissue explant, comprising: (a) synthesizing siNA molecules of the invention, which can be chemically-modified, wherein one of the siNA strands includes a sequence complementary to RNA of the EGFR genes; (b) introducing the siNA molecules into a cell of the tissue explant derived from a particular organism under conditions suitable to modulate the expression of the EGFR genes in the tissue explant, and (c) optionally introducing the tissue explant back into the organism the tissue was derived from or into another organism under conditions suitable to modulate the expression of the EGFR genes in that organism.

In one embodiment, the invention features a method of modulating the expression of a EGFR gene in an organism, comprising: (a) synthesizing a siNA molecule of the invention, which can be chemically-modified, wherein one of the siNA strands includes a sequence complementary to RNA of the EGFR gene; and (b) introducing the siNA molecule into the organism under conditions suitable to modulate the expression of the EGFR gene in the organism.

In another embodiment, the invention features a method of modulating the expression of more than one EGFR gene in an organism, comprising: (a) synthesizing siNA molecules of the invention, which can be chemically-modified, wherein one of the siNA strands includes a sequence complementary to RNA of the EGFR genes; and (b) introducing the siNA molecules into the organism under conditions suitable to modulate the expression of the EGFR genes in the organism.

The siNA molecules of the invention can be designed to inhibit EGFR gene expression through RNAi targeting of a variety of RNA molecules. In one embodiment, the siNA molecules of the invention are used to target various RNAs corresponding to a target gene. Non-limiting examples of such RNAs include messenger RNA (mRNA), alternate RNA splice variants of target gene(s), post-transcriptionally modified RNA of target gene(s), pre-mRNA of target gene(s). If alternate splicing produces a family of transcipts that are distinguished by usage of appropriate exons, the instant invention can be used to inhibit gene expression through the appropriate exons to specifically inhibit or to distinguish among the functions of gene family members. For example, a protein that contains an alternatively spliced transmembrane domain can be expressed in both membrane bound and secreted forms. Use of the invention to target the exon containing the transmembrane domain can be used to determine the functional consequences of pharmaceutical targeting of membrane bound as opposed to the secreted form of the protein. Non-limiting examples of applications of the invention relating to targeting these RNA molecules include therapeutic pharmaceutical applications, pharmaceutical discovery applications, molecular diagnostic and gene function applications, and gene mapping, for example using single nucleotide polymorphism mapping with siNA molecules of the invention. Such applications can be implemented using known gene sequences or from partial sequences available from an expressed sequence tag (EST).

In another embodiment, the siNA molecules of the invention are used to target conserved sequences corresponding to a gene family or gene families such as EGFR genes HER1, HER2, HER3, and/or HER4. As such, siNA molecules targeting multiple EGFR targets can provide increased therapeutic effect. In addition, siNA can be used to characterize pathways of gene function in a variety of applications. For example, the present invention can be used to inhibit the activity of target gene(s) in a pathway to determine the function of uncharacterized gene(s) in gene function analysis, mRNA function analysis, or translational analysis. The invention can be used to determine potential target gene pathways involved in various diseases and conditions toward pharmaceutical development. The invention can be used to understand pathways of gene expression involved in development, such as prenatal development, postnatal development and/or aging.

In one embodiment, siNA molecule(s) and/or methods of the invention are used to inhibit the expression of gene(s) that encode RNA referred to by Genbank Accession, for example EGFR genes such as HER1 (for example Genbank Accession No. NM_{—}005228), HER2 (for example Genbank Accession No. NM_{—}004448), HER3 (for example Genbank Accession No. NM_{—}001982), and HER4 (for example Genbank Accession No. NM_{—}005235). In another embodiment, siNA molecule(s) and/or methods of the invention are used to target RNA sequence(s) referred to by Genbank Accession number, for example EGFR genes such as HER1 (for example Genbank Accession No. NM_{—}005228), HER2 (for example Genbank Accession No. NM_{—}004448), HER3 (for example Genbank Accession No. NM_{—}001982), and HER4 (for example Genbank Accession No. NM_{—}005235). Such sequences are readily obtained using these Genbank Accession numbers.

In one embodiment, the invention features a method comprising: (a) generating a randomized library of siNA constructs having a predetermined complexity, such as of 4^{N}, where N represents the number of base paired nucleotides in each of the siNA construct strands (eg. for a siNA construct having 21-nucleotide sense and antisense strands with 19 base pairs, the complexity would be 4¹⁹); and (b) assaying the siNA constructs of (a) above, under conditions suitable to determine RNAi target sites within the target HER2 RNA sequence. In another embodiment, the siNA molecules of (a) have strands of a fixed length, for example about 23 nucleotides in length. In yet another embodiment, the siNA molecules of (a) are of differing length, for example having strands of about 19 to about 25 (e.g., about 19, 20, 21, 22, 23, 24, or 25) nucleotides in length. In yet another embodiment, the assay can comprise a reconstituted in vitro siNA assay as described in Example 8 herein. In another embodiment, the assay can comprise a cell culture system in which target RNA is expressed. In another embodiment, fragments of HER2 RNA are analyzed for detectable levels of cleavage, for example by gel electrophoresis, northern blot analysis, or RNAse protection assays, to determine the most suitable target site(s) within the target HER2 RNA sequence. In another embodiment, the target HER2 RNA sequence can be obtained as is known in the art, for example, by cloning and/or transcription for in vitro systems, and by cellular expression in in vivo systems.

In another embodiment, the invention features a method comprising: (a) analyzing the sequence of a RNA target encoded by an HER2 gene; (b) synthesizing one or more sets of siNA molecules having sequence complementary to one or more regions of the RNA of (a); and (c) assaying the siNA molecules of (b) under conditions suitable to determine RNAi targets within the target RNA sequence. In another embodiment, the siNA molecules of (b) have strands of a fixed length, for example about 23 nucleotides in length. In yet another embodiment, the siNA molecules of (b) are of differing length, for example having strands of about 19 to about 25 (e.g., about 19, 20, 21, 22, 23, 24, or 25) nucleotides in length. In yet another embodiment, the assay can comprise a reconstituted in vitro siNA assay as described in Example 8 herein. In another embodiment, the assay can comprise a cell culture system in which target RNA is expressed. Fragments of HER2 RNA are analyzed for detectable levels of cleavage, for example by gel electrophoresis, northern blot analysis, or RNAse protection assays, to determine the most suitable target site(s) within the target HER2 RNA sequence. The target HER2 RNA sequence can be obtained as is known in the art, for example, by cloning and/or transcription for in vitro systems, and by expression in in vivo systems.

By “target site” is meant a sequence within a target RNA that is “targeted” for cleavage mediated by a siNA construct which contains sequences within its antisense region that are complementary to the target sequence.

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

In one embodiment, the invention features a composition comprising a siNA molecule of the invention, which can be chemically-modified, in a pharmaceutically acceptable carrier or diluent. In another embodiment, the invention features a pharmaceutical composition comprising siNA molecules of the invention, which can be chemically-modified, targeting one or more genes in a pharmaceutically acceptable carrier or diluent. In another embodiment, the invention features a method for treating or preventing a disease or condition in a patient, comprising administering to the patient a composition of the invention under conditions suitable for the treatment or prevention of the disease or condition in the patient, alone or in conjunction with one or more other therapeutic compounds. In yet another embodiment, the invention features a method for reducing or preventing tissue rejection in a patient comprising administering to the patient a composition of the invention under conditions suitable for the reduction or prevention of tissue rejection in the patient.

In another embodiment, the invention features a method for validating a EGFR gene target, comprising: (a) synthesizing a siNA molecule of the invention, which can be chemically-modified, wherein one of the siNA strands includes a sequence complementary to RNA of a EGFR target gene; (b) introducing the siNA molecule into a cell, tissue, or organism under conditions suitable for modulating expression of the EGFR target gene in the cell, tissue, or organism; and (c) determining the function of the gene by assaying for any phenotypic change in the cell, tissue, or organism.

In one embodiment, the invention features a kit containing a siNA molecule of the invention, which can be chemically-modified, that can be used to modulate the expression of a EGFR target gene in a cell, tissue, or organism. In another embodiment, the invention features a kit containing more than one siNA molecule of the invention, which can be chemically-modified, that can be used to modulate the expression of more than one EGFR target gene in a cell, tissue, or organism.

In one embodiment, the invention features a cell containing one or more siNA molecules of the invention, which can be chemically-modified. In another embodiment, the cell containing a siNA molecule of the invention is a mammalian cell. In yet another embodiment, the cell containing a siNA molecule of the invention is a human cell.

In one embodiment, the synthesis of a siNA molecule of the invention, which can be chemically-modified, comprises: (a) synthesis of two complementary strands of the siNA molecule; (b) annealing the two complementary strands together under conditions suitable to obtain a double-stranded siNA molecule. In another embodiment, synthesis of the two complementary strands of the siNA molecule is by solid phase oligonucleotide synthesis. In yet another embodiment, synthesis of the two complementary strands of the siNA molecule is by solid phase tandem oligonucleotide synthesis.

In one embodiment, the invention features a method for synthesizing a siNA duplex molecule comprising: (a) synthesizing a first oligonucleotide sequence strand of the siNA molecule, wherein the first oligonucleotide sequence strand comprises a cleavable linker molecule that can be used as a scaffold for the synthesis of the second oligonucleotide sequence strand of the siNA; (b) synthesizing the second oligonucleotide sequence strand of siNA on the scaffold of the first oligonucleotide sequence strand, wherein the second oligonucleotide sequence strand further comprises a chemical moiety than can be used to purify the siNA duplex; (c) cleaving the linker molecule of (a) under conditions suitable for the two siNA oligonucleotide strands to hybridize and form a stable duplex; and (d) purifying the siNA duplex utilizing the chemical moiety of the second oligonucleotide sequence strand. In another embodiment, cleavage of the linker molecule in (c) above takes place during deprotection of the oligonucleotide, for example under hydrolysis conditions using an alkylamine base such as methylamine. In another embodiment, the method of synthesis comprises solid phase synthesis on a solid support such as controlled pore glass (CPG) or polystyrene, wherein the first sequence of (a) is synthesized on a cleavable linker, such as a succinyl linker, using the solid support as a scaffold. The cleavable linker in (a) used as a scaffold for synthesizing the second strand can comprise similar reactivity as the solid support derivatized linker, such that cleavage of the solid support derivatized linker and the cleavable linker of (a) takes place concomitantly. In another embodiment, the chemical moiety of (b) that can used to isolate the attached oligonucleotide sequence comprises a trityl group, for example a dimethoxytrityl group, which can be employed in a trityl-on synthesis strategy as described herein. In yet another embodiment, the chemical moiety, such as a dimethoxytrityl group, is removed during purification, for example using acidic conditions.

In a further embodiment, the method for siNA synthesis is a solution phase synthesis or hybrid phase synthesis wherein both strands of the siNA duplex are synthesized in tandem using a cleavable linker attached to the first sequence which acts a scaffold for synthesis of the second sequence. Cleavage of the linker under conditions suitable for hybridization of the separate siNA sequence strands results in formation of the double-stranded siNA molecule.

In another embodiment, the invention features a method for synthesizing a siNA duplex molecule comprising: (a) synthesizing one oligonucleotide sequence strand of the siNA molecule, wherein the sequence comprises a cleavable linker molecule that can be used as a scaffold for the synthesis of another oligonucleotide sequence; (b) synthesizing a second oligonucleotide sequence having complementarity to the first sequence strand on the scaffold of (a), wherein the second sequence comprises the other strand of the double-stranded siNA molecule and wherein the second sequence further comprises a chemical moiety than can be used to isolate the attached oligonucleotide sequence; (c) purifying the product of (b) utilizing the chemical moiety of the second oligonucleotide sequence strand under conditions suitable for isolating the full-length sequence comprising both siNA oligonucleotide strands connected by the cleavable linker; and (d) under conditions suitable for the two siNA oligonucleotide strands to hybridize and form a stable duplex. In another embodiment, cleavage of the linker molecule in (c) above takes place during deprotection of the oligonucleotide, for example under hydrolysis conditions. In another embodiment, cleavage of the linker molecule in (c) above takes place after deprotection of the oligonucleotide. In another embodiment, the method of synthesis comprises solid phase synthesis on a solid support such as controlled pore glass (CPG) or polystyrene, wherein the first sequence of (a) is synthesized on a cleavable linker, such as a succinyl linker, using the solid support as a scaffold. The cleavable linker in (a) used as a scaffold for synthesizing the second strand can comprise similar reactivity or differing reactivity as the solid support derivatized linker, such that cleavage of the solid support derivatized linker and the cleavable linker of (a) takes place either concomitantly or sequentially. In another embodiment, the chemical moiety of (b) that can used to isolate the attached oligonucleotide sequence comprises a trityl group, for example a dimethoxytrityl group.

In another embodiment, the invention features a method for making a double-stranded siNA molecule in a single synthetic process, comprising: (a) synthesizing an oligonucleotide having a first and a second sequence, wherein the first sequence is complementary to the second sequence, and the first oligonucleotide sequence is linked to the second sequence via a cleavable linker, and wherein a terminal 5′-protecting group, for example a 5′-O-dimethoxytrityl group (5′-O-DMT) remains on the oligonucleotide having the second sequence; (b) deprotecting the oligonucleotide whereby the deprotection results in the cleavage of the linker joining the two oligonucleotide sequences; and (c) purifying the product of (b) under conditions suitable for isolating the double-stranded siNA molecule, for example using a trityl-on synthesis strategy as described herein.

In one embodiment, the invention features siNA constructs that mediate RNAi against EGFR, wherein the siNA construct comprises one or more chemical modifications, for example one or more chemical modifications having Formulae I, II, III, IV, V, VI or VII that increases the nuclease resistance of the siNA construct.

In another embodiment, the invention features a method for generating siNA molecules with increased nuclease resistance comprising (a) introducing nucleotides having any of Formulae I-VII into a siNA molecule, and (b) assaying the siNA molecule of step (a) under conditions suitable for isolating siNA molecules having increased nuclease resistance.

In one embodiment, the invention features siNA constructs that mediate RNAi against EGFR, wherein the siNA construct comprises one or more chemical modifications described herein that modulates the binding affinity between the sense and antisense strands of the siNA construct.

In another embodiment, the invention features a method for generating siNA molecules with increased binding affinity between the sense and antisense strands of the siNA molecule comprising (a) introducing nucleotides having any of Formulae I-VII into a siNA molecule, and (b) assaying the siNA molecule of step (a) under conditions suitable for isolating siNA molecules having increased binding affinity between the sense and antisense strands of the siNA molecule.

In one embodiment, the invention features siNA constructs that mediate RNAi against EGFR, wherein the siNA construct comprises one or more chemical modifications described herein that modulates the binding affinity between the antisense strand of the siNA construct and a complementary target RNA sequence within a cell.

In another embodiment, the invention features a method for generating siNA molecules with increased binding affinity between the antisense strand of the siNA molecule and a complementary target RNA sequence, comprising (a) introducing nucleotides having any of Formulae I-VII into a siNA molecule, and (b) assaying the siNA molecule of step (a) under conditions suitable for isolating siNA molecules having increased binding affinity between the antisense strand of the siNA molecule and a complementary target RNA sequence.

In one embodiment, the invention features siNA constructs that mediate RNAi against EGFR, wherein the siNA construct comprises one or more chemical modifications described herein that modulate the polymerase activity of a cellular polymerase capable of generating additional endogenous siNA molecules having sequence homology to the chemically-modified siNA construct.

In another embodiment, the invention features a method for generating siNA molecules capable of mediating increased polymerase activity of a cellular polymerase capable of generating additional endogenous siNA molecules having sequence homology to the chemically-modified siNA molecule comprising (a) introducing nucleotides having any of Formulae I-VII into a siNA molecule, and (b) assaying the siNA molecule of step (a) under conditions suitable for isolating siNA molecules capable of mediating increased polymerase activity of a cellular polymerase capable of generating additional endogenous siNA molecules having sequence homology to the chemically-modified siNA molecule.

In one embodiment, the invention features chemically-modified siNA constructs that mediate RNAi against EGFR in a cell, wherein the chemical modifications do not significantly effect the interaction of siNA with a target RNA molecule and/or proteins or other factors that are essential for RNAi in a manner that would decrease the efficacy of RNAi mediated by such siNA constructs.

In another embodiment, the invention features a method for generating siNA molecules with improved RNAi activity against EGFR, comprising (a) introducing nucleotides having any of Formulae I-VII into a siNA molecule, and (b) assaying the siNA molecule of step (a) under conditions suitable for isolating siNA molecules having improved RNAi activity.

In yet another embodiment, the invention features a method for generating siNA molecules with improved RNAi activity against an EGFR target RNA, comprising (a) introducing nucleotides having any of Formulae I-VII into a siNA molecule, and (b) assaying the siNA molecule of step (a) under conditions suitable for isolating siNA molecules having improved RNAi activity against the target RNA.

In one embodiment, the invention features siNA constructs that mediate RNAi against EGFR, wherein the siNA construct comprises one or more chemical modifications described herein that modulates the cellular uptake of the siNA construct.

In another embodiment, the invention features a method for generating siNA molecules against EGFR with improved cellular uptake, comprising (a) introducing nucleotides having any of Formulae I-VII into a siNA molecule, and (b) assaying the siNA molecule of step (a) under conditions suitable for isolating siNA molecules having improved cellular uptake.

In one embodiment, the invention features siNA constructs that mediate RNAi against EGFR, wherein the siNA construct comprises one or more chemical modifications described herein that increases the bioavailability of the siNA construct, for example by attaching polymeric conjugates such as polyethyleneglycol or equivalent conjugates that improve the pharmacokinetics of the siNA construct, or by attaching conjugates that target specific tissue types or cell types in vivo. Non-limiting examples of such conjugates are described in Vargeese et al., U.S. Ser. No. 60/311,865 incorporated by reference herein.

In one embodiment, the invention features a method for generating siNA molecules of the invention with improved bioavailability, comprising (a) introducing a conjugate into the structure of a siNA molecule, and (b) assaying the siNA molecule of step (a) under conditions suitable for isolating siNA molecules having improved bioavailability. Such conjugates can include ligands for cellular receptors such as peptides derived from naturally occurring protein ligands, protein localization sequences including cellular ZIP code sequences, antibodies, nucleic acid aptamers, vitamins and other co-factors such as folate and N-acetylgalactosamine, polymers such as polyethyleneglycol (PEG), phospholipids, polyamines such as spermine or spermidine, and others.

In another embodiment, the invention features a method for generating siNA molecules of the invention with improved bioavailability, comprising (a) introducing an excipient formulation to a siNA molecule, and (b) assaying the siNA molecule of step (a) under conditions suitable for isolating siNA molecules having improved bioavailability. Such excipients include polymers such as cyclodextrines, lipids, cationic lipids, polyamines, phospholipids, and others.

In another embodiment, the invention features a method for generating siNA molecules of the invention with improved bioavailability, comprising (a) introducing nucleotides having any of Formulae I-VII into a siNA molecule, and (b) assaying the siNA molecule of step (a) under conditions suitable for isolating siNA molecules having improved bioavailability.

In another embodiment, polyethylene glycol (PEG) can be covalently attached to siNA compounds of the present invention. The attached PEG can be any molecular weight, preferably from about 2,000 to about 50,000 daltons (Da).

The present invention can be used alone or as a component of a kit having at least one of the reagents necessary to carry out the in vitro or in vivo introduction of RNA to test samples and/or subjects. For example, preferred components of the kit include the siNA and a vehicle that promotes introduction of the siNA. Such a kit can also include instructions to allow a user of the kit to practice the invention.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid moleule” as used herein refers to any nucleic acid molecule capable of mediating RNA interference “RNAi” or gene silencing; see for example Bass, 2001, Nature, 411, 428-429; Elbashir et al., 2001, Nature, 411, 494-498; and Kreutzer et al., International PCT Publication No. WO 00/44895; Zemicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. Non limiting examples of siRNA molecules of the invention are shown in FIG. 10. For example the siNA can be a double-stranded polynucleotide molecule comprising self-complementary sense and antisense regions, wherein the antisense region comprises complementarity to a target nucleic acid molecule. The siNA can be a single-stranded hairpin polynucleotide having self-complementary sense and antisense regions, wherein the antisense region comprises complementarity to a target nucleic acid molecule. The siNA can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active siNA capable of mediating RNAi. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not contain any ribonucleotides (e.g., nucleotides having a 2′-OH group). The modified short interfering nucleic acid molecules of the invention can also be referred to as short interfering modified oligonucleotides ““siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA, short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing.

By “modulate” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

By “inhibit” it is meant that the activity of a gene expression product or level of RNAs or equivalent RNAs encoding one or more gene products is reduced below that observed in the absence of the nucleic acid molecule of the invention. In one embodiment, inhibition with a siNA molecule preferably is below that level observed in the presence of an inactive or attenuated molecule that is unable to mediate an RNAi response. In another embodiment, inhibition of gene expression with the siNA molecule of the instant invention is greater in the presence of the siNA molecule than in its absence.

By “gene” or “target gene” is meant, a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. Non-limiting examples of plants include monocots, dicots, or gymnosperms. Non-limiting examples of animals include vertebrates or invertebrates. Non-limiting examples of fungi include molds or yeasts.

By “EGFR” as used herein is meant, any epidermal growth factor receptor, such as HER1 (for example encoded by Genbank Accession No. NM_{—}005228), HER2 (for example encoded by Genbank Accession No. NM_{—}004448), HER3 (for example encoded by Genbank Accession No. NM_{—}001982), and HER4 (for example encoded by Genbank Accession No. NM_{—}005235).

By “EGFR proteins” is meant, protein receptor or a mutant protein derivative thereof, having epidermal growth factor receptor activity, for example, having the ability to bind an epidermal growth factor and/or having tyrosine kinase activity.

By “highly conserved sequence region” is meant, a nucleotide sequence of one or more regions in a target gene does not vary significantly from one generation to the other or from one biological system to the other.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The siNA molecules of the invention represent a novel therapeutic approach to treat a variety of pathologic indications, such as cancer, including but not limited to breast, lung, prostate, colorectal, brain, esophageal, bladder, pancreatic, cervical, head and neck, and ovarian cancer, melanoma, lymphoma, glioma, multidrug resistant cancers, and any other diseases or conditions that are related to or will respond to the levels of EGFR in a cell or tissue, alone or in combination with other therapies. The reduction of EGFR expression (specifically EGFR gene RNA levels) and thus reduction in the level of the respective protein relieves, to some extent, the symptoms of the disease or condition.

In one embodiment of the present invention, each sequence of a siNA molecule of the invention is independently 18 to 24 nucleotides in length, in specific embodiments about 18, 19, 20, 21, 22, 23, or 24 nucleotides in length. In another embodiment, the siNA duplexes of the invention independently comprise between about 17 and about 23 base pairs. In yet another embodiment, siNA molecules of the invention comprising hairpin or circular structures are about 35 to about 55 nucleotides in length, or about 38 to about 44 nucleotides in length and comprising 16-22 base pairs. Exemplary siNA molecules of the invention are shown in Tables I-V. Exemplary synthetic siNA molecules of the invention are shown in Table II, III and V and/or FIGS. 12-14.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

The siNA molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Tables I-V and/or FIGS. 12-14. Examples of such nucleic acid molecules consist essentially of sequences defined in these tables and figures.

In another aspect, the invention provides mammalian cells containing one or more siNA molecules of this invention. The one or more siNA molecules can independently be targeted to the same or different sites.

By “RNA” is meant a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “patient” is meant an organism, which is a donor or recipient of explanted cells or the cells themselves. “Patient” also refers to an organism to which the nucleic acid molecules of the invention can be administered. In one embodiment, a patient is a mammal or mammalian cells. In another embodiment, a patient is a human or human cells.

The term “phosphorothioate” as used herein refers to an internucleotide linkage having Formula I, wherein Z and/or W comprise a sulfur atom. Hence, the term phosphorothioate refers to both phosphorothioate and phosphorodithioate internucleotide linkages.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

The term “acyclic nucleotide” as used herein refers to any nucleotide having an acyclic ribose sugar, for example where any of the ribose carbons (C1, C2, C3, C4, or C5), are independently or in combination absent from the nucleotide.

The nucleic acid molecules of the instant invention, individually, or in combination or in conjunction with other drugs, can be used to treat diseases or conditions discussed herein. For example, to treat a particular disease or condition, the siNA molecules can be administered to a patient or can be administered to other appropriate cells evident to those skilled in the art, individually or in combination with one or more drugs under conditions suitable for the treatment.

In a further embodiment, the siNA molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a siNA molecule of the invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

In one embodiment, the invention features an expression vector comprising a nucleic acid sequence encoding at least one siNA molecule of the invention, in a manner that allows expression of the siNA molecule. For example, the vector can contain sequence(s) encoding both strands of a siNA molecule comprising a duplex. The vector can also contain sequence(s) encoding a single nucleic acid molecule that is self complementary and thus forms a siNA molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi: 10.1038/mn725.

In another embodiment, the invention features a mammalian cell, for example, a human cell, including an expression vector of the invention.

In yet another embodiment, the expression vector of the invention comprises a sequence for a siNA molecule having complementarity to a RNA molecule referred to by a Genbank Accession numbers, for example Genbank Accession No. NM_{—}005228 (HER1), Genbank Accession No. NM_{—}004448 (HER2), Genbank Accession No. NM_{—}001982 (HER3), and Genbank Accession No. NM_{—}005235 (HER4).

In one embodiment, an expression vector of the invention comprises a nucleic acid sequence encoding two or more siNA molecules, which can be the same or different.

In another aspect of the invention, siNA molecules that interact with target RNA molecules and down-regulate gene encoding target RNA molecules (for example target RNA molecules referred to by Genbank Accession numbers herein) are expressed from transcription units inserted into DNA or RNA vectors. The recombinant vectors can be DNA plasmids or viral vectors. siNA expressing viral vectors can be constructed based on, but not limited to, adeno-associated virus, retrovirus, adenovirus, or alphavirus. The recombinant vectors capable of expressing the siNA molecules can be delivered as described herein, and persist in target cells. Alternatively, viral vectors can be used that provide for transient expression of siNA molecules. Such vectors can be repeatedly administered as necessary. Once expressed, the siNA molecules bind and down-regulate gene function or expression via RNA interference (RNAi). Delivery of siNA expressing vectors can be systemic, such as by intravenous or intramuscular administration, by administration to target cells ex-planted from a patient followed by reintroduction into the patient, or by any other means that would allow for introduction into the desired target cell.

By “vectors” is meant any nucleic acid- and/or viral-based technique used to deliver a desired nucleic acid.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DESCRIPTION OF THE PREFERRED EMBODIMENTS

First the drawings will be described briefly.

### Drawings

FIG. 1 shows a non-limiting example of a scheme for the synthesis of siNA molecules. The complementary siNA sequence strands, strand 1 and strand 2, are synthesized in tandem and are connected by a cleavable linkage, such as a nucleotide succinate or abasic succinate, which can be the same or different from the cleavable linker used for solid phase synthesis on a solid support. The synthesis can be either solid phase or solution phase, in the example shown, the synthesis is a solid phase synthesis. The synthesis is performed such that a protecting group, such as a dimethoxytrityl group, remains intact on the terminal nucleotide of the tandem oligonucleotide. Upon cleavage and deprotection of the oligonucleotide, the two siNA strands spontaneously hybridize to form a siNA duplex, which allows the purification of the duplex by utilizing the properties of the terminal protecting group, for example by applying a trityl on purification method wherein only duplexes/oligonucleotides with the terminal protecting group are isolated.

FIG. 2 shows a MALDI-TOV mass spectrum of a purified siNA duplex synthesized by a method of the invention. The two peaks shown correspond to the predicted mass of the separate siNA sequence strands. This result demonstrates that the siNA duplex generated from tandem synthesis can be purified as a single entity using a simple trityl-on purification methodology.

FIG. 3 shows a non-limiting example of HER2 protein in SK-BR-3 cells mediated siNA targeting HER2 mRNA site 2344. SK-BR-3 cells were transfected with 0.39-25 nM siNA (RPI#28266/28267) or the inverted control (RPI#28268/28269) as indicated and cationic lipid (4 μg/mL). HER2 protein levels were measured 48 h post-treatment by ELISA. The ratio of HER2 protein over cell density (MTS assay) was determined for each treatment group and results are reported as normalized HER2 protein after treatment with lipid alone, active siNA or inverted control relative to untreated (UNT) cells. Results are reported as the mean of duplicate samples±1 SD.

FIG. 4 shows a non-limiting example of reduction of HER2 mRNA in SK-BR-3 cells mediated by siNA targeting HER2 mRNA site 2344. SK-BR-3 cells were transfected with 0.39-25 nM siNA (RPI#28266/28267) or the inverted control (RPI#28268/28269) as indicated and cationic lipid (4 μg/mL). HER2 mRNA levels were measured 24 h post-treatment by real time RT-PCR. The ratio of HER2 mRNA over 36B4 mRNA was determined for each treatment group and results are reported as normalized HER2 mRNA after treatment with lipid alone, active siNA or inverted control relative to untreated (UNT) cells. Results are reported as the mean of triplicate samples±SD.

FIG. 5 shows a non-limiting example of antiproliferative activity of either unmodified (RPI#28268/28269) or chemically-modified (RPI#29991/29990) siNAs targeting HER2 site 2344 in SK-BR-3 cells. SK-BR-3 cells were transfected with 6.25-50 nM siNA or inverted controls (RPI#28268/28269) and (RPI#29997/29999) as indicated and cationic lipid (4 μg/mL) on days one and three. Cell proliferation was determined 96 h after treatment with lipid alone, active siNAs or inverted controls relative to untreated (UNT) cells. Results are reported as the mean of triplicate samples±SD.

FIG. 6 shows a non-limiting example of reduction of HER2 protein in SK-OV-3 cells mediated by siNA targeting HER2 mRNA site 2344. SK-BR-3 cells were transfected with 0.39-25 nM siNA (RPI#28266/28267) or the inverted control (RPI#28268/28269) as indicated and cationic lipid (4 μg/mL). HER2 protein levels were measured 48 h post-treatment by ELISA. The ratio of HER2 protein over cell density (MTS assay) was determined for each treatment group and results are reported as normalized HER2 protein after treatment with lipid alone, active siNA or inverted control relative to untreated (UNT) cells. Results are reported as the mean of duplicate samples±SD.

FIG. 7 shows a non-limiting example of reduction of HER2 mRNA in SK-OV-3 cells mediated by siNA targeting HER2 mRNA site 2344. SK-BR-3 cells were transfected with 0.39-25 nM siNA (RPI#28266/28267) or the inverted control (RPI#28268/28269) as indicated and cationic lipid (4 μg/mL). HER2 mRNA levels were measured 24 h post-treatment by real time RT-PCR. The ratio of HER2 mRNA over 36B4 mRNA was determined for each treatment group and results are reported as normalized HER2 mRNA after treatment with lipid alone, active siNA or inverted control relative to untreated (UNT) cells. Results are reported as the mean of triplicate samples±SD.

FIG. 8 shows a non-limiting example of reduction of HER2 mRNA in SK-OV-3 cells mediated by chemically-modified siNAs that target HER2 mRNA site 2344. SK-BR-3 cells were transfected with 6.25 or 25 nM unmodified siNA (RPI#28266/28267) or the inverted control (RPI#28268/28269) as well as sets of chemically-modified siNAs as indicated and cationic lipid (4 μg/mL). A particular modified sense strand (RPI#29991) was mixed with each of four possible antisense strands (RPI#s 29990, 29994, 29995 or 29993) and cells were treated with these four sets. HER2 mRNA levels were measured 24 h post-treatment by real time RT-PCR. The ratio of HER2 mRNA over 36B4 mRNA was determined for each treatment group and results are reported as normalized HER2 mRNA after treatment with lipid alone, active siNA or inverted control, and modified sets of siNAs relative to untreated (UNT) cells. Results are reported as the mean of triplicate samples±SD.

FIG. 9 shows a non-limiting example of reduction of HER2 mRNA in SK-OV-3 cells mediated by chemically-modified siNAs that target HER2 mRNA site 2344. SK-BR-3 cells were transfected with 6.25 or 25 nM unmodified siNA (RPI#28266/28267) or the inverted control (RPI#28268/28269) as well as sets of chemically-modified siNAs as indicated and cationic lipid (4 μg/mL). A particular modified sense strand (RPI#29989) was mixed with each of four possible antisense strands (RPI#s 29990, 29994, 29995 or 29993) and cells were treated with these four sets. HER2 mRNA levels were measured 24 h post-treatment by real time RT-PCR. The ratio of HER2 mRNA over 36B4 mRNA was determined for each treatment group and results are reported as normalized HER2 mRNA after treatment with lipid alone, active siNA or inverted control, and modified sets of siNAs relative to untreated (UNT) cells. Results are reported as the mean of triplicate samples ±SD.

FIG. 10 shows a non-limiting example of reduction of HER2 mRNA in SK-OV-3 cells mediated by chemically-modified siNAs that target HER2 mRNA site 2344. SK-BR-3 cells were transfected with 6.25 or 25 nM unmodified siNA (RPI#28266/28267) or the inverted control (RPI#28268/28269) as well as sets of chemically-modified siNAs as indicated and cationic lipid (4 μg/mL). A particular modified sense strand (RPI#29992) was mixed with each of four possible antisense strands (RPI#s 29990, 29994, 29995 or 29993) and cells were treated with these four sets. HER2 mRNA levels were measured 24 h post-treatment by real time RT-PCR. The ratio of HER2 mRNA over 36B4 mRNA was determined for each treatment group and results are reported as normalized HER2 mRNA after treatment with lipid alone, active siNA or inverted control, and modified sets of siNAs relative to untreated (UNT) cells. Results are reported as the mean of triplicate samples±SD.

FIG. 11 shows a non-limiting proposed mechanistic representation of target RNA degradation involved in RNAi. Double-stranded RNA (dsRNA), which is generated by RNA dependent RNA polymerase (RdRP) from foreign single-stranded RNA, for example viral, transposon, or other exogenous RNA, activates the DICER enzyme which in turn generates siNA duplexes having terminal phosphate groups (P). An active siNA complex forms which recognizes a target RNA, resulting in degradation of the target RNA by the RISC endonuclease complex or in the synthesis of additional RNA by RNA dependent RNA polymerase (RdRP), which can activate DICER and result in additional siNA molecules, thereby amplifying the RNAi response.

FIGS. 12A-F shows non-limiting examples of chemically-modified siNA constructs of the present invention. In the figure, N stands for any nucleotide (adenosine, guanosine, cytosine, uridine, or optionally thymidine, for example thymidine can be substituted in the overhanging regions designated by parenthesis (N N). Various modifications are shown for the sense and antisense strands of the siNA constructs.

FIG. 12A The sense strand comprises 21 nucleotides having four phosphorothioate 5′ and 3′-terminal internucleotide linkages, wherein the two terminal 3′-nucleotides are optionally base paired and wherein all pyrimidine nucleotides that may be present are 2′-O-methyl modified nucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complementary to the target RNA sequence, and having one 3′-terminal phosphorothioate internucleotide linkage and four 5′-terminal phosphorothioate internucleotide linkages and wherein all pyrimidine nucleotides that may be present are 2′-deoxy-2′-fluoro modified nucleotides except for (N N) nucleotides, which can comprise naturally occurring ribonucleotides, deoxynucleotides, universal bases, or other chemical modifications described herein.

FIG. 12B The sense strand comprises 21 nucleotides wherein the two terminal 3′-nucleotides are optionally base paired and wherein all pyrimidine nucleotides that may be present are 2′-O-methyl modified nucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complementary to the target RNA sequence, and wherein all pyrimidine nucleotides that may be present are 2′-deoxy-2′-fluoro modified nucleotides except for (N N) nucleotides, which can comprise naturally occurring ribonucleotides, deoxynucleotides, universal bases, or other chemical modifications described herein.

FIG. 12C The sense strand comprises 21 nucleotides having 5′- and 3′-terminal cap moieties wherein the two terminal 3′-nucleotides are optionally base paired and wherein all pyrimidine nucleotides that may be present are 2′-O-methyl modified nucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complementary to the target RNA sequence, and wherein all pyrimidine nucleotides that may be present are 2′-deoxy-2′-fluoro modified nucleotides except for (N N) nucleotides, which can comprise naturally occurring ribonucleotides, deoxynucleotides, universal bases, or other chemical modifications described herein.

FIG. 12D The sense strand comprises 21 nucleotides having five phosphorothioate 5′ and 3′-terminal internucleotide linkages, wherein the two terminal 3′-nucleotides are optionally base paired and wherein all nucleotides are ribonucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complementary to the target RNA sequence, and having one 3′-terminal phosphorothioate internucleotide linkage and five 5′-terminal phosphorothioate internucleotide linkages and wherein all nucleotides are ribonucleotides except for (N N) nucleotides, which can comprise naturally occurring ribonucleotides, deoxynucleotides, universal bases, or other chemical modifications described herein.

FIG. 12E The sense strand comprises 21 nucleotides wherein the two terminal 3′-nucleotides are optionally base paired and wherein all pyrimidine nucleotides that may be present are 2′-O-methyl nucleotides except for (N N) nucleotides, which can comprise naturally occurring ribonucleotides, deoxynucleotides, universal bases, or other chemical modifications described herein. The antisense strand comprises 21 nucleotides all having phosphorothioate internucleotide linkages, wherein the two terminal 3′-nucleotides are optionally complementary to the target RNA sequence, and wherein all nucleotides are ribonucleotides except for (N N) nucleotides, which can comprise naturally occurring ribonucleotides, deoxynucleotides, universal bases, or other chemical modifications described herein.

FIG. 12F The sense strand comprises 21 nucleotides having 5′- and 3′-terminal cap moieties, wherein the two terminal 3′-nucleotides are optionally base paired and wherein all pyrimidine nucleotides that may be present are 2′-O-methyl nucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complementary to the target RNA sequence, and having one 3′-terminal phosphorothioate internucleotide linkage and wherein all pyrimidine nucleotides that may be present are 2′-deoxy-2′-fluoro nucleotides except for (N N) nucleotides, which can comprise naturally occurring ribonucleotides, deoxynucleotides, universal bases, or other chemical modifications described herein. The antisense strand of constructs A-F comprise sequence complementary to target RNA sequence of the invention.

FIGS. 13A-F shows non-limiting examples of specific chemically-modified siNA sequences of the invention. FIGS. 13A-F applies the chemical modifications described in FIGS. 12A-F to an HER2 siNA sequence.

FIGS. 14A-F shows non-limiting examples of specific chemically-modified siNA sequences of the invention. FIGS. 14A-F applies the chemical modifications described in FIGS. 12A-F to an HER1 siNA sequence.

FIG. 15 shows non-limiting examples of different siNA constructs of the invention. The examples shown (constructs 1, 2, and 3) have 19 representative base pairs, however, different embodiments of the invention include any number of base pairs described herein. Bracketed regions represent nucleotide overhangs, for example comprising between about 1, 2, 3, or 4 nucleotides in length, preferably about 2 nucleotides. Constructs 1 and 2 can be used independently for RNAi activity. Construct 2 can comprise a polynucleotide or non-nucleotide linker, which can optionally be designed as a biodegradable linker. In one embodiment, the loop structure shown in construct 2 can comprise a biodegradable linker that results in the formation of construct 1 in vivo and/or in vitro. In another example, construct 3 can be used to generate construct 2 under the same principle wherein a linker is used to generate the active siNA construct 2 in vivo and/or in vitro, which can optionally utilize another biodegradable linker to generate the active siNA construct 1 in vivo and/or in vitro. As such, the stability and/or activity of the siNA constructs can be modulated based on the design of the siNA construct for use in vivo or in vitro and/or in vitro.

FIGS. 16A-C is a diagrammatic representation of a scheme utilized in generating an expression cassette to generate siNA hairpin constructs.

FIG. 16A A DNA oligomer is synthesized with a 5′-restriction site (R1) sequence followed by a region having sequence identical (sense region of siNA) to a predetermined HER2 target seqeunce, wherein the sense region comprises, for example, about 19, 20, 21, or 22 nucleotides (N) in length, which is followed by a loop sequence of defined sequence (X), comprising, for example, between about 3 and 10 nucleotides.

FIG. 16B The synthetic construct is then extended by DNA polymerase to generate a hairpin structure having self complementary sequence that will result in a siNA transcript having specificity for an HER2 target sequence and having self complementary sense and antisense regions.

FIG. 16C The construct is heated (for example to about 95° C.) to linearize the sequence, thus allowing extension of a complementary second DNA strand using a primer to the 3′-restriction sequence of the first strand. The double-stranded DNA is then inserted into an appropriate vector for expression in cells. The construct can be designed such that a 3′-overhang results from the transcription, for example by engineering restriction sites and/or utilizing a poly-U termination region as described in Paul et al., 2002, Nature Biotechnology, 29, 505-508.

FIGS. 17A-C is a diagrammatic representation of a scheme utilized in generating an expression cassette to generate double-stranded siNA constructs.

FIG. 17A A DNA oligomer is synthesized with a 5′-restriction (R1) site sequence followed by a region having sequence identical (sense region of siNA) to a predetermined HER2 target seqeunce, wherein the sense region comprises, for example, about 19, 20, 21, or 22 nucleotides (N) in length, and which is followed by a 3′-restriction site (R2) which is adjacent to a loop sequence of defined sequence (X).

FIG. 17B The synthetic construct is then extended by DNA polymerase to generate a hairpin structure having self complementary sequence.

FIG. 17C The construct is processed by restriction enzymes specific to R1 and R2 to generate a double-stranded DNA which is then inserted into an appropriate vector for expression in cells. The transcription cassette is designed such that a U6 promoter region flanks each side of the dsDNA which generates the separate sense and antisense strands of the siNA. Poly T termination sequences can be added to the constructs to generate U overhangs in the resulting transcript.

FIGS. 18A-E is a diagrammatic representation of a method used to determine target sites for siNA mediated RNAi within a particular target nucleic acid sequence, such as messenger RNA.

FIG. 18A A pool of siNA oligonucleotides are synthesized wherein the antisense region of the siNA constructs has complementarity to target sites across the target nucleic acid sequence, and wherein the sense region comprises sequence complementary to the antisense region of the siNA.

FIGS. 18B-C The sequences are pooled and are inserted into vectors such that transfection of a vector into cells results in the expression of the siNA (FIG. 18C).

FIG. 18D Cells are sorted based on phenotypic change that is associated with modulation of the target nucleic acid sequence.

FIG. 18E The siNA is isolated from the sorted cells and is sequenced to identify efficacious target sites within the target nucleic acid sequence.

FIG. 19 shows non-limiting examples of different stabilization chemistries (1-10) that can be used, for example, to stabilize the 3′-end of siNA sequences of the invention, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6) 3′-glyceryl; (7) [3′-5′]-3′-deoxyribonucleotide; (8) [3′-3′]-deoxyribonucleotide; (9) [5′-2′]-deoxyribonucleotide; and (10) [5-3′]-dideoxyribonucleotide. In addition to modified and unmodified backbone chemistries indicated in the figure, these chemistries can be combined with different backbone modifications as described herein, for example, backbone modifications having Formula I. In addition, the 2′-deoxy nucleotide shown 5′ to the terminal modifications shown can be another modified or unmodified nucleotide or non-nucleotide described herein, for example modifications having Formulae II, III, IV, V, or VI.

Mechanism of action of Nucleic Acid Molecules of the Invention

The discussion that follows discusses the proposed mechanism of RNA interference mediated by short interfering RNA as is presently known, and is not meant to be limiting and is not an admission of prior art. Applicant demonstrates herein that chemically-modified short interfering nucleic acids possess similar or improved capacity to mediate RNAi as do siRNA molecules and are expected to possess improved stability and activity in vivo; therefore, this discussion is not meant to be limiting only to siRNA and can be applied to siNA as a whole. By “improved capacity to mediate RNAi” is meant to include RNAi activity measured in vitro and/or in vivo where the RNAi activity is a reflection of both the ability of the siNA to mediate RNAi and the stability of the siRNAs of the invention. In this invention, the product of these activities can be increased in vitro and/or in vivo compared to an all RNA siRNA or an siNA containing a plurality of ribonucleotides. In some cases, the activity or stability of the siNA molecule can be decreased (i.e., less than ten-fold), but the overall activity of the siNA molecule is enhanced, in vitro and/or in vivo.

RNA interference refers to the process of sequence specific post-transcriptional gene silencing in animals mediated by short interfering nucleic acid (siNA) or short interfering RNAs (siRNA) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double-stranded RNAs (dsRNA) derived from viral infection or the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21 to about 23 nucleotides in length and comprise about 19 base pair duplexes. Dicer has also been implicated in the excision of about 21 and about 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single-stranded RNA having sequence homologous to the siRNA. Cleavage of the target RNA takes place in the middle of the region complementary to the guide sequence of the siRNA duplex (Elbashir et al., 2001, Genes Dev., 15, 188).

Short interfering RNA mediated RNAi has been studied in a variety of systems. Fire et al, 1998, Nature, 391, 806, were the first to observe RNAi in C. Elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two di-nucleotide 3′-terminal nucleotide overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J, 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs may occur in vivo.

Synthesis of Nucleic acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., individual siNA oligonucleotide sequences or siNA sequences synthesized in tandem) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides or 2′-deoxy-2′-fluoro nucleotides. Table VI outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA including certain siNA molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table VI outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA·3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT Publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

The siNA molecules of the invention can also be synthesized via a tandem synthesis methodology as described in Example 1 herein, wherein both siNA strands are synthesized as a contiguous oligonucleotide sequence separated by a cleavable linker which is subsequently cleaved to provide separate siNA sequences that hybridize and permit purification of the siNA duplex. The tandem synthesis of siNA as described herein can be readily adapted to both multiwell/multiplate synthesis platforms such as 96 well or similarly larger multi-well platforms. The tandem synthesis of siNA as described herein can also be readily adapted to large scale synthesis platforms employing batch reactors, synthesis columns and the like.

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). siNA constructs can be purified by gel electrophoresis using general methods or can be purified by high pressure liquid chromatography (HPLC; see Wincott et al., supra, the totality of which is hereby incorporated herein by reference) and re-suspended in water.

In another aspect of the invention, siNA molecules of the invention are expressed from transcription units inserted into DNA or RNA vectors. The recombinant vectors can be DNA plasmids or viral vectors. siNA expressing viral vectors can be constructed based on, but not limited to, adeno-associated virus, retrovirus, adenovirus, or alphavirus. The recombinant vectors capable of expressing the siNA molecules can be delivered as described herein, and persist in target cells. Alternatively, viral vectors can be used that provide for transient expression of siNA molecules.

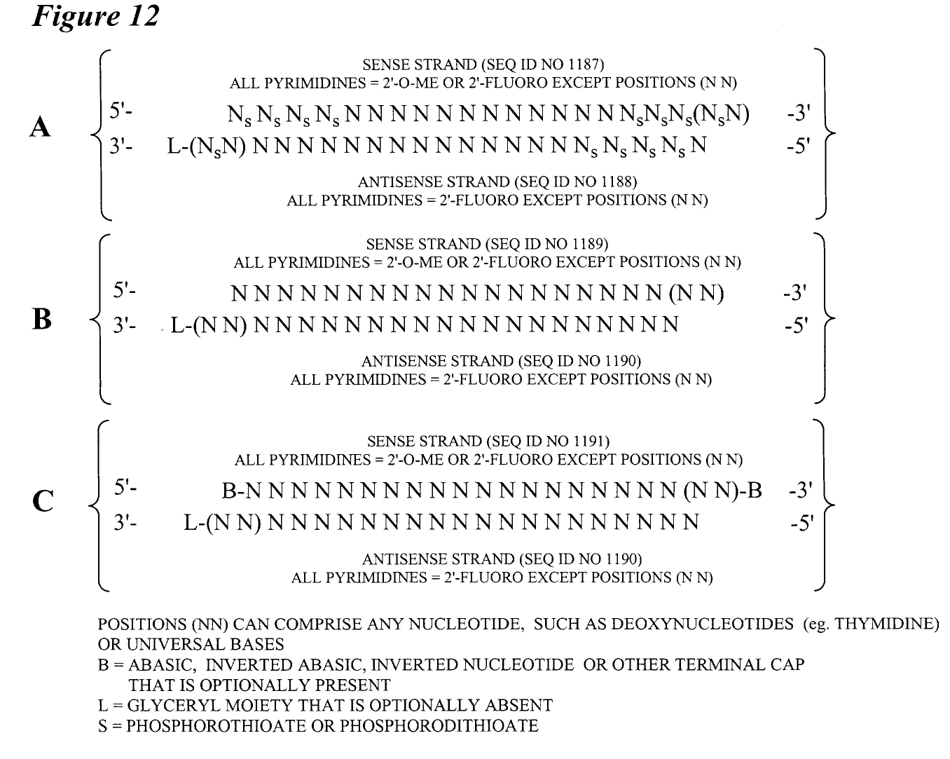

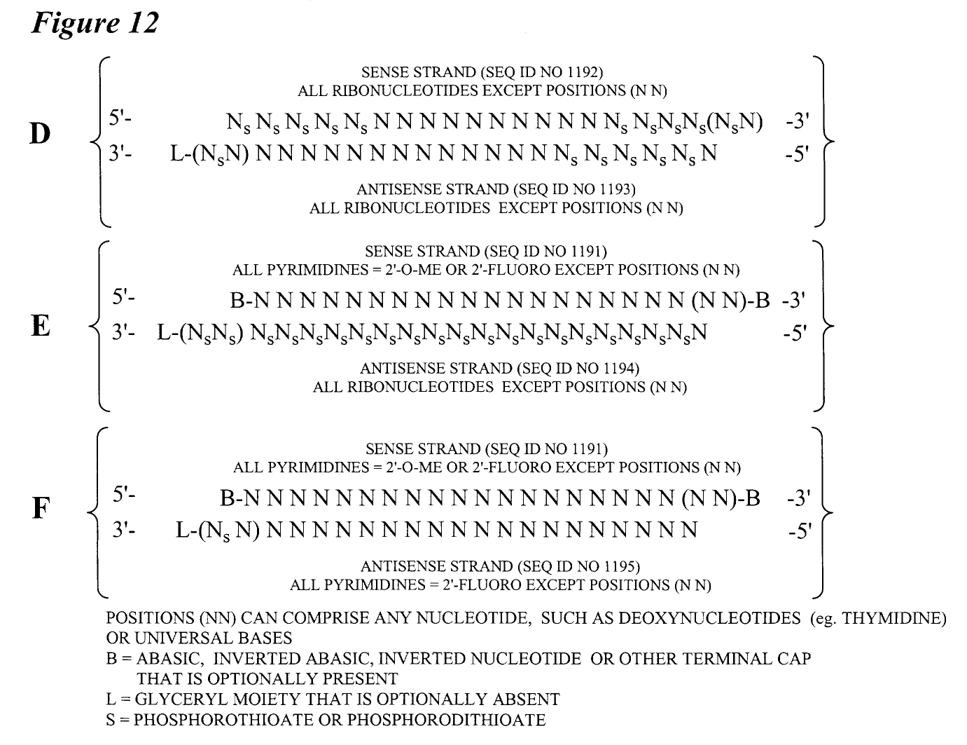

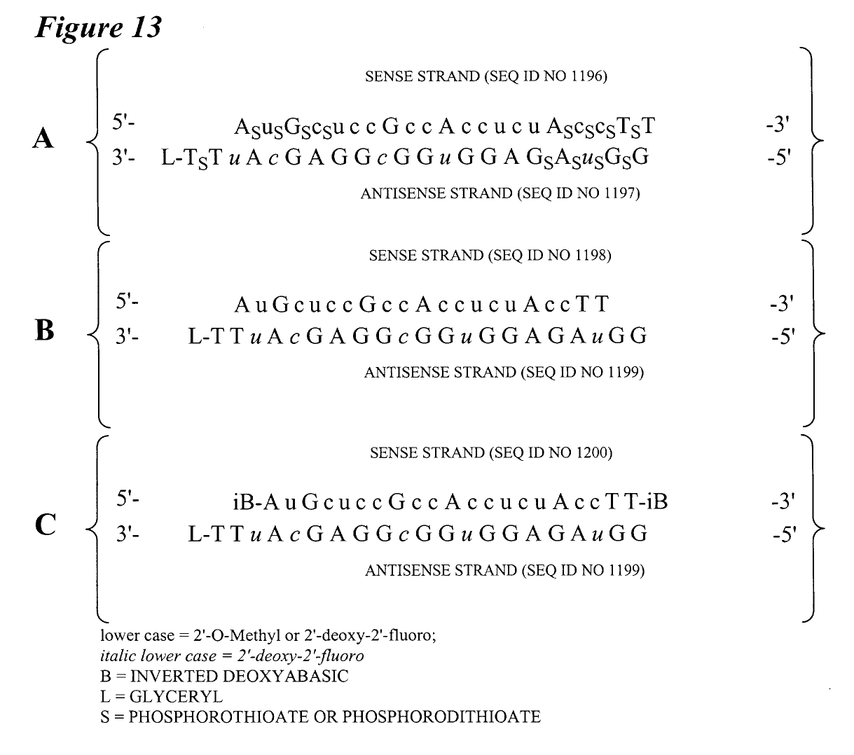

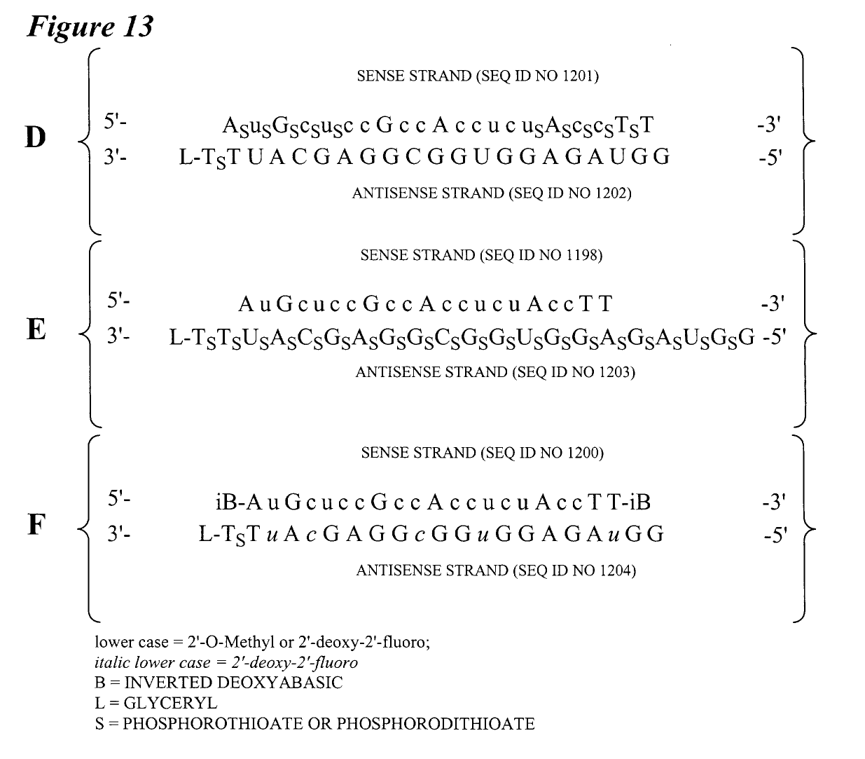

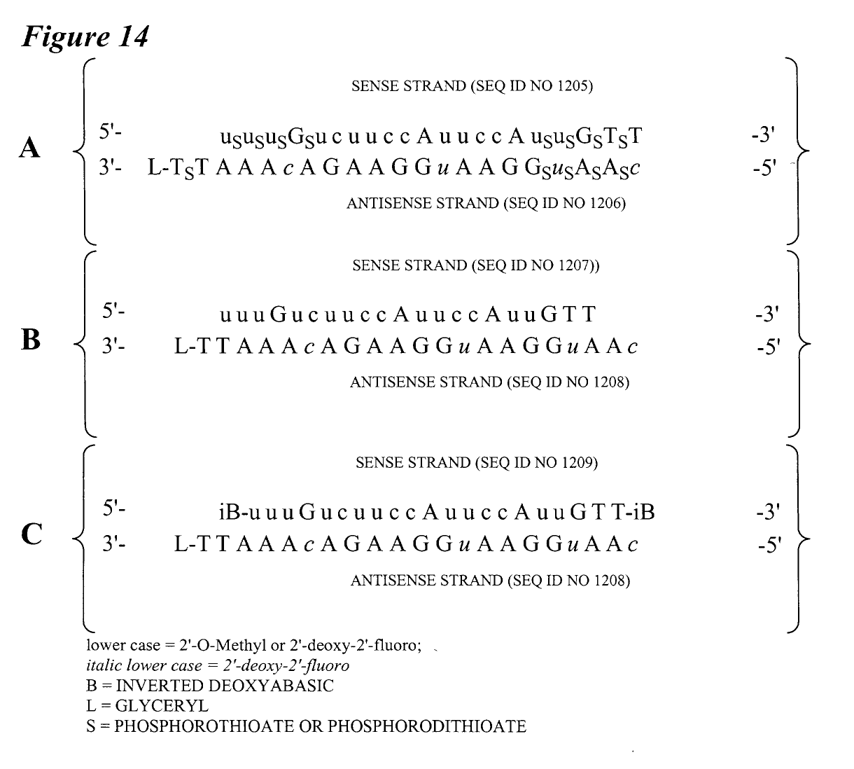

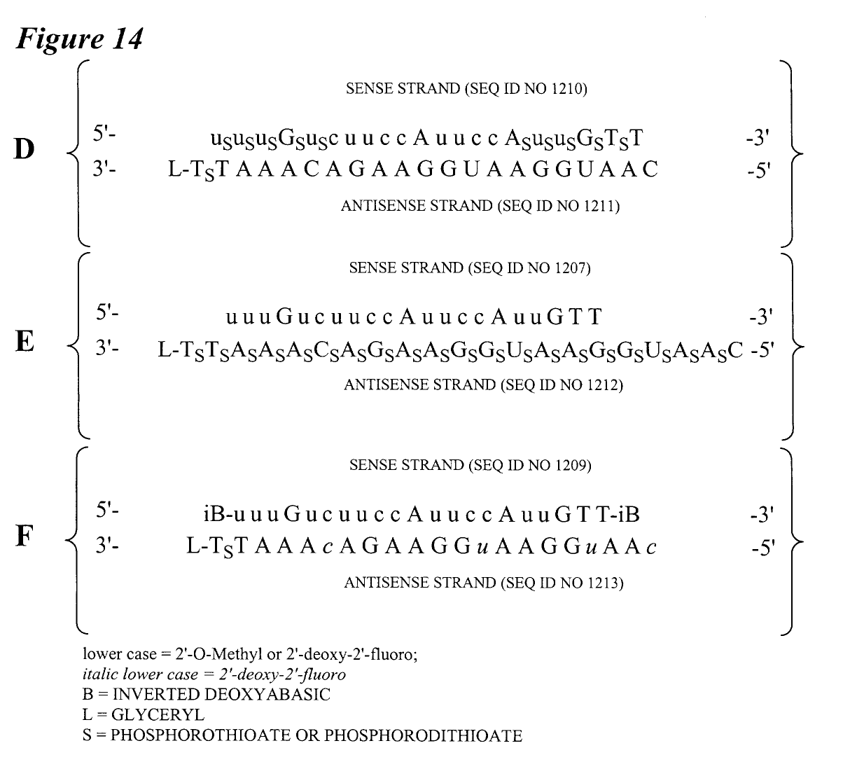

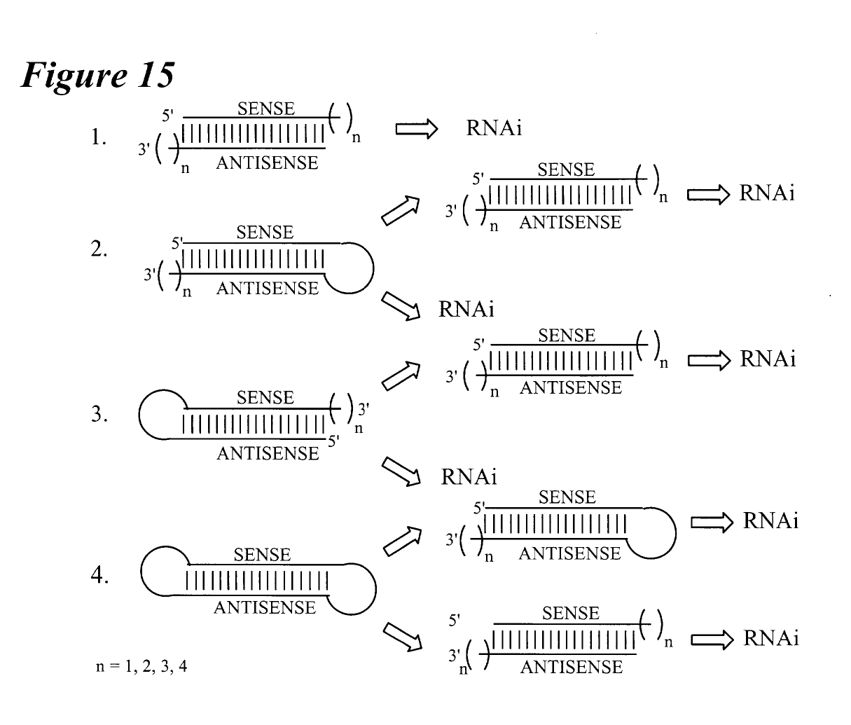

The sequences of the siNA constructs that are chemically synthesized, useful in this study, are shown in Table II, III, V and/or FIGS. 12-14. Similarly, the siNA construct sequences listed in the Tables can be formed of ribonucleotides or other nucleotides or non-nucleotides as described herein.

Optimizing Activity of the nucleic acid molecule of the invention.

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-O-allyl, and/or 2′-H, nucleotide base modifications (for a review see Usman and Cedergren, 1992, TIBS., 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into nucleic acid molecules without modulating catalysis, and are incorporated by reference herein. In view of such teachings, similar modifications can be used as described herein to modify the siNA nucleic acid molecules of the instant invention so long as the ability of siNA to promote RNAi is cells is not significantly inhibited.

While chemical modification of oligonucleotide internucleotide linkages with phosphorothioate, phosphorothioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

Short interfering nucleic acid (siNA) molecules having chemical modifications that maintain or enhance activity are provided. Such a nucleic acid is also generally more resistant to nucleases than an unmodified nucleic acid. Accordingly, the in vitro and/or in vivo activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., 1995, Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability, as described above.

In one embodiment, nucleic acid molecules of the invention include one or more G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands. In another embodiment, nucleic acid molecules of the invention include one or more LNA “locked nucleic acid” nucleotides such as a 2′, 4′-C mythylene bicyclo nucleotide (see for example Wengel et al., International PCT Publication No. WO 00/66604 and WO 99/14226).

In another embodiment, the invention features conjugates and/or complexes of siNA molecules of the invention. Such conjugates and/or complexes can be used to facilitate delivery of siNA molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically-modified nucleotides, for example, 2′-O-methyl, 2′-fluoro, 2′-amino, 2′-O-amino, 2′-C-allyl, 2′-O-allyl, and other 2′-modified or base-modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active siNA molecules either alone or in combination with othe molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

Therapeutic nucleic acid molecules (e.g., siNA molecules) delivered exogenously optimally are stable within cells until reverse trascription of the RNA has been modulated long enough to reduce the levels of the RNA transcript. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another embodiment, siNA molecules having chemical modifications that maintain or enhance enzymatic activity of proteins involved in RNAi are provided. Such nucleic acids are also generally more resistant to nucleases than unmodified nucleic acids. Thus, in vitro and/or in vivo the activity should not be significantly lowered.

Use of the nucleic acid-based molecules of the invention will lead to better treatment of the disease progression by affording the possibility of combination therapies (e.g., multiple siNA molecules targeted to different genes; nucleic acid molecules coupled with known small molecule modulators; or intermittent treatment with combinations of molecules, including different motifs and/or other chemical or biological molecules). The treatment of patients with siNA molecules can also include combinations of different types of nucleic acid molecules, such as enzymatic nucleic acid molecules (ribozymes), allozymes, antisense molecules, 2,5-A oligoadenylate, decoys, aptamers etc.

In another aspect a siNA molecule of the invention comprises one or more 5′ and/or a 3′-cap structure, for example on only the sense siNA strand, antisense siNA strand, or both siNA strands.

By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and can help in delivery and/or localization within a cell. The cap can be present at the 5′-terminus (5′-cap) or at the 3′-terminal (3′-cap) or can be present on both termini. In non-limiting examples: the 5′-cap is selected from the group comprising inverted abasic residue (moiety); 4,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

An “alkyl” group refers to a saturated aliphatic hydrocarbon, including straight-chain, branched-chain, and cyclic alkyl groups. Preferably, the alkyl group has 1 to 12 carbons. More preferably, it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups that are unsaturated hydrocarbon groups containing at least one carbon-carbon double bond, including straight-chain, branched-chain, and cyclic groups. Preferably, the alkenyl group has 1 to 12 carbons. More preferably, it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups that have an unsaturated hydrocarbon group containing at least one carbon-carbon triple bond, including straight-chain, branched-chain, and cyclic groups. Preferably, the alkynyl group has 1 to 12 carbons. More preferably, it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group that has at least one ring having a conjugated pi electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents.

In one embodiment, the invention features modified siNA molecules, with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, uracil joined to the 1′ carbon of β-D-ribo-furanose.

By “modified nucleoside” is meant any nucleotide base that contains a modification in the chemical structure of an unmodified nucleotide base, sugar and/or phosphate.

In connection with 2′-modified nucleotides as described for the present invention, by “amino” is meant 2′—NH₂ or 2′—O—NH_{2,} which can be modified or unmodified. Such modified groups are described, for example, in Eckstein et al., U.S. Pat. No. 5,672,695 and Matulic-Adamic et al., U.S. Pat. No. 6,248,878, which are both incorporated by reference in their entireties.

Various modifications to nucleic acid siNA structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Administration of Nucleic Acid Molecules

A siNA molecule of the invention can be adapted for use to treat, for example, cancer and any other indications that can respond to the level of EGFR in a cell or tissue, alone or in combination with other therapies. For example, a siNA molecule can comprise a delivery vehicle, including liposomes, for administration to a subject, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akbtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a patient.

Thus, the invention features a pharmaceutical composition comprising one or more nucleic acid(s) of the invention in an acceptable carrier, such as a stabilizer, buffer, and the like. The polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes that lead to systemic absorption include, without limitation: intravenous, subcutaneous, intraperitoneal, inhalation, oral, intrapulmonary and intramuscular. Each of these administration routes exposes the siNA molecules of the invention to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, DF et al, 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

In one embodiment, the invention comprises compositions suitable for administering nucleic acid molecules of the invention to specific cell types, such as hepatocytes. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention.

Alternatively, certain siNA molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45. Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856.

In another aspect of the invention, RNA molecules of the present invention can be expressed from transcription units (see for example Couture et al., 1996, TIG., 12, 510) inserted into DNA or RNA vectors. The recombinant vectors can be DNA plasmids or viral vectors. siNA expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pat. Nos. 5,902,880 and 6,146,886). The recombinant vectors capable of expressing the siNA molecules can be delivered as described above, and persist in target cells. Alternatively, viral vectors can be used that provide for transient expression of nucleic acid molecules. Such vectors can be repeatedly administered as necessary. Once expressed, the siNA molecule interacts with the target mRNA and generates an RNAi response. Delivery of siNA molecule expressing vectors can be systemic, such as by intravenous or intra-muscular administration, by administration to target cells ex-planted from a patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one siNA molecule of the instant invention. The expression vector can encode one or both strands of a siNA duplex, or a single self-complementary strand that self hybridizes into a siNA duplex. The nucleic acid sequences encoding the siNA molecules of the instant invention can be operably linked in a manner that allows expression of the siNA molecule (see for example Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725).

In another aspect, the invention features an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siNA molecules of the instant invention; wherein said sequence is operably linked to said initiation region and said termination region, in a manner that allows expression and/or delivery of the siNA molecule. The vector can optionally include an open reading frame (ORF) for a protein operably linked on the 5′ side or the 3′-side of the sequence encoding the siNA of the invention; and/or an intron (intervening sequences).

Transcription of the siNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J, 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siNA in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736. The above siNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising a nucleic acid sequence encoding at least one of the siNA molecules of the invention, in a manner that allows expression of that siNA molecule. The expression vector comprises in one embodiment; a) a transcription initiation region; b) a transcription termination region; and c) a nucleic acid sequence encoding at least one strand of the siNA molecule; wherein the sequence is operably linked to the initiation region and the termination region, in a manner that allows expression and/or delivery of the siNA molecule.

In another embodiment the expression vector comprises: a) a transcription initiation region; b) a transcription termination region; c) an open reading frame; and d) a nucleic acid sequence encoding at least one strand of a siNA molecule, wherein the sequence is operably linked to the 3′-end of the open reading frame; and wherein the sequence is operably linked to the initiation region, the open reading frame and the termination region, in a manner that allows expression and/or delivery of the siNA molecule. In yet another embodiment the expression vector comprises: a) a transcription initiation region; b) a transcription termination region; c) an intron; and d) a nucleic acid sequence encoding at least one siNA molecule; wherein the sequence is operably linked to the initiation region, the intron and the termination region, in a manner which allows expression and/or delivery of the nucleic acid molecule.

In another embodiment, the expression vector comprises: a) a transcription initiation region; b) a transcription termination region; c) an intron; d) an open reading frame; and e) a nucleic acid sequence encoding at least one strand of a siNA molecule, wherein the sequence is operably linked to the 3′-end of the open reading frame; and wherein the sequence is operably linked to the initiation region, the intron, the open reading frame and the termination region, in a manner which allows expression and/or delivery of the siNA molecule.

### EXAMPLES

The following are non-limiting examples showing the selection, isolation, synthesis and activity of nucleic acids of the instant invention.

### Example 1

### Tandem Synthesis of siNA Constructs

Exemplary siNA molecules of the invention are synthesized in tandem using a cleavable linker, for example a succinyl-based linker. Tandem synthesis as described herein is followed by a one-step purification process that provides RNAi molecules in high yield. This approach is highly amenable to siNA synthesis in support of high throughput RNAi screening, and can be readily adapted to multi-column or multi-well synthesis platforms.

After completing a tandem synthesis of an siNA oligo and its compliment in which the 5′-terminal dimethoxytrityl (5′-O-DMT) group remains intact (trityl on synthesis), the oligonucleotides are deprotected as described above. Following deprotection, the siNA sequence strands are allowed to spontaneously hybridize. This hybridization yields a duplex in which one strand has retained the 5′-O-DMT group while the complementary strand comprises a terminal 5′-hydroxyl. The newly formed duplex to behaves as a single molecule during routine solid-phase extraction purification (Trityl-On purification) even though only one molecule has a dimethoxytrityl group. Because the strands form a stable duplex, this dimethoxytrityl group (or an equivalent group, such as other trityl groups or other hydrophobic moieties) is all that is required to purify the pair of oligos, for example by using a C18 cartridge.

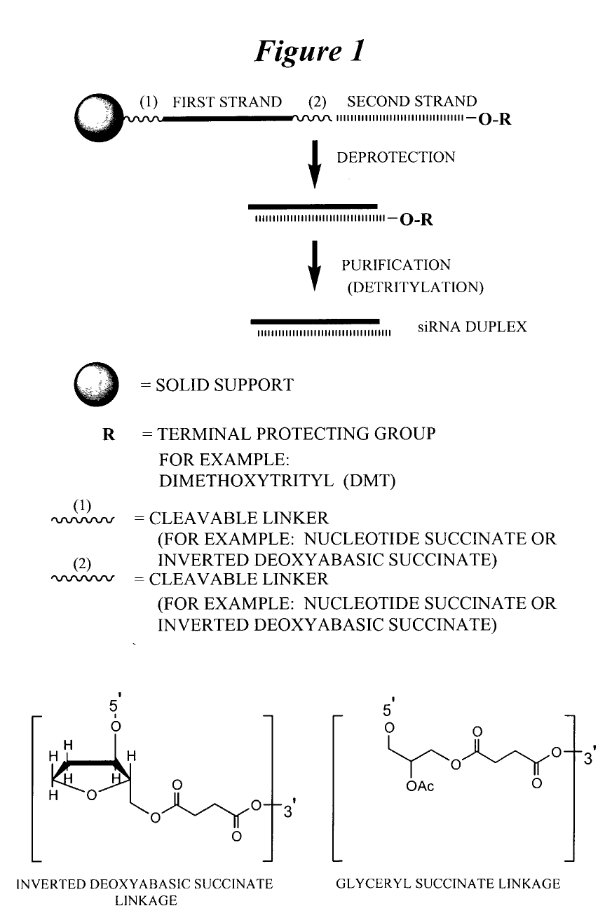

Standard phosphoramidite synthesis chemistry is used up to point of introducing a tandem linker, such as an inverted deoxy abasic succinate or glyceryl succinate linker (see FIG. 1) or an equivalent cleavable linker. A non-limiting example of linker coupling conditions that can be used includes a hindered base such as diisopropylethylamine (DIPA) and/or DMAP in the presence of an activator reagent such as Bromotripyrrolidinophosphoniumhexaflurorophosphate (PyBrOP). After the linker is coupled, standard synthesis chemistry is utilized to complete synthesis of the second sequence leaving the terminal the 5′-O-DMT intact. Following synthesis, the resulting oligonucleotide is deprotected according to the procedures described herein and quenched with a suitable buffer, for example with 50 mM NaOAc or 1.5M NH₄H₂CO₃.

Purification of the siNA duplex can be readily accomplished using solid phase extraction, for example using a Waters C18 SepPak 1 g cartridge conditioned with 1 column volume (CV) of acetonitrile, 2 CV H2O, and 2 CV 50 mM NaOAc. The sample is loaded and then washed with 1 CV H2O or 50 mM NaOAc. Failure sequences are eluted with 1 CV 14% ACN (Aqueous with 50 mM NaOAc and 50 mM NaCl). The column is then washed, for example with 1 CV H2O followed by on-column detritylation, for example by passing 1 CV of 1% aqueous trifluoroacetic acid (TFA) over the column, then adding a second CV of 1% aqueous TFA to the column and allowing to stand for approx. 10 minutes. The remaining TFA solution is removed and the column washed with H2O followed by 1 CV 1M NaCl and additional H2O. The siNA duplex product is then eluted, for example using 1 CV 20% aqueous CAN.

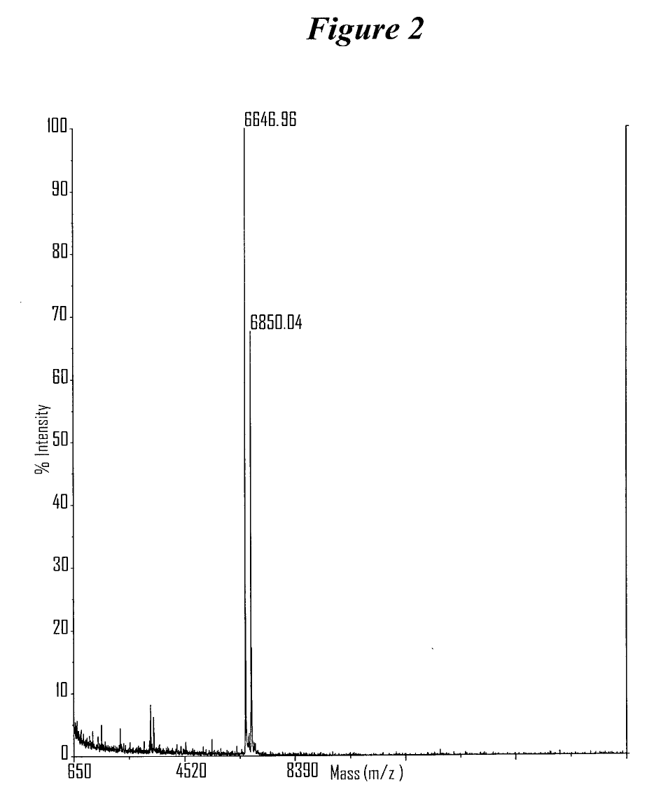

FIG. 2 provides an example of MALDI-TOV mass spectrometry analysis of a purified siNA construct in which each peak corresponds to the calculated mass of an individual siNA strand of the siNA duplex. The same purified siNA provides three peaks when analyzed by capillary gel electrophoresis (CGE), one peak presumably corresponding to the duplex siNA, and two peaks presumably corresponding to the separate siNA sequence strands. Ion exchange HPLC analysis of the same siNA contract only shows a single peak. Testing of the purified siNA construct using a luciferase reporter assay described below demonstrated the same RNAi activity compared to siNA constructs generated from separately synthesized oligonucleotide sequence strands.

### Example 2

### Identification of Potential siNA Target Sites in any RNA Sequence

The sequence of an RNA target of interest, such as a viral or human mRNA transcript, is screened for target sites, for example by using a computer folding algorithm. In a non-limiting example, the sequence of a gene or RNA gene transcript derived from a database, such as Genbank, is used to generate siNA targets having complimentarity to the target. Such sequences can be obtained from a database, or can be determined experimentally as known in the art. Target sites that are known, for example, those target sites determined to be effective target sites based on studies with other nucleic acid molecules, for example ribozymes or antisense, or those targets known to be associated with a disease or condition such as those sites containing mutations or deletions, can be used to design siNA molecules targeting those sites as well. Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siNA molecules for efficacy, for example by using in vitro RNA cleavage assays, cell culture, or animal models. In a non-limiting example, anywhere from 1 to 1000 target sites are chosen within the transcript based on the size of the siNA contruct to be used. High throughput screening assays can be developed for screening siNA molecules using methods known in the art, such as with multi-well or multi-plate assays to determine efficient reduction in target gene expression.

### Example 3

### Selection of siNA Molecule Target Sites in a RNA

The following non-limiting steps can be used to carry out the selection of siNAs targeting a given gene sequence or transcipt.

1. The target sequence is parsed in silico into a list of all fragments or subsequences of a particular length, for example 23 nucleotide fragments, contained within the target sequence. This step is typically carried out using a custom Perl script, but commercial sequence analysis programs such as Oligo, MacVector, or the GCG Wisconsin Package can be employed as well.

2. In some instances the siNAs correspond to more than one target sequence; such would be the case for example in targeting many different strains of a viral sequence, for targeting different transcipts of the same gene, targeting different transcipts of more than one gene, or for targeting both the human gene and an animal homolog. In this case, a subsequence list of a particular length is generated for each of the targets, and then the lists are compared to find matching sequences in each list. The subsequences are then ranked according to the number of target sequences that contain the given subsequence; the goal is to find subsequences that are present in most or all of the target sequences. Alternately, the ranking can indentify subsequences that are unique to a target sequence, such as a mutant target sequence. Such an approach would enable the use of siNA to target specifically the mutant sequence and not effect the expression of the normal sequence.

3. In some instances the siNA subsequences are absent in one or more sequences while present in the desired target sequence; such would be the case if the siNA targets a gene with a paralogous family member that is to remain untargeted. As in case 2 above, a subsequence list of a particular length is generated for each of the targets, and then the lists are compared to find sequences that are present in the target gene but are absent in the untargeted paralog.

4. The ranked siNA subsequences can be further analyzed and ranked according to GC content. A preference can be given to sites containing 30-70% GC, with a further preference to sites containing 40-60% GC.

5. The ranked siNA subsequences can be further analyzed and ranked according to self-folding and internal hairpins. Weaker internal folds are preferred; strong hairpin structures are to be avoided.

6. The ranked siNA subsequences can be further analyzed and ranked according to whether they have runs of GGG or CCC in the sequence. GGG (or even more Gs) in either strand can make oligonucleotide synthesis problematic, so it is avoided whenever better sequences are available. CCC is searched in the target strand because that will place GGG in the antisense strand.

7. The ranked siNA subsequences can be further analyzed and ranked according to whether they have the dinucleotide UU (uridine dinucleotide) on the 3′ end of the sequence, and/or AA on the 5′ end of the sequence (to yield 3′ UU on the antisense sequence). These sequences allow one to design siNA molecules with terminal TT thymidine dinucleotides.

8. Four or five target sites are chosen from the ranked list of subsequences as described above. For example, in subsequences having 23 nucleotides, the right 21 nucleotides of each chosen 23-mer subsequence are then designed and synthesized for the upper (sense) strand of the siNA duplex, while the reverse complement of the left 21 nucleotides of each chosen 23-mer subsequence are then designed and synthesized for the lower (antisense) strand of the siNA duplex. If terminal TT residues are desired for the sequence (as described in paragraph 7), then the two 3′ terminal nucleotides of both the sense and antisense strands are replaced by TT prior to synthesizing the oligos.

9. The siNA molecules are screened in an in vitro, cell culture or animal model system to identify the most active siNA molecule or the most preferred target site within the target RNA sequence.

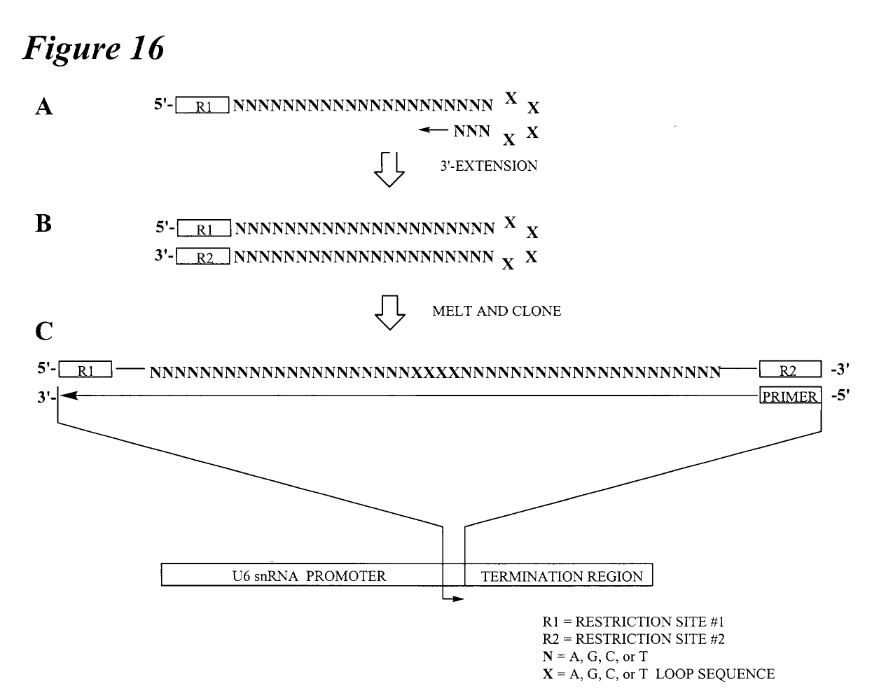

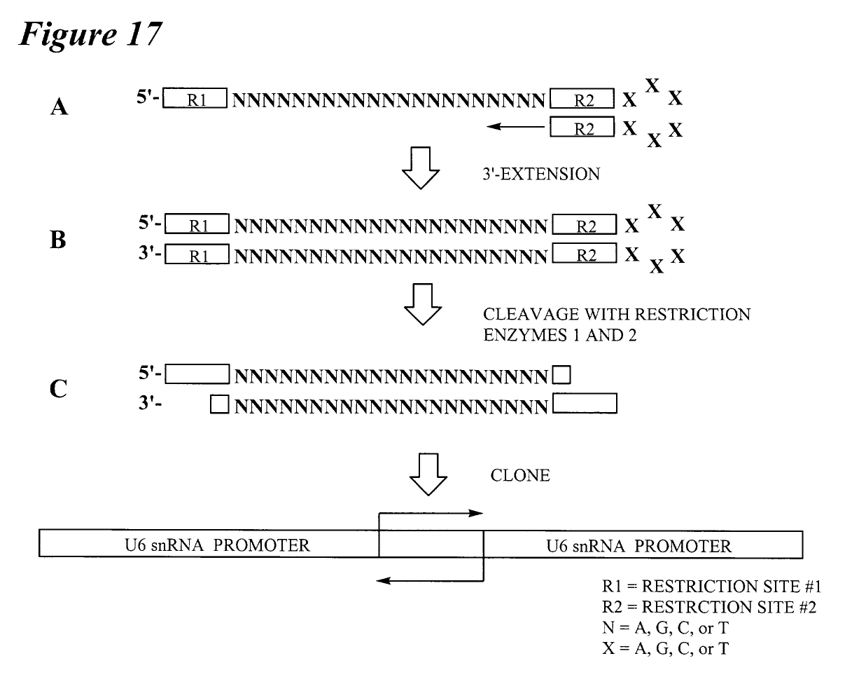

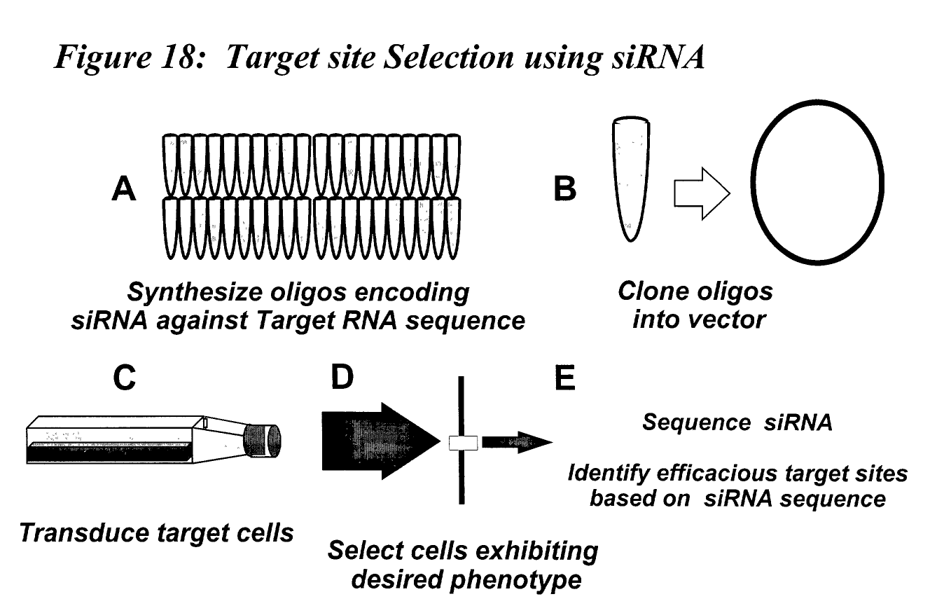

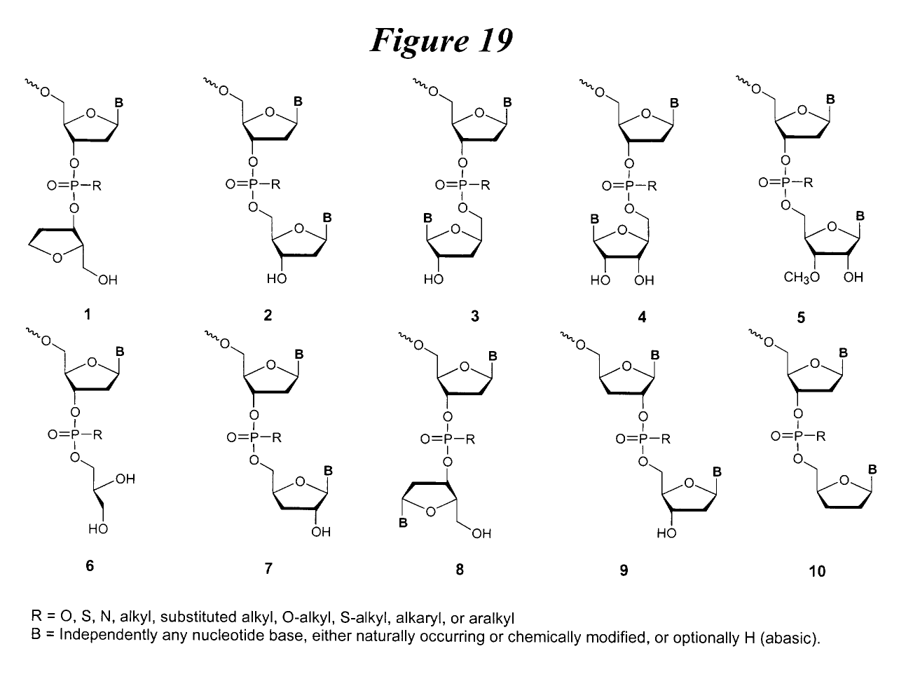

In an alternate approach, a pool of siNA constructs specific to an EGFR (e.g., HER1, HER2) target sequence is used to screen for target sites in cells expressing EGFR RNA. The general strategy used in this approach is shown in FIG. 18. Cells expressing EGFR (e.g., HER1, HER2) are transfected with the pool of siNA constructs and cells that demonstrate a phenotype associated with EGFR (e.g., HER1, HER2) inhibition are sorted. The pool of siNA constructs can be expressed from transcription cassettes inserted into appropriate vectors (see for example FIG. 16 and FIG. 17). Cells in which EGFR (e.g., HER1, HER2) expression is decreased due to siNA treatment demonstrate a phenotypic change, for example decreased production of EGFR (e.g., HER1, HER2) RNA or protein(s) compared to untreated cells or cells treated with a control siNA. The siNA from cells demonstrating a positive phenotypic change (e.g., decreased EGFR EGFR (e.g., HER1, HER2) RNA or protein), are sequenced to determine the most suitable target site(s) within the target RNA sequence.

### Example 4

### EGFR Targeted siNA Design

siNA target sites were chosen by analyzing sequences of the EGFR (e.g., HER1, HER2) RNA target and optionally prioritizing the target sites on the basis of folding (structure of any given sequence analyzed to determine siNA accessibility to the target). siNA molecules were designed that could bind each target and are optionally individually analyzed by computer folding to assess whether the siNA molecule can interact with the target sequence. Varying the length of the siNA molecules can be chosen to optimize activity. Generally, a sufficient number of complementary nucleotide bases are chosen to bind to, or otherwise interact with, the target RNA, but the degree of complementarity can be modulated to accommodate siNA duplexes or varying length or base composition. By using such methodologies, siNA molecules can be designed to target sites within any known RNA sequence, for example those RNA sequences corresponding to the any gene transcript.

### Example 5

### Chemical Synthesis and Purification of siNA

siNA molecules can be designed to interact with various sites in the RNA message, for example target sequences within the RNA sequences described herein. The sequence of one strand of the siNA molecule(s) are complementary to the target site sequences described above. The siNA molecules can be chemically synthesized using methods described herein. Inactive siNA molecules that are used as control sequences can be synthesized by scrambling the sequence of the siNA molecules such that it is not complementary to the target sequence.

### Example 6

### In Vivo Models Used to Evaluate the Down-Regulation of EGFR Gene Expression

Nucleic acid molecules targeted to the human EGFR RNA are designed and synthesized as described above. These nucleic acid molecules can be tested for cleavage activity in vivo, for example, using the procedures described below. A variety of endpoints have been used in cell culture models to evaluate EGFR-mediated effects after treatment with anti-EGFR agents. Phenotypic endpoints include inhibition of cell proliferation, apoptosis assays and reduction of EGFR protein expression. Because overexpression of EGFR is directly associated with increased proliferation of tumor cells, a proliferation endpoint for cell culture assays is preferably used as a primary screen. There are several methods by which this endpoint can be measured. Following treatment of cells with nucleic acid molecules, cells are allowed to grow (typically 5 days) after which either the cell viability, the incorporation of [³H] thymidine into cellular DNA and/or the cell density can be measured. The assay of cell density is well-known to those skilled in the art and can, for example, be performed in a 96-well format using commercially available fluorescent nucleic acid stains (such as Syto® 13 or CyQuant®) or the ability of live cells to reduce MTS to formazon (Promega, Madison, Wis.). For example, the MTS assay is described herein.

As a secondary, confirmatory endpoint, a nucleic acid-mediated decrease in the level of EGFR RNA and/or EGFR protein expression can be evaluated using methods known in the art, such as RT-PCR, Northern blot, ELISA, Western blot, and immunoprecipitation analyses, to name a few techniques.

Validation of Cell Lines and Ribozyme Treatment Conditions

Two human cell lines (SKBR-3 and SKOV-3) that are known to express medium to high levels of EGFR protein are considered for nucleic acid screening. In order to validate these cell lines for EGFR-mediated sensitivity, both cell lines are treated with an EGFR specific antibody, for example mAB IMC-C225 (ImClone) and its effect on cell proliferation is determined. mAB is added to cells at concentrations ranging from 0-8 μM in medium containing either no serum (OptiMem), 0.1% or 0.5% FBS and efficacy is determined via cell proliferation. Inhibition of proliferation (˜50%) in both cell lines after addition of mAB at 0.5 nM in medium containing 0.1% or no FBS, indicates that both cell lines are sensitive to an anti-EGFR agent (mAB) and supports their use in experiments testing anti-EGFR nucleic acid molecules.

Prior to nucleic acid screening, the choice of the optimal lipid(s) and conditions for nucleic acid delivery is determined empirically for each cell line. Applicant has established a panel of cationic lipids (lipids as described in PCT application WO99/05094) that can be used to deliver nucleic acids to cultured cells and are useful for cell proliferation assays that are typically 3-5 days in length. Additional description of useful lipids is provided above, and those skilled in the art are also familiar with a variety of lipids that can be used for delivery of oligonucleotide to cells in culture. Initially, this panel of lipid delivery vehicles is screened in SKBR-3 and SKOV-3 cells using previously established control oligonucleotides. Specific lipids and conditions for optimal delivery are selected for each cell line based on these screens. These conditions are used to deliver EGFR specific nucleic acids to cells for primary (inhibition of cell proliferation) and secondary (decrease in EGFR RNA/protein) efficacy endpoints.

Primary Screen: Inhibition of Cell Proliferation

Nucleic acid screens were performed using an automated, high throughput 96-well cell proliferation assay. Cell proliferation was measured over a 5-day treatment period using the MTS assay for determining cell density. The growth of cells treated with siNA/lipid complexes was compared to untreated cells, lipid treatment alone, and to cells treated with a inverted control sequence. Inverted controls can no longer bind to the target site due to a reversal of the native sequence. These controls are used to determine non-specific inhibition of cell growth caused by nucleic acid chemistry. The growth of cells treated with siNA/lipid complexes was compared to untreated cells, lipid treatment alone, and to cells treated with an inverted control sequence. Lead nucleic acids are chosen from the primary screen based on their ability to inhibit cell proliferation in a specific manner. Dose response assays are carried out on these leads and a subset are advanced into a secondary screen using a reduction in the level of EGFR protein and/or RNA as an endpoint.

Secondary Screen: Decrease in EGFR Protein and/or RNA

A secondary screen that measures the effect of anti-EGFR nucleic acids on EGFR protein and/or RNA levels is used to affirm preliminary findings. A EGFR ELISA for both SKBR-3 and SKOV-3 cells can been established and made available for use as an additional endpoint. In addition, a real time RT-PCR assay (TaqMan assay) has been developed to assess EGFR RNA reduction. Dose response activity of nucleic acid molecules of the instant invention can be used to assess both EGFR protein and RNA reduction endpoints.

siNA Mechanism Assays

A TaqMan® assay for measuring the siNA-mediated decrease in EGFR RNA has been established. This assay is based on PCR technology and can measure in real time the production of EGFR mRNA relative to a standard cellular mRNA such as 36B4. This RNA assay is used to establish proof that lead siNAs are working through an RNA cleavage mechanism and result in a decrease in the level of EGFR mRNA, thus leading to a decrease in cell surface EGFR protein receptors and a subsequent decrease in tumor cell proliferation.

Animal Models

Evaluating the efficacy of anti-HER2 agents in animal models is an important prerequisite to human clinical trials. As in cell culture models, the most HER2 sensitive mouse tumor xenografts are those derived from human carcinoma cells that express high levels of HER2 protein. In a recent study, nude mice bearing human vulvar (A431), lung (A549 and SK-LC-16 NSCL and LX-1) and prostate (PC-3 and TSU-PRI) xenografts were sensitive to the anti-HER2 tyrosine kinase inhibitor ZD1839 (Iressa), resulting in a partial regression of A431 tumor growth, 70-80% inhibition of tumor growth (A549, SKLC-16, TSU-PRI and PC-3 tumors), and 50-55% inhibition against the LX-1 tumor at a 150 mg kg dose (ip, every 3-4 days×4), (Sirotnak et al., 2000, Clin. Cancer Res., 6, 4885-48892). This same study compared the efficacy of ZD1839 alone or in combination with the commonly used chemotherapeutics, cisplatin, carboplatin, paclitaxel, docetaxel, edatrexate, gemcitabine, vinorelbine. When used in combination with certain chemotherapeutic agents, most notably cisplatin, carboplatin, paclitaxel, docetaxel, and edatrexate, marked response was observed compared to treatment with these agents alone, resulting in partial or complete regression in some cases. The above studies provide evidence that inhibition of HER2 expression by anti-HER2 agents causes inhibition of tumor growth in animals.

Animal Model Development

Tumor cell lines (SKBR-3 and SKOV-3) are characterized to establish their growth curves in mice. These cell lines are implanted into both nude and SCID mice and primary tumor volumes are measured 3 times per week. Growth characteristics of these tumor lines using a Matrigel implantation format can also be established. The use of other cell lines that have been engineered to express high levels of EGFR can also be used in the described studies. The tumor cell line(s) and implantation method that supports the most consistent and reliable tumor growth is used in animal studies testing the lead EGFR nucleic acid(s). Nucleic acids are administered by daily subcutaneous injection or by continuous subcutaneous infusion from Alzet mini osmotic pumps beginning 3 days after tumor implantation and continuing for the duration of the study. Group sizes of at least 10 animals are employed. Efficacy is determined by statistical comparison of tumor volume of nucleic acid-treated animals to a control group of animals treated with saline alone. Because the growth of these tumors is generally slow (45-60 days), an initial endpoint is the time in days it takes to establish an easily measurable primary tumor (i.e. 50-100 mm³) in the presence or absence of nucleic acid treatment.

EGFR Protein Levels for Patient Screening and as a Potential Endpoint

Because elevated EGFR levels can be detected in several cancers, cancer patients can be pre-screened for elevated EGFR prior to admission to initial clinical trials testing an anti-EGFR nucleic acid. Initial EGFR levels can be determined (by ELISA) from tumor biopsies or resected tumor samples. During clinical trials, it may be possible to monitor circulating EGFR protein by ELISA. Evaluation of serial blood/serum samples over the course of the anti-EGFR nucleic acid treatment period could be useful in determining early indications of efficacy.

### Example 7

### RNAi Mediated Inhibition of HER2 Expression

Unmodified and chemically-modified (see Table II) siNAs against HER2 site 2344 were tested for the ability to reduce endogenous HER2 RNA and protein in the HER2 overexpressing breast cancer cell line SK-BR-3. Additionally, siNAs were tested for the ability to inhibit proliferation of SK-BR-3 cells. Further, unmodified and additional chemically-modified siNAs (see Table II) against HER2 site 2344 were tested for the ability to reduce endogenous HER2 RNA in the HER2 overexpressing ovarian cancer cell line SK-OV-3.

SK-BR-3 cells were maintained in McCoy's medium (GIBCO/BRL, Bethesda, Md.) supplemented with 10% fetal bovine serum, L-glutamine (2 mM), bovine insulin (10 μg/mL). SK-OV-3 cells were maintained in EMEM medium (GIBCO/BRL, Bethesda, Md.) supplemented with 10% fetal bovine serum.

Cells were seeded in 96-well plates at a density of 7,500 and 5,000 cells/well for SK-BR-3 and SK-OV-3 cells, respectivelyin 100 μL of growth medium and incubated at 37° C. under 5% CO₂ for 24 h. Transfection of siNAs or inverted controls for RNA and protein endpoints was achieved by the following method: a 5×mixture of siNA (1.95-250 nM) and a cationic lipid formulation (20 μg/mL) was made in 150 μL of growth medium. siNA/lipid complexes were allowed to form for 20 min at 37° C. under 5% CO₂. A 25 μL aliquot of 5×siNA/lipid complexes was then added to treatment wells containing 100 μL of medium, resulting in a 1×final concentration of siNA (0.39-50 nM) and lipid (4 μg/mL). siNA/lipid complexes were left on cells for 24 h (RNA endpoint) or 48 h (protein endpoint).

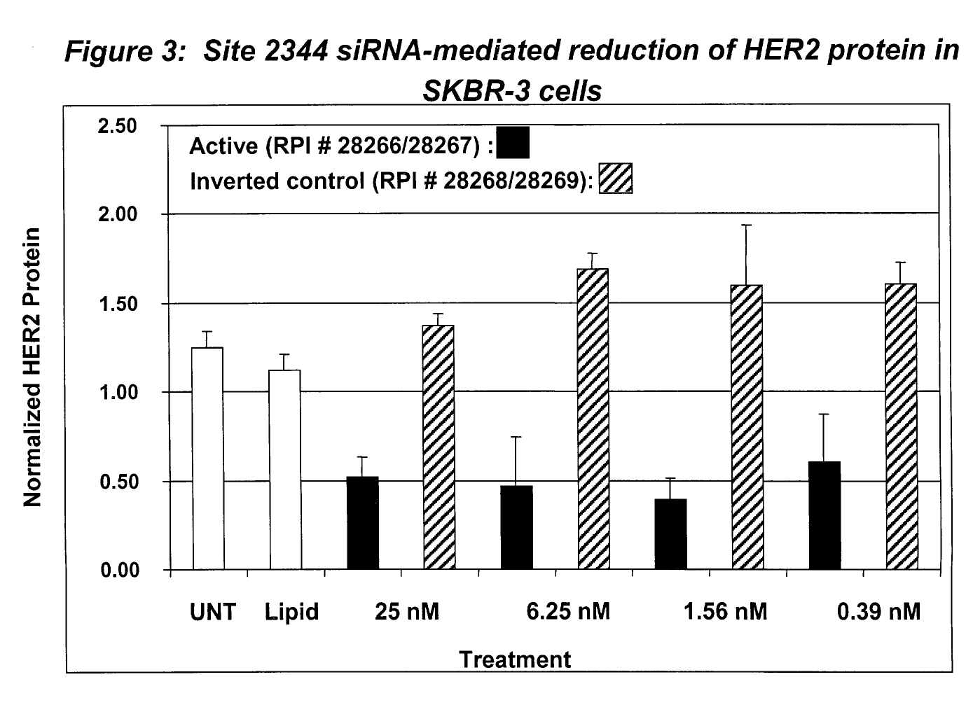

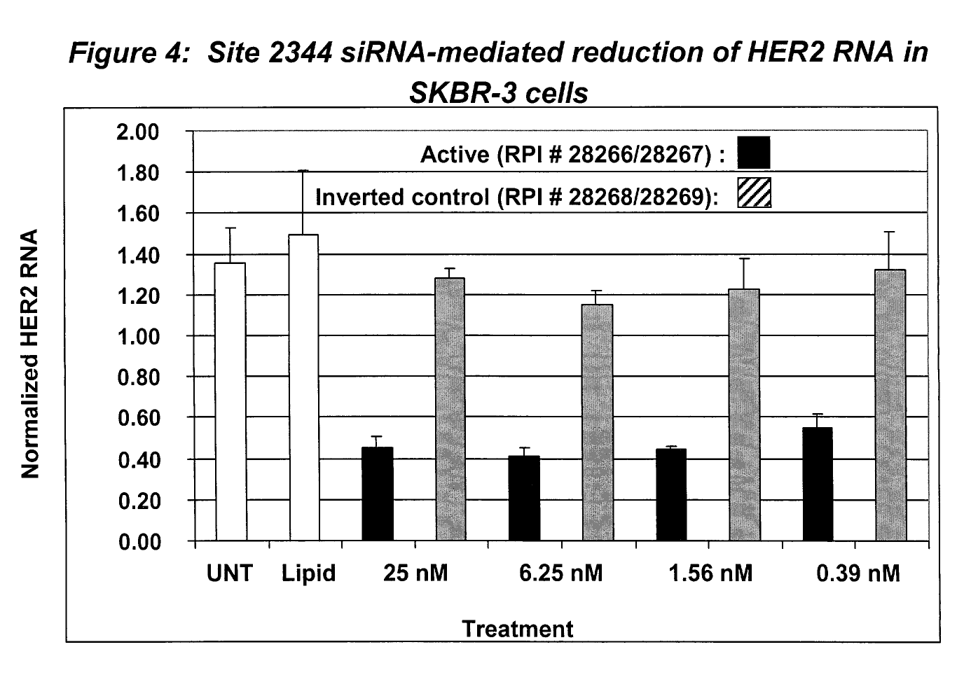

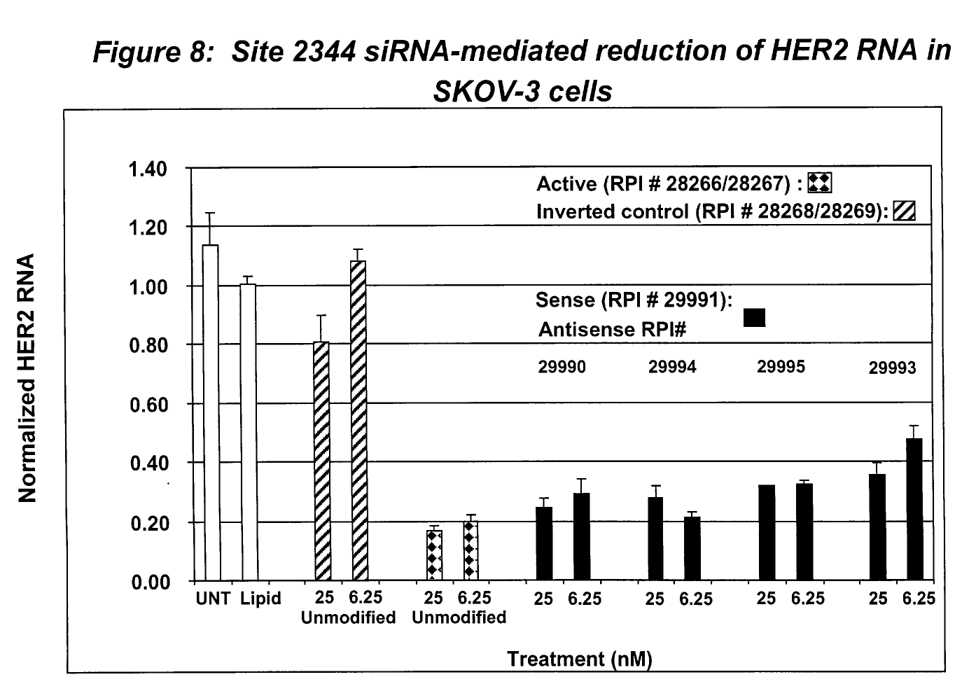

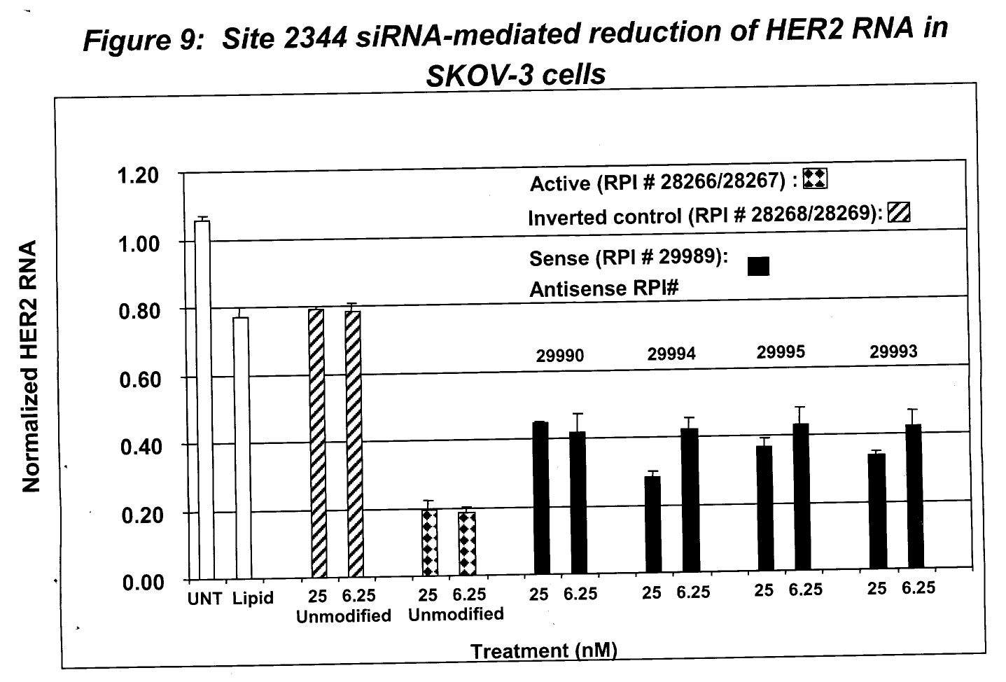

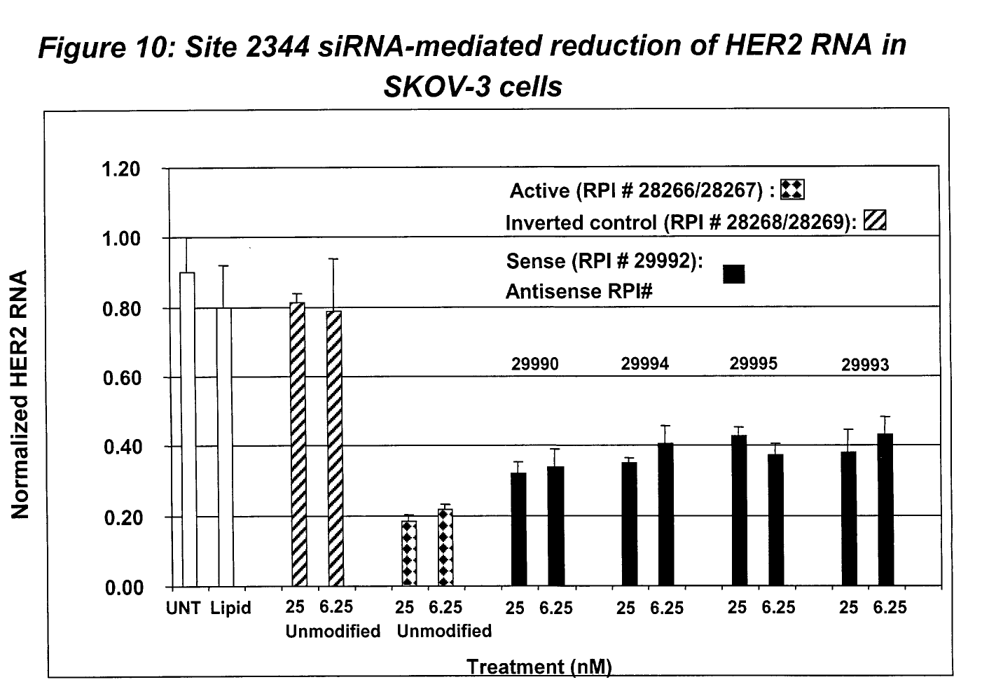

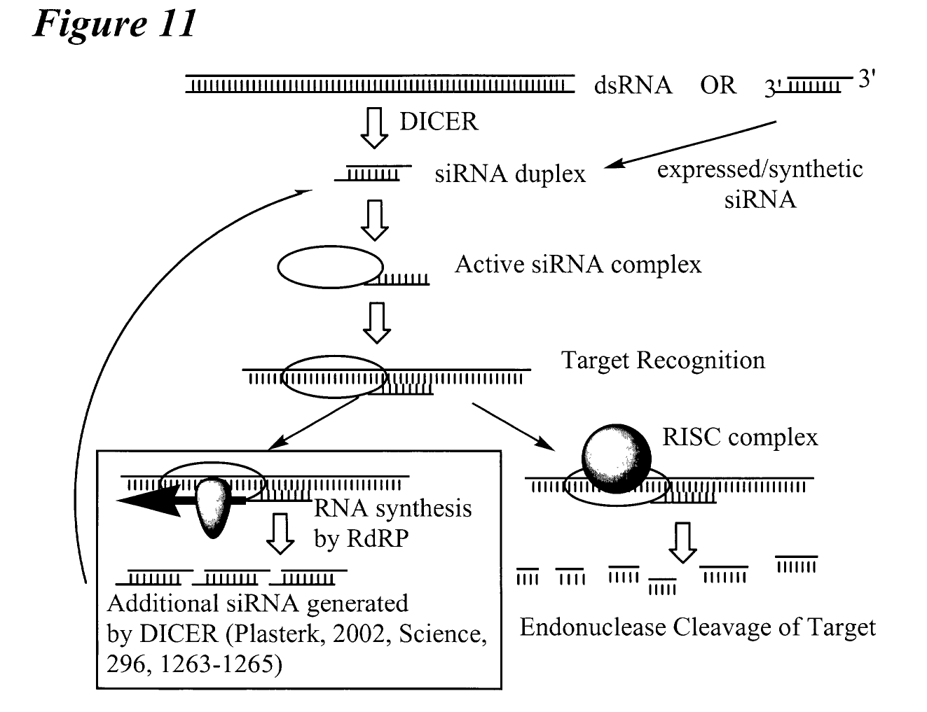

Total RNA was purified from transfected cells at 24 h post-treatment. Real time RT-PCR (Taqman assay) was performed on purified RNA samples using separate primer/probe sets for target HER2 mRNA or control 36B4 RNA. 36B4 RNA levels were used to normalize for differences in well to well sample recovery. RT-PCR conditions were: 30 min at 48° C., 10 min at 95° C., followed by 40 cycles of 15 sec at 95° C. and 1 min at 60° C. Reactions were performed on an ABI Prism 7700 sequence detector. Results for all RNA siNA constructs are shown in FIGS. 3 and 4, whereas results for chemically-modified siNA constructs compared to all RNA (unmodified) constructs are shown in FIGS. 8-10 as the average of triplicate treatments±SD.

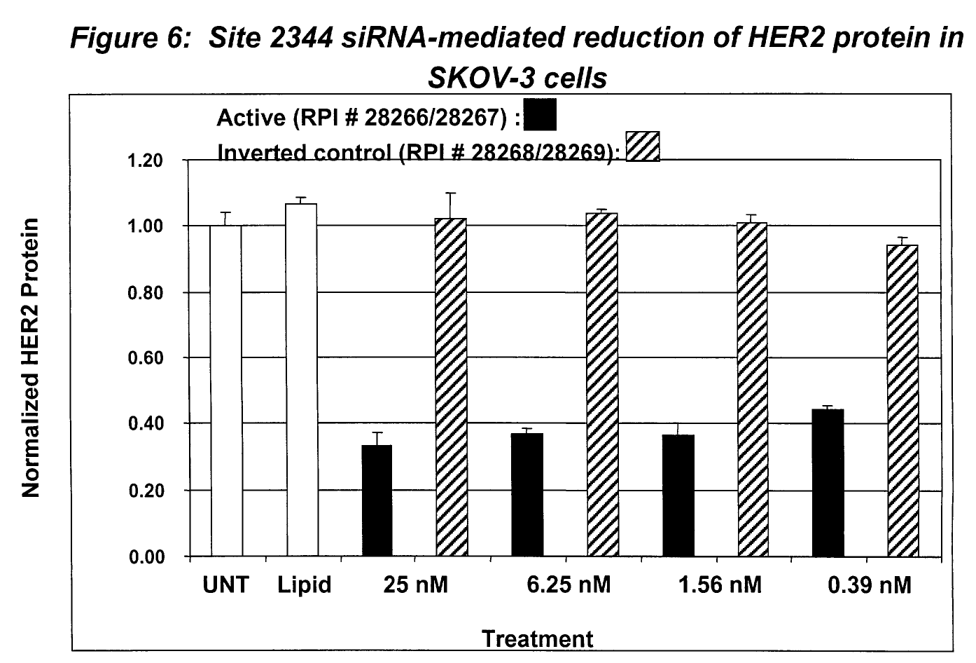

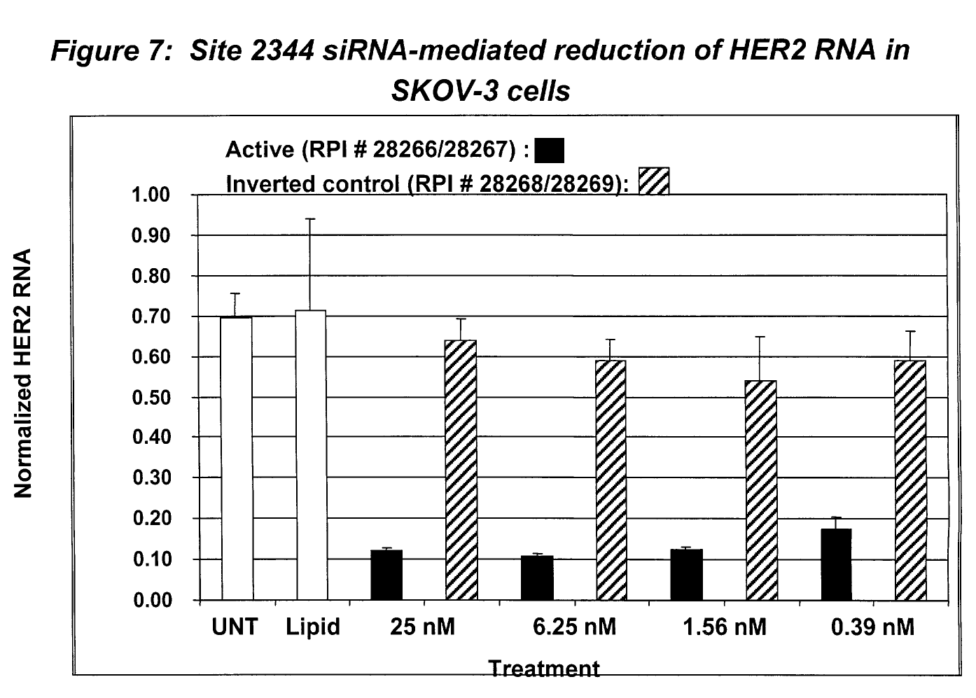

HER2 protein levels were determined by ELISA 48 h post-treatment. HER2 protein levels were normalized to cell number (MTS assay) to control for differences in well to well sample recovery. Results are shown in FIGS. 6 and 7 as the average of duplicate treatments±SD.

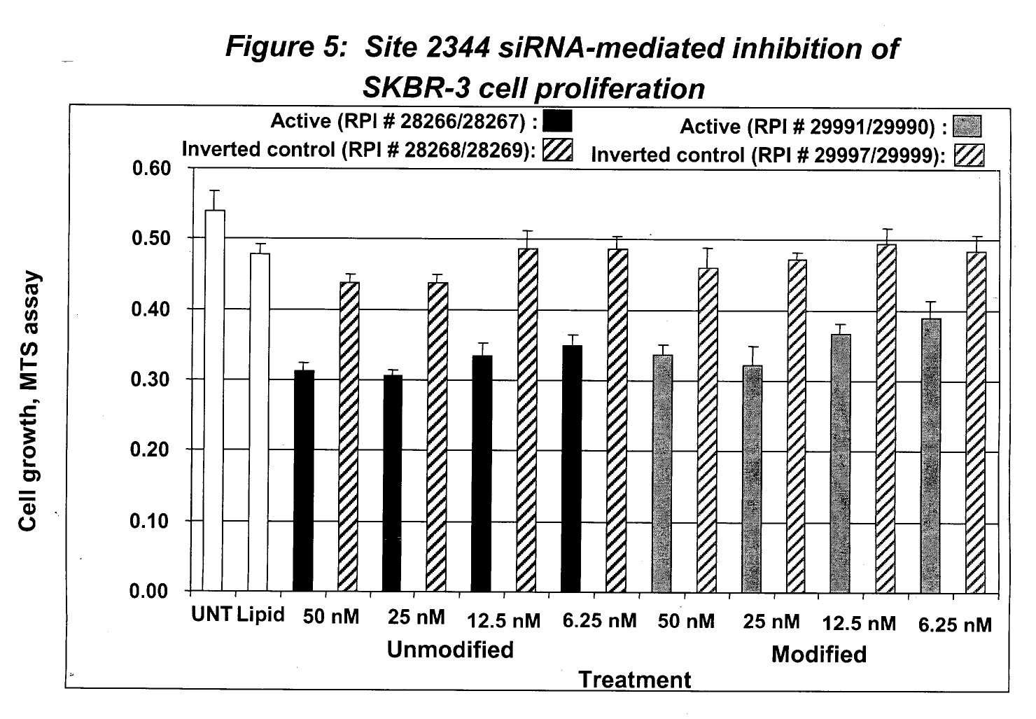

Transfection of siNAs for proliferation assays was the same as above except for the following changes. Short pulse transfection and multiple dosing was used, at 24 h post-plating 5×siNA/lipid complexes were added to and left on cells for 4 h then removed and replaced with growth medium. Final concentration of siNA and inverted controls was 6.25-50 nM. A second dose of siNA/lipid was added at 72 h post-plating and once again replaced with growth medium after 4 h of treatment. Inhibition of cell growth was determined by MTS assay at 48, 72 and 96 h post-treatment. Data for the 96 h point is shown in FIG. 5. Results are shown as the average of triplicate treatments±SD. As shown in FIG. 5, significant inhibition of proliferation is observed using both all RNA and chemically-modified siNA constructs targeting HER2 site 2344 in SKBR-3 cells.

### Example 8

### RNAi In Vitro Assay to Assess siNA Activity

An in vitro assay that recapitulates RNAi in a cell free system is used to evaluate siNA constructs targeting EGFR (e.g., HER1, HER2) RNA targets. The assay comprises the system described by Tuschl et al, 1999, Genes and Development, 13, 3191-3197 and Zamore et al., 2000, Cell, 101, 25-33 adapted for use with HER2 target RNA. A Drosophila extract derived from syncytial blastoderm is used to reconstitute RNAi activity in vitro. Target RNA is generated via in vitro transcription from an appropriate EGFR (e.g., HER1, HER2) expressing plasmid using T7 RNA polymerase or via chemical synthesis as described herein. Sense and antisense siNA strands (for example 20 uM each) are annealed by incubation in buffer (such as 100 mM potassium acetate, 30 mM HEPES-KOH, pH 7.4, 2 mM magnesium acetate) for 1 min. at 90° C. followed by 1 hour at 37° C., then diluted in lysis buffer (for example 100 mM potassium acetate, 30 mM HEPES-KOH at pH 7.4, 2mM magnesium acetate). Annealing can be monitored by gel electrophoresis on an agarose gel in TBE buffer and stained with ethidium bromide. The Drosophila lysate is prepared using zero to two hour old embryos from Oregon R flies collected on yeasted molasses agar that are dechorionated and lysed. The lysate is centrifuged and the supernatant isolated. The assay comprises a reaction mixture containing 50% lysate [vol/vol], RNA (10-50 pM final concentration), and 10% [vol/vol] lysis buffer containing siNA (10 nM final concentration). The reaction mixture also contains 10 mM creatine phosphate, 10 ug.ml creatine phosphokinase, 100 um GTP, 100 uM UTP, 100 uM CTP, 500 uM ATP, 5 mM DTT, 0.1 U/uL RNasin (Promega), and 100 uM of each amino acid. The final concentration of potassium acetate is adjusted to 100 mM. The reactions are pre-assembled on ice and preincubated at 25° C. for 10 minutes before adding RNA, then incubated at 25° C. for an additional 60 minutes. Reactions are quenched with 4 volumes of 1.25×Passive Lysis Buffer (Promega). Target RNA cleavage is assayed by RT-PCR analysis or other methods known in the art and are compared to control reactions in which siNA is omitted from the reaction.

Alternately, internally-labeled target RNA for the assay is prepared by in vitro transcription in the presence of [a-³²p] CTP, passed over a G 50 Sephadex column by spin chromatography and used as target RNA without further purification. Optionally, target RNA is 5′-³²P-end labeled using T4 polynucleotide kinase enzyme. Assays are performed as described above and target RNA and the specific RNA cleavage products generated by RNAi are visualized on an autoradiograph of a gel. The percentage of cleavage is determined by Phosphor Imager® quantitation of bands representing intact control RNA or RNA from control reactions without siNA and the cleavage products generated by the assay.

In one embodiment, this assay is used to determine target sites the EGFR (e.g., HER1, HER2) RNA target for siNA mediated RNAi cleavage, wherein a plurality of siNA constructs are screened for RNAi mediated cleavage of the EGFR (e.g., HER1, HER2) RNA target, for example by analyzing the assay reaction by electrophoresis of labeled target RNA, or by northern blotting, as well as by other methodology well known in the art.

### Example 9

### Diagnostic Uses

The siNA molecules of the invention can be used in a variety of diagnostic applications, such as in identifying molecular targets such as RNA in a variety of applications, for example, in clinical, industrial, environmental, agricultural and/or research settings. Such diagnostic use of siNA molecules involves utilizing reconstituted RNAi systems, for example using cellular lysates or partially purified cellular lysates. siNA molecules of this invention can be used as diagnostic tools to examine genetic drift and mutations within diseased cells or to detect the presence of endogenous or exogenous, for example viral, RNA in a cell. The close relationship between siNA activity and the structure of the target RNA allows the detection of mutations in any region of the molecule, which alters the base-pairing and three-dimensional structure of the target RNA. By using multiple siNA molecules described in this invention, one can map nucleotide changes, which are important to RNA structure and function in vitro, as well as in cells and tissues. Cleavage of target RNAs with siNA molecules can be used to inhibit gene expression and define the role (essentially) of specified gene products in the progression of disease or infection. In this manner, other genetic targets can be defined as important mediators of the disease. These experiments will lead to better treatment of the disease progression by affording the possibility of combination therapies (e.g., multiple siNA molecules targeted to different genes, siNA molecules coupled with known small molecule inhibitors, or intermittent treatment with combinations siNA molecules and/or other chemical or biological molecules). Other in vitro uses of siNA molecules of this invention are well known in the art, and include detection of the presence of mRNAs associated with a disease, infection, or related condition. Such RNA is detected by determining the presence of a cleavage product after treatment with a siNA using standard methodologies, for example fluorescence resonance emission transfer (FRET).

In a specific example, siNA molecules that can cleave only wild-type or mutant forms of the target RNA are used for the assay. The first siNA molecules is used to identify wild-type RNA present in the sample and the second siNA molecules will be used to identify mutant RNA in the sample. As reaction controls, synthetic substrates of both wild-type and mutant RNA will be cleaved by both siNA molecules to demonstrate the relative siNA efficiencies in the reactions and the absence of cleavage of the “non-targeted” RNA species. The cleavage products from the synthetic substrates will also serve to generate size markers for the analysis of wild-type and mutant RNAs in the sample population. Thus each analysis will require two siNA molecules, two substrates and one unknown sample which will be combined into six reactions. The presence of cleavage products will be determined using an RNase protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., disease related or infection related) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations that are not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE 1

HER2 siNA and Target Sequences

| | | | | | | | | |
|---|---|---|---|---|---|---|---|---|
| | | Seq | | | Seq | | | Seq |
| Pos | Target Sequence | ID | UPos | Upper seq | ID | Lpos | Lower seq | ID |

| | | | | | | | | |
|---|---|---|---|---|---|---|---|---|
| 1 | AAGGGGAGGUAACCCUGGC | 1 | 1 | AAGGGGAGGUAACCCUGGC | 1 | 23 | GCCAGGGUUACCUCCCCUU | 250 |
| 19 | CCCCUUUGGUCGGGGCCCC | 2 | 19 | CCCCUUUGGUCGGGGCCCC | 2 | 41 | GGGGCCCCGACCXAAGGGG | 251 |
| 37 | CGGGCAGCCGCGCGCCCCU | 3 | 37 | CGGGCAGCCGCGCGCCCCU | 3 | 59 | AGGGGCGCGCGGCUGCCCG | 252 |
| 55 | UUCCCACGGGGCCCUUUAC | 4 | 55 | UUCCCACGGGGCCCUUUAC | 4 | 77 | GUAAAGGGCCCCGUGGGAA | 253 |
| 73 | CUGCGCCGCGOGCCCGGCC | 5 | 73 | CUGCGCCGCGCGCCCGGCC | 5 | 95 | GGCCGGGCGCGCGGCGCAG | 254 |
| 91 | CCCCACCCCUCGCAGCACC | 6 | 91 | CCCCACCCCUCGCAGCACC | 6 | 113 | GGUGCUGCGAGGGGUGGGG | 255 |
| 109 | CCCGCGCCCCGCGCCCUCC | 7 | 109 | CCCGCGCCCCGCGCCCUCC | 7 | 131 | GGAGGGCGCGGGGCGCGGG | 256 |
| 127 | CCAGCCGGGUCCAGCCGGA | 8 | 127 | CCAGCCGGGUCCAGCCGGA | 8 | 149 | UCCGGCUGGACCCGGCUGG | 257 |
| 145 | AGCCAUGGGGCCGGAGCCG | 9 | 145 | AGCCAUGGGGCCGGAGCCG | 9 | 167 | CGGCUCCGGCCCCAUGGCU | 258 |
| 163 | GCAGUGAGCACCAUGGAGC | 10 | 163 | GCAGUGAGCACCAUGGAGC | 10 | 185 | GCUCCAUGGUGCUCACUGC | 259 |
| 181 | CUGGCGGCCUUGUGCCGCU | 11 | 181 | CUGGCGGCCUUGUGCCGCU | 11 | 203 | AGCGGCACAAGGCCGCCAG | 260 |
| 199 | UGGGGGCUCCUCCUCGCCC | 12 | 199 | UGGGGGCUCCUCCUCGCCC | 12 | 221 | GGGCGAGGAGGAGCCCCCA | 261 |
| 217 | CUCUUGCCCCCCGGAGCCG | 13 | 217 | CUCUUGCCCCCCGGAGCCG | 13 | 239 | CGGCUCCGGGGGGCAAGAG | 262 |
| 235 | GCGAGCACCCAAGUGUGCA | 14 | 235 | GCGAGCACCCAAGUGUGCA | 14 | 257 | UGCACACUUGGGUGCUCGC | 263 |
| 253 | ACCGGCACAGACAUGAAGC | 15 | 253 | ACCGGCACAGACAUGAAGC | 15 | 275 | GCUUCAUGUCUGUGCCGGU | 264 |
| 271 | CUGCGGCUCCCUGCCAGUC | 16 | 271 | CUGCGGCUCCCUGCCAGUC | 16 | 293 | GACUGGCAGGGAGCCGCAG | 265 |
| 289 | CCCGAGACCCACCUGGACA | 17 | 289 | CCCGAGACCCACCUGGACA | 17 | 311 | UGUCCAGGUGGGUCUCGGG | 266 |
| 307 | AUGCUCCGCCACCUCUACC | 18 | 307 | AUGCUCCGCCACCUCUACC | 18 | 329 | GGUAGAGGUGGCGGAGCAU | 267 |
| 325 | CAGGGCUGCCAGGUGGUGC | 19 | 325 | CAGGGCUGCCAGGUGGUGC | 19 | 347 | GCACCACCUGGCAGCCCUG | 268 |
| 343 | CAGGGAAACCUGGAACUCA | 20 | 343 | CAGGGCAACCUGGAACUCA | 20 | 365 | UGAGUUCCAGGUUUCCCUG | 269 |
| 361 | ACCUACCUGCCCACCAAUG | 21 | 361 | ACCUACCUGCCCACCAAUG | 21 | 383 | CAUUGGUGGGCAGGUAGGU | 270 |
| 379 | GCCAGCCUGUCCUUCCUGC | 22 | 379 | GCCAGCCUGUCCUUCCUGC | 22 | 401 | GCAGGAAGGACAGGCUGGC | 271 |
| 397 | CAGGAUAUCCAGGAGGUGC | 23 | 397 | CAGGAUAUCCAGGAGGUGC | 23 | 419 | GCACCUCCUGGAUAUCCUG | 272 |
| 415 | CAGGGCUACGUGCUCAUCG | 24 | 415 | CAGGGCUACGUGCUCAUCG | 24 | 437 | CGAUGAGCACGUAGCCCUG | 273 |
| 433 | GCUCACAACCAAGUGAGGC | 25 | 433 | GCUCACAACCAAGUGAGGC | 25 | 455 | GCCUCACUUGGUUGUGAGC | 274 |
| 451 | CAGGUCCCACUGCAGAGGC | 26 | 451 | CAGGUCCCACUGCAGAGGC | 26 | 473 | GCCUCUGCAGUGGGACCUG | 275 |
| 469 | CUGCGGAUUGUGCGAGGCA | 27 | 469 | CUGCGGAUUGUGCGAGGCA | 27 | 491 | UGCCUCGCACAAUCCGCAG | 276 |
| 487 | ACCCAGCUCUUUGAGGACA | 28 | 487 | ACCCAGCUCUUUGAGGACA | 28 | 509 | UGUCCUCAAAGAGCUGGGU | 277 |
| 505 | AACUAUGCCCUGGCCGUGC | 29 | 505 | AACUAUGCCCUGGCCGUGC | 29 | 527 | GCACGGCCAGGGCAUAGUU | 278 |
| 523 | CUAGACAAUGGAGACCCGC | 30 | 523 | CUAGACAAUGGAGACCCGC | 30 | 545 | GCGGGUCUCCAUUGUCUAG | 279 |
| 541 | CUGAACAAUACCACCCCUG | 31 | 541 | CUGAACAAUACCACCCCUG | 31 | 563 | CAGGGGUGGUAUUGUUCAG | 280 |
| 559 | GUCACAGGGGCCUCCCCAG | 32 | 559 | GUCACAGGGGCCUCCCCAG | 32 | 581 | CUGGGGAGGCCCCUGUGAC | 281 |
| 577 | GGAGGCCUGCGGGAGCUGC | 33 | 577 | GGAGGCCUGCGGGAGCUGC | 33 | 599 | GCAGCUCCCGCAGGCCUCC | 282 |
| 595 | CAGCUUCGAAGCCUCACAG | 34 | 595 | CAGCUUCGAAGCCUCACAG | 34 | 617 | CUGUGAGGCUUCGAAGCUG | 283 |
| 613 | GAGAUCUUGAAAGGAGGGG | 35 | 613 | GAGAUCUUGAAAGGAGGGG | 35 | 635 | CCCCUCCUUUCAAGAUCUC | 284 |
| 631 | GUCUUGAUCCAGCGGAACC | 36 | 631 | GUCUUGAUCCAGCGGAACC | 36 | 653 | GGUUCCGCUGGAUCAAGAC | 285 |
| 649 | CCCCAGCUCUGCUACCAGG | 37 | 649 | CCCCAGCUCUGCUACCAGG | 37 | 671 | CCUGGUAGCAGAGCUGGGG | 286 |
| 667 | GACACGAUUUUGUGGAAGG | 38 | 667 | GACACGAUUUUGUGGAAGG | 38 | 689 | CCUUCCACAAAAUCGUGUC | 287 |
| 685 | GACAUCUUCCACAAGAACA | 39 | 685 | GACAUCUUCCACAAGAACA | 39 | 707 | UGUUCUUGUGGAAGAUGUC | 288 |
| 703 | AACCAGCUGGCUCUCACAC | 40 | 703 | AACCAGCUGGCUCUCACAC | 40 | 725 | GUGUGAGAGCCAGCUGGUU | 289 |
| 721 | CUGAUAGACACCAACCGCU | 41 | 721 | CUGAUAGACACCAACCGCU | 41 | 743 | AGCGGUUGGUGUCUAUCAG | 290 |
| 739 | UCUCGGGCCUGCCACCCCU | 42 | 739 | UCUCGGGCCUGCCACCCCU | 42 | 761 | AGGGGUGGCAGGCCCGAGA | 291 |
| 757 | UGUUCUCCGAUGUGUAAGG | 43 | 757 | UGUUCUCCGAUGUGUAAGG | 43 | 779 | CCUUACACAUCGGAGAACA | 292 |
| 775 | GGCUCCCGCUGCUGGGGAG | 44 | 775 | GGCUCCCGCUGCUGGGGAG | 44 | 797 | CUCCCCAGCAGCGGGAGCC | 293 |
| 793 | GAGAGUUCUGAGGAUUGUC | 45 | 793 | GAGAGUUCUGAGGAUUGUC | 45 | 815 | GACAAUCCUCAGAACUCUC | 294 |
| 811 | CAGAGCCUGACGCGCACUG | 46 | 811 | CAGAGCCUGACGCGCACUG | 46 | 833 | CAGUGOGOGUCAGGOUCUG | 295 |
| 829 | GUCUGUGCCGGUGGCUGUG | 47 | 829 | GUCUGUGCCGGUGGCUGUG | 47 | 851 | CACAGCCACCGGCACAGAC | 296 |
| 847 | GCCCGCUGCAAGGGGCCAC | 48 | 847 | GCCCGCUGCAAGGGGCCAC | 48 | 869 | GUGGCCCCUUGCAGCGGGC | 297 |
| 865 | CUGCCCACUGACUGCUGCC | 49 | 865 | CUGCCCACUGACUGCUGCC | 49 | 887 | GGCAGCAGUCAGUGGGCAG | 298 |
| 883 | CAUGAGCAGUGUGCUGCCG | 50 | 883 | CAUGAGCAGUGUGCUGCCG | 50 | 905 | CGGCAGCACACUGCUCAUG | 299 |
| 901 | GGCUGCACGGGCCCCAAGC | 51 | 901 | GGCUGCACGGGCCCCAAGC | 51 | 923 | GCUUGGGGCCCGUGCAGCC | 300 |
| 919 | CACUCUGACUGCCUGGCCU | 52 | 919 | CACUCUGACUGCCUGGCCU | 52 | 941 | AGGCCAGGCAGUCAGAGUG | 301 |
| 937 | UGCCUCCACUUCAACCACA | 53 | 937 | UGCCUCCACUUCAACCACA | 53 | 959 | UGUGGUUGAAGUGGAGGCA | 302 |
| 955 | AGUGGCAUCUGUGAGCUGC | 54 | 955 | AGUGGCAUCUGUGAGCUGC | 54 | 977 | GCAGCUCACAGAUGCCACU | 303 |
| 973 | CACUGCCCAGCCCUGGUCA | 55 | 973 | CACUGCCCAGCCCUGGUCA | 55 | 995 | UGACCAGGGCUGGGCAGUG | 304 |
| 991 | ACCUACAACACAGACACGU | 56 | 991 | ACCUACAACACAGACACGU | 56 | 1013 | ACGUGUCUGUGUUGUAGGU | 305 |
| 1009 | UUUGAGUCCAUGCCCAAUC | 57 | 1009 | UUUGAGUCCAUGCCCAAUC | 57 | 1031 | GAUUGGGCAUGGACUCAAA | 306 |
| 1027 | CCCGAGGGCCGGUAUACAU | 58 | 1027 | CCCGAGGGCCGGUAUACAU | 58 | 1049 | AUGUAUACCGGCCCUCGGG | 307 |
| 1045 | UUCGGCGCCAGCUGUGUGA | 59 | 1045 | UUCGGCGCCAGCUGUGUGA | 59 | 1067 | UCACACAGCUGGCGCCGAA | 308 |
| 1063 | ACUGCCUGUCCCUACAACU | 60 | 1063 | ACUGCCUGUCCCUACAACU | 60 | 1085 | AGUUGUAGGGACAGGCAGU | 309 |
| 1081 | UACCUUUCUACGGACGUGG | 61 | 1081 | UACCUUUCUACGGACGUGG | 61 | 1103 | CCACGUCCGUAGAAAGGUA | 310 |
| 1099 | GGAUCCUGCACCCUCGUCU | 62 | 1099 | GGAUCCUGCACCCUCGUCU | 62 | 1121 | AGACGAGGGUGCAGGAUCC | 311 |
| 1117 | UGCCCCCUGCACAACCAAG | 63 | 1117 | UGCCCCCUGCACAACCAAG | 63 | 1139 | CUUGGUUGUGCAGGGGGCA | 312 |
| 1135 | GAGGUGACAGCAGAGGAUG | 64 | 1135 | GAGGUGACAGCAGAGGAUG | 64 | 1157 | CAUCCUCUGCUGUCACCUC | 313 |
| 1153 | GGAACACAGCGGUGUGAGA | 65 | 1153 | GGAACACAGCGGUGUGAGA | 65 | 1175 | UCUCACACCGCUGUGUUCC | 314 |
| 1171 | AAGUGCAGCAAGCCCUGUG | 66 | 1171 | AAGUGCAGCAAGCCCUGUG | 66 | 1193 | CACAGGGCUUGCUGCACUU | 315 |
| 1189 | GCCCGAGUGUGCUAUGGUC | 67 | 1189 | GCCCGAGUGUGCUAUGGUC | 67 | 1211 | GACCAUAGCACACUCGGGC | 316 |
| 1207 | CUGGGCAUGGAGCACUUGC | 68 | 1207 | CUGGGCAUGGAGCACUUGC | 68 | 1229 | GCAAGUGCUCCAUGCCCAG | 317 |
| 1225 | CGAGAGGUGAGGGCAGUUA | 69 | 1225 | CGAGAGGUGAGGGCAGUUA | 69 | 1247 | UAACUGCCCUCACCUCUCG | 318 |
| 1243 | ACCAGUGCCAAUAUCCAGG | 70 | 1243 | ACCAGUGCCAAUAUCCAGG | 70 | 1265 | CCUGGAUAUUGGCACUGGU | 319 |
| 1261 | GAGUUUGCUGGCUGCAAGA | 71 | 1261 | GAGUUUGCUGGCUGCAAGA | 71 | 1283 | UCUUGCAGCCAGCAAACUC | 320 |
| 1279 | AAGAUCUUUGGGAGCCUGG | 72 | 1279 | AAGAUCUUUGGGAGCCUGG | 72 | 1301 | CCAGGCUCCCAAAGAUCUU | 321 |
| 1297 | GCAUUUCUGCCGGAGAGCU | 73 | 1297 | GCAUUUCUGCCGGAGAGCU | 73 | 1319 | AGCUCUCCGGCAGAAAUGC | 322 |
| 1315 | UUUGAUGGGGACCCAGCCU | 74 | 1315 | UUUGAUGGGGACCCAGCCU | 74 | 1337 | AGGCUGGGUCCCCAUCAAA | 323 |
| 1333 | UCCAACACUGCCCCGCUCC | 75 | 1333 | UCCAACACUGCCCCGCUCC | 75 | 1355 | GGAGCGGGGCAGUGUUGGA | 324 |
| 1351 | CAGCCAGAGCAGCUCCAAG | 76 | 1351 | CAGCCAGAGCAGCUCCAAG | 76 | 1373 | CUUGGAGCUGCUCUGGCUG | 325 |
| 1369 | GUGUUUGAGACUCUGGAAG | 77 | 1369 | GUGUUUGAGACUCUGGAAG | 77 | 1391 | CUUCCAGAGUCUCAAACAC | 326 |
| 1387 | GAGAUCACAGGUUACCUAU | 78 | 1387 | GAGAUCACAGGUUACCUAU | 78 | 1409 | AUAGGUAACCUGUGAUCUC | 327 |
| 1405 | UACAUCUCAGCAUGGCCGG | 79 | 1405 | UACAUCUCAGCAUGGCCGG | 79 | 1427 | CCGGCCAUGCUGAGAUGUA | 328 |
| 1423 | GACAGCCUGGCUGACCUCA | 80 | 1423 | GACAGCCUGCCUGACCUCA | 80 | 1445 | UGAGGUCAGGCAGGCUGUC | 329 |
| 1441 | AGCGUCUUCCAGAACCUGC | 81 | 1441 | AGCGUCUUCCAGAACCUGC | 81 | 1463 | GCAGGUUCUGGAAGACGCU | 330 |
| 1459 | CAAGUAAUCCGGGGACGAA | 82 | 1459 | CAAGUAAUCCGGGGACGAA | 82 | 1481 | UUCGUCCCCGGAUUACUUG | 331 |
| 1477 | AUUCUGCACAAUGGCGCCU | 83 | 1477 | AUUCUGCACAAUGGCGCCU | 83 | 1499 | AGGCGCCAUUGUGCAGAAU | 332 |
| 1495 | UACUCGCUGACCCUGCAAG | 84 | 1495 | UACUCGCUGACCCUGCAAG | 84 | 1517 | CUUGCAGGGUCAGCGAGUA | 333 |
| 1513 | GGGCUGGGCAUCAGCUGGC | 85 | 1513 | GGGCUGGGCAUCAGCUGGC | 85 | 1535 | GCCAGCUGAUGCCCAGCCC | 334 |
| 1531 | CUGGGGCUGCGCUCACUGA | 86 | 1531 | CUGGGGCUGCGCUCACUGA | 86 | 1553 | UCAGUGAGCGCAGCCCCAG | 335 |
| 1549 | AGGGAACUGGGCAGUGGAC | 87 | 1549 | AGGGAACUGGGCAGUGGAC | 87 | 1571 | GUCCACUGCCCAGUUCCCU | 336 |
| 1567 | CUGGCCCUCAUCCACCAUA | 88 | 1567 | CUGGCCCUCAUCCACCAUA | 88 | 1589 | UAUGGUGGAUGAGGGCCAG | 337 |
| 1585 | AACACCCACCUCUGCUUCG | 89 | 1585 | AACACCCACCUCUGCUUCG | 89 | 1607 | CGAAGCAGAGGUGGGUGUU | 338 |
| 1603 | GUGCACACGGUGCCCUGGG | 90 | 1603 | GUGCACACGGUGCCCUGGG | 90 | 1625 | CCCAGGGCACCGUGUGCAC | 339 |
| 1621 | GACCAGCUCUUUCGGAACC | 91 | 1621 | GACCAGCUCUUUCGGAACC | 91 | 1643 | GGUUCCGAAAGAGCUGGUC | 340 |
| 1639 | CCGCACCAAGCUCUGCUCC | 92 | 1639 | CCGCACCAAGCUCUGCUCC | 92 | 1661 | GGAGCAGAGCUUGGUGCGG | 341 |
| 1657 | CACACUGCCAACCGGCCAG | 93 | 1657 | CACACUGCCAACCGGCCAG | 93 | 1679 | CUGGCCGGUUGGCAGUGUG | 342 |
| 1675 | GAGGACGAGUGUGUGGGCG | 94 | 1675 | GAGGACGAGUGUGUGGGCG | 94 | 1697 | CGCCCACACACUCGUCCUC | 343 |
| 1693 | GAGGGCCUGGCCUGCCACC | 95 | 1693 | GAGGGCCUGGCCUGCCACC | 95 | 1715 | GGUGGCAGGCCAGGCCCUC | 344 |
| 1711 | CAGCUGUGCGCCCGAGGGC | 96 | 1711 | CAGCUGUGCGCCCGAGGGC | 96 | 1733 | GCCCUCGGGCGCACAGCUG | 345 |
| 1729 | CACUGCUGGGGUCCAGGGC | 97 | 1729 | CACUGCUGGGGUCCAGGGC | 97 | 1751 | GCCCUGGACCCCAGCAGUG | 346 |
| 1747 | CCCACCCAGUGUGUCAACU | 98 | 1747 | CCCACCCAGUGUGUCAACU | 98 | 1769 | AGUUGACACACUGGGUGGG | 347 |
| 1765 | UGCAGCCAGUUCCUUCGGG | 99 | 1765 | UGCAGCCAGUUCCUUCGGG | 99 | 1787 | CCCGAAGGAACUGGCUGCA | 348 |
| 1783 | GGCCAGGAGUGCGUGGAGG | 100 | 1783 | GGCCAGGAGUGCGUGGAGG | 100 | 1805 | CCUCCACGCACUCCUGGCC | 349 |
| 1801 | GAAUGCCGAGUACUGCAGG | 101 | 1801 | GAAUGCCGAGUACUGCAGG | 101 | 1823 | CCUGCAGUACUCGGCAUUC | 350 |
| 1819 | GGGCUCCCCAGGGAGUAUG | 102 | 1819 | GGGCUCCCCAGGGAGUAUG | 102 | 1841 | CAUACUCCCUGGGGAGCCC | 351 |
| 1837 | GUGAAUGCCAGGCACUGUU | 103 | 1837 | GUGAAUGCCAGGCACUGUU | 103 | 1859 | AACAGUGCCUGGCAUUCAC | 352 |
| 1855 | UUGCCGUGCCACCCUGAGU | 104 | 1855 | UUGCCGUGCCACCCUGAGU | 104 | 1877 | ACUCAGGGUGGCACGGCAA | 353 |
| 1873 | UGUCAGCCCCAGAAUGGCU | 105 | 1873 | UGUCAGCCCCAGAAUGGCU | 105 | 1895 | AGCCAUUCUGGGGCUGACA | 354 |
| 1891 | UCAGUGACCUGUUUUGGAC | 106 | 1891 | UCAGUGACCUGUUUUGGAC | 106 | 1913 | GUCCAAAACAGGUCACUGA | 355 |
| 1909 | CCGGAGGCUGACCAGUGUG | 107 | 1909 | CCGGAGGCUGACCAGUGUG | 107 | 1931 | CACACUGGUCAGCCUCCGG | 356 |
| 1927 | GUGGCCUGUGCCCACUAUA | 108 | 1927 | GUGGCCUGUGCCCACUAUA | 108 | 1949 | UAUAGUGGGCACAGGCCAC | 357 |
| 1945 | AAGGACCCUCCCUUCUGCG | 109 | 1945 | AAGGACCCUCCCUUCUGCG | 109 | 1967 | CGCAGAAGGGAGGGUCCUU | 358 |
| 1963 | GUGGCCCGCUGCCCCAGCG | 110 | 1963 | GUGGCCCGCUGCCCCAGCG | 110 | 1985 | CGCUGGGGCAGCGGGCCAC | 359 |
| 1981 | GGUGUGAAACCUGACCUCU | 111 | 1981 | GGUGUGAAACCUGACCUCU | 111 | 2003 | AGAGGUCAGGUUUCACACC | 360 |
| 1999 | UCCUACAUGOCCAUCUGGA | 112 | 1999 | UCCUACAUGCCCAUCUGGA | 112 | 2021 | UCCAGAUGGGCAUGUAGGA | 361 |
| 2017 | AAGUUUCCAGAUGAGGAGG | 113 | 2017 | AAGUUUCCAGAUGAGGAGG | 113 | 2039 | CCUCCUCAUCUGGAAACUU | 362 |
| 2035 | GGCGCAUGCCAGCCUUGCC | 114 | 2035 | GGCGCAUGCCAGCCUUGCC | 114 | 2057 | GGCAAGGCUGGCAUGCGCC | 363 |
| 2053 | CCCAUCAACUGCACCCACU | 115 | 2053 | CCCAUCAACUGCACCCACU | 115 | 2075 | AGUGGGUGCAGUUGAUGGG | 364 |
| 2071 | UCCUGUGUGGACCUGGAUG | 116 | 2071 | UCCUGUGUGGACCUGGAUG | 116 | 2093 | CAUCCAGGUCCACACAGGA | 365 |
| 2089 | GACAAGGGCUGCCCCGCCG | 117 | 2089 | GACAAGGGCUGCCCCGCCG | 117 | 2111 | CGGCGGGGCAGCCCUUGUC | 366 |
| 2107 | GAGCAGAGAGCCAGCCCUC | 118 | 2107 | GAGCAGAGAGCCAGCCCUC | 118 | 2129 | GAGGGCUGGCUCUCUGCUC | 367 |
| 2125 | CUGACGUCCAUCAUCUCUG | 119 | 2125 | CUGACGUCCAUCAUCUCUG | 119 | 2147 | CAGAGAUGAUGGACGUCAG | 368 |
| 2143 | GCGGUGGUUGGCAUUCUGC | 120 | 2143 | GCGGUGGUUGGCAUUCUGC | 120 | 2165 | GCAGAAUGCCAACCACCGC | 369 |
| 2161 | CUGGUCGUGGUCUUGGGGG | 121 | 2161 | CUGGUCGUGGUCUUGGGGG | 121 | 2183 | CCCCCAAGACCACGACCAG | 370 |
| 2179 | GUGGUCUUUGGGAUCCUCA | 122 | 2179 | GUGGUCUUUGGGAUCCUCA | 122 | 2201 | UGAGGAUCCCAAAGACCAC | 371 |
| 2197 | AUCAAGCGACGGCAGCAGA | 123 | 2197 | AUCAAGCGACGGCAGCAGA | 123 | 2219 | UCUGCUGCCGUCGCUUGAU | 372 |
| 2215 | AAGAUCCGGAAGUACACGA | 124 | 2215 | AAGAUCCGGAAGUACACGA | 124 | 2237 | UCGUGUACUUCCGGAUCUU | 373 |
| 2233 | AUGCGGAGACUGCUGCAGG | 125 | 2233 | AUGCGGAGACUGCUGCAGG | 125 | 2255 | CCUGCAGCAGUCUCCGCAU | 374 |
| 2251 | GAAACGGAGCUGGUGGAGC | 126 | 2251 | GAAACGGAGCUGGUGGAGC | 126 | 2273 | GCUCCACCAGCUCCGUUUC | 375 |
| 2269 | CCGCUGACACCUAGCGGAG | 127 | 2269 | CCGCUGACACCUAGCGGAG | 127 | 2291 | CUCCGCUAGGUGUCAGCGG | 376 |
| 2287 | GCGAUGCCCAACCAGGCGC | 128 | 2287 | GCGAUGCCCAACCAGGCGC | 128 | 2309 | GCGCCUGGUUGGGCAUCGC | 377 |
| 2305 | CAGAUGCGGAUCCUGAAAG | 129 | 2305 | CAGAUGCGGAUCCUGAAAG | 129 | 2327 | CUUUCAGGAUCCGCAUCUG | 378 |
| 2323 | GAGACGGAGCUGAGGkAGG | 130 | 2323 | GAGACGGAGCUGAGGAAGG | 130 | 2345 | CCUUCCUCAGCUCCGUCUC | 379 |
| 2341 | GUGAAGGUGCUUGGAUCUG | 131 | 2341 | GUGAAGGUGCUUGGAUCUG | 131 | 2363 | CAGAUCCAAGCACCUUCAC | 380 |
| 2359 | GGCGCUUUUGGCACAGUCU | 132 | 2359 | GGCGCUUUUGGCACAGUCU | 132 | 2381 | AGACUGUGCCAAAAGCGCC | 381 |
| 2377 | UACAAGGGCAUCUGGAUCC | 133 | 2377 | UACAAGGGCAUCUGGAUCC | 133 | 2399 | GGAUCCAGAUGCCCUUGUA | 382 |
| 2395 | CCUGAUGGGGAGAAUGUGA | 134 | 2395 | CCUGAUGGGGAGAAUGUGA | 134 | 2417 | UCACAUUCUCCCCAUCAGG | 383 |
| 2413 | AAAAUUCCAGUGGCCAUCA | 135 | 2413 | AAAAUUCCAGUGGCCAUCA | 135 | 2435 | UGAUGGCCACUGGAAUUUU | 384 |
| 2431 | AAAGUGUUGAGGGAAACA | 136 | 2431 | AAAGUGUUGAGGGAAAACA | 136 | 2453 | UGUUUUCCCUCAACACUUU | 385 |
| 2449 | ACAUCCCCCAAAGCCAACA | 137 | 2449 | ACAUCCCCCAAAGCCAACA | 137 | 2471 | UGUUGGCUUUGGGGGAUGU | 386 |
| 2467 | AAAGAAAUCUUAGACGAAG | 138 | 2467 | AAAGAAAUCUUAGACGAAG | 138 | 2489 | CUUCGUCUAAGAUUUCUUU | 387 |
| 2485 | GCAUACGUGAUGGCUGGUG | 139 | 2485 | GCAUACGUGAUGGCUGGUG | 139 | 2507 | CACCAGCCAUCACGUAUGC | 388 |
| 2503 | GUGGGCUCCCCAUAUGUCU | 140 | 2503 | GUGGGCUCCCCAUAUGUCU | 140 | 2525 | AGACAUAUGGGGAGCCCAC | 389 |
| 2521 | UCCCGCCUUCUGGGCAUCU | 141 | 2521 | UCCCGCCUUCUGGGCAUCU | 141 | 2543 | AGAUGCCCAGAAGGCGGGA | 390 |
| 2539 | UGCCUGACAUCCACGGUGC | 142 | 2539 | UGCCUGACAUCCACGGUGC | 142 | 2561 | GCACCGUGGAUGUCAGGCA | 391 |
| 2557 | CAGCUGGUGACACAGCUUA | 143 | 2557 | CAGCUGGUGACACAGCUUA | 143 | 2579 | UAAGCUGUGUCACCAGCUG | 392 |
| 2575 | AUGCCCUAUGGCUGCCUCU | 144 | 2575 | AUGCCCUAUGGCUGCCUCU | 144 | 2597 | AGAGGCAGCCAUAGGGCAU | 393 |
| 2593 | UUAGACCAUGUCCGGGAAA | 145 | 2593 | UUAGACCAUGUCCGGGAAA | 145 | 2615 | UUUCCCGGACAUGGUCUAA | 394 |
| 2611 | AACCGCGGACGCCUGGGCU | 146 | 2611 | AACCGCGGACGCCUGGGCU | 146 | 2633 | AGCCCAGGCGUCCGCGGUU | 395 |
| 2629 | UCCCAGGACCUGCUGAACU | 147 | 2629 | UCCCAGGACCUGCUGAACU | 147 | 2651 | AGUUCAGCAGGUCCUGGGA | 396 |
| 2647 | UGGUGUAUGCAGAUUGCCA | 148 | 2647 | UGGUGUAUGCAGAUUGCCA | 148 | 2669 | UGGCAAUCUGCAUACACCA | 397 |
| 2665 | AAGGGGAUGAGCUACCUGG | 149 | 2665 | AAGGGGAUGAGCUACCUGG | 149 | 2687 | CCAGGUAGCUCAUCCCCUU | 398 |
| 2683 | GAGGAUGUGCGGCUCGUAC | 150 | 2683 | GAGGAUGUGCGGCUOGUAC | 150 | 2705 | GUACGAGCCGCACAUCCUC | 399 |
| 2701 | CACAGGGACUUGGCCGCUC | 151 | 2701 | CACAGGGACUUGGCCGCUC | 151 | 2723 | GAGCGGCCAAGUCCCUGUG | 400 |
| 2719 | CGGPACGUGCUGGUCAAGA | 152 | 2719 | CGGAACGUGCUGGUCAAGA | 152 | 2741 | UCUUGACCAGCACGUUCCG | 401 |
| 2737 | AGUCCCAACCAUGUCAAAA | 153 | 2737 | AGUCCCAACCAUGUCAAAA | 153 | 2759 | UUUUGACAUGGUUGGGACU | 402 |
| 2755 | AUUACAGACUUCGGGCUGG | 154 | 2755 | AUUACAGACUUCGGGCUGG | 154 | 2777 | CCAGCCCGAAGUCUGUAAU | 403 |
| 2773 | GCUCGGCUGCUGGACAUUG | 155 | 2773 | GCUCGGCUGCUGGACAUUG | 155 | 2795 | CAAUGUCCAGCAGCCGAGC | 404 |
| 2791 | GACGAGACAGAGUACCAUG | 156 | 2791 | GACGAGACAGAGUACCAUG | 156 | 2813 | CAUGGUACUCUGUCUOGUC | 405 |
| 2809 | GCAGAUGGGGGCAAGGUGC | 157 | 2809 | GCAGAUGGGGGCAAGGUGC | 157 | 2831 | GCACCUUGCCCCCAUCUGC | 406 |
| 2827 | CCCAUCAAGUGGAUGGCGC | 158 | 2827 | CCCAUCAAGUGGAUGGCGC | 158 | 2849 | GCGCCAUCCACUUGAUGGG | 407 |
| 2845 | CUGGAGUCCAUUCUCCGCC | 159 | 2845 | CUGGAGUCCAUUCUCCGCC | 159 | 2867 | GGCGGAGAAUGGACUCCAG | 408 |
| 2863 | CGGCGGUUCACCCACCAGA | 160 | 2863 | CGGCGGUUCACCCACCAGA | 160 | 2885 | UCUGGUGGGUGAACCGCCG | 409 |
| 2881 | AGUGAUGUGUGGAGUUAUG | 161 | 2881 | AGUGAUGUGUGGAGUUAUG | 161 | 2903 | CAUAACUCCACACAUCACU | 410 |
| 2899 | GGUGUGACUGUGUGGGAGC | 162 | 2899 | GGUGUGACUGUGUGGGAGC | 162 | 2921 | GCUCCCACACAGUCACACC | 411 |
| 2917 | CUGAUGACUUUUGGGGCCA | 163 | 2917 | CUGAUGACUUUUGGGGCCA | 163 | 2939 | UGGCCCCAAAAGUCAUCAG | 412 |
| 2935 | AAACCUUACGAUGGGAUCC | 164 | 2935 | AAACCUUACGAUGGGAUCC | 164 | 2957 | GGAUCCCAUCGUAAGGUUU | 413 |
| 2953 | CCAGCCCGGGAGAUCCCUG | 165 | 2953 | CCAGCCCGGGAGAUCCCUG | 165 | 2975 | CAGGGAUCUCCCGGGCUGG | 414 |
| 2971 | GACCUGCUGGAAAAGGGGG | 166 | 2971 | GACCUGCUGGAAAAGGGGG | 166 | 2993 | CCCCCUUUUCCAGCAGGUC | 415 |
| 2989 | GAGCGGCUGCCCCAGCCCC | 167 | 2989 | GAGCGGCUGCCCCAGCCCC | 167 | 3011 | GGGGCUGGGGCAGCCGCUC | 416 |
| 3007 | CCCAUCUGCACCAUUGAUG | 168 | 3007 | CCCAUCUGCACCAUUGAUG | 168 | 3029 | CAUCAAUGGUGCAGAUGGG | 417 |
| 3025 | GUCUACAUGAUCAUGGUCA | 169 | 3025 | GUCUACAUGAUCAUGGUCA | 169 | 3047 | UGACCAUGAUCAUGUAGAC | 418 |
| 3043 | AAAUGUUGGAUGAUUGACU | 170 | 3043 | AAAUGUUGGAUGAUUGACU | 170 | 3065 | AGUCAAUCAUCCAACAUUU | 419 |
| 3061 | UCUGAAUGUCGGCCAAGAU | 171 | 3061 | UCUGAAUGUCGGCCAAGAU | 171 | 3083 | AUCUUGGCCGACAUUCAGA | 420 |
| 3079 | UUCCGGGAGUUGGUGUCUG | 172 | 3079 | UUCCGGGAGUUGGUGUCUG | 172 | 3101 | CAGACACCAACUCCCGGAA | 421 |
| 3097 | GAAUUCUCCCGCAUGGCCA | 173 | 3097 | GAAUUCUCCCGCAUGGCCA | 173 | 3119 | UGGCCAUGCGGGAGAAUUC | 422 |
| 3115 | AGGGACCCCCAGCGCUUUG | 174 | 3115 | AGGGACCCCCAGCGCUUUG | 174 | 3137 | CAAAGCGCUGGGGGUCCCU | 423 |
| 3133 | GUGGUCAUCCAGAAUGAGG | 175 | 3133 | GUGGUCAUCCAGAAUGAGG | 175 | 3155 | CCUCAUUCUGGAUGACCAC | 424 |
| 3151 | GACUUGGGCCCAGCCAGUC | 176 | 3151 | GACUUGGGCCCAGCCAGUC | 176 | 3173 | GACUGGCUGGGCCCAAGUC | 425 |
| 3169 | CCCUUGGACAGCACCUUCU | 177 | 3169 | CCCUUGGACAGCACCUUCU | 177 | 3191 | AGAAGGUGCUGUCCAAGGG | 426 |
| 3187 | UACCGCUCACUGCUGGAGG | 178 | 3187 | UACCGCUCACUGCUGGAGG | 178 | 3209 | CCUCCAGCAGUGAGCGGUA | 427 |
| 3205 | GACGAUGACAUGGGGGACC | 179 | 3205 | GACGAUGACAUGGGGGACC | 179 | 3227 | GGUCCCCCAUGUCAUCGUC | 428 |
| 3223 | CUGGUGGAUGCUGAGGAGU | 180 | 3223 | CUGGUGGAUGCUGAGGAGU | 180 | 3245 | ACUCCUCAGCAUCCACCAG | 429 |
| 3241 | UAUCUGGUACCCCAGCAGG | 181 | 3241 | UAUCUGGUACCCCAGCAGG | 181 | 3263 | CCUGCUGGGGUACCAGAUA | 430 |
| 3259 | GGCUUCUUCUGUCCAGACC | 182 | 3259 | GGCUUCUUCUGUCCAGACC | 182 | 3281 | GGUCUGGACAGAAGAAGCC | 431 |
| 3277 | CCUGCCCCGGGCGCUGGGG | 183 | 3277 | CCUGCCCCGGGCGCUGGGG | 183 | 3299 | CCCCAGCGCCCGGGGCAGG | 432 |
| 3295 | GGCAUGGUCCACCACAGGC | 184 | 3295 | GGCAUGGUCCACCACAGGC | 184 | 3317 | GCCUGUGGUGGACCAUGCC | 433 |
| 3313 | CACCGCAGCUCAUCUACCA | 185 | 3313 | CACCGCAGCUCAUCUACCA | 185 | 3335 | UGGUAGAUGAGCUGCGGUG | 434 |
| 3331 | AGGAGUGGCGGUGGGGACC | 186 | 3331 | AGGAGUGGCGGUGGGGACC | 186 | 3353 | GGUCCCCACCGCCACUCCU | 435 |
| 3349 | CUGACACUAGGGCUGGAGC | 187 | 3349 | CUGACACUAGGGCUGGAGC | 187 | 3371 | GCUCCAGCCCUAGUGUCAG | 436 |
| 3367 | CCCUCUGAAGAGGAGGCCC | 188 | 3367 | CCCUCUGAAGAGGAGGCCC | 188 | 3389 | GGGCCUCCUCUUCAGAGGG | 437 |
| 3385 | CCCAGGUCUCCACUGGCAC | 189 | 3385 | CCCAGGUCUCCACUGGCAC | 189 | 3407 | GUGCCAGUGGAGACCUGGG | 438 |
| 3403 | CCCUCCGAAGGGGCUGGCU | 190 | 3403 | CCCUCCGAAGGGGCUGGCU | 190 | 3425 | AGCCAGCCCCUUCGGAGGG | 439 |
| 3421 | UCCGAUGUAUUUGAUGGUG | 191 | 3421 | UCCGAUGUAUUUGAUGGUG | 191 | 3443 | CACCAUCPAAUACAUCGGA | 440 |
| 3439 | GACCUGGGAAUGGGGGCAG | 192 | 3439 | GACCUGGGAAUGGGGGCAG | 192 | 3461 | CUGCCCCCAUUCCCAGGUC | 441 |
| 3457 | GCCAAGGGGCUGCAAAGCC | 193 | 3457 | GCCAAGGGGCUGCAAAGCC | 193 | 3479 | GGCUUUGCAGCCCCUUGGC | 442 |
| 3475 | CUCCCCACACAUGACCCCA | 194 | 3475 | CUCCCCACACAUGACCCCA | 194 | 3497 | UGGGGUCAUGUGUGGGGAG | 443 |
| 3493 | AGCCCUCUACAGCGGUACA | 195 | 3493 | AGCCCUCUACAGCGGUACA | 195 | 3515 | UGUACCGCUGUAGAGGGCU | 444 |
| 3511 | AGUGAGGACCOCACAGUAC | 196 | 3511 | AGUGAGGACCCCACAGUAC | 196 | 3533 | GUACUGUGGGGUCCUCACU | 445 |
| 3529 | CCCCUGCCCUCUGAGACUG | 197 | 3529 | CCCCUGCCCUCUGAGACUG | 197 | 3551 | CAGUCUCAGAGGGCAGGGG | 446 |
| 3547 | GAUGGCUACGUUGCCCCCC | 198 | 3547 | GAUGGCUACGUUGCCCCCC | 198 | 3569 | GGGGGGCAACGUAGCCAUC | 447 |
| 3565 | CUGACCUGCAGCCCCCAGC | 199 | 3565 | CUGACCUGCAGCCCCCAGC | 199 | 3587 | GCUGGGGGCUGCAGGUCAG | 448 |
| 3583 | CCUGAAUAUGUGAACCAGC | 200 | 3583 | CCUGAAUAUGUGAACCAGC | 200 | 3605 | GCUGGUUCACAUAUUCAGG | 449 |
| 3601 | CCAGAUGUUCGGCCCCAGC | 201 | 3601 | CCAGAUGUUCGGCCCCAGC | 201 | 3623 | GCUGGGGCCGAACAUCUGG | 450 |
| 3619 | CCCCCUUCGCCCCGAGAGG | 202 | 3619 | CCCCCUUCGCCCCGAGAGG | 202 | 3641 | CCUCUCGGGGCGAAGGGGG | 451 |
| 3637 | GGCCCUCUGCCUGCUGCCC | 203 | 3637 | GGCCCUCUGCCUGCUGCCC | 203 | 3659 | GGGCAGCAGGCAGAGGGCC | 452 |
| 3655 | CGACCUGCUGGUGCCACUC | 204 | 3655 | CGACCUGCUGGUGCCACUC | 204 | 3677 | GAGUGGCACCAGCAGGUCG | 453 |
| 3673 | CUGGAAAGGCCCAAGACUC | 205 | 3673 | CUGGAAAGGCCCAAGACUC | 205 | 3695 | GAGUCUUGGGCCUUUCCAG | 454 |
| 3691 | CUCUCCCCAGGGAAGAAUG | 206 | 3691 | CUCUCCCCAGGGAAGAAUG | 206 | 3713 | CAUUCUUCCCUGGGGAGAG | 455 |
| 3709 | GGGGUCGUCAAAGACGUUU | 207 | 3709 | GGGGUCGUCAAAGACGUUU | 207 | 3731 | AAACGUCUUUGACGACCCC | 456 |
| 3727 | UUUGCCUUUGGGGGUGCCG | 208 | 3727 | UUUGCCUUUGGGGGUGCCG | 208 | 3749 | CGGCACCCCCkAAGGCAAA | 457 |
| 3745 | GUGGAGAACCCCGAGUACU | 209 | 3745 | GUGGAGAACCCCGAGUACU | 209 | 3767 | AGUACUCGGGGUUCUCCAC | 458 |
| 3763 | UUGACACCCCAGGGAGGAG | 210 | 3763 | UUGACACCCCAGGGAGGAG | 210 | 3785 | CUCCUCCCUGGGGUGUCAA | 459 |
| 3781 | GCUGCCCCUCAGCCCCACC | 211 | 3781 | GCUGCCCCUCAGCCCCACC | 211 | 3803 | GGUGGGGCUGAGGGGCAGC | 460 |
| 3799 | CCUCCUCCUGCCUUCAGCC | 212 | 3799 | CCUCCUCCUGCCUUCAGCC | 212 | 3821 | GGCUGAAGGCAGGAGGAGG | 461 |
| 3817 | CCAGCCUUCGACAACCUCU | 213 | 3817 | CCAGCCUUCGACAACCUCU | 213 | 3839 | AGAGGUUGUCGAAGGCUGG | 462 |
| 3835 | UAUUACUGGGACCAGGACC | 214 | 3835 | UAUUACUGGGACCAGGACC | 214 | 3857 | GGUCCUGGUCCCAGUAAUA | 463 |
| 3853 | CCACCAGAGCGGGGGGCUC | 215 | 3853 | CCACCAGAGCGGGGGGCUC | 215 | 3875 | GAGCCCCCCGCUCUGGUGG | 464 |
| 3871 | CCACCCAGCACCUUCAAAG | 216 | 3871 | CCACCCAGCACCUUCAAAG | 216 | 3893 | CUUUGAAGGUGCUGGGUGG | 465 |
| 3889 | GGGACACCUACGGCAGAGA | 217 | 3889 | GGGACACCUACGGCAGAGA | 217 | 3911 | UCUCUGCCGUAGGUGUCCC | 466 |
| 3907 | AACCCAGAGUACCUGGGUC | 218 | 3907 | AACCCAGAGUACCUGGGUC | 218 | 3929 | GACCCAGGUACUCUGGGUU | 467 |
| 3925 | CUGGACGUGCCAGUGUGAA | 219 | 3925 | CUGGACGUGCCAGUGUGAA | 219 | 3947 | UUCACACUGGCACGUCCAG | 468 |
| 3943 | ACCAGAAGGCCAAGUCCGC | 220 | 3943 | ACCAGAAGGCCAAGUCCGC | 220 | 3965 | GCGGACUUGGCCUUCUGGU | 469 |
| 3961 | CAGAAGCCCUGAUGUGUCC | 221 | 3961 | CAGAAGCCCUGAUGUGUCC | 221 | 3983 | GGACACAUCAGGGCUUCUG | 470 |
| 3979 | CUCAGGGAGCAGGGAAGGC | 222 | 3979 | CUCAGGGAGCAGGGAAGGC | 222 | 4001 | GCCUUCCCUGCUCCCUGAG | 471 |
| 3997 | CCUGACUUCUGCUGGCAUC | 223 | 3997 | CCUGACUUCUGCUGGCAUC | 223 | 4019 | GAUGCCAGCAGAAGUCAGG | 472 |
| 4015 | CAAGAGGUGGGAGGGCCCU | 224 | 4015 | CAAGAGGUGGGAGGGCCCU | 224 | 4037 | AGGGCCCUCCCACCUCUUG | 473 |
| 4033 | UCCGACCACUUCCAGGGGA | 225 | 4033 | UCCGACCACUUCCAGGGGA | 225 | 4055 | UCCCCUGGAAGUGGUCGGA | 474 |
| 4051 | AACCUGCCAUGCCAGGAAC | 226 | 4051 | AACCUGCCAUGCCAGGAAC | 226 | 4073 | GUUCCUGGCAUGGCAGGUU | 475 |
| 4069 | CCUGUCCUAAGGAACCUUC | 227 | 4069 | CCUGUCCUAAGGAACCUUC | 227 | 4091 | GAAGGUUCCUUAGGACAGG | 476 |
| 4087 | CCUUCCUGCUUGAGUUCCC | 228 | 4087 | CCUUCCUGCUUGAGUUCCC | 228 | 4109 | GGGAACUCAAGCAGGAAGG | 477 |
| 4105 | CAGAUGGCUGGAAGGGGUC | 229 | 4105 | CAGAUGGCUGGAAGGGGUC | 229 | 4127 | GACCCCUUCCAGCCAUCUG | 478 |
| 4123 | CCAGCCUCGUUGGAAGAGG | 230 | 4123 | CCAGCCUCGUUGGAAGAGG | 230 | 4145 | CCUCUUCCAACGAGGCUGG | 479 |
| 4141 | GAACAGCACUGGGGAGUCU | 231 | 4141 | GAACAGCACUGGGGAGUCU | 231 | 4163 | AGACUCCCCAGUGCUGUUC | 480 |
| 4159 | UUUGUGGAUUCUGAGGCCC | 232 | 4159 | UUUGUGGAUUCUGAGGCCC | 232 | 4181 | GGGCCUCAGAAUCCACkAA | 481 |
| 4177 | CUGCCCAAUGAGACUCUAG | 233 | 4177 | CUGCCCAAUGAGACUCUAG | 233 | 4199 | CUAGAGUCUCAUUGGGCAG | 482 |
| 4195 | GGGUCCAGUGGAUGCCACA | 234 | 4195 | GGGUCCAGUGGAUGCCACA | 234 | 4217 | UGUGGCAUCCACUGGACCC | 483 |
| 4213 | AGCCCAGCUUGGCCCUUUC | 235 | 4213 | AGCCCAGCUUGGCCCUUUC | 235 | 4235 | GAAAGGGCCAAGCUGGGCU | 484 |
| 4231 | CCUUCCAGAUCCUGGGUAC | 236 | 4231 | CCUUCCAGAUCCUGGGUAC | 236 | 4253 | GUACCCAGGAUCUGGAAGG | 485 |
| 4249 | CUGAAAGCCUUAGGGAAGC | 237 | 4249 | CUGAAAGCCUUAGGGAAGC | 237 | 4271 | GCUUCCCUAAGGCUUUCAG | 486 |
| 4267 | CUGGCCUGAGAGGGGAAGC | 238 | 4267 | CUGGCCUGAGAGGGGAAGC | 238 | 4289 | GCUUCCCCUCUCAGGCCAG | 487 |
| 4285 | CGGCCCUAAGGGAGUGUCU | 239 | 4285 | CGGCCCUAAGGGAGUGUCU | 239 | 4307 | AGACACUCCCUUAGGGCCG | 488 |
| 4303 | UAAGAACAAAAGCGACCCA | 240 | 4303 | UAAGAACAAAAGCGACCCA | 240 | 4325 | UGGGUCGCUUUUGUUCUUA | 489 |
| 4321 | AUUCAGAGACUGUCCCUGA | 241 | 4321 | AUUCAGAGACUGUCCCUGA | 241 | 4343 | UCAGGGACAGUCUCUGAAU | 490 |
| 4339 | AAACCUAGUACUGCCCCCC | 242 | 4339 | AAACCUAGUACUGCCCCCC | 242 | 4361 | GGGGGGCAGUACUAGGUUU | 491 |
| 4357 | CAUGAGGAAGGAACAGCAA | 243 | 4357 | CAUGAGGAAGGAACAGCAA | 243 | 4379 | UUGCUGUUCCUUCCUCAUG | 492 |
| 4375 | AUGGUGUCAGUAUCCAGGC | 244 | 4375 | AUGGUGUCAGUAUCCAGGC | 244 | 4397 | GCCUGGAUACUGACACCAU | 493 |
| 4393 | CUUUGUACAGAGUGCUUUU | 245 | 4393 | CUUUGUACAGAGUGCUUUU | 245 | 4415 | AAAAGCACUCUGUACAAAG | 494 |
| 4411 | UCUGUUUAGUUUUUACUUU | 246 | 4411 | UCUGUUUAGUUUUUACUUU | 246 | 4433 | AAAGUAAAAACUAAACAGA | 495 |
| 4429 | UUUUUGUUUUGUUUUUUUA | 247 | 4429 | UUUUUGUUUUGUUUUUUUA | 247 | 4451 | UAAAAAACAAAACAAAAAA | 496 |
| 4447 | AAGAUGAAAUAAAGACCCA | 248 | 4447 | AAAGAUGAAAUAAAGACCC | 248 | 4469 | GGGUCUUUAUUUCAUCUUU | 497 |
| 4455 | AAUAAAGACCCAGGGGGAG | 249 | 4455 | AAUAAAGACCCAGGGGGAG | 249 | 4477 | CUCCCCCUGGGUCUUUAUU | 498 |

HSERB2R (X03363)

TABLE II

+HZ,1 HER2 Synthetic siNA constructs

| | | | |
|---|---|---|---|
| | | | SEQ |
| RPI# | Aliases | Sequence | ID# |

| | | | |
|---|---|---|---|
| 25245 | RPI 17763 Her2Neu AS as siNA Str 2 | BUCCAUGGUGOUCACUGOGGOUB | 499 |
| | (antisense) | | |
| 25246 | RPI 17763 Her2Neu AS as siNA Str 1 | BAGCCGCAGUGAGCACCAUGGAB | 500 |
| | (sense) | | |
| 25247 | RPI 17763 Her2Neu AS as siNA Str 1 | BAGGUACCACGAGUGACGCCGAB | 501 |
| | (sense) Inverted control | | |
| 25248 | RPI 17763 Her2Neu AS as siNA Str 1 | BUCGGCGUCACUCGUGGUACCUB | 502 |
| | (sense) Inverted control compliment | | |
| 25822 | RPI 17763 Her2Neu AS as siNA Str 2 | UCCAUGGUGCUCACUGCGGCUUU | 503 |
| | (antisense)+2U overhang | | |
| 25823 | RPI 17763 Her2Neu AS as siNA Str 1 | AGCCGCAGUGAGCACCAUGGAUU | 504 |
| | (sense)+2U overhang | | |
| 25842 | RPI 17763 Her2Neu AS as siNA Str 2 | BUCCAUGGUGCUCACUGCGGCUUUB | 505 |
| | (antisense)+2U overhang | | |
| 25843 | RPI 17763 Her2Neu AS as siNA Str 1 | BAGCCGCAGUGAGCACCAUGGAUUB | 506 |
| | (sense)+2U overhang | | |
| 28262 | Her2.1.sense Str1 | UGGGGUCGUCAAAGACGUUTT | 507 |
| 28263 | Her2.1.antisense Str2 | AACGUCUUUGACGACCCCATT | 508 |
| 28264 | Her2.1.sense Str1 inverted | UUGCAGAAACUGCUGGGGUTT | 509 |
| 28265 | Her2.1.antisense Str2 inverted | ACCCCAGCAGUUUCUGCAATT | 510 |
| 28266 | Her2.2.sense Str1 | GGUGCUUGGAUCUGGCGCUTT | 511 |
| 28267 | Her2.2.antisense Str2 | AGCGCCAGAUCCAAGCACCTT | 512 |
| 28268 | Her2.2.sense Str1 inverted | UCGCGGUCUAGGUUCGUGGTT | 513 |
| 28269 | Her2.2.antisense Str2 inverted | CCACGAACCUAGACCGCGATT | 514 |
| 28270 | Her2.3.sense Str1 | GAUCUUUGGGAGCCUGGCATT | 515 |
| 28271 | Her2.3.antisense Str2 | UGCCAGGCUCCCAAAGAUCTT | 516 |
| 28272 | Her2.3.sense Str1 inverted | ACGGUCCGAGGGUUUCUAGTT | 517 |
| 28273 | Her2.3.antisense Str2 inverted | CUAGAAACCCUCGGACCGUTT | 518 |
| 29989 | Her2.2.sense Str1 (site 2344) | G_{S}G_{S}u_{S}G_{S}cuuGGAucuGGcG_{S}csu_{S}T_{S}T | 519 |
| 29990 | Her2.2.antisense Str2 | A_{S}G_{S}C_{S}G_{S}C_{S}CAGAUCCAAGCACCT_{S}T | 520 |
| 29991 | Her2.2.sense Str1 (site 2344) | G_{S}G_{S}U_{S}G_{S}C_{S}UUGGAUCUGGCGCUT_{S}T | 521 |
| 29992 | Her2.2.sense Str1 (site 2344) | G_{S}G_{S}u_{S}G_{S}cuuGGAucuGGcGcuTTB | 522 |
| 29993 | Her2.2.antisense Str2 | A_{S}G_{S}C_{S}G_{S}C_{S}C_{S}A_{S}G_{S}A_{S}U_{S}C_{S}C_{S}A_{S}A_{S}G_{S}C_{S}A_{S}C_{S}C_{S}T_{S}T | 523 |
| 29994 | Her2.2.antisense Str2 | A_{S}G_{S}C_{S}G_{S}C_{S}C_{S}A_{S}G_{S}A_{S}U_{S}CCAAGCACCT_{S}T | 524 |
| 29995 | Her2.2.antisense Str2 | A_{S}G_{S}C_{S}G_{S}C_{S}C_{S}A_{S}G_{S}A_{S}U_{S}C_{S}C_{S}A_{S}A_{S}GCACCT_{S}T | 525 |
| 29996 | Her2.2.sense Str1 inverted | u_{S}c_{S}G_{S}c_{S}GGucuAGGuucGu_{S}G_{S}G_{S}T_{S}AT | 526 |
| 29997 | Her2.2.sense Str1 inverted | U_{S}C_{S}G_{S}C_{S}G_{S}GUCUAGGUUCGUGGT_{S}T | 527 |
| 29998 | Her2.2.sense Str1 inverted | u_{S}c_{S}G_{S}c_{S}GGucuAGGuucGuGGTTB | 528 |
| 29999 | Her2.2.antisense Str2 inverted | C_{S}C_{S}A_{S}C_{S}G_{S}AACCUAGACCGCGAT_{S}T | 529 |
| 30000 | Her2.2.antisense Str2 inverted | C_{S}C_{S}A_{S}C_{S}G_{S}A_{S}A_{S}C_{S}C_{S}U_{S}A_{S}G_{S}A_{S}C_{S}C_{S}G_{S}C_{S}G_{S}A_{S}T_{S}T | 530 |
| 30001 | Her2.2.antisense Str2 inverted | C_{S}C_{S}A_{S}C_{S}G_{S}A_{S}A_{S}C_{S}C_{S}U_{S}AGACCGCGAT_{S}T | 531 |
| 30002 | Her2.2.antisense Str2 inverted | C_{S}C_{S}A_{S}C_{S}G_{S}A_{S}A_{S}C_{S}C_{S}U_{S}A_{S}G_{S}A_{S}C_{S}CGCGAT_{S}T | 532 |

UPPER CASE =ribonucleotide
Lower case =2′-O-methyl nucleotide
Underline =2′-deoxy-2′-amino nucleotide
Italic =2′-deoxy-2′-fluro nucleotide
T =thymidine
B =inverted deoxy abasic succinate linker
B =inverted deoxy abasic
X =universal base (5-nitroindole)
Z =universal base (3-nitropyrrole)
S =phosphorothioate internucleotide linkage
U =5-bromodeoxyuridine

TABLE III

HER2 Synthetic Sequences with all RNA counterparts and Target Sequences

| | | | | | |
|---|---|---|---|---|---|
| Target | | Seq | | | |
| Pos | Target | ID | Aliases | Sequence | Seq ID |

| | | | | | |
|---|---|---|---|---|---|
| 986 | CGUGUCUGUGUUGUAGGUGACCA | 533 | HER2:988U21 siNA | GUCACCUACAACA0AGACACG | 537 |
| 1882 | AAACAGGUCACUGAGCCAUUCUG | 534 | HER2:1884U21 siN | AGAAUGGCUOAGUGACOUGUUU | 538 |
| 3128 | CUCAUUCUGGAUGACCACAAAGC | 535 | HER2:3130U21 siNA | UUUGUGGUCAUCCAGAAUGAG | 539 |
| 3877 | GUAGGUGUCCCUUUGAAGGUGCU | 536 | HER2:3879U21 siNA | CACCUUCAAAGGGACACCUAC | 540 |
| 986 | CGUGUCUGUGUUGUAGGUGACCA | 533 | HER2:1006L21 siNA (988C) | UGUCUGUGUUGUAGGUGACCA | 541 |
| 1882 | AAACAGGUCACUGAGCCAUUCUG | 534 | HER2:1902L21 siNA (1884C) | ACAGGUCACUGAGCCAUUCUG | 542 |
| 3128 | CUCAUUCUGGAUGACCACAAAGC | 535 | HER2:3148L21 siNA (3130C) | CAUUCUGGAUGACCACXAAGC | 543 |
| 3877 | GUAGGUGUCCCUUUGAAGGUGCU | 536 | HER2:3897L21 siNA (3879C) | AGGUGUCCCUUUGAAGGUGCU | 544 |
| 986 | CGUGUCUGUGUUGUAGGUGACCA | 533 | HER2:988U21 siNA stab4 | B GucAccuAcAAcAcAGAcAcG B | 545 |
| 1882 | AAACAGGUCACUGAGCCAUUCUG | 534 | HER2:1884U21 siNA stab4 | B GAAuGGcucAGuGAccuGuuu B | 546 |
| 3128 | CUCAUUCUGGAUGACCACAAAGC | 535 | HER2:3130U21 siNA stab4 | B uuuGuGGucAuccAGAAuGAG B | 547 |
| 3877 | GUAGGUGUCCCUUUGAAGGUGCU | 536 | HER2:3879U21 siNA stab4 | B cAccuucAAAGGGAcAccuAc B | 548 |
| 986 | CGUGUCUGUGUUGUAGGUGACCA | 533 | HER2:1006L21 siNA (988C) stab5 | uGucuGuGuuGuAGGuGAcTsT | 549 |
| 1882 | AAACAGGUCACUGAGCCAUUCUG | 534 | HER2:1902L21 siNA (1884C) stab5 | AcAGGucAcuGAGccAuucTsT | 550 |
| 3128 | CUCAUUCUGGAUGA0CACAAAGC | 535 | HER2:3148L21 siNA (3130C) stab5 | cAuucuGGAuGAccAcAAATsT | 551 |
| 3877 | GUAGGUGUCCCUUUGAAGGUGCU | 536 | HER2:3897L21 siNA (3879C) stab5 | AGGuGucccuuuGAAGGuGTsT | 552tz,1/64 |

A = Adenosine
G = Guanosine
C = Cytidine
U = Uridine
T = Thymidine
u = 2′-deoxy-2′-fluoro uridine
c = 2′-deoxy-2′-fluoro cytidine
B = inverted deoxy abasic ribose
L = glyceryl moiety
S = phosphorothioate internucleotide linkage

TABLE IV

EGFR (HER1) siNA and Target Sequences

| | | | | | | | | |
|---|---|---|---|---|---|---|---|---|
| | | Seq | | | Seq | | | Seq |
| Pos | Target Sequence | ID | UPos | Upper seq | ID | LPos | Lower seq | ID |

| | | | | | | | | |
|---|---|---|---|---|---|---|---|---|
| 3 | CGCGCUGCGCCGGAGUCCC | 553 | 3 | CGCGCUGCGCCGGAGUCCC | 553 | 21 | GGGACUCCGGCGCAGCGCG | 860 |
| 21 | CGAGCUAGCCCCGGCGCCG | 554 | 21 | CGAGCUAGCCCCGGCGCCG | 554 | 39 | CGGCGCCGGGGCUAGCUCG | 861 |
| 39 | GCCGCCGCCCAGACCGGAC | 555 | 39 | GCCGCCGCCCAGACCGGAC | 555 | 57 | GUCCGGUCUGGGCGGCGGC | 862 |
| 57 | CGACAGGCCACCUCGUCGG | 556 | 57 | CGACAGGCCACCUCGUCGG | 556 | 75 | CCGACGAGGUGGCCUGUCG | 863 |
| 75 | GCGUCCGCCCGAGUCCCCG | 557 | 75 | GCGUCCGCCCGAGUCCCCG | 557 | 93 | CGGGGACUCGGGCGGACGC | 864 |
| 93 | GCCUCGCCGCCAACGCCAC | 558 | 93 | GCCUCGCCGCCAACGCCAC | 558 | 111 | GUGGCGUUGGCGGCGAGGC | 865 |
| 111 | CAACCACCGCGCACGGCCC | 559 | 111 | CAACCACCGCGCACGGCCC | 559 | 129 | GGGCCGUGCGCGGUGGUUG | 866 |
| 129 | CCCUGACUCCGUCCAGUAU | 560 | 129 | CCCUGACUCCGUCCAGUAU | 560 | 147 | AUACUGGACGGAGUCAGGG | 867 |
| 147 | UUGAUCGGGAGAGCCGGAG | 561 | 147 | UUGAUCGGGAGAGCCGGAG | 561 | 165 | CUCCGGCUCUCCCGAUCAA | 868 |
| 165 | GCGAGCUCUUCGGGGAGCA | 562 | 165 | GCGAGCUCUUCGGGGAGCA | 562 | 183 | UGCUCCCCGAAGAGCUCGC | 869 |
| 183 | AGCGAUGCGACCCUCCGGG | 563 | 183 | AGCGAUGCGACCCUCCGGG | 563 | 201 | CCCGGAGGGUCGCAUCGCU | 870 |
| 201 | GACGGCCGGGGCAGCGCUC | 564 | 201 | GACGGCCGGGGCAGCGCUC | 564 | 219 | GAGCGCUGCCCCGGCCGUC | 871 |
| 219 | CCUGGCGCUGCUGGCUGCG | 565 | 219 | CCUGGCGCUGCUGGCUGCG | 565 | 237 | CGCAGCCAGCAGCGCCAGG | 872 |
| 237 | GCUCUGCCCGGCGAGUCGG | 566 | 237 | GCUCUGCCCGGCGAGUCGG | 566 | 255 | CCGACUCGCCGGGCAGAGC | 873 |
| 255 | GGCUCUGGAGGAAAAGAAA | 567 | 255 | GGCUCUGGAGGAAAAGAAA | 567 | 273 | UUUCUUUUCCUCCAGAGCC | 874 |
| 273 | AGUUUGCCAAGGCACGAGU | 568 | 273 | AGUUUGCCAAGGCACGAGU | 568 | 291 | ACUCGUGCCUUGGCAAACU | 875 |
| 291 | UAACAAGCUCACGCAGUUG | 569 | 291 | UAACAAGCUCACGCAGUUG | 569 | 309 | CAACUGCGUGAGCUUGUUA | 876 |
| 309 | GGGCACUUUUGAAGAUCAU | 570 | 309 | GGGCACUUUUGAAGAUCAU | 570 | 327 | AUGAUCUUCAAAAGUGCCC | 877 |
| 327 | UUUUCUCAGCCUCCAGAGG | 571 | 327 | UUUUCUCAGCCUCCAGAGG | 571 | 345 | CCUCUGGAGGCUGAGAAAA | 878 |
| 345 | GAUGUUCAAUAACUGUGAG | 572 | 345 | GAUGUUCAAUAACUGUGAG | 572 | 363 | CUCACAGUUAUUGAACAUC | 879 |
| 363 | GGUGGUCCUUGGGAAUUUG | 573 | 363 | GGUGGUCCUUGGGAAUUUG | 573 | 381 | CAAAUUCCCAAGGACCACC | 880 |
| 381 | GGAAAUUACCUAUGUGCAG | 574 | 381 | GGAAAUUACCUAUGUGCAG | 574 | 399 | CUGCACAUAGGUAAUUUCC | 881 |
| 399 | GAGGAAUUAUGAUCUUUCC | 575 | 399 | GAGGAAUUAUGAUCUUUCC | 575 | 417 | GGAAAGAUCAUAAUUCCUC | 882 |
| 417 | CUUCUUAAAGACCAUCCAG | 576 | 417 | CUUCUUAAAGACCAUCCAG | 576 | 435 | CUGGAUGGUCUUUAAGAAG | 883 |
| 435 | GGAGGUGGCUGGUUAUGUC | 577 | 435 | GGAGGUGGCUGGUUAUGUC | 577 | 453 | GACAUAACCAGCCACCUCC | 884 |
| 453 | CCUCAUUGCCCUCAACACA | 578 | 453 | CCUCAUUGCCCUCAACACA | 578 | 471 | UGUGUUGAGGGCAAUGAGG | 885 |
| 471 | AGUGGAGCGAAUUCCUUUG | 579 | 471 | AGUGGAGCGAAUUCCUUUG | 579 | 489 | CAAAGGAAUUCGCUCCACU | 886 |
| 489 | GGAAAACCUGCAGAUCAUC | 580 | 489 | GGAAAACCUGCAGAUCAUC | 580 | 507 | GAUGAUCUGCAGGUUUUCC | 887 |
| 507 | CAGAGGAAAUAUGUACUAC | 581 | 507 | CAGAGGAAAUAUGUACUAC | 581 | 525 | GUAGUACAUAUUUCCUCUG | 888 |
| 525 | CGAAAAUUCCUAUGCCUUA | 582 | 525 | CGAAAAUUCCUAUGCCUUA | 582 | 543 | UAAGGCAUAGGAAUUUUCG | 889 |
| 543 | AGCAGUCUUAUCUAACUAU | 583 | 543 | AGCAGUCUUAUCUAACUAU | 583 | 561 | AUAGUUAGAUAAGACUGCU | 890 |
| 561 | UGAUGCAAAUAAAACCGGA | 584 | 561 | UGAUGCAAAUAAAACCGGA | 584 | 579 | UCCGGUUUUAUUUGCAUCA | 891 |
| 579 | ACUGAAGGAGCUGCCCAUG | 585 | 579 | ACUGAAGGAGCUGCCCAUG | 585 | 597 | CAUGGGCAGCUCCUUCAGU | 892 |
| 597 | GAGAAAUUUACAGGAAAUC | 586 | 597 | GAGAAAUUUACAGGAAAUC | 586 | 615 | GAUUUCCUGUAAAUUUCUC | 893 |
| 615 | CCUGCAUGGCGCCGUGCGG | 587 | 615 | CCUGCAUGGCGCCGUGCGG | 587 | 633 | CCGCACGGCGCCAUGCAGG | 894 |
| 633 | GUUCAGCAACAACCCUGCC | 588 | 633 | GUUCAGCAACAACCCUGCC | 588 | 651 | GGCAGGGUUGUUGCUGAAC | 895 |
| 651 | CCUGUGCAACGUGGAGAGC | 589 | 651 | CCUGUGCAACGUGGAGAGC | 589 | 669 | GCUCUCCACGUUGCACAGG | 896 |
| 669 | CAUCCAGUGGCGGGACAUA | 590 | 669 | CAUCCAGUGGCGGGACAUA | 590 | 687 | UAUGUCCCGCCACUGGAUG | 897 |
| 687 | AGUCAGCAGUGACUUUCUC | 591 | 687 | AGUCAGCAGUGACUUUCUC | 591 | 705 | GAGAAAGUCACUGCUGACU | 898 |
| 705 | CAGCAACAUGUCGAUGGAC | 592 | 705 | CAGCAACAUGUCGAUGGAC | 592 | 723 | GUCCAUCGACAUGUUGCUG | 899 |
| 723 | CUUCCAGAACCACCUGGGC | 593 | 723 | CUUCCAGAACCACCUGGGC | 593 | 741 | GCCCAGGUGGUUCUGGAAG | 900 |
| 741 | CAGCUGCCAAAAGUGUGAU | 594 | 741 | CAGCUGCCAAAAGUGUGAU | 594 | 759 | AUCACACUUUUGGCAGCUG | 901 |
| 759 | UCCAAGCUGUCCCAAUGGG | 595 | 759 | UCCAAGCUGUCCCAAUGGG | 595 | 777 | CCCAUUGGGACAGCUUGGA | 902 |
| 777 | GAGCUGCUGGGGUGCAGGA | 596 | 777 | GAGCUGCUGGGGUGCAGGA | 596 | 795 | UCCUGCACCCCAGCAGCUC | 903 |
| 795 | AGAGGAGAACUGCCAGAAA | 597 | 795 | AGAGGAGAACUGCCAGAAA | 597 | 813 | UUUCUGGCAGUUCUCCUCU | 904 |
| 813 | ACUGACCAAAAUCAUCUGU | 598 | 813 | ACUGACCAAAAUCAUCUGU | 598 | 831 | ACAGAUGAUUUUGGUCAGU | 905 |
| 831 | UGCCCAGCAGUGCUCCGGG | 599 | 831 | UGCCCAGCAGUGCUCCGGG | 599 | 849 | CCCGGAGCACUGCUGGGCA | 906 |
| 849 | GCGCUGCCGUGGCAAGUCC | 600 | 849 | GCGCUGCCGUGGCAAGUCC | 600 | 867 | GGACUUGCCACGGCAGCGC | 907 |
| 867 | CCCCAGUGACUGOUGCCAC | 601 | 867 | CCCCAGUGACUGCUGCCAC | 601 | 885 | GUGGCAGCAGUCACUGGGG | 908 |
| 885 | CAACCAGUGUGCUGCAGGC | 602 | 885 | CAACCAGUGUGCUGCAGGC | 602 | 903 | GCCUGCAGCACACUGGUUG | 909 |
| 903 | CUGCACAGGCCCCCGGGAG | 603 | 903 | CUGCACAGGCCCCCGGGAG | 603 | 921 | CUCCCGGGGGCCUGUGCAG | 910 |
| 921 | GAGCGACUGCCUGGUCUGC | 604 | 921 | GAGCGACUGCCUGGUCUGC | 604 | 939 | GCAGACCAGGCAGUCGCUC | 911 |
| 939 | CCGCAAAUUCCGAGACGAA | 605 | 939 | CCGCAAAUUCCGAGACGAA | 605 | 957 | UUCGUCUCGGAAUUUGCGG | 912 |
| 957 | AGCCACGUGCAAGGACACC | 606 | 957 | AGCCACGUGCAAGGACACC | 606 | 975 | GGUGUCCUUGCACGUGGCU | 913 |
| 975 | CUGCCCCCCACUCAUGCUC | 607 | 975 | CUGCCCCCCACUCAUGCUC | 607 | 993 | GAGCAUGAGUGGGGGGCAG | 914 |
| 993 | CUACAACCCCACCACGUAC | 608 | 993 | CUACAACCCCACCACGUAC | 608 | 1011 | GUACGUGGUGGGGUUGUAG | 915 |
| 1011 | CCAGAUGGAUGUGAACCCC | 609 | 1011 | CCAGAUGGAUGUGAACCCC | 609 | 1029 | GGGGUUCACAUCCAUCUGG | 916 |
| 1029 | CGAGGGCAAAUACAGCUUU | 610 | 1029 | CGAGGGCAAAUACAGCUUU | 610 | 1047 | AAAGCUGUAUUUGCCCUCG | 917 |
| 1047 | UGGUGCCACCUGCGUGAAG | 611 | 1047 | UGGUGCCACCUGCGUGAAG | 611 | 1065 | CUUCACGCAGGUGGCACCA | 918 |
| 1065 | GAAGUGUCCCCGUAAUUAU | 612 | 1065 | GAAGUGUCCCCGUAAUUAU | 612 | 1083 | AUAAUUACGGGGACACUUC | 919 |
| 1083 | UGUGGUGACAGAUCACGGC | 613 | 1083 | UGUGGUGACAGAUCACGGC | 613 | 1101 | GCCGUGAUCUGUCACCACA | 920 |
| 1101 | CUCGUGCGUCCGAGCCUGU | 614 | 1101 | CUCGUGCGUCCGAGCCUGU | 614 | 1119 | ACAGGCUCGGACGCACGAG | 921 |
| 1119 | UGGGGCCGACAGCUAUGAG | 615 | 1119 | UGGGGCCGACAGCUAUGAG | 615 | 1137 | CUCAUAGCUGUCGGCCCCA | 922 |
| 1137 | GAUGGAGGAAGACGGCGUC | 616 | 1137 | GAUGGAGGAAGACGGCGUC | 616 | 1155 | GACGCCGUCUUCCUCCAUC | 923 |
| 1155 | CCGCAAGUGUAAGAAGUGC | 617 | 1155 | CCGCAAGUGUAAGAAGUGC | 617 | 1173 | GCACUUCUUACACUUGCGG | 924 |
| 1173 | CGAAGGGCCUUGCCGCAAA | 618 | 1173 | CGAAGGGCCUUGCCGCAAA | 618 | 1191 | UUUGCGGCAAGGCCCUUCG | 925 |
| 1191 | AGUGUGUAACGGAAUAGGU | 619 | 1191 | AGUGUGUAACGGAAUAGGU | 619 | 1209 | ACCUAUUCCGUUACACACU | 926 |
| 1209 | UAUUGGUGAAUUUAAAGAC | 620 | 1209 | UAUUGGUGAAUUUAAAGAC | 620 | 1227 | GUCUUUAAAUUCACCAAUA | 927 |
| 1227 | CUCACUCUCCAUAAAUGCU | 621 | 1227 | CUCACUCUCCAUAAAUGCU | 621 | 1245 | AGCAUUUAUGGAGAGUGAG | 928 |
| 1245 | UACGAAUAUUAAACACUUC | 622 | 1245 | UACGAAUAUUAAACACUUC | 622 | 1263 | GAAGUGUUUAAUAUUCGUA | 929 |
| 1263 | CAAAAACUGCACCUCCAUC | 623 | 1263 | CAAAAACUGCACCUCCAUC | 623 | 1281 | GAUGGAGGUGCAGUUUUUG | 930 |
| 1281 | CAGUGGCGAUCUCCACAUC | 624 | 1281 | CAGUGGCGAUCUCCACAUC | 624 | 1299 | GAUGUGGAGAUCGCCACUG | 931 |
| 1299 | CCUGCCGGUGGCAUUUAGG | 625 | 1299 | CCUGCCGGUGGCAUUUAGG | 625 | 1317 | CCUAAAUGCCACCGGCAGG | 932 |
| 1317 | GGGUGACUCCUUCACACAU | 626 | 1317 | GGGUGACUCCUUCACACAU | 626 | 1335 | AUGUGUGAAGGAGUCACCC | 933 |
| 1335 | UACUCCUCCUCUGGAUCCA | 627 | 1335 | UACUCCUCCUCUGGAUCCA | 627 | 1353 | UGGAUCCAGAGGAGGAGUA | 934 |
| 1353 | ACAGGAACUGGAUAUUCUG | 628 | 1353 | ACAGGAACUGGAUAUUCUG | 628 | 1371 | CAGAAUAUCCAGUUCCUGU | 935 |
| 1371 | GAAAACCGUAAAGGAAAUC | 629 | 1371 | GAAAACCGUAAAGGAAAUC | 629 | 1389 | GAUUUCCUUUACGGUUUUC | 936 |
| 1389 | CACAGGGUUUUUGCUGAUU | 630 | 1389 | CACAGGGUUUUUGCUGAUU | 630 | 1407 | AAUCAGCAAAAACCCUGUG | 937 |
| 1407 | UCAGGCUUGGCCUGAAAAC | 631 | 1407 | UCAGGCUUGGCCUGAAAAC | 631 | 1425 | GUUUUCAGGCCAAGCCUGA | 938 |
| 1425 | CAGGACGGACCUCCAUGCC | 632 | 1425 | CAGGACGGACCUCCAUGCC | 632 | 1443 | GGCAUGGAGGUCCGUCCUG | 939 |
| 1443 | CUUUGAGAACCUAGAAAUC | 633 | 1443 | CUUUGAGAACCUAGAAAUC | 633 | 1461 | GAUUUCUAGGUUCUCAAAG | 940 |
| 1461 | CAUACGCGGCAGGACCAAG | 634 | 1461 | CAUACGCGGCAGGACCAAG | 634 | 1479 | CUUGGUCCUGCCGCGUAUG | 941 |
| 1479 | GCAACAUGGUCAGUUUUCU | 635 | 1479 | GCAACAUGGUCAGUUUUCU | 635 | 1497 | AGAAAACUGACCAUGUUGC | 942 |
| 1497 | UCUUGCAGUCGUCAGCCUG | 636 | 1497 | UCUUGCAGUCGUCAGCCUG | 636 | 1515 | CAGGCUGACGACUGCAAGA | 943 |
| 1515 | GAACAUAACAUCCUUGGGA | 637 | 1515 | GAACAUAACAUCCUUGGGA | 637 | 1533 | UCCCAAGGAUGUUAUGUUC | 944 |
| 1533 | AUUACGCUCCCUCAAGGAG | 638 | 1533 | AUUACGCUCCCUCAAGGAG | 638 | 1551 | CUCCUUGAGGGAGCGUAAU | 945 |
| 1551 | GAUAAGUGAUGGAGAUGUG | 639 | 1551 | GAUAAGUGAUGGAGAUGUG | 639 | 1569 | CACAUCUCCAUCACUUAUC | 946 |
| 1569 | GAUAAUUUCAGGAAACAAA | 640 | 1569 | GAUAAUUUCAGGAAACAAA | 640 | 1587 | UUUGUUUCCUGAAAUUAUC | 947 |
| 1587 | AAAUUUGUGCUAUGCAAAU | 641 | 1587 | AAAUUUGUGCUAUGCAAAU | 641 | 1605 | AUUUGCAUAGCACAAAUUU | 948 |
| 1605 | UACAAUAAACUGGAAAAAA | 642 | 1605 | UACAAUAAACUGGAAAAAA | 642 | 1623 | UUUUUUCCAGUUUAUUGUA | 949 |
| 1623 | ACUGUUUGGGACCUCCGGU | 643 | 1623 | ACUGUUUGGGACCUCCGGU | 643 | 1641 | ACCGGAGGUCCCAAACAGU | 950 |
| 1641 | UCAGAAAACCAAAAUUAUA | 644 | 1641 | UCAGAAAACCAAAAUUAUA | 644 | 1659 | UAUAAUUUUGGUUUUCUGA | 951 |
| 1659 | AAGCAACAGAGGUGAAAAC | 645 | 1659 | AAGCAACAGAGGUGAAAAC | 645 | 1677 | GUUUUCACCUCUGUUGCUU | 952 |
| 1677 | CAGCUGCAAGGCCACAGGC | 646 | 1677 | CAGCUGCAAGGCCACAGGC | 646 | 1695 | GCCUGUGGCCUUGCAGCUG | 953 |
| 1695 | CCAGGUCUGCCAUGCCUUG | 647 | 1695 | CCAGGUCUGCCAUGCCUUG | 647 | 1713 | CAAGGCAUGGCAGACCUGG | 954 |
| 1713 | GUGCUCCCCCGAGGGCUGC | 648 | 1713 | GUGCUCCCCCGAGGGCUGC | 648 | 1731 | GCAGCCCUCGGGGGAGCAC | 955 |
| 1731 | CUGGGGCCCGGAGCCCAGG | 649 | 1731 | CUGGGGCCCGGAGCCCAGG | 649 | 1749 | CCUGGGCUCCGGGCCCCAG | 956 |
| 1749 | GGACUGCGUCUCUUGCCGG | 650 | 1749 | GGACUGCGUCUCUUGCCGG | 650 | 1767 | CCGGCAAGAGACGCAGUCC | 957 |
| 1767 | GAAUGUCAGCCGAGGCAGG | 651 | 1767 | GAAUGUCAGCCGAGGCAGG | 651 | 1785 | CCUGCCUCGGCUGACAUUC | 958 |
| 1785 | GGAAUGCGUGGACAAGUGC | 652 | 1785 | GGAAUGCGUGGACAAGUGC | 652 | 1803 | GCACUUGUCCACGCAUUCC | 959 |
| 1803 | CAAGCUUCUGGAGGGUGAG | 653 | 1803 | CAAGCUUCUGGAGGGUGAG | 653 | 1821 | CUCACCCUCCAGAAGCUUG | 960 |
| 1821 | GCCAAGGGAGUUUGUGGAG | 654 | 1821 | GCCAAGGGAGUUUGUGGAG | 654 | 1839 | CUCCACAAACUCCCUUGGC | 961 |
| 1839 | GAACUCUGAGUGCAUACAG | 655 | 1839 | GAACUCUGAGUGCAUACAG | 655 | 1857 | CUGUAUGCACUCAGAGUUC | 962 |
| 1857 | GUGCCACCCAGAGUGCCUG | 656 | 1857 | GUGCCACCCAGAGUGCCUG | 656 | 1875 | CAGGCACUCUGGGUGGCAC | 963 |
| 1875 | GCCUCAGGCCAUGAACAUC | 657 | 1875 | GCCUCAGGCCAUGAACAUC | 657 | 1893 | GAUGUUCAUGGCCUGAGGC | 964 |
| 1893 | CACCUGCACAGGACGGGGA | 658 | 1893 | CACCUGCACAGGACGGGGA | 658 | 1911 | UCCCCGUCCUGUGCAGGUG | 965 |
| 1911 | ACCAGACAACUGUAUCCAG | 659 | 1911 | ACCAGACAACUGUAUCCAG | 659 | 1929 | CUGGAUACAGUUGUCUGGU | 966 |
| 1929 | GUGUGCCCACUACAUUGAC | 660 | 1929 | GUGUGCCCACUACAUUGAC | 660 | 1947 | GUCAAUGUAGUGGGCACAC | 967 |
| 1947 | CGGCCCCCACUGCGUCAAG | 661 | 1947 | CGGCCCCCACUGCGUCAAG | 661 | 1965 | CUUGACGCAGUGGGGGCCG | 968 |
| 1965 | GACCUGCCCGGCAGGAGUC | 662 | 1965 | GACCUGCCCGGCAGGAGUC | 662 | 1983 | GACUCCUGCCGGGCAGGUC | 969 |
| 1983 | CAUGGGAGAAAACAACACC | 663 | 1983 | CAUGGGAGAAAACAACACC | 663 | 2001 | GGUGUUGUUUUCUCCCAUG | 970 |
| 2001 | CCUGGUCUGGAAGUACGCA | 664 | 2001 | CCUGGUCUGGAAGUACGCA | 664 | 2019 | UGCGUACUUCCAGACCAGG | 971 |
| 2019 | AGACGCCGGCCAUGUGUGC | 665 | 2019 | AGACGCCGGCCAUGUGUGC | 665 | 2037 | GCACACAUGGCCGGCGUCU | 972 |
| 2037 | CCACCUGUGCCAUCCAAAC | 666 | 2037 | CCACCUGUGCCAUCCAAAC | 666 | 2055 | GUUUGGAUGGCACAGGUGG | 973 |
| 2055 | CUGCACCUACGGAUGCACU | 667 | 2055 | CUGCACCUACGGAUGCACU | 667 | 2073 | AGUGCAUCCGUAGGUGCAG | 974 |
| 2073 | UGGGCCAGGUCUUGAAGGC | 668 | 2073 | UGGGCCAGGUCUUGAAGGC | 668 | 2091 | GCCUUCAAGACCUGGCCCA | 975 |
| 2091 | CUGUCCAACGAAUGGGCCU | 669 | 2091 | CUGUCCAACGAAUGGGCCU | 669 | 2109 | AGGCCCAUUCGUUGGACAG | 976 |
| 2109 | UAAGAUCCCGUCCAUCGCC | 670 | 2109 | UAAGAUCCCGUCCAUCGCC | 670 | 2127 | GGCGAUGGACGGGAUCUUA | 977 |
| 2127 | CACUGGGAUGGUGGGGGCC | 671 | 2127 | CACUGGGAUGGUGGGGGCC | 671 | 2145 | GGCCCCCACCAUCCCAGUG | 978 |
| 2145 | CCUCCUCUUGCUGCUGGUG | 672 | 2145 | CCUCCUCUUGCUGCUGGUG | 672 | 2163 | CACCAGCAGCAAGAGGAGG | 979 |
| 2163 | GGUGGCCCUGGGGAUCGGC | 673 | 2163 | GGUGGCCCUGGGGAUCGGC | 673 | 2181 | GCCGAUCCCCAGGGCCACC | 980 |
| 2181 | CCUCUUCAUGCGAAGGCGC | 674 | 2181 | CCUCUUCAUGCGAAGGCGC | 674 | 2199 | GCGCCUUCGCAUGAAGAGG | 981 |
| 2199 | CCACAUCGUUCGGAAGCGC | 675 | 2199 | CCACAUCGUUCGGAAGCGC | 675 | 2217 | GCGCUUCCGAACGAUGUGG | 982 |
| 2217 | CACGCUGCGGAGGCUGCUG | 676 | 2217 | CACGCUGCGGAGGCUGCUG | 676 | 2235 | CAGCAGCCUCCGCAGCGUG | 983 |
| 2235 | GCAGGAGAGGGAGCUUGUG | 677 | 2235 | GCAGGAGAGGGAGCUUGUG | 677 | 2253 | CACAAGCUCCCUCUCCUGC | 984 |
| 2253 | GGAGCCUCUUACACCCAGU | 678 | 2253 | GGAGCCUCUUACACCCAGU | 678 | 2271 | ACUGGGUGUAAGAGGCUCC | 985 |
| 2271 | UGGAGAAGCUCCCAACCAA | 679 | 2271 | UGGAGAAGCUCCCAACCAA | 679 | 2289 | UUGGUUGGGAGCUUCUCCA | 986 |
| 2289 | AGCUCUCUUGAGGAUCUUG | 680 | 2289 | AGCUCUCUUGAGGAUCUUG | 680 | 2307 | CAAGAUCCUCAAGAGAGCU | 987 |
| 2307 | GAAGGAAACUGAAUUCAAA | 681 | 2307 | GAAGGAAACUGAAUUCAAA | 681 | 2325 | UUUGAAUUCAGUUUCCUUC | 988 |
| 2325 | AAAGAUCAAAGUGCUGGGC | 682 | 2325 | AAAGAUCAAAGUGCUGGGC | 682 | 2343 | GCCCAGCACUUUGAUCUUU | 989 |
| 2343 | CUCCGGUGCGUUCGGCACG | 683 | 2343 | CUCCGGUGCGUUCGGCACG | 683 | 2361 | CGUGCCGAACGCACCGGAG | 990 |
| 2361 | GGUGUAUAAGGGACUCUGG | 684 | 2361 | GGUGUAUAAGGGACUCUGG | 684 | 2379 | CCAGAGUCCCUUAUACACC | 991 |
| 2379 | GAUCCCAGAAGGUGAGAAA | 685 | 2379 | GAUCCCAGAAGGUGAGAAA | 685 | 2397 | UUUCUCACCUUCUGGGAUC | 992 |
| 2397 | AGUUAAAAUUCCCGUCGCU | 686 | 2397 | AGUUAAAAUUCCCGUCGCU | 686 | 2415 | AGCGACGGGAAUUUUAACU | 993 |
| 2415 | UAUCAAGGAAUUAAGAGAA | 687 | 2415 | UAUCAAGGAAUUAAGAGAA | 687 | 2433 | UUCUCUUAAUUCCUUGAUA | 994 |
| 2433 | AGCAACAUCUCCGAAAGCC | 688 | 2433 | AGCAACAUCUCCGAAAGCC | 688 | 2451 | GGCUUUCGGAGAUGUUGCU | 995 |
| 2451 | CAACAAGGAAAUCCUCGAU | 689 | 2451 | CAACAAGGAAAUCCUCGAU | 689 | 2469 | AUCGAGGAUUUCCUUGUUG | 996 |
| 2469 | UGAAGCCUACGUGAUGGCC | 690 | 2469 | UGAAGCCUACGUGAUGGCC | 690 | 2487 | GGCCAUCACGUAGGCUUCA | 997 |
| 2487 | CAGCGUGGACAACCCCCAC | 691 | 2487 | CAGCGUGGACAACCCCCAC | 691 | 2505 | GUGGGGGUUGUCCACGCUG | 998 |
| 2505 | CGUGUGCCGCCUGCUGGGC | 692 | 2505 | CGUGUGCCGCCUGCUGGGC | 692 | 2523 | GCCCAGCAGGCGGCACACG | 999 |
| 2523 | CAUCUGCCUCACCUCCACC | 693 | 2523 | CAUCUGCCUCACCUCCACC | 693 | 2541 | GGUGGAGGUGAGGCAGAUG | 1000 |
| 2541 | CGUGCAACUCAUCACGCAG | 694 | 2541 | CGUGCAACUCAUCACGCAG | 694 | 2559 | CUGCGUGAUGAGUUGCACG | 1001 |
| 2559 | GCUCAUGCCCUUCGGCUGC | 695 | 2559 | GCUCAUGCCCUUCGGCUGC | 695 | 2577 | GCAGCCGAAGGGCAUGAGC | 1002 |
| 2577 | CCUCCUGGACUAUGUCCGG | 696 | 2577 | CCUCCUGGACUAUGUCCGG | 696 | 2595 | CCGGACAUAGUCCAGGAGG | 1003 |
| 2595 | GGAACACAAAGACAAUAUU | 697 | 2595 | GGAACACAAAGACAAUAUU | 697 | 2613 | AAUAUUGUCUUUGUGUUCC | 1004 |
| 2613 | UGGCUCCCAGUACCUGCUC | 698 | 2613 | UGGCUCCCAGUACCUGCUC | 698 | 2631 | GAGCAGGUACUGGGAGCCA | 1005 |
| 2631 | CAACUGGUGUGUGCAGAUC | 699 | 2631 | CAACUGGUGUGUGCAGAUC | 699 | 2649 | GAUCUGCACACACCAGUUG | 1006 |
| 2649 | CGCAAAGGGCAUGAACUAC | 700 | 2649 | CGCAAAGGGCAUGAACUAC | 700 | 2667 | GUAGUUCAUGCCCUUUGCG | 1007 |
| 2667 | CUUGGAGGACCGUCGCUUG | 701 | 2667 | CUUGGAGGACCGUCGCUUG | 701 | 2685 | AGAGCGACGGUCCUCCAAG | 1008 |
| 2685 | GGUGCACCGCGACCUGGCA | 702 | 2685 | GGUGCACCGCGACCUGGCA | 702 | 2703 | UGCCAGGUCGCGGUGCACC | 1009 |
| 2703 | AGCCAGGAACGUACUGGUG | 703 | 2703 | AGCCAGGAACGUACUGGUG | 703 | 2721 | CACCAGUACGUUCCUGGCU | 1010 |
| 2721 | GAAAACACCGCAGCAUGUC | 704 | 2721 | GAAAACACCGCAGCAUGUC | 704 | 2739 | GACAUGCUGCGGUGUUUUC | 1011 |
| 2739 | CAAGAUCACAGAUUUUGGG | 705 | 2739 | CAAGAUCACAGAUUUUGGG | 705 | 2757 | CCCAAAAUCUGUGAUCUUG | 1012 |
| 2757 | GCUGGCCAAACUGCUGGGU | 706 | 2757 | GCUGGCCAAACUGCUGGGU | 706 | 2775 | ACCCAGCAGUUUGGCCAGC | 1013 |
| 2775 | UGCGGAAGAGAAAGAAUAC | 707 | 2775 | UGCGGAAGAGAAAGAAUAC | 707 | 2793 | GUAUUCUUUCUCUUCCGCA | 1014 |
| 2793 | CCAUGCAGAAGGAGGCAAA | 708 | 2793 | CCAUGCAGAAGGAGGCAAA | 708 | 2811 | UUUGCCUCCUUCUGCAUGG | 1015 |
| 2811 | AGUGCCUAUCAAGUGGAUG | 709 | 2811 | AGUGCCUAUCAAGUGGAUG | 709 | 2829 | CAUCCACUUGAUAGGCACU | 1016 |
| 2829 | GGCAUUGGAAUCAAUUUUA | 710 | 2829 | GGCAUUGGAAUCAAUUUUA | 710 | 2847 | UAAAAUUGAUUCCAAUGCC | 1017 |
| 2847 | ACACAGAAUCUAUACCCAC | 711 | 2847 | ACACAGAAUCUAUACCCAC | 711 | 2865 | GUGGGUAUAGAUUCUGUGU | 1018 |
| 2865 | CCAGAGUGAUGUCUGGAGC | 712 | 2865 | CCAGAGUGAUGUCUGGAGC | 712 | 2883 | GCUCCAGACAUCACUCUGG | 1019 |
| 2883 | CUACGGGGUGACCGUUUGG | 713 | 2883 | CUACGGGGUGACCGUUUGG | 713 | 2901 | CCAAACGGUCACCCCGUAG | 1020 |
| 2901 | GGAGUUGAUGACCUUUGGA | 714 | 2901 | GGAGUUGAUGACCUUUGGA | 714 | 2919 | UCCAAAGGUCAUCAACUCC | 1021 |
| 2919 | AUCCAAGCCAUAUGACGGA | 715 | 2919 | AUCCAAGCCAUAUGACGGA | 715 | 2937 | UCCGUCAUAUGGCUUGGAU | 1022 |
| 2937 | AAUCCCUGCCAGCGAGAUC | 716 | 2937 | AAUCCCUGCCAGCGAGAUC | 716 | 2955 | GAUCUCGCUGGCAGGGAUU | 1023 |
| 2955 | CUCCUCCAUCCUGGAGAAA | 717 | 2955 | CUCCUCCAUCCUGGAGAAA | 717 | 2973 | UUUCUCCAGGAUGGAGGAG | 1024 |
| 2973 | AGGAGAACGCCUCCCUCAG | 718 | 2973 | AGGAGAACGCCUCCCUCAG | 718 | 2991 | CUGAGGGAGGCGUUCUCCU | 1025 |
| 2991 | GCCACCCAUAUGUACCAUC | 719 | 2991 | GCCACCCAUAUGUACCAUC | 719 | 3009 | GAUGGUACAUAUGGGUGGC | 1026 |
| 3009 | CGAUGUCUACAUGAUCAUG | 720 | 3009 | CGAUGUCUACAUGAUCAUG | 720 | 3027 | CAUGAUCAUGUAGACAUCG | 1027 |
| 3027 | GGUCAAGUGCUGGAUGAUA | 721 | 3027 | GGUCAAGUGCUGGAUGAUA | 721 | 3045 | UAUCAUCCAGCACUUGACC | 1028 |
| 3045 | AGACGCAGAUAGUCGCCCA | 722 | 3045 | AGACGCAGAUAGUCGCCCA | 722 | 3063 | UGGGCGACUAUCUGCGUCU | 1029 |
| 3063 | AAAGUUCCGUGAGUUGAUC | 723 | 3063 | AAAGUUCCGUGAGUUGAUC | 723 | 3081 | GAUCAACUCACGGAACUUU | 1030 |
| 3081 | CAUCGAAUUCUCCAAAAUG | 724 | 3081 | CAUCGAAUUCUCCAAAAUG | 724 | 3099 | CAUUUUGGAGAAUUCGAUG | 1031 |
| 3099 | GGCCCGAGACCCCCAGCGC | 725 | 3099 | GGCCCGAGACCCCCAGCGC | 725 | 3117 | GCGCUGGGGGUCUCGGGCC | 1032 |
| 3117 | CUACCUUGUCAUUCAGGGG | 726 | 3117 | CUACCUUGUCAUUCAGGGG | 726 | 3135 | CCCCUGAAUGACAAGGUAG | 1033 |
| 3135 | GGAUGAAAGAAUGCAUUUG | 727 | 3135 | GGAUGAAAGAAUGCAUUUG | 727 | 3153 | CAAAUGCAUUCUUUCAUCC | 1034 |
| 3153 | GCCAAGUCCUACAGACUCC | 728 | 3153 | GCCAAGUCCUACAGACUCC | 728 | 3171 | GGAGUCUGUAGGACUUGGC | 1035 |
| 3171 | CAACUUCUACCGUGCCCUG | 729 | 3171 | CAACUUCUACCGUGCCCUG | 729 | 3189 | CAGGGCACGGUAGAAGUUG | 1036 |
| 3189 | GAUGGAUGAAGAAGACAUG | 730 | 3189 | GAUGGAUGAAGAAGACAUG | 730 | 3207 | CAUGUCUUCUUCAUCCAUC | 1037 |
| 3207 | GGACGACGUGGUGGAUGCC | 731 | 3207 | GGACGACGUGGUGGAUGCC | 731 | 3225 | GGCAUCCACCACGUCGUCC | 1038 |
| 3225 | CGACGAGUACCUCAUCCCA | 732 | 3225 | CGACGAGUACCUCAUCCCA | 732 | 3243 | UGGGAUGAGGUACUCGUCG | 1039 |
| 3243 | ACAGCAGGGCUUCUUCAGC | 733 | 3243 | ACAGCAGGGCUUCUUCAGC | 733 | 3261 | GCUGAAGAAGCCCUGCUGU | 1040 |
| 3261 | CAGCCCCUCCACGUCACGG | 734 | 3261 | CAGCCCCUCCACGUCACGG | 734 | 3279 | CCGUGACGUGGAGGGGCUG | 1041 |
| 3279 | GACUCCCCUCCUGAGCUCU | 735 | 3279 | GACUCCCCUCCUGAGCUCU | 735 | 3297 | AGAGCUCAGGAGGGGAGUC | 1042 |
| 3297 | UCUGAGUGCAACCAGCAAC | 736 | 3297 | UCUGAGUGCAACCAGCAAC | 736 | 3315 | GUUGCUGGUUGCACUCAGA | 1043 |
| 3315 | CAAUUCCACCGUGGCUUGC | 737 | 3315 | CAAUUCCACCGUGGCUUGC | 737 | 3333 | GCAAGCCACGGUGGAAUUG | 1044 |
| 3333 | CAUUGAUAGAAAUGGGCUG | 738 | 3333 | CAUUGAUAGAAAUGGGCUG | 738 | 3351 | CAGCCCAUUUCUAUCAAUG | 1045 |
| 3351 | GCAAAGCUGUCCCAUCAAG | 739 | 3351 | GCAAAGCUGUCCCAUCAAG | 739 | 3369 | CUUGAUGGGACAGCUUUGC | 1046 |
| 3369 | GGAAGACAGCUUCUUGCAG | 740 | 3369 | GGAAGACAGCUUCUUGCAG | 740 | 3387 | CUGCAAGAAGCUGUCUUCC | 1047 |
| 3387 | GCGAUACAGCUCAGACCCC | 741 | 3387 | GCGAUACAGCUCAGACCCC | 741 | 3405 | GGGGUCUGAGCUGUAUCGC | 1048 |
| 3405 | CACAGGCGCCUUGACUGAG | 742 | 3405 | CACAGGCGCCUUGACUGAG | 742 | 3423 | CUCAGUCAAGGCGCCUGUG | 1049 |
| 3423 | GGACAGCAUAGACGACACC | 743 | 3423 | GGACAGCAUAGACGACACC | 743 | 3441 | GGUGUCGUCUAUGCUGUCC | 1050 |
| 3441 | CUUCCUCCCAGUGCCUGAA | 744 | 3441 | CUUCCUCCCAGUGCCUGAA | 744 | 3459 | UUCAGGCACUGGGAGGAAG | 1051 |
| 3459 | AUACAUAAACCAGUCCGUU | 745 | 3459 | AUACAUAAACCAGUCCGUU | 745 | 3477 | AACGGACUGGUUUAUGUAU | 1052 |
| 3477 | UCCCAAAAGGCCCGCUGGC | 746 | 3477 | UCCCAAAAGGCCCGCUGGC | 746 | 3495 | GCCAGCGGGCCUUUUGGGA | 1053 |
| 3495 | CUCUGUGCAGAAUCCUGUC | 747 | 3495 | CUCUGUGCAGAAUCCUGUC | 747 | 3513 | GACAGGAUUCUGCACAGAG | 1054 |
| 3513 | CUAUCACAAUCAGCCUCUG | 748 | 3513 | CUAUCACAAUCAGCCUCUG | 748 | 3531 | CAGAGGCUGAUUGUGAUAG | 1055 |
| 3531 | GAACCCCGCGCCCAGCAGA | 749 | 3531 | GAACCCCGCGCCCAGCAGA | 749 | 3549 | UCUGCUGGGCGCGGGGUUC | 1056 |
| 3549 | AGACCCACACUACCAGGAC | 750 | 3549 | AGACCCACACUACCAGGAC | 750 | 3567 | GUCCUGGUAGUGUGGGUCU | 1057 |
| 3567 | CCCCCACAGCACUGCAGUG | 751 | 3567 | CCCCCACAGCACUGCAGUG | 751 | 3585 | CACUGCAGUGCUGUGGGGG | 1058 |
| 3585 | GGGCAACCCCGAGUAUCUC | 752 | 3585 | GGGCAACCCCGAGUAUCUC | 752 | 3603 | GAGAUACUCGGGGUUGCCC | 1059 |
| 3603 | CAACACUGUCCAGCCCACC | 753 | 3603 | CAACACUGUCCAGCCCACC | 753 | 3621 | GGUGGGCUGGACAGUGUUG | 1060 |
| 3621 | CUGUGUCAACAGCACAUUC | 754 | 3621 | CUGUGUCAACAGCACAUUC | 754 | 3639 | GAAUGUGCUGUUGACACAG | 1061 |
| 3639 | CGACAGCCCUGCCCACUGG | 755 | 3639 | CGACAGCCCUGCCCACUGG | 755 | 3657 | CCAGUGGGCAGGGCUGUCG | 1062 |
| 3657 | GGCCCAGAAAGGCAGCCAC | 756 | 3657 | GGCCCAGAAAGGCAGCCAC | 756 | 3675 | GUGGCUGCCUUUCUGGGCC | 1063 |
| 3675 | CCAAAUUAGCCUGGACAAC | 757 | 3675 | CCAAAUUAGCCUGGACAAC | 757 | 3693 | GUUGUCCAGGCUAAUUUGG | 1064 |
| 3693 | CCCUGACUACCAGCAGGAC | 758 | 3693 | CCCUGACUACCAGCAGGAC | 758 | 3711 | GUCCUGCUGGUAGUCAGGG | 1065 |
| 3711 | CUUCUUUCCCAAGGAAGCC | 759 | 3711 | CUUCUUUCCCAAGGAAGCC | 759 | 3729 | GGCUUCCUUGGGAXAGAAG | 1066 |
| 3729 | CAAGCCAAAUGGCAUCUUU | 760 | 3729 | CAAGCCAAAUGGCAUCUUU | 760 | 3747 | AAAGAUGCCAUUUGGCUUG | 1067 |
| 3747 | UAAGGGCUCCACAGCUGAA | 761 | 3747 | UAAGGGCUCCACAGCUGAA | 761 | 3765 | UUCAGCUGUGGAGCCCUUA | 1068 |
| 3765 | AAAUGCAGAAUACCUAAGG | 762 | 3765 | AAAUGCAGAAUACCUAAGG | 762 | 3783 | CCUUAGGUAUUCUGCAUUU | 1069 |
| 3783 | GGUCGCGCCACAAAGCAGU | 763 | 3783 | GGUCGCGCCACAAAGCAGU | 763 | 3801 | ACUGCUUUGUGGCGCGACC | 1070 |
| 3801 | UGAAUUUAUUGGAGCAUGA | 764 | 3801 | UGAAUUUAUUGGAGCAUGA | 764 | 3819 | UCAUGCUCCAAUAAAUUCA | 1071 |
| 3819 | ACCACGGAGGAUAGUAUGA | 765 | 3819 | ACCACGGAGGAUAGUAUGA | 765 | 3837 | UCAUACUAUCCUCCGUGGU | 1072 |
| 3837 | AGCCCUAAAAAUCCAGACU | 766 | 3837 | AGCCCUAAAAAUCCAGACU | 766 | 3855 | AGUCUGGAUUUUUAGGGCU | 1073 |
| 3855 | UCUUUCGAUACCCAGGACC | 767 | 3855 | UCUUUCGAUACCCAGGACC | 767 | 3873 | GGUCCUGGGUAUCGAAAGA | 1074 |
| 3873 | CAAGCCACAGCAGGUCCUC | 768 | 3873 | CAAGCCACAGCAGGUCCUC | 768 | 3891 | GAGGACCUGCUGUGGCUUG | 1075 |
| 3891 | CCAUCCCAACAGCCAUGCC | 769 | 3891 | CCAUCCCAACAGCCAUGCC | 769 | 3909 | GGCAUGGCUGUUGGGAUGG | 1076 |
| 3909 | CCGCAUUAGCUCUUAGACC | 770 | 3909 | CCGCAUUAGCUCUUAGACC | 770 | 3927 | GGUCUAAGAGCUAAUGCGG | 1077 |
| 3927 | CCACAGACUGGUUUUGCAA | 771 | 3927 | CCACAGACUGGUUUUGCAA | 771 | 3945 | UUGCAAAACCAGUCUGUGG | 1078 |
| 3945 | ACGUUUACACCGACUAGCC | 772 | 3945 | ACGUUUACACCGACUAGCC | 772 | 3963 | GGCUAGUCGGUGUAAACGU | 1079 |
| 3963 | CAGGAAGUACUUCCACCUC | 773 | 3963 | CAGGAAGUACUUCCACCUC | 773 | 3981 | GAGGUGGAAGUACUUCCUG | 1080 |
| 3981 | CGGGCACAUUUUGGGAAGU | 774 | 3981 | CGGGCACAUUUUGGGAAGU | 774 | 3999 | ACUUCCCAAAAUGUGCCCG | 1081 |
| 3999 | UUGCAUUCCUUUGUCUUCA | 775 | 3999 | UUGCAUUCCUUUGUCUUCA | 775 | 4017 | UGAAGACAAAGGAAUGCAA | 1082 |
| 4017 | AAACUGUGAAGCAUUUACA | 776 | 4017 | AAACUGUGAAGCAUUUACA | 776 | 4035 | UGUAAAUGCUUCACAGUUU | 1083 |
| 4035 | AGAAACGCAUCCAGCAAGA | 777 | 4035 | AGAAACGCAUCCAGCAAGA | 777 | 4053 | UCUUGCUGGAUGCGUUUCU | 1084 |
| 4053 | AAUAUUGUCCCUUUGAGCA | 778 | 4053 | AAUAUUGUCCCUUUGAGCA | 778 | 4071 | UGCUCAAAGGGACAAUAUU | 1085 |
| 4071 | AGAAAUUUAUCUUUCAAAG | 779 | 4071 | AGAAAUUUAUCUUUCAAAG | 779 | 4089 | CUUUGAAAGAUAAAUUUCU | 1086 |
| 4089 | GAGGUAUAUUUGAAAAAAA | 780 | 4089 | GAGGUAUAUUUGAAAAAAA | 780 | 4107 | UUUUUUUCAAAUAUACCUC | 1087 |
| 4107 | AAAAAAAAAGUAUAUGUGA | 781 | 4107 | AAAAAAAAAGUAUAUGUGA | 781 | 4125 | UCACAUAUACUUUUUUUUU | 1088 |
| 4125 | AGGAUUUUUAUUGAUUGGG | 782 | 4125 | AGGAUUUUUAUUGAUUGGG | 782 | 4143 | CCCAAUCAAUAAAAAUCCU | 1089 |
| 4143 | GGAUCUUGGAGUUUUUCAU | 783 | 4143 | GGAUCUUGGAGUUUUUCAU | 783 | 4161 | AUGAAAAACUCCAAGAUCC | 1090 |
| 4161 | UUGUCGCUAUUGAUUUUUA | 784 | 4161 | UUGUCGCUAUUGAUUUUUA | 784 | 4179 | UAAAAAUCAAUAGCGACAA | 1091 |
| 4179 | ACUUCAAUGGGCUCUUCCA | 785 | 4179 | ACUUCAAUGGGCUCUUCCA | 785 | 4197 | UGGAAGAGCCCAUUGAAGU | 1092 |
| 4197 | AACAAGGAAGAAGCUUGCU | 786 | 4197 | AACAAGGAAGAAGCUUGCU | 786 | 4215 | AGCAAGCUUCUUCCUUGUU | 1093 |
| 4215 | UGGUAGCACUUGCUACCCU | 787 | 4215 | UGGUAGCACUUGCUACCCU | 787 | 4233 | AGGGUAGCAAGUGCUACCA | 1094 |
| 4233 | UGAGUUCAUCCAGGCCCAA | 788 | 4233 | UGAGUUCAUCCAGGCCCAA | 788 | 4251 | UUGGGCCUGGAUGAACUCA | 1095 |
| 4251 | ACUGUGAGCAAGGAGCACA | 789 | 4251 | ACUGUGAGCAAGGAGCACA | 789 | 4269 | UGUGCUCCUUGCUCACAGU | 1096 |
| 4269 | AAGCCACAAGUCUUCCAGA | 790 | 4269 | AAGCCACAAGUCUUCCAGA | 790 | 4287 | UCUGGAAGACUUGUGGCUU | 1097 |
| 4287 | AGGAUGCUUGAUUCCAGUG | 791 | 4287 | AGGAUGCUUGAUUCCAGUG | 791 | 4305 | CACUGGAAUCAAGCAUCCU | 1098 |
| 4305 | GGUUCUGCUUCAAGGCUUC | 792 | 4305 | GGUUCUGCUUCAAGGCUUC | 792 | 4323 | GAAGCCUUGAAGCAGAACC | 1099 |
| 4323 | CCACUGCAAAACACUAAAG | 793 | 4323 | CCACUGCAAAACACUAAAG | 793 | 4341 | CUUUAGUGUUUUGCAGUGG | 1100 |
| 4341 | GAUCCAAGAAGGCCUUCAU | 794 | 4341 | GAUCCAAGAAGGCCUUCAU | 794 | 4359 | AUGAAGGCCUUCUUGGAUC | 1101 |
| 4359 | UGGCCCCAGCAGGCCGGAU | 795 | 4359 | UGGCCCCAGCAGGCCGGAU | 795 | 4377 | AUCCGGCCUGCUGGGGCCA | 1102 |
| 4377 | UCGGUACUGUAUCAAGUCA | 796 | 4377 | UCGGUACUGUAUCAAGUCA | 796 | 4395 | UGACUUGAUACAGUACCGA | 1103 |
| 4395 | AUGGCAGGUACAGUAGGAU | 797 | 4395 | AUGGCAGGUACAGUAGGAU | 797 | 4413 | AUCCUACUGUACCUGCCAU | 1104 |
| 4413 | UAAGCCACUCUGUCCCUUC | 798 | 4413 | UAAGCCACUCUGUCCCUUC | 798 | 4431 | GAAGGGACAGAGUGGCUUA | 1105 |
| 4431 | CCUGGGCAAAGAAGAAACG | 799 | 4431 | CCUGGGCAAAGAAGAAACG | 799 | 4449 | CGUUUCUUCUUUGCCCAGG | 1106 |
| 4449 | GGAGGGGAUGAAUUCUUCC | 800 | 4449 | GGAGGGGAUGAAUUCUUCC | 800 | 4467 | GGAAGAAUUCAUCCCCUCC | 1107 |
| 4467 | CUUAGACUUACUUUUGUAA | 801 | 4467 | CUUAGACUUACUUUUGUAA | 801 | 4485 | UUACAAAAGUAAGUCUAAG | 1108 |
| 4485 | AAAAUGUCCCCACGGUACU | 802 | 4485 | AAAAUGUCCCCACGGUACU | 802 | 4503 | AGUACCGUGGGGACAUUUU | 1109 |
| 4503 | UUACUCCCCACUGAUGGAC | 803 | 4503 | UUACUCCCCACUGAUGGAC | 803 | 4521 | GUCCAUCAGUGGGGAGUAA | 1110 |
| 4521 | CCAGUGGUUUCCAGUCAUG | 804 | 4521 | CCAGUGGUUUCCAGUCAUG | 804 | 4539 | CAUGACUGGAAACCACUGG | 1111 |
| 4539 | GAGCGUUAGACUGACUUGU | 805 | 4539 | GAGCGUUAGACUGACUUGU | 805 | 4557 | ACAAGUCAGUCUAACGCUC | 1112 |
| 4557 | UUUGUCUUCCAUUCCAUUG | 806 | 4557 | UUUGUCUUCCAUUCCAUUG | 806 | 4575 | CAAUGGAAUGGAAGACAAA | 1113 |
| 4575 | GUUUUGAAACUCAGUAUGC | 807 | 4575 | GUUUUGAAACUCAGUAUGC | 807 | 4593 | GCAUACUGAGUUUCAAAAC | 1114 |
| 4593 | CCGCCCCUGUCUUGCUGUC | 808 | 4593 | CCGCCCCUGUCUUGCUGUC | 808 | 4611 | GACAGCAAGACAGGGGCGG | 1115 |
| 4611 | CAUGAAAUCAGCAAGAGAG | 809 | 4611 | CAUGAAAUCAGCAAGAGAG | 809 | 4629 | CUCUCUUGCUGAUUUCAUG | 1116 |
| 4629 | GGAUGACACAUCAAAUAAU | 810 | 4629 | GGAUGACACAUCAAAUAAU | 810 | 4647 | AUUAUUUGAUGUGUCAUCC | 1117 |
| 4647 | UAACUCGGAUUCCAGCCCA | 811 | 4647 | UAACUCGGAUUCCAGCCCA | 811 | 4665 | UGGGCUGGAAUCCGAGUUA | 1118 |
| 4665 | ACAUUGGAUUCAUCAGCAU | 812 | 4665 | ACAUUGGAUUCAUCAGCAU | 812 | 4683 | AUGCUGAUGAAUCCAAUGU | 1119 |
| 4683 | UUUGGACCAAUAGCCCACA | 813 | 4683 | UUUGGACCAAUAGCCCACA | 813 | 4701 | UGUGGGCUAUUGGUCCAAA | 1120 |
| 4701 | AGCUGAGAAUGUGGAAUAC | 814 | 4701 | AGCUGAGAAUGUGGAAUAC | 814 | 4719 | GUAUUCCACAUUCUCAGCU | 1121 |
| 4719 | CCUAAGGAUAACACCGCUU | 815 | 4719 | CCUAAGGAUAACACCGCUU | 815 | 4737 | AAGCGGUGUUAUCCUUAGG | 1122 |
| 4737 | UUUGUUCUCGCAAAAACGU | 816 | 4737 | UUUGUUCUCGCAAAAACGU | 816 | 4755 | ACGUUUUUGCGAGAACAAA | 1123 |
| 4755 | UAUCUCCUAAUUUGAGGCU | 817 | 4755 | UAUCUCCUAAUUUGAGGCU | 817 | 4773 | AGCCUCAAAUUAGGAGAUA | 1124 |
| 4773 | UCAGAUGAAAUGCAUCAGG | 818 | 4773 | UCAGAUGAAAUGCAUCAGG | 818 | 4791 | CCUGAUGCAUUUCAUCUGA | 1125 |
| 4791 | GUCCUUUGGGGCAUAGAUC | 819 | 4791 | GUCCUUUGGGGCAUAGAUC | 819 | 4809 | GAUCUAUGCCCCAAAGGAC | 1126 |
| 4809 | CAGAAGACUACAAAAAUGA | 820 | 4809 | CAGAAGACUACAAAAAUGA | 820 | 4827 | UCAUUUUUGUAGUCUUCUG | 1127 |
| 4827 | AAGCUGCUCUGAAAUCUCC | 821 | 4827 | AAGCUGCUCUGAAAUCUCC | 821 | 4845 | GGAGAUUUCAGAGCAGCUU | 1128 |
| 4845 | CUUUAGCCAUCACCCCAAC | 822 | 4845 | CUUUAGCCAUCACCCCAAC | 822 | 4863 | GUUGGGGUGAUGGCUAAAG | 1129 |
| 4863 | CCCCCCAAAAUUAGUUUGU | 823 | 4863 | CCCCCCAAAAUUAGUUUGU | 823 | 4881 | ACAAACUAAUUUUGGGGGG | 1130 |
| 4881 | UGUUACUUAUGGXAGAUAG | 824 | 4881 | UGUUACUUAUGGAAGAUAG | 824 | 4899 | CUAUCUUCCAUAAGUAACA | 1131 |
| 4899 | GUUUUCUCCUUUUACUUCA | 825 | 4899 | GUUUUCUCCUUUUACUUCA | 825 | 4917 | UGAAGUAAAAGGAGAAAAC | 1132 |
| 4917 | ACUUCAAAAGCUUUUUACU | 826 | 4917 | ACUUCAAAAGCUUUUUACU | 826 | 4935 | AGUAAAAAGCUUUUGAAGU | 1133 |
| 4935 | UCAAAGAGUAUAUGUUCCC | 827 | 4935 | UCAAAGAGUAUAUGUUCCC | 827 | 4953 | GGGAACAUAUACUCUUUGA | 1134 |
| 4953 | CUCCAGGUCAGCUGCCCCC | 828 | 4953 | CUCCAGGUCAGCUGCCCCC | 828 | 4971 | GGGGGCAGCUGACCUGGAG | 1135 |
| 4971 | CAAACCCCCUCCUUACGCU | 829 | 4971 | CAAACCCCCUCCUUACGCU | 829 | 4989 | AGCGUAAGGAGGGGGUUUG | 1136 |
| 4989 | UUUGUCACACAAAAAGUGU | 830 | 4989 | UUUGUCACACAAAAAGUGU | 830 | 5007 | ACACUUUUUGUGUGACAAA | 1137 |
| 5007 | UCUCUGCCUUGAGUCAUCU | 831 | 5007 | UCUCUGCCUUGAGUCAUCU | 831 | 5025 | AGAUGACUCAAGGCAGAGA | 1138 |
| 5025 | UAUUCAAGCACUUACAGCU | 832 | 5025 | UAUUCAAGCACUUACAGCU | 832 | 5043 | AGCUGUAAGUGCUUGAAUA | 1139 |
| 5043 | UCUGGCCACAACAGGGCAU | 833 | 5043 | UCUGGCCACAACAGGGCAU | 833 | 5061 | AUGCCCUGUUGUGGCCAGA | 1140 |
| 5061 | UUUUACAGGUGCGAAUGAC | 834 | 5061 | UUUUACAGGUGCGAAUGAC | 834 | 5079 | GUCAUUCGCACCUGUAAAA | 1141 |
| 5079 | CAGUAGCAUUAUGAGUAGU | 835 | 5079 | CAGUAGCAUUAUGAGUAGU | 835 | 5097 | ACUACUCAUAAUGCUACUG | 1142 |
| 5097 | UGUGAAUUCAGGUAGUAAA | 836 | 5097 | UGUGAAUUCAGGUAGUAAA | 836 | 5115 | UUUACUACCUGAAUUCACA | 1143 |
| 5115 | AUAUGAAACUAGGGUUUGA | 837 | 5115 | AUAUGAAACUAGGGUUUGA | 837 | 5133 | UOAAACCCUAGUUUCAUAU | 1144 |
| 5133 | AAAUUGAUAAUGCUUUCAC | 838 | 5133 | AAAUUGAUAAUGCUUUCAC | 838 | 5151 | GUGAAAGCAUUAUCAAUUU | 1145 |
| 5151 | CAACAUUUGCAGAUGUUUU | 839 | 5151 | CAACAUUUGCAGAUGUUUU | 839 | 5169 | AAAACAUCUGCAAAUGUUG | 1146 |
| 5169 | UAGAAGGAAAAAAGUUCCU | 840 | 5169 | UAGAAGGAAAAAAGUUCCU | 840 | 5187 | AGGAACUUUUUUCCUUCUA | 1147 |
| 5187 | UUCCUAAAAUAAUUUCUCU | 841 | 5187 | UUCCUAAAAUAAUUUCUCU | 841 | 5205 | AGAGAAAUUAUUUUAGGAA | 1148 |
| 5205 | UACAAUUGGAAGAUUGGAA | 842 | 5205 | UACAAUUGGAAGAUUGGAA | 842 | 5223 | UUCCAAUCUUCCAAUUGUA | 1149 |
| 5223 | AGAUUCAGCUAGUUAGGAG | 843 | 5223 | AGAUUCAGCUAGUUAGGAG | 843 | 5241 | CUCCUAACUAGCUGAAUCU | 1150 |
| 5241 | GCCCAUUUUUUCCUAAUCU | 844 | 5241 | GCCCAUUUUUUCCUAAUCU | 844 | 5259 | AGAUUAGGAAAAAAUGGGC | 1151 |
| 5259 | UGUGUGUGCCCUGUAACCU | 845 | 5259 | UGUGUGUGCCCUGUAACCU | 845 | 5277 | AGGUUACAGGGCACACACA | 1152 |
| 5277 | UGACUGGUUAACAGCAGUC | 846 | 5277 | UGACUGGUUAACAGCAGUC | 846 | 5295 | GACUGCUGUUAACOAGUCA | 1153 |
| 5295 | CCUUUGUAAACAGUGUUUU | 847 | 5295 | CCUUUGUAAACAGUGUUUU | 847 | 5313 | AAAACACUGUUUACAAAGG | 1154 |
| 5313 | UAAACUCUCCUAGUCAAUA | 848 | 5313 | UAAACUCUCCUAGUCAAUA | 848 | 5331 | UAUUGACUAGGAGAGUUUA | 1155 |
| 5331 | AUCCACCCCAUCCAAUUUA | 849 | 5331 | AUCCACCCCAUCCAAUUUA | 849 | 5349 | UAAAUUGGAUGGGGUGGAU | 1156 |
| 5349 | AUCAAGGAAGAAAUGGUUC | 850 | 5349 | AUCAAGGAAGAAAUGGUUC | 850 | 5367 | GAACCAUUUCUUCCUUGAU | 1157 |
| 5367 | CAGAAAAUAUUUUCAGCCU | 851 | 5367 | CAGAAAAUAUUUUCAGCCU | 851 | 5385 | AGGCUGAAAAUAUUUUCUG | 1158 |
| 5385 | UACAGUUAUGUUCAGUCAC | 852 | 5385 | UACAGUUAUGUUCAGUCAC | 852 | 5403 | GUGACUGAAOAUAACUGUA | 1159 |
| 5403 | CACACACAUACAAAAUGUU | 853 | 5403 | CACACACAUACAAAAUGUU | 853 | 5421 | AACAUUUUGUAUGUGUGUG | 1160 |
| 5421 | UCCUUUUGCUUUUAAAGUA | 854 | 5421 | UCCUUUUGCUUUUAAAGUA | 854 | 5439 | UACUUUAAAAGCAAAAGGA | 1161 |
| 5439 | AAUUUUUGACUCCCAGAUC | 855 | 5439 | AAUUUUUGACUCCCAGAUC | 855 | 5457 | GAUCUGGGAGUCAAAAAUU | 1162 |
| 5457 | CAGUCAGAGCCCCUACAGC | 856 | 5457 | CAGUCAGAGCCCCUACAGC | 856 | 5475 | GCUGUAGGGGCUCUGACUG | 1163 |
| 5475 | CAUUGUUAAGAAAGUAUUU | 857 | 5475 | CAUUGUUAAGAAAGUAUUU | 857 | 5493 | AAAUACUUUCUUAACAAUG | 1164 |
| 5493 | UGAUUUUUGUCUCAAUGAA | 858 | 5493 | UGAUUUUUGUCUCAAUGAA | 858 | 5511 | UUCAUUGAGACAAAAAUCA | 1165 |
| 5511 | AAAUAAAACUAUAUUCAUU | 859 | 5511 | AAAUAAAACUAUAUUCAUU | 859 | 5529 | AAUGAAUAUAGUUUUAUUU | 1166 |

NM_005228 Homo sapiens epidermal growth factor receptor (erythroblastic leukemia viral (v-erb-b) oncogene homolog, avian) (EGFR), mRNA

TABLE V
EGFR (HER1) Synthetic Sequences with all RNA counterparts and Target Sequences

| | | | | | |
|---|---|---|---|---|---|
| Target | | Seq | | | |
| Pos | Target | ID | Aliases | Sequence | Seq ID |

| | | | | | |
|---|---|---|---|---|---|
| 799 | UUGGUCAGUUUCUGGCAGUUCUC | 1167 | EGFR:801U21 siNA | GAACUGCCAGAAACUGACCAA | 1171 |
| 1380 | GCAAAAACCCUGUGAUUUCCUUU | 1168 | EGFR:1382U21 sINA | AGGAAAUCACAGGGUUUUUGC | 1172 |
| 3064 | UCGAUGAUCAACUCACGGAACUU | 1169 | EGFR:3066U21 siNA | GUUCCGUGAGUUGAUCAUCGA | 1173 |
| 3152 | GUUGGAGUCUGUAGGACUUGGCA | 1170 | EGFR:3154U21 siNA | CCAAGUCCUACAGACUCCAAC | 1174 |
| 799 | UUGGUCAGUUUCUGGCAGUUCUC | 1167 | EGFR:819L21 siNA (801C) | GGUCAGUUUCUGGCAGUUCUC | 1175 |
| 1380 | GCAAAAACCCUGUGAUUUCCUUU | 1168 | EGFR:1400L21 siNA (1382C) | AAMACCCUGUGAUUUCCUUU | 1176 |
| 3064 | UCGAUGAUCAACUCACGGAACUU | 1169 | EGFR:3084L21 siNA (3066C) | GAUGAUCAACUCACGGAACUU | 1177 |
| 3152 | GUUGGAGUCUGUAGGACUUGGCA | 1170 | EGFR:3172L21 sINA (3154C) | UGGAGUCUGUAGGACUUGGCA | 1178 |
| 799 | UUGGUCAGUUUCUGGCAGUUCUC | 1167 | EGFR:801U21 siNAstab4 | B GAAcuGccAGAAACuGAccAA B | 1179 |
| 1380 | GCAAAAACCCUGUGAUUUCCUUU | 1168 | EGFR:1382U21 siNAstab4 | B AGGAAAucAcAGGGuuuuuGC B | 1180 |
| 3064 | UCGAUGAUCAACUCACGGAACUU | 1169 | EGFR:3066U21 siNAstab4 | B GuuccGuGAGuuGAucAucGA B | 1181 |
| 3152 | GUUGGAGUCUGUAGGACUUGGCA | 1170 | EGFR:3154U21 siNAstab4 | B ccAAGuccuAcAGAcuccAAc B | 1182 |
| 799 | UUGGUCAGUUUCUGGCAGUUCUC | 1167 | EGFR:819L21 siNA (801C) stab5 | GgucAGuuucuGGcAGuucTsT L | 1183 |
| 1380 | GCAAAAACCCUGUGAUUUCCUUU | 1168 | EGFR:1400L21 siNA (1382C) stab5 | AAAAAcccuGuGAuuuccuTsT L | 1184 |
| 3064 | UCGAUGAUCAACUCACGGAACUU | 1169 | EGFR:3084L21 siNA (3066C) stab5 | GAuGAucAAcucAcGGAAcTsT L | 1185 |
| 3152 | GUUGGAGUCUGUAGGACUUGGCA | 1170 | EGFR:3172121 siNA (3154C) stab5 | uGGAGucuGuAGGAcuuGGTsT L | 1186 |

A = Adenosine
G = Guanosine
C = Cytidine
U = Uridine
T = Thymidine
u = 2′-deoxy-2′-fluoro uridine
c = 2′-deoxy-2′-fluoro cytidine
B = inverted deoxy abasic ribose
L = glyceryl moiety
S = phosphorothioate internucleotide linkage

TABLE VI
A. 2.5 μmol Synthesis Cycle ABI 394 Instrument

| | | | | Wait Time* | | Wait Time* | | Wait Time* | |
|---|---|---|---|---|---|---|---|---|---|
| Reagent | Equivalents | Amount | | DNA | | 2′-O-methyl | | RNA | |
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |
| Acetonitrile | NA | 6.67 | mL | NA | | NA | | NA | |

B. 0.2 μmol Synthesis Cycle ABI 394 Instrument

| | | | | Wait Time* | | Wait Time* | | Wait Time* | |
|---|---|---|---|---|---|---|---|---|---|
| Reagent | Equivalents | Amount | | DNA | | 2′-O-methyl | | RNA | |
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |
| Acetonitrile | NA | 2.64 | mL | NA | | NA | | NA | |

C. 0.2 μmol Synthesis Cycle 96 well Instrument

| | Equivalents: DNA/ | Amount: DNA/ | | Wait Time* | | Wait Time* | | Wait Time* | |
|---|---|---|---|---|---|---|---|---|---|
| Reagent | 2′-O-methyl/Ribo | 2′-O-methyl/Ribo | | DNA | | 2′-O-methyl | | Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |
| Acetonitrile | NA | 1150/1150/1150 | μL | NA | | NA | | NA | |

*Wait time does not include contact time during delivery.
*Tandem synthesis utilizes double coupling of linker molecule

What we claim is:

1. A short interfering nucleic acid (siNA) molecule that down regulates expression of an EGFR gene by RNA interference.

2. The siNA molecule of claim 1, wherein the EGFR gene comprises HER1 sequence, HER2 sequence, HER3 sequence, HER4 sequence, or a combination thereof.

3. The siNA molecule of claim 1, wherein said siNA molecule is adapted for use to treat cancer.

4. The siNA molecule of claim 1, wherein said siNA molecule comprises a sense region and an antisense region and wherein said antisense region comprises sequence complementary to an RNA sequence encoding EGFR and the sense region comprises sequence complementary to the antisense region.

5. The siNA molecule of claim 4, wherein said siNA molecule is assembled from two nucleic acid fragments wherein one fragment comprises the sense region and the second fragment comprises the antisense region of said siNA molecule.

6. The siNA molecule of claim 5, wherein said sense region and antisense region are covalently connected via a linker molecule.

7. The siNA molecule of claim 6, wherein said linker molecule is a polynucleotide linker.

8. The siNA molecule of claim 6, wherein said linker molecule is a non-nucleotide linker.

9. The siNA molecule of claim 1, wherein the siNA molecule comprises sequence having any of SEQ ID NOs. 1-1213.

10. The siNA molecule of claim 4, wherein said sense region comprises a 3′-terminal overhang and said antisense region comprises a 3′-terminal overhang.

11. The siNA molecule of claim 10, wherein said 3′-terminal overhangs each comprise about 2 nucleotides.

12. The siNA molecule of claim 10, wherein said antisense region 3′-terminal nucleotide overhang is complementary to RNA encoding EGFR.

13. The siNA molecule of claim 4, wherein said sense region comprises one or more 2′-O-methyl modified pyrimidine nucleotides.

14. The siNA molecule of claim 4, wherein said sense region comprises a terminal cap moiety at the 5′-end, 3′-end, or both 5′ and 3′ ends of said sense region.

15. The siNA molecule of claim 4, wherein said antisense region comprises one or more 2′-deoxy-2′-fluoro modified pyrimidine nucleotides.

16. The siNA molecule of claim 4, wherein said antisense region comprises a phosphorothioate internucleotide linkage at the 3′ end of said antisense region.

17. The siNA molecule of claim 4, wherein said antisense region comprises between about one and about five phosphorothioate internucleotide linkages at the 5′ end of said antisense region.

18. The siNA molecule of claim 10, wherein said 3′-terminal nucleotide overhangs comprise ribonucleotides that are chemically-modified at a nucleic acid sugar, base, or backbone.

19. The siNA molecule of claim 10, wherein said 3′-terminal nucleotide overhangs comprise deoxyribonucleotides that are chemically-modified at a nucleic acid sugar, base, or backbone.

20. The siNA molecule of claim 10, wherein said 3′-terminal nucleotide overhangs comprise one or more universal base ribonucleotides.

21. The siNA molecule of claim 10, wherein said 3′-terminal nucleotide overhangs comprise one or more acyclic nucleotides.

22. The siNA molecule of claim 10, wherein said 3′-terminal nucleotide overhangs comprise nucleotides comprising internucleotide linkages having Formula I: 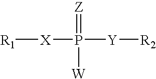
wherein each R1 and R2 is independently any nucleotide, non-nucleotide, or polynucleotide which can be naturally occurring or chemically-modified, each X and Y is independently O, S, N, alkyl, or substituted alkyl, each Z and W is independently O, S, N, alkyl, substituted alkyl, O-alkyl, S-alkyl, alkaryl, or aralkyl.

23. The siNA molecule of claim 10, wherein said 3′-terminal nucleotide overhangs comprise nucleotides or non-nucleotides having Formula II: 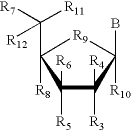
wherein each R3, R4, R5, R6, R7, R8, R10, R11 and R12 is independently H, OH, alkyl, substituted alkyl, alkaryl or aralkyl, F, C1, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO₂, NO₂, N3, NH_{2,} aminoalkyl, aminoacid, aminoacyl, ONH_{2,} O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base or any other non-naturally occurring base that can be complementary or non-complementary to EGFR RNA or a non-nucleosidic base or any other non-naturally occurring universal base that can be complementary or non-complementary to EGFR RNA.

24. The siNA molecule of claim 10, wherein said antisense region 3′-terminal nucleotide overhang comprise a 3′-terminal substituted polyalkyl moiety having Formula VII: 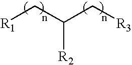
wherein each n is independently an integer from 1 to 12, R1, R2 and R3 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I, and either R1, R2 or R3 serve as points of attachment to the siNA molecule of the invention.

25. An expression vector comprising a nucleic acid sequence encoding at least one siNA molecule of claim 1 in a manner that allows expression of the nucleic acid molecule.

26. A mammalian cell comprising an expression vector of claim 25.

27. The mammalian cell of claim 26, wherein said mammalian cell is a human cell.

28. The expression vector of claim 25, wherein said at least one siNA molecule comprises a sense region and an antisense region and wherein said antisense region comprises sequence complementary to an RNA sequence encoding EGFR and the sense region comprises sequence complementary to the antisense region.

29. The expression vector of claim 28, wherein said at least one siNA molecule comprises two distinct strands having complementarity sense and antisense regions.

30. The expression vector of claim 28, wherein said at least one siNA molecule comprises a single-strand having complementary sense and antisense regions.

31. The siNA molecule of claim 4, wherein any pyrimidine nucleotides present in said sense region comprise 2′-deoxy-2′-fluoro pyrimidine nucleotides and wherein any purine nucleotides present in said sense region comprise 2′-deoxy purine nucleotides.

32. The siNA molecule of claim 31, wherein any nucleotides comprising a 3′-terminal nucleotide overhang that are present in said sense region are 2′-deoxy nucleotides.

33. The siNA molecule of claim 4, wherein any pyrimidine nucleotides present in said antisense region comprise 2′-deoxy-2′-fluoro pyrimidine nucleotides and wherein any purine nucleotides present in said antisense region comprise 2′-O-methyl purine nucleotides.

34. The siNA molecule of claim 33, wherein any nucleotides comprising a 3′-terminal nucleotide overhang that are present in said antisense region are 2′-deoxy nucleotides.
